(19) United States
(12) Patent Application Publication
(10) Pub. No.: US 2024/0408032 A1
(43) Pub. Date: Dec. 12, 2024

(54) LIPID-BASED RNA FORMULATIONS SUITABLE FOR THERAPY

(71) Applicant: BioNTech SE, Mainz (DE)

(72) Inventors: Hossam HEFESHA, Mainz (DE); Heinrich HAAS, Mainz (DE); Ferdia BATES, Mainz (DE); Christian HOTZ, Mainz (DE); Katalin KARIKÓ, Mainz (DE)

(21) Appl. No.: 18/689,727

(22) PCT Filed: Sep. 9, 2022
(86) PCT No.: PCT/EP2022/075168

§ 371 Date: Mar. 6, 2024

(30) Foreign Application Priority Data
Sep. 10, 2021 (WO) PCT/EP2021/074976

(51) Int. Cl. A61K 9/51; A61K 9/00; A61K 38/19; A61K 38/20; A61K 38/21

(52) U.S. Cl. CPC A61K 9/513; A61K 9/0019; A61K 9/5123; A61K 9/5192; A61K 38/193; A61K 38/208; A61K 38/212

(57) ABSTRACT

The present disclosure relates to RNA particles comprising phospholipids with a phosphatidylserine head group for delivering RNA to target tissues after administration, in particular after parenteral, intratumoral, or peritumoral administration, and compositions comprising such RNA particles. The present disclosure also relates to methods for preparing RNA particles described herein.

### TECHNICAL FIELD

The present disclosure relates to RNA particles comprising phospholipids with a phosphatidylserine head group for delivering RNA to target tissues after administration, in particular after parenteral, intratumoral, or peritumoral administration, and compositions comprising such RNA particles. The present disclosure also relates to methods for preparing RNA particles described herein. The RNA particles in some embodiments comprise single-stranded RNA such as mRNA which encodes a peptide or polypeptide of interest, such as a pharmaceutically active peptide or polypeptide. The RNA is taken up by cells of a target tissue and the RNA is translated into the encoded peptide or polypeptide, which may exhibit its physiological activity. The methods, processes, and compositions described herein are suitable for use in a manner that complies with the requirements for pharmaceutical products, more specifically that complies with the requirements for GMP manufacturing and the requirements for the quality of pharmaceutical products.

### BACKGROUND

The use of RNA to deliver foreign genetic information into target cells offers an attractive alternative to DNA. The advantages of RNA include transient expression and non-transforming character. RNA does not require nucleus infiltration for expression and moreover cannot integrate into the host genome, thereby eliminating the risk of oncogenesis.

RNA can be delivered to a target through different vehicles, based mostly upon cationic polymers or lipids, which combine with the RNA to form nanoparticles. The nanoparticles are intended to protect the RNA from degradation, enable delivery of the RNA to the target site and facilitate cellular uptake and processing by the target cells. The efficiency of RNA delivery depends, in part, on the molecular composition of the nanoparticle and can be influenced by numerous parameters, including particle size, formulation, and charge or grafting with molecular moieties, such as polyethylene glycol (PEG) or other ligands.

There is a need of providing formulations for the delivery of biologically active RNA to a target tissue where the delivered RNA is efficiently translated into the peptide or polypeptide it codes for. The inventors surprisingly found that the RNA particle formulations described herein fulfill the above mentioned requirements.

### SUMMARY

The present invention generally provides RNA particles comprising a phospholipid with a phosphatidylserine head group. The inventors found that RNA particle formulations containing negatively charged phospholipids with a phosphatidylserine head group can be manufactured in a robust and reproducible manner. They are suitable as RNA delivery systems and display superior biological characteristics in comparison to systems without phosphatidylserine.

In order to overcome electrostatic repulsion with the RNA, at certain stages of manufacturing, the pH may be lowered to reduce or completely remove the negative charge of the phosphatidylserine lipids. Subsequently, for administration, the particles may be restored to a pH closer to the physiological range. In this way, the particles keep their stability while retaining their advantageous biological activity.

The RNA particle formulations described herein are useful as RNA delivery vehicles for in vivo application. In addition, the particles can be manufactured in a GMP-compliant manner with relatively easy and straightforward protocols. This has removed undesired processing steps required for the manufacturing of other lipid based nanoparticle products (diafiltration, extrusion, etc.). The final product is suitable for pharmaceutical application.

In some aspects, the invention provides an RNA particle comprising:
(i) RNA,
(ii) at least one cationic or cationically ionizable lipid, and
(iii) at least one phosphatidylserine.

In some embodiments, the cationic or cationically ionizable lipid comprises a head group which includes at least one nitrogen atom which is positively charged or capable of being protonated, preferably under physiological conditions.

In some embodiments, the cationic or cationically ionizable lipid is selected from the group consisting of N,N-dimethyl-2,3-dioleyloxypropylamine (DODMA), 1,2-dimyristyloxypropyl-3-dimethyl-hydroxy ethyl ammonium bromide (DMRIE), N,N-dioleyl-N,N-dimethylammonium chloride (DODAC), 1,2-dioleoyl-3-dimethylammonium-propane (DODAP), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), (6Z,9Z,28Z,31Z)-heptatriaconta-6,9,28,31-tetraen-19-yl 4-(dimethylamino)butanoate (D-Lin-MC3-DMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), and 4-((di((9Z,12Z)-octadeca-9,12-dien-1-yl)amino)oxy)-N,N-dimethyl-4-oxobutan-1-amine (DPL-14) or a mixture thereof.

In some embodiments, the phosphatidylserine comprises dioleoylphosphatidylserine (DOPS), 1,2-dioctanoyl-sn-glycero-3-phospho-L-serine (08:0 PS), 1,2-didecanoyl-sn-glycero-3-phospho-L-serine (10:0 PS), 1,2-dilauroyl-sn-glycero-3-phospho-L-serine (12:0 PS), dimyristoylphosphatidylserine (DMPS), dipalmitoylphosphatidylserine (DPPS), 1,2-diheptadecanoyl-sn-glycero-3-phospho-L-serine (17:0 PS), 1,2-distearoyl-sn-glycero-3-phospho-L-serine (18:0 PS), 1-palmitoyl-2-oleoyl-sn-glycero-3-phospho-L-serine (POPS), 1-stearoyl-2-oleoyl-sn-glycero-3-phospho-L-serine (SOPS), 1-palmitoyl-2-linoleoyl-sn-glycero-3-phospho-L-serine (PLenPS), 1-palmitoyl-2-arachidonoyl-sn-glycero-3-phospho-L-serine (16:0-20:4 PS), 1-palmitoyl-2-docosahexaenoyl-sn-glycero-3-phospho-L-serineor (16:0-22:6 PS) 1-stearoyl-2-linoleoyl-sn-glycero-3-phospho-L-serine (18:0-18:2 PS), 1-stearoyl-2-arachidonoyl-sn-glycero-3-phospho-L-serine (18:0-20:4 PS), 1-stearoyl-2-docosahexaenoyl-sn-glycero-3-phospho-L-serine (18:0-22:6 PS), 1,2-dilinoleoyl-sn-glycero-3-phospho-L-serine (18:2 PS), 1,2-diarachidonoyl-sn-glycero-3-phospho-L-serine (20:4 PS), 1,2-didocosahexaenoyl-sn-glycero-3-phospho-L-serine (22:6 PS), 1-tridecanoyl-sn-glycero-3-phospho-L-serine (13:0 Lyso PS), 1-(10Z-heptadecenoyl)-2-hydroxy-sn-glycero-3-[phospho-L-serine] (17:1 Lyso PS), 1-palmitoyl-2-hydroxy-sn-glycero-3-phospho-L-serine (16:0 Lyso PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), brain PS, brain lyso PS, soy PS, or a mixture thereof.

In some embodiments, the RNA particle further comprises at least one additional lipid.

In some embodiments, the additional lipid is selected from the group consisting of neutral lipids, steroids, and combinations thereof.

In some embodiments, the additional lipid comprises a neutral lipid.

In some embodiments, the additional lipid comprises a phospholipid.

In some embodiments, the phospholipid is selected from the group consisting of phosphatidylcholines, phosphatidylethanolamines, phosphatidylglycerols, phosphatidic acids, and sphingomyelins.

In some embodiments, the phospholipid is selected from the group consisting of dioleoylphosphatidylethanolamine (DOPE), distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

In some embodiments, the additional lipid comprises cholesterol or a cholesterol derivative.

In some embodiments, the additional lipid comprises a mixture of a phospholipid and cholesterol or a cholesterol derivative.

In some embodiments, the additional lipid comprises DOPE, cholesterol, or a mixture of DOPE and cholesterol.

In some embodiments, the cationic or cationically ionizable lipid comprises from about 20 mol % to about 95 mol % of the total lipid present in the particle.

In some embodiments, the phosphatidylserine comprises from about 2 mol % to about 70 mol % of the total lipid present in the particle.

In some embodiments, the additional lipid comprises from about 0 mol % to about 80 mol % of the total lipid present in the particle.

In some embodiments, the RNA particle comprises a cationic or cationically ionizable lipid, a phosphatidylserine, and an additional lipid.

In some embodiments, the additional lipid comprises a neutral lipid, a steroid or a mixture thereof.

In some embodiments, the molar ratio of cationic or cationically ionizable lipid, phosphatidylserine, and additional lipid is 2-9.5:0.2-7:0.2-8.

In some embodiments, the molar ratio of cationic or cationically ionizable lipid, phosphatidylserine, and additional lipid is about 6:about 1:about 1.

In some embodiments, the molar ratio of cationic or cationically ionizable lipid, phosphatidylserine, and additional lipid is about 6:about 1:about 1.2-1.8.

In some embodiments, the molar ratio of cationic or cationically ionizable lipid, phosphatidylserine, and additional lipid is such as about 6:about 1:about 1.2.

In some embodiments, the molar ratio of cationic or cationically ionizable lipid, phosphatidylserine, and additional lipid is about 6:about 1:about 1.7.

In some embodiments, the molar ratio of cationic or cationically ionizable lipid, phosphatidylserine, and additional lipid is about 6:about 1:about 1.8.

In some embodiments, the RNA particle comprises a cationic or cationically ionizable lipid, DOPS, and DOPE.

In some embodiments, the molar ratio of cationic or cationically ionizable lipid, DOPS, and DOPE is 2-9.5:0.2-7:0.2-8.

In some embodiments, the molar ratio of cationic or cationically ionizable lipid, DOPS, and DOPE is about 6:about 1:about 1.

In some embodiments, the molar ratio of cationic or cationically ionizable lipid, DOPS, and DOPE is about 6:about 1:about 1.2-1.8.

In some embodiments, the molar ratio of cationic or cationically ionizable lipid, DOPS, and DOPE is about 6:about 1:about 1.2.

In some embodiments, the molar ratio of cationic or cationically ionizable lipid, DOPS, and DOPE is about 6:about 1:about 1.7.

In some embodiments, the molar ratio of cationic or cationically ionizable lipid, DOPS, and DOPE is about 6:about 1:about 1.8.

In some embodiments, the RNA particle comprises DODMA, DOPS, and DOPE.

In some embodiments, the molar ratio of DODMA, DOPS, and DOPE is 2-9.5:0.2-7:0.2-8.

In some embodiments, the molar ratio of DODMA, DOPS, and DOPE is about 6:about 1:about 1.

In some embodiments, the molar ratio of DODMA, DOPS, and DOPE is about 6:about 1:about 1.2-1.8.

In some embodiments, the molar ratio of DODMA, DOPS, and DOPE is about 6:about 1:about 1.2.

In some embodiments, the molar ratio of DODMA, DOPS, and DOPE is about 6:about 1:about 1.7.

In some embodiments, the molar ratio of DODMA, DOPS, and DOPE is about 6:about 1:about 1.8.

In some aspects, the invention provides an RNA particle comprising:
(i) RNA,
(ii) at least one cationic or cationically ionizable lipid,
(iii) a first phospholipid which is a phosphatidylserine, and
(iv) a second phospholipid.

In some embodiments, the cationic or cationically ionizable lipid comprises DODMA.

In some embodiments, the phosphatidylserine comprises DOPS.

In some embodiments, the second phospholipid comprises DOPE.

In some embodiments, the cationic or cationically ionizable lipid comprises from about 20 mol % to about 95 mol % of the total lipid present in the particle.

In some embodiments, the phosphatidylserine comprises from about 2 mol % to about 70 mol % of the total lipid present in the particle.

In some embodiments, the second phospholipid comprises from about 2 mol % to about 80 mol % of the total lipid present in the particle.

In some embodiments, the molar ratio of cationic or cationically ionizable lipid, phosphatidylserine, and second phospholipid is 2-9.5:0.2-7:0.2-8.

In some embodiments, the molar ratio of cationic or cationically ionizable lipid, phosphatidylserine, and second phospholipid is about 6:about 1:about 1.

In some embodiments, the molar ratio of cationic or cationically ionizable lipid, phosphatidylserine, and second phospholipid is about 6:about 1:about 1.2-1.8.

In some embodiments, the molar ratio of cationic or cationically ionizable lipid, phosphatidylserine, and second phospholipid is about 6:about 1:about 1.2.

In some embodiments, the molar ratio of cationic or cationically ionizable lipid, phosphatidylserine, and second phospholipid is about 6:about 1:about 1.7.

In some embodiments, the molar ratio of cationic or cationically ionizable lipid, phosphatidylserine, and second phospholipid is about 6:about 1:about 1.8.

In some aspects, the invention provides an RNA particle comprising (i) RNA, (ii) N,N-dimethyl-2,3-dioleyloxypropylamine (DODMA) and (iii) dioleoylphosphatidylserine (DOPS).

In some embodiments, the RNA particle further comprises dioleoylphosphatidylethanolamine (DOPE).

In some embodiments, the DODMA comprises from about 20 mol % to about 95 mol % of the total lipid present in the particle.

In some embodiments, the DOPS comprises from about 2 mol % to about 70 mol % of the total lipid present in the particle.

In some embodiments, the DOPE comprises from about 2 mol % to about 80 mol % of the total lipid present in the particle.

In some embodiments, the molar ratio of DODMA:DOPE:DOPS is 2-9.5:0.2-8:0.2-7.

In some embodiments, the molar ratio of DODMA:DOPE:DOPS is 5.5-6.5:0.9-1.1:0.9-1.1.

In some embodiments, the molar ratio of DODMA:DOPE:DOPS is about 6:about 1:about 1.

In some embodiments, the molar ratio of DODMA:DOPE:DOPS is about 6:about 1.2-1.8:about 1.

In some embodiments, the molar ratio of DODMA:DOPE:DOPS is about 6:about 1.2:about 1.

In some embodiments, the molar ratio of DODMA:DOPE:DOPS is about 6:about 1.7:about 1.

In some embodiments, the molar ratio of DODMA:DOPE:DOPS is about 6:about 1.8:about 1.

In some embodiments, the RNA particle of all aspects disclosed herein comprises a non-ionic amphiphilic organic compound. In some embodiments, the level of non-ionic amphiphilic organic compound present in an RNA particle (or composition comprising an RNA particle) is at least about 5 mol % of the total lipid present in the particle or composition, or at least about 0.15 mM. In some embodiments, the level of non-ionic amphiphilic organic compound present in an RNA particle (or composition comprising an RNA particle) is at least about 20 mol % of the total lipid present in the particle or composition, or at least about 0.6 mM.

In some embodiments, the non-ionic amphiphilic organic compound is a surfactant. In some embodiments, the level of surfactant present in an RNA particle (or composition comprising an RNA particle) is at least about 5 mol % of the total lipid present in the particle or composition, or at least about 0.15 mM. In some embodiments, the level of surfactant present in an RNA particle (or composition comprising an RNA particle) is at least about 20 mol % of the total lipid present in the particle or composition, or at least about 0.6 mM.

In some embodiments, the non-ionic amphiphilic organic compound is a poly(ethylene glycol) (PEG) surfactant.

In some embodiments, the non-ionic amphiphilic organic compound comprises a poly(ethylene glycol) (PEG) chain linked to a single hydrophobic chain.

In some embodiments, the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof. In some embodiments, the non-ionic amphiphilic organic compound comprises a polyoxyethylene sorbitan fatty acid ester. In some embodiments, the level of polyoxyethylene sorbitan fatty acid ester present in an RNA particle (or composition comprising an RNA particle) is at least about 5 mol % of the total lipid present in the particle or composition, or at least about 0.15 mM. In some embodiments, the level of polyoxyethylene sorbitan fatty acid ester present in an RNA particle (or composition comprising an RNA particle) is at least about 20 mol % of the total lipid present in the particle or composition, or at least about 0.6 mM. In some embodiments, the non-ionic amphiphilic organic compound comprises a polysorbate. In some embodiments, the level of polysorbate present in an RNA particle (or composition comprising an RNA particle) is at least about 5 mol % of the total lipid present in the particle or composition, or at least about 0.15 mM. In some embodiments, the level of polysorbate present in an RNA particle (or composition comprising an RNA particle) is at least about 20 mol % of the total lipid present in the particle or composition, or at least about 0.6 mM. In some embodiments, the non-ionic amphiphilic organic compound comprises polysorbate 20. In some embodiments, the level of polysorbate 20 present in an RNA particle (or composition comprising an RNA particle) is at least about 5 mol % of the total lipid present in the particle or composition, or at least about 0.15 mM. In some embodiments, the level of polysorbate 20 present in an RNA particle (or composition comprising an RNA particle) is at least about 20 mol % of the total lipid present in the particle or composition, or at least about 0.6 mM.

In some embodiments, the RNA particle does not comprise a sterol (e.g., a cholesterol or a cholesterol derivative). In some embodiments, the RNA particle does not comprise cholesterol. In some embodiments, the RNA particle does not comprise a cholesterol derivative. In some embodiments, the RNA particle does not comprise a steroid.

In some embodiments, the RNA particle does not comprise PEG. In some embodiments, the RNA particle does not comprise a cationic or cationically ionizable lipid that is conjugated to PEG. In some embodiments, the RNA particle does not comprise a phospholipid that is conjugated to PEG.

In some embodiments, the RNA is mRNA or saRNA.

In some embodiments, the particle is a lipid nanoparticle (LNP) or a lipoplex particle (LPX).

In some embodiments, the particle has a size of from about 30 nm to about 500 nm. In some embodiments, the particle has a size of from about 30 nm to about 100 nm. In some embodiments, the particle has a size of from about 100 nm to about 200 nm. In some embodiments, the particle has a size of from about 200 nm to about 300 nm. In some embodiments, the particle has a size of from about 300 nm to about 400 nm. In some embodiments, the particle has a size of from about 400 nm to about 500 nm. In some embodiments, the particle has a size of about 30 nm. In some embodiments, the particle has a size of about 50 nm. In some embodiments, the particle has a size of about 75 nm. In some embodiments, the particle has a size of about 100 nm. In some embodiments, the particle has a size of about 125 nm. In some embodiments, the particle has a size of about 175 nm. In some embodiments, the particle has a size of about 200 nm. In some embodiments, the particle has a size of about 250 nm.

In some embodiments, the particle has a size of about 300 nm. In some embodiments, the particle has a size of about 350 nm. In some embodiments, the particle has a size of about 400 nm. In some embodiments, the particle has a size of about 450 nm. In some embodiments, the particle has a size of about 500 nm.

In some embodiments, mRNA particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 n, from about 150 nm to about 200 n, from about 200 nm to about 1000 n, from about 200 nm to about 800 nm, from about 200 nm to about 700 n, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm.

In some embodiments, the particle is a non-viral particle.

In some embodiments, the RNA is of unimolecular or multimolecular species.

In some embodiments, the RNA is coding RNA.

In some embodiments, the RNA comprises a mixture of mRNAs that encode different peptides or polypeptides. In some embodiments, the mixture of mRNAs comprises 2, 3, or 4 mRNAs that encode different peptides or polypeptides. In some embodiments, the mixture of mRNAs comprises 2 mRNAs that encode 2 different peptides or polypeptides. In some embodiments, the mixture of mRNAs comprises 3 mRNAs that encode 3 different peptides or polypeptides. In some embodiments, the mixture of mRNAs comprises 4 mRNAs that encode 4 different peptides or polypeptides. In some embodiments, the mixture of mRNAs comprises mRNAs that encode different peptides or polypeptides, wherein the same weight of each of the mRNAs is present. In some embodiments, the mixture of mRNAs comprises 4 mRNAs that encode 4 different peptides or polypeptides, wherein the mRNAs are present in a 1:1:1:1 weight ratio.

In some embodiments, the RNA encodes one or more peptides or polypeptides selected from the group consisting of cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

In some embodiments, the peptides or polypeptides are cytokines. In some embodiments, the RNA comprises or consists of RNA encoding an IL-12sc protein, RNA encoding an IL-15 sushi protein, RNA encoding an IFNα protein, and RNA encoding a GM-CSF protein. In some embodiments, the RNA comprises or consists of RNA encoding an IL-12sc protein, RNA encoding an IL-15 sushi protein, RNA encoding an IFNα protein, and RNA encoding a GM-CSF protein, wherein each RNA is in a 1:1:1:1 (w/w/w/w) ratio.

In some embodiments:
i. each RNA is in a 1:1:1:1 (w/w/w/w) ratio; and/or
ii. each RNA comprises a modified nucleoside in place of each uridine; and/or
iii. each RNA comprises a modified nucleoside in place of each uridine, wherein the modified nucleoside is independently selected from pseudouridine (ψ), N1-methyl-pseudouridine (m1ψ), and 5-methyl-uridine (m5U); and/or
iv. each RNA comprises the 5′ cap m₂^{7,3′-O}Gppp(m₁^{2′-O})ApG or 3′-O-Me-m⁷G(5′)ppp(5′)G; and/or
v. each RNA comprises a 5′ UTR comprising a nucleotide sequence selected from the group consisting of SEQ ID NOs: 2 and 4, or a nucleotide sequence having at least 99%, 98%, 97%, 96%, 95%, 90%, 85%, or 80% identity to a nucleotide sequence selected from the group consisting of SEQ ID NOs: 2 and 4; and/or
vi. each RNA comprises a 3′ UTR comprising the nucleotide sequence of SEQ ID NO: 6, or a nucleotide sequence having at least 99%, 98%, 97%, 96%, 95%, 90%, 85%, or 80% identity to the nucleotide sequence of SEQ ID NO: 6; and/or
vii. each RNA comprises a poly-A tail, wherein the poly-A tail comprises at least 100 nucleotides, and wherein the poly-A tail comprises or consists of the poly-A tail shown in SEQ ID NO: 27.

In some embodiments:
i. the RNA encoding IL-12sc protein comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
ii. the RNA encoding an IL-15 sushi protein comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
iii. the RNA encoding an IFNα protein comprises the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
iv. the RNA encoding a GM-CSF protein comprises the nucleotide sequence of SEQ ID NO: 10, or a nucleotide sequence having at least 99%, 98%, 97%, 96%, 95%, 90%, 85%, or 80% identity to the nucleotide sequence of SEQ ID NO: 10.

In some embodiments:
i. the IL-12sc protein comprises the amino acid sequence of SEQ ID NO: 11, or an amino acid sequence having at least 99%, 98%, 97%, 96%, 95%, 90%, 85%, or 80% identity to the amino acid sequence of SEQ ID NO: 11; and/or
ii. the IL-15 sushi protein comprises the amino acid sequence of SEQ ID NO: 21, or an amino acid sequence having at least 99%, 98%, 97%, 96%, 95%, 90%, 85%, or 80% identity to the amino acid sequence of SEQ ID NO: 21; and/or
iii. the IFNα protein comprises the amino acid sequence of SEQ ID NO: 16, or an amino acid sequence having at least 99%, 98%, 97%, 96%, 95%, 90%, 85%, or 80% identity to the amino acid sequence of SEQ ID NO: 16; and/or
iv. the GM-CSF protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

In some embodiments, the RNA particle described herein is obtainable by a method comprising the steps:
(i) preparing an acidified lipid solution in an organic solvent;
(ii) adding the acidified lipid solution to an aqueous phase to produce a liposome colloid; and
(iii) adding the liposome colloid to an aqueous solution comprising RNA and a non-ionic amphiphilic organic compound to produce the RNA lipid particle.

In some embodiments, the particle is a lipoplex particle (LPX). In some embodiments, the level of polysorbate present in the LPX particle is at least about 5 mol % of the total lipid present in the particle.

In some embodiments, the RNA particle described herein is obtainable by a method comprising the steps:
(i) preparing an acidified lipid solution in an organic solvent; and
(ii) adding the acidified lipid solution to an aqueous solution comprising RNA and a non-ionic amphiphilic organic compound to produce the RNA lipid particle.

In some embodiments, the particle is a lipid nanoparticle (LNP). In some embodiments, the level of polysorbate present in the LNP particle is at least about 5 mol % of the total lipid present in the particle.

In some embodiments, the organic solvent comprises an alcohol. In some embodiments, the alcohol is ethanol.

In some embodiments, the non-ionic amphiphilic organic compound comprises from about 0.1 mol % to about 30 mol % of the total lipid. In some embodiments, the non-ionic amphiphilic organic compound comprises more than about 5 mol % of the total lipid.

In some embodiments, the non-ionic amphiphilic organic compound comprises from about 5 mol % to about 25 mol % of the total lipid. In some embodiments, the level of non-ionic amphiphilic organic compound is at least about 5 mol % of the total lipid present in the particle.

In some aspects, the invention provides a composition comprising one or more RNA particles or a plurality of RNA particles disclosed herein.

In some embodiments, the composition comprises an aqueous solution.

In some embodiments, the particles and/or the aqueous solution comprises a non-ionic amphiphilic organic compound. In some embodiments, the non-ionic amphiphilic organic compound is in the particles. In some embodiments, the non-ionic amphiphilic organic compound is in the aqueous solution.

In some embodiments, the non-ionic amphiphilic organic compound is present in the particle and the aqueous solution.

In some embodiments, the non-ionic amphiphilic organic compound is a surfactant.

In some embodiments, the non-ionic amphiphilic organic compound is a poly(ethylene glycol) (PEG) surfactant.

In some embodiments, the non-ionic amphiphilic organic compound comprises a poly(ethylene glycol) (PEG) chain linked to a single hydrophobic chain.

In some embodiments, the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof. In some embodiments, the non-ionic amphiphilic organic compound comprises a polyoxyethylene sorbitan fatty acid ester. In some embodiments, the non-ionic amphiphilic organic compound comprises a polysorbate. In some embodiments, the polysorbate is polysorbate 20.

In some embodiments, the non-ionic amphiphilic organic compound comprises from about 0.1 mol % to about 50 mol %, from about 1 mol % to about 50 mol %, from about 1 mol % to about 40 mol %, from about 1 mol % to about 30 mol %, from about 1 mol % to about 20 mol %, from about 1 mol % to about 15 mol %, from about 1 mol % to about 10 mol %, from about 1 mol % to about 7.5 mol %, or from about 2 mol % to about 5 mol % of the total lipid (including lipid-like material, e.g., non-ionic amphiphilic organic compound) present in the composition. In some embodiments, the non-ionic amphiphilic organic compound comprises about 3 mol % of the total lipid present in the composition. In some embodiments, the non-ionic amphiphilic organic compound comprises at least about 3 mol % of the total lipid present in the composition. In some embodiments, the non-ionic amphiphilic organic compound comprises at least about 5 mol % of the total lipid present in the composition. In some embodiments, the non-ionic amphiphilic organic compound comprises at least about 10 mol % of the total lipid present in the composition. In some embodiments, the non-ionic amphiphilic organic compound comprises at least about 15 mol % of the total lipid present in the composition. In some embodiments, the non-ionic amphiphilic organic compound comprises at least about 20 mol % of the total lipid present in the composition. In some embodiments, the non-ionic amphiphilic organic compound comprises at least about 25 mol % of the total lipid present in the composition. In some embodiments, the non-ionic amphiphilic organic compound comprises less than about 30 mol % of the total lipid present in the composition. In some embodiments, the non-ionic amphiphilic organic compound comprises from about 5 mol % to about 30 mol % of the total lipid present in the composition. In some embodiments, the non-ionic amphiphilic organic compound comprises from about 5 mol % to about 25 mol % of the total lipid present in the composition. In some embodiments, the non-ionic amphiphilic organic compound comprises from about 5 mol % to about 20 mol % of the total lipid present in the composition. In some embodiments, the non-ionic amphiphilic organic compound comprises from about 5 mol % to about 15 mol % of the total lipid present in the composition. In some embodiments, the non-ionic amphiphilic organic compound comprises from about 5 mol % to about 10 mol % of the total lipid present in the composition. In some embodiments, the non-ionic amphiphilic organic compound comprises from about 10 mol % to about 25 mol % of the total lipid present in the composition. In some embodiments, the non-ionic amphiphilic organic compound comprises from about 15 mol % to about 30 mol % of the total lipid present in the composition. In some embodiments, the non-ionic amphiphilic organic compound comprises from about 15 mol % to about 25 mol % of the total lipid present in the composition. In some embodiments, the non-ionic amphiphilic organic compound comprises from about 20 mol % to about 25 mol % of the total lipid present in the composition.

In some embodiments, a composition comprises a non-ionic amphiphilic organic compound at a level of at least about 0.15 mM. In some embodiments, a composition comprises a non-ionic amphiphilic organic compound at a level of at least about 0.2 mM. In some embodiments, a composition comprises a non-ionic amphiphilic organic compound at a level of at least about 0.25 mM. In some embodiments, a composition comprises a non-ionic amphiphilic organic compound at a level of at least about 0.3 mM. In some embodiments, a composition comprises a non-ionic amphiphilic organic compound at a level of at least about 0.35 mM. In some embodiments, a composition comprises a non-ionic amphiphilic organic compound at a level of at least about 0.4 mM. In some embodiments, a composition comprises a non-ionic amphiphilic organic compound at a level of at least about 0.45 mM. In some embodiments, a composition comprises a non-ionic amphiphilic organic compound at a level of at least about 0.5 mM. In some embodiments, a composition comprises a non-ionic amphiphilic organic compound at a level of at least about 0.55 mM. In some embodiments, a composition comprises a non-ionic amphiphilic organic compound at a level of at least about 0.6 mM. In some embodiments, a composition comprises a non-ionic amphiphilic organic compound at a level of at least about 0.7 mM. In some embodiments, a composition comprises a non-ionic amphiphilic organic compound at a level of at least about 0.8 mM. In some embodiments, a composition comprises a non-ionic amphiphilic organic compound at a level of at least about 0.9 mM. In some embodiments, a composition comprises a non-ionic amphiphilic organic compound at a level of at least about 1.0 mM. In some embodiments, a composition comprises a non-ionic amphiphilic organic compound at a level of at least about 1.1 mM. In some embodiments, a composition comprises a non-ionic amphiphilic organic compound at a level of at least about 1.2 mM. In some embodiments, a composition comprises a non-ionic amphiphilic organic compound at a level of at least about 1.3 mM. In some embodiments, a composition comprises a non-ionic amphiphilic organic compound at a level of at least about 1.4 mM. In some embodiments, a composition comprises a non-ionic amphiphilic organic compound at a level of at least about 1.5 mM. In some embodiments, a composition comprises a non-ionic amphiphilic organic compound at a level of at least about 2.0 mM. In some embodiments, a composition comprises a non-ionic amphiphilic organic compound at a level of at least about 2.5 mM. In some embodiments, a composition comprises a non-ionic amphiphilic organic compound at a level of at least about 3.0 mM. In some embodiments, a composition comprises a non-ionic amphiphilic organic compound at a level of at least about 4.0 mM. In some embodiments, a composition comprises a non-ionic amphiphilic organic compound at a level of at least about 5.0 mM.

In some embodiments, a composition comprises a non-ionic amphiphilic organic compound at a level of from about 0.15 mM to about 0.3 mM. In some embodiments, a composition comprises a non-ionic amphiphilic organic compound at a level of from about 0.3 mM to about 0.6 mM. In some embodiments, a composition comprises a non-ionic amphiphilic organic compound at a level of from about 0.6 mM to about 0.75 mM. In some embodiments, a composition comprises a non-ionic amphiphilic organic compound at a level of from about 0.75 mM to about 1.0 mM. In some embodiments, a composition comprises a non-ionic amphiphilic organic compound at a level of from about 1.0 mM to about 1.25 mM. In some embodiments, a composition comprises a non-ionic amphiphilic organic compound at a level of from about 1.25 mM to about 1.5 mM. In some embodiments, a composition comprises a non-ionic amphiphilic organic compound at a level of from about 1.75 mM to about 2.0 mM. In some embodiments, a composition comprises a non-ionic amphiphilic organic compound at a level of from about 2.0 mM to about 2.25 mM. In some embodiments, a composition comprises a non-ionic amphiphilic organic compound at a level of from about 2.25 mM to about 2.5 mM. In some embodiments, a composition comprises a non-ionic amphiphilic organic compound at a level of from about 2.5 mM to about 2.75 mM. In some embodiments, a composition comprises a non-ionic amphiphilic organic compound at a level of from about 2.75 mM to about 3.0 mM. In some embodiments, a composition comprises a non-ionic amphiphilic organic compound at a level of from about 3.0 mM to about 3.5 mM. In some embodiments, a composition comprises a non-ionic amphiphilic organic compound at a level of from about 4.0 mM to about 4.5 mM. In some embodiments, a composition comprises a non-ionic amphiphilic organic compound at a level of from about 4.5 mM to about 5.0 mM.

In some embodiments, the composition comprises DODMA, DOPE, DOPS, and polysorbate 20.

In some embodiments, the composition comprises DODMA, DOPE, DOPS, and polysorbate 20, wherein the molar ratio of DODMA:DOPE:DOPS:polysorbate 20 is 5.5-6.5:0.9-1.1:0.9-1.1:1.5-2.5.

In some embodiments, the molar ratio of DODMA:DOPE:DOPS:polysorbate 20 is about 6:about 1:about 1:about 2.

In some embodiments, the molar ratio of DODMA:DOPE:DOPS:polysorbate 20 is about 6:about 1.2-1.8:about 1:about 2.

In some embodiments, the molar ratio of DODMA:DOPE:DOPS:polysorbate 20 is about 6:about 1.2:about 1:about 2.

In some embodiments, the molar ratio of DODMA:DOPE:DOPS:polysorbate 20 is about 6:about 1.7:about 1:about 2.

In some embodiments, the molar ratio of DODMA:DOPE:DOPS:polysorbate 20 is about 6:about 1.8:about 1:about 2.

In some embodiments, the composition comprises a non-ionic amphiphilic organic compound at a level of at least about 0.15 mM. In some embodiments, the composition comprises a non-ionic amphiphilic organic compound at a level of at least about 0.3 mM. In some embodiments, the composition comprises a non-ionic amphiphilic organic compound at a level of at least about 0.6 mM.

In some embodiments, the composition comprises DODMA at a level of at least about 1.8 mM, DOPE at a level of at least about 0.3 mM, and DOPS at a level of at least about 0.3 mM. In some embodiments, the composition comprises DODMA at a level of about 1.8 mM, DOPE at a level of about 0.3 mM, and DOPS at a level of about 0.3 mM.

In some embodiments, the lipids in the composition consist of DODMA, DOPE, DOPS, and polysorbate 20.

In some embodiments, the particles are LPX particles.

In some embodiments, the LPX particles have an N/P ratio from about 3 to about 5, and wherein the pH of the composition is from about 7 to about 7.5. In some embodiments, the LPX particles have an N/P ratio from about 3 to about 5, and wherein the pH of the composition is from about 5.0 to about 6.0, optionally from about 5.2 to about 5.8, optionally about 5.5. In some embodiments, the pH is adjusted prior to administration to from about 6.5 to about 7.0, optionally from about 6.7 to about 6.8.

In some embodiments, the LPX particles have an N/P ratio of about 3.

In some embodiments, the RNA comprises RNA encoding an IL-12sc protein, RNA encoding an IL-15 sushi protein, RNA encoding an IFNα protein, and RNA encoding a GM-CSF protein.

In some embodiments, the composition does not comprise a sterol (e.g., a cholesterol or a cholesterol derivative). In some embodiments, the composition does not comprise cholesterol. In some embodiments, the composition does not comprise a cholesterol derivative. In some embodiments, the composition does not comprise a steroid.

In some embodiments, the composition does not comprise PEG. In some embodiments, the composition does not comprise a cationic or cationically ionizable lipid that is conjugated to PEG. In some embodiments, the composition does not comprise a phospholipid that is conjugated to PEG. In some embodiments, the composition does not comprise a lipid with less than 4 PEG chains. In some embodiments, the composition does not comprise a lipid with less than 3 PEG chains. In some embodiments, the composition does not comprise a lipid with less than 2 PEG chains. In some embodiments, the composition does not comprise a lipid with only 1 PEG chain.

In some embodiments, the composition does not comprise a compound comprising a poly(ethylene glycol) (PEG) chain and more than one hydrophobic chains. In some embodiments, the composition does not comprise a compound comprising a poly(ethylene glycol) (PEG) chain and two hydrophobic chains.

In some embodiments, the composition comprises a buffer such as HAc, sodium citrate (NaOCit), sodium acetate (NAOAc), NaCl, Na₂HPO₄, KH₂PO₄, or any combination thereof. In some embodiments, the composition comprises citrate buffer, optionally citrate buffer and sodium chloride. In some embodiments, the composition comprises a cryoprotectant such as sucrose. In some embodiments, the composition comprises HAc, NaOCit, NAOAc, NaCl, Na₂HPO₄, KH₂PO₄, and sucrose. In some embodiments, the composition comprises, consists essentially of, or consists of RNA, DODMA, DOPE, DOPS, polysorbate 20, HAc, NaOCit, NAOAc, NaCl, KCl, Na₂HPO₄, KH₂PO₄, sucrose, and water. In some embodiments, the composition comprises, consists essentially of, or consists of RNA, from 1.0 to 1.5 mg/ml DODMA, from 0.2 to 0.3 mg/ml DOPE, from 0.2 to 0.3 mg/ml DOPS, from 0.5 to 1.0 mg/ml polysorbate 20, from 0.01 to 0.05 mg/ml HAc, from 0.2 to 0.3 mg/ml NaOCit, from 0.2 to 0.5 mg/ml NAOAc, from 0.1 to 10 mg/ml NaCl, from 0 to 0.5 mg/ml KCl, 0 to 1.5 mg/ml Na₂HPO₄, from 0 to 0.5 mg/ml KH₂PO₄, from 75 to 125 mg/ml sucrose, and water. In some embodiments, the composition comprises, consists essentially of, or consists of RNA, about 1.24 or about 1.12 mg/ml DODMA, about 0.25 or about 0.225 mg/ml DOPE, about 0.27 or about 0.24 mg/ml DOPS, about 0.82 or about 0.74 mg/ml polysorbate 20, about 0.03 or about 0.027 mg/ml HAc, about 0.26 or about 0.23 mg/ml NaOCit, about 0.41 or about 0.37 mg/ml NAOAc, about 100 or about 90 mg/ml NaCl, 0 or about 0.2 mg/ml KCl, 0 or about 1.42 mg/ml Na₂HPO₄, 0 or about 0.24 mg/ml KH₂PO₄, from 75 to 125 mg/ml sucrose, and water.

In some aspects, the invention provides a method for delivering RNA to cells of a subject, the method comprising administering to a subject one or more RNA particles or a plurality of RNA particles or a composition disclosed herein.

In some aspects, the invention provides a method for delivering a therapeutic peptide or polypeptide to a subject, the method comprising administering to a subject one or more RNA particles or a plurality of RNA particles or a composition disclosed herein, wherein the RNA encodes the therapeutic peptide or polypeptide.

In some aspects, the invention provides a method for treating or preventing a disease in a subject, the method comprising administering to a subject one or more RNA particles or a plurality of RNA particles or a composition disclosed herein, wherein delivering the RNA to cells of the subject is beneficial in treating or preventing the disease.

In some aspects, the invention provides a method for treating or preventing a disease in a subject, the method comprising administering to a subject one or more RNA particles or a plurality of RNA particles or a composition disclosed herein, wherein the RNA encodes a therapeutic peptide or polypeptide and wherein delivering the therapeutic peptide or polypeptide to the subject is beneficial in treating or preventing the disease.

In some embodiments, the one or more RNA particles or plurality of RNA particles is administered in a pharmaceutically effective amount. In some embodiments, the composition is administered in a pharmaceutically effective amount.

In some embodiments of all aspects disclosed herein, administration is by intravenous, intratumoral, or peritumoral injection. In some embodiments, the administration is by intravenous injection. In some embodiments, the administration is by intratumoral injection. In some embodiments, the injection is by peritumoral injection.

In some embodiments of all aspects disclosed herein, the subject is a mammal.

In some embodiments, the mammal is a human.

In further aspects, the invention provides a method of producing RNA lipid particles comprising at least one phosphatidylserine, the method comprising the step of preparing a lipid solution in an organic solvent comprising the at least one phosphatidylserine wherein the lipid solution is acidified.

In some embodiments, the organic solvent comprises an alcohol.

In some embodiments, the alcohol is ethanol.

In some embodiments, the lipid solution has an acidic pH so as to render the at least one phosphatidylserine electroneutral.

In some embodiments, the lipid solution further comprises a cationically ionizable lipid and the acidic pH of the lipid solution renders the cationically ionizable lipid protonated.

In some embodiments, the method further comprises adding the lipid solution to an aqueous phase to produce a liposome colloid.

In some embodiments, the method further comprises adding the liposome colloid to an aqueous solution comprising the RNA to produce the RNA lipid particles.

In some embodiments, the aqueous solution comprising the RNA comprises citrate buffer, optionally citrate buffer and sodium chloride.

In some embodiments, the RNA lipid particles are lipoplex particles (LPX).

In some of these embodiments, where RNA lipid particles are LPX, the liposome colloid and the aqueous solution comprising the RNA are mixed at a flow rate of at least 200 mL/min, optionally at a flow rate of 300-400 mL/min, preferably at a flow rate of 300-360 mL/min.

In some embodiments, the method further comprises mixing the lipid solution with an aqueous solution comprising the RNA to produce the RNA lipid particles.

In some embodiments, the RNA lipid particles are lipid nanoparticles (LNP).

In some of these embodiments, where RNA lipid particles are LNP, the lipid solution and the aqueous solution comprising the RNA are mixed at a flow rate of about 2 to about 12 mL/min, optionally at a flow rate of about 4 to about 10 mL/min, preferably at a flow rate of about 6 to about 10 mL/min.

In some embodiments, the lipid solution and/or the RNA solution comprises a non-ionic amphiphilic organic compound.

In some embodiments, the method further comprises freezing the RNA lipid particles at −80° C. for storage.

In a further aspect, the invention provides a method of producing RNA lipid particles, the method comprising the step of preparing a lipid solution in an organic solvent and mixing the lipid solution with an aqueous solution comprising the RNA to produce the RNA lipid particles, wherein a non-ionic amphiphilic organic compound is present in the lipid solution or in the RNA solution, or both.

In some embodiments, the organic solvent comprises an alcohol.

In some embodiments, the alcohol is ethanol.

In some embodiments, the non-ionic amphiphilic organic compound comprises from about 0.1 mol % to about 50 mol %, from about 1 mol % to about 50 mol %, from about 1 mol % to about 40 mol %, from about 1 mol % to about 30 mol %, from about 1 mol % to about 20 mol %, from about 1 mol % to about 15 mol %, from about 1 mol % to about 10 mol %, from about 1 mol % to about 7.5 mol %, or from about 2 mol % to about 5 mol % of the total lipid (including lipid-like material, e.g., non-ionic amphiphilic organic compound) present in the lipid solution and/or the RNA solution. In some embodiments, the lipid solution and/or the RNA solution comprises a mol fraction of non-ionic amphiphilic organic compound from about 0.1 mol % to about 30 mol %. In some embodiments, the non-ionic amphiphilic organic compound comprises about 3 mol % of the total lipid present. In some embodiments, the non-ionic amphiphilic organic compound comprises at least about 3 mol % of the total lipid present. In some embodiments, the non-ionic amphiphilic organic compound comprises at least about 5 mol % of the total lipid present. In some embodiments, the non-ionic amphiphilic organic compound comprises at least about 10 mol % of the total lipid present. In some embodiments, the non-ionic amphiphilic organic compound comprises at least about 15 mol % of the total lipid present. In some embodiments, the non-ionic amphiphilic organic compound comprises at least about 20 mol % of the total lipid present. In some embodiments, the non-ionic amphiphilic organic compound comprises at least about 25 mol % of the total lipid present. In some embodiments, the non-ionic amphiphilic organic compound comprises less than about 30 mol % of the total lipid present. In some embodiments, the non-ionic amphiphilic organic compound comprises from about 5 mol % to about 30 mol % of the total lipid present. In some embodiments, the non-ionic amphiphilic organic compound comprises from about 5 mol % to about 25 mol % of the total lipid present. In some embodiments, the non-ionic amphiphilic organic compound comprises from about 5 mol % to about 20 mol % of the total lipid present. In some embodiments, the non-ionic amphiphilic organic compound comprises from about 5 mol % to about 15 mol % of the total lipid present. In some embodiments, the non-ionic amphiphilic organic compound comprises from about 5 mol % to about 10 mol % of the total lipid present. In some embodiments, the non-ionic amphiphilic organic compound comprises from about 10 mol % to about 25 mol % of the total lipid present. In some embodiments, the non-ionic amphiphilic organic compound comprises from about 15 mol % to about 30 mol % of the total lipid present. In some embodiments, the non-ionic amphiphilic organic compound comprises from about 15 mol % to about 25 mol % of the total lipid present. In some embodiments, the non-ionic amphiphilic organic compound comprises from about 20 mol % to about 25 mol % of the total lipid present.

In some embodiments, a composition comprises a non-ionic amphiphilic organic compound at a level of at least about 0.15 mM. In some embodiments, a composition comprises a non-ionic amphiphilic organic compound at a level of at least about 0.2 mM. In some embodiments, a composition comprises a non-ionic amphiphilic organic compound at a level of at least about 0.25 mM. In some embodiments, a composition comprises a non-ionic amphiphilic organic compound at a level of at least about 0.3 mM.

In some embodiments, a composition comprises a non-ionic amphiphilic organic compound at a level of at least about 0.35 mM. In some embodiments, a composition comprises a non-ionic amphiphilic organic compound at a level of at least about 0.4 mM. In some embodiments, a composition comprises a non-ionic amphiphilic organic compound at a level of at least about 0.45 mM. In some embodiments, a composition comprises a non-ionic amphiphilic organic compound at a level of at least about 0.5 mM. In some embodiments, a composition comprises a non-ionic amphiphilic organic compound at a level of at least about 0.55 mM. In some embodiments, a composition comprises a non-ionic amphiphilic organic compound at a level of at least about 0.6 mM. In some embodiments, a composition comprises a non-ionic amphiphilic organic compound at a level of at least about 0.7 mM. In some embodiments, a composition comprises a non-ionic amphiphilic organic compound at a level of at least about 0.8 mM. In some embodiments, a composition comprises a non-ionic amphiphilic organic compound at a level of at least about 0.9 mM. In some embodiments, a composition comprises a non-ionic amphiphilic organic compound at a level of at least about 1.0 mM. In some embodiments, a composition comprises a non-ionic amphiphilic organic compound at a level of at least about 1.1 mM. In some embodiments, a composition comprises a non-ionic amphiphilic organic compound at a level of at least about 1.2 mM. In some embodiments, a composition comprises a non-ionic amphiphilic organic compound at a level of at least about 1.3 mM. In some embodiments, a composition comprises a non-ionic amphiphilic organic compound at a level of at least about 1.4 mM. In some embodiments, a composition comprises a non-ionic amphiphilic organic compound at a level of at least about 1.5 mM. In some embodiments, a composition comprises a non-ionic amphiphilic organic compound at a level of at least about 2.0 mM. In some embodiments, a composition comprises a non-ionic amphiphilic organic compound at a level of at least about 2.5 mM. In some embodiments, a composition comprises a non-ionic amphiphilic organic compound at a level of at least about 3.0 mM. In some embodiments, a composition comprises a non-ionic amphiphilic organic compound at a level of at least about 4.0 mM. In some embodiments, a composition comprises a non-ionic amphiphilic organic compound at a level of at least about 5.0 mM.

In some embodiments, a composition comprises a non-ionic amphiphilic organic compound at a level of from about 0.15 mM to about 0.3 mM. In some embodiments, a composition comprises a non-ionic amphiphilic organic compound at a level of from about 0.3 mM to about 0.6 mM. In some embodiments, a composition comprises a non-ionic amphiphilic organic compound at a level of from about 0.6 mM to about 0.75 mM. In some embodiments, a composition comprises a non-ionic amphiphilic organic compound at a level of from about 0.75 mM to about 1.0 mM. In some embodiments, a composition comprises a non-ionic amphiphilic organic compound at a level of from about 1.0 mM to about 1.25 mM. In some embodiments, a composition comprises a non-ionic amphiphilic organic compound at a level of from about 1.25 mM to about 1.5 mM. In some embodiments, a composition comprises a non-ionic amphiphilic organic compound at a level of from about 1.75 mM to about 2.0 mM. In some embodiments, a composition comprises a non-ionic amphiphilic organic compound at a level of from about 2.0 mM to about 2.25 mM. In some embodiments, a composition comprises a non-ionic amphiphilic organic compound at a level of from about 2.25 mM to about 2.5 mM. In some embodiments, a composition comprises a non-ionic amphiphilic organic compound at a level of from about 2.5 mM to about 2.75 mM. In some embodiments, a composition comprises a non-ionic amphiphilic organic compound at a level of from about 2.75 mM to about 3.0 mM. In some embodiments, a composition comprises a non-ionic amphiphilic organic compound at a level of from about 3.0 mM to about 3.5 mM. In some embodiments, a composition comprises a non-ionic amphiphilic organic compound at a level of from about 4.0 mM to about 4.5 mM. In some embodiments, a composition comprises a non-ionic amphiphilic organic compound at a level of from about 4.5 mM to about 5.0 mM.

In some embodiments, a composition comprising RNA particles comprises the components specified in the following table:

| | Phase during | | |
|---|---|---|---|
| Components | manufacturing | Concentration | |
| RNA | RNA phase | 0.22 mg/mL | |
| | | (total RNA) | |
| HEPES | | 0.26 | mg/mL |
| EDTA | | 0.003 | mg/mL |
| NaCl | | 0.13 | mg/mL |
| Sodium citrate | | 0.12 | mg/mL |
| Citric acid | | 0.12 | mg/mL |
| DODMA | Liposome phase | 1.23 | mg/mL |
| DOPE | | 0.25 | mg/mL |
| DOPS | | 0.27 | mg/mL |
| Polysorbate 20 | | 0.81 | mg/mL |
| Acetic acid | | 0.03 | mg/mL |
| Sucrose | Storage matrix | 100 | mg/mL |
| Sodium acetate | | 0.41 | mg/mL |

| | | |
|---|---|---|
| Water for injection | Intermediate dilution | q.s. (e.g. 1 mL) |

In some embodiments, a composition comprising RNA particles comprises the components specified in the following table:

| Components | Concentration | |
|---|---|---|
| RNA | 0.44 mg/mL | |
| | (total RNA) | |
| DODMA | 2.48 | mg/mL |
| DOPE | 0.50 | mg/mL |
| DOPS | 0.54 | mg/mL |
| Polysorbate 20 | 1.64 | mg/mL |
| Acetic acid | 0.05 | mg/mL |
| Sucrose | 100 | mg/mL |
| TriSodium citrate dihydrate | 0.18 | mg/mL |
| Citric acid monohydrate | 0.19 | mg/mL |
| NaCl | 0.19 | mg/mL |
| HEPES | 0.53 | mg/mL |
| EDTA | 0.006 | mg/mL |
| Sodium acetate trihydrate | 0.68 | mg/mL |

In a further aspect, the invention provides a method of producing a composition comprising RNA lipoplex particles comprising at least one phosphatidylserine, the method comprising:
i) preparing an acidified aqueous colloid comprising liposomes, wherein the liposomes comprise at least one phosphatidylserine, wherein the acidified aqueous colloid comprising liposomes comprises from 10 to 30 mM acetic acid; and
ii) mixing the acidified aqueous colloid comprising liposomes with an aqueous solution comprising RNA;
   thereby producing the composition comprising RNA lipoplex particles,
   wherein the RNA lipoplex particles and the liposomes comprise dioleoylphosphatidylserine (DOPS); N,N-dimethyl-2,3-dioleyloxypropylamine (DODMA); and dioleoylphosphatidylethanolamine (DOPE).

In some embodiments, the molar ratio of DODMA:DOPE:DOPS is 5.5-6.5:0.9-1.1:0.9-1.1.

In some embodiments, the molar ratio of DODMA:DOPE:DOPS is about 6:about 1:about 1.

In some embodiments, the molar ratio of DODMA:DOPE:DOPS is about 6:about 1.2-1.8:about 1.

In some embodiments, the molar ratio of DODMA:DOPE:DOPS is about 6:about 1.2:about 1.

In some embodiments, the molar ratio of DODMA:DOPE:DOPS is about 6:about 1.7:about 1.

In some embodiments, the molar ratio of DODMA:DOPE:DOPS is about 6:about 1.8:about 1.

In some embodiments, the acidified aqueous colloid comprising liposomes has a pH of between about 4.5 and about 6.5, such as between about 5.0 and about 6.0, or between about 5.2 and about 5.5.

In some embodiments, the acidified aqueous colloid comprising liposomes comprises from 15 mM to 25 mM acetic acid.

In some embodiments, the composition comprises a non-ionic amphiphilic organic compound, optionally which is a surfactant. In some embodiments, the composition comprises a polyoxyethylene sorbitan ester, optionally a polysorbate, preferably polysorbate 20. In some embodiments, the non-ionic amphiphilic organic compound or the polyoxyethylene sorbitan ester (e.g., polysorbate 20) comprises from about 0.1 mol % to about 30 mol % of the total lipid present in the composition, optionally more than about 5 mol % of the total lipid present in the composition, preferably from about 5 mol % to about 25 mol % of the total lipid present in the composition.

In some embodiments, the composition comprises DODMA, DOPE, DOPS, and polysorbate 20.

In some embodiments, the composition comprises DODMA, DOPE, DOPS, and polysorbate 20, wherein the molar ratio of DODMA:DOPE:DOPS:polysorbate 20 is 5.5-6.5:0.9-1.1:0.9-1.1:1.5-2.5.

In some embodiments, the molar ratio of DODMA:DOPE:DOPS:polysorbate 20 is about 6:about 1:about 1:about 2.

In some embodiments, the molar ratio of DODMA:DOPE:DOPS:polysorbate 20 is about 6:about 1.2-1.8:about 1:about 2.

In some embodiments, the molar ratio of DODMA:DOPE:DOPS:polysorbate 20 is about 6:about 1.2:about 1:about 2.

In some embodiments, the molar ratio of DODMA:DOPE:DOPS:polysorbate 20 is about 6:about 1.7:about 1:about 2.

In some embodiments, the molar ratio of DODMA:DOPE:DOPS:polysorbate 20 is about 6:about 1.8:about 1:about 2.

In some embodiments, the aqueous solution comprising RNA does not comprise sodium chloride (NaCl).

In some embodiments, the acidified aqueous colloid comprising liposomes and the aqueous solution comprising RNA are mixed at a flow rate of at least 200 mL/min, optionally at a flow rate of 300-400 mL/min, preferably at a flow rate of 300-360 mL/min.

In some embodiments, the concentration of RNA in the aqueous solution comprising RNA, at the point of mixing with the acidified aqueous colloid comprising liposomes, is less than 0.5 mg/mL, optionally 0.1-0.5 mg/mL, preferably less than 0.3 mg/mL, optionally 0.1-0.25 mg/mL.

In some embodiments, the acidified aqueous colloid comprising liposomes is produced by a method comprising:
i) preparing a lipid solution comprising the at least one phosphatidylserine in an organic phase and injecting said lipid solution into an acidified aqueous phase; or
ii) preparing a lipid film by dehydration of a lipid solution comprising the at least one phosphatidylserine in an organic solvent and rehydrating the lipid film in an acidified aqueous phase.

In some embodiments, the organic solvent comprises or is an alcohol or acetone. In some embodiments, the organic solvent is ethanol, acetone, isopropyl alcohol (IPA) or tert-butanol.

In some embodiments, the lipid solution comprises DODMA and the acidic pH of the acidified aqueous colloid comprising liposomes renders the DODMA protonated.

In some embodiments, the method further comprises performing an ultrafiltration/diafiltration (UF/DF) step on the composition comprising RNA lipoplex particles. In some embodiments, said step increases the concentration of the RNA lipoplex particles as measured by the concentration of RNA.

In some embodiments, the method further comprises adding a dilution solution to the composition comprising RNA lipoplex particles. In some embodiments, the concentration of RNA lipoplex particles following addition of the dilution solution is less than 0.3 mg/mL, optionally 0.1-0.25 mg/mL.

In some embodiments, the dilution solution comprises at least 10% sucrose, at least 20% sucrose, or preferably at least 30% sucrose.

In some embodiments, the dilution solution comprises a buffer at pH 5.0-6.5, optionally at pH 5.1-6.0, optionally at pH 5.2-5.5, optionally at pH 5.3 or pH 5.5.

In some embodiments, the buffer is a histidine buffer. In some embodiments, the buffer comprises at least 20 mM histidine buffer.

In some embodiments, the RNA LPX particles have an N/P ratio from about 3 to about 5. In some embodiments, the RNA LPX particles have an N/P ratio of about 3.

In some embodiments, the diluted composition is suitable for administration to a human patient by injection, optionally im or iv injection, for example after thawing and without further dilution.

In a further aspect, the invention provides a composition comprising RNA-LPX particles comprising at least one phosphatidylserine, said composition being obtainable by the method of producing a composition comprising RNA lipoplex particles comprising at least one phosphatidylserine disclosed herein.

In some aspects, the invention provides the particles and compositions, e.g., pharmaceutical compositions, described herein for use in the treatments or methods of treatment described herein.

### BRIEF DESCRIPTION OF THE FIGURES

FIGS. 1A and 1B: (A): Overview of the PS-LNP Manufacturing Process used herein. (B): Overview of the PS-LPX Manufacturing Process used herein.

FIGS. 2A-2C. Luciferase activities in Balb/c mouse model. The in vitro-transcribed (IVT) mRNA was formulated with either PS-LNP1, PS-LNP2, or F12 lipoplex (Kranz et al. 2016, Nature 534: 396) as a control. 20 μg was injected into mice by intravenous (i.v.) route. Luciferin, substrate of luciferase was injected intraperitoneal at 4 and 24 hours following delivery of the formulated mRNA and luciferase activity was detected using in vivo imaging system (IVIS). FIG. 2A shows lateral and ventral images of injected mice at 4 and 24 hours. FIGS. 2B and 2C show total flux values in liver (FIG. 2B) and spleen (FIG. 2C).

FIGS. 3A and 3B. 24 hours ex-vivo luciferase activities in Balb/c mouse model organs. The IVT mRNA was formulated with either PS-LNP1, PS-LNP2 or F12 lipoplex (Kranz et al. 2016, Nature 534: 396) as a control. 20 μg was injected into mice by intravenous (i.v.) route. After 24 hours, the mice were sacrificed and the organs were removed and luciferase activity was detected using in vivo imaging system (IVIS). FIG. 3A shows images of lung, liver, spleen, and kidney. FIG. 3B show total flux values in liver and spleen.

FIGS. 4A-4C. Luciferase activities in B6 mouse model. The IVT mRNA was formulated with ether PS-LNP1, PS-LNP2, or F12 lipoplex (Kranz et al. 2016, Nature 534: 396) as a control and 20 μg was injected into mice by intravenous (i.v.) route. Luciferin, substrate of luciferase, was injected intraperitoneal at 4 and 24 hours following delivery of the formulated mRNA and luciferase activity was detected using in vivo imaging system (IVIS). FIG. 4A shows lateral and ventral images of injected mice at 4 and 24 hours. FIGS. 4B and 4C shows total flux values in liver (FIG. 4B) and spleen (FIG. 4C).

FIGS. 5A and B. 24 hours ex-vivo luciferase activities in B6 mouse model organs. The IVT mRNA was formulated with either PS-LNP1, PS-LNP2, or F12 lipoplex (Kranz et al. 2016, Nature 534: 396) as a control. 20 μg was injected into mice by intravenous (i.v.) route. After 24 hours, the mice were sacrificed and the organs were removed and luciferase activity was detected using in vivo imaging system (IVIS). FIG. 5A shows images of lung, liver, spleen, and kidney. FIG. 5B show total flux values in liver and spleen.

FIG. 6. Luciferase activities in mice injected with in vitro-transcribed (IVT) firefly luciferase-encoding mRNA formulated with phosphatidylserine PS-LNP1 or PS-LNP2 or PS-LNP3 and delivered via different application routes . . . 3 μg was injected into mice by intradermal (i.d.), intramuscular (i.m.) or intravenous (i.v.) routes. Luciferin, substrate of luciferase, was injected intraperitoneal at 6, 24, 48, 72 hours and at day 6 following delivery of the formulated mRNA and luciferase activity was detected using in vivo imaging system (IVIS).

FIG. 7. Plasma erythropoietin (EPO) levels in mice injected with in vitro-transcribed (IVT) murine EPO (mEPO)-encoding mRNA formulated with phosphatidylserine PS-LNP1 or PS-LNP2 and delivered via different application routes.

FIGS. 8A and 8B. Luciferase activity in HepG2 cells transfected with in vitro-transcribed (IVT) luciferase mRNA formulated with phosphatidylserine PS-LNP1 and PS-LNP26. F12 lipoplex (Kranz et al. 2016, Nature 534: 396) and TransIT were used as an internal and commercial control, respectively. Experiment was performed in triplicates. Results are shown in linear (FIG. 8A) and logarithmic scale (FIG. 8B).

FIGS. 9A-9C. Mice were injected intratumorally with preparations of two different mRNAs encoding for firefly luciferase and IL-12sc, respectively, in the indicated formulations, PS-LNP1 and PS-LNP2, to facilitate transfection and target translation. PS-LNP1 particle size, PDI, zeta potential, and EE % were 203 nm, 0.14, −8.19 mV, and 84, respectively. PS-LNP2 particle size, PDI, zeta potential, and EE % were 362 nm, 0.26, −30.52 mV, and 93, respectively. The N/P ratio was 3. Luciferase protein activity was evaluated 6 h post injection (p.i.) by bioluminescent imaging at the target anatomical site (tumor; FIG. 9A) as well as off-target locations (FIG. 9B), the latter reflecting signals from spleen and liver. A value around 1×10⁵ p/s can be considered base-line. Target-to-off target ratios indicate the extent of specific delivery to the tumor by means of formulation. IL-12 is a secreted cytokine and was determined by MSD ELISA in serum withdrawn at 6 h p.i. (FIG. 9C).

FIGS. 10A-10F. Mice were injected intratumorally with preparations of two different mRNAs encoding for firefly luciferase and IL-12sc, respectively, in the indicated formulations to facilitate transfection and target translation. Luciferase protein activity was evaluated 6 and 24 h p.i. by bioluminescent imaging at the target anatomical site (tumor) as well as off-target organs. BLI ratio indicates the extent of specific delivery to the tumor at 6 h p.i. as opposed to off-target organs by means of the formulation. IL-12 was determined by MSD ELISA in mouse serum withdrawn at 6 h p.i. FIG. 10A shows tumor BLI at 6 h; FIG. 10B shows tumor BLI at 24 h; FIG. 10C shows off-target BLI at 6 h; FIG. 10D shows target/off-target ratios; FIG. 10E shows serum IL-12 cytokine levels; FIG. 10F shows total flux measurements.

FIGS. 11A-11B. Mice were injected intratumorally with preparations of two different mRNAs encoding for firefly luciferase and IL-12sc, respectively, in the indicated formulations to facilitate transfection and target translation. Luciferase protein activity was evaluated 6, 24 and 48 h p.i. by bioluminescent imaging at the target anatomical site (tumor) as well as off-target organs. FIG. 11A shows images of tumors at 6 h p.i., and FIG. 11B shows total flux measurements up to 48 h p.i.

FIGS. 12A-12C. Mice were injected intratumorally with an mRNA encoding for firefly luciferase in the indicated formulations to facilitate transfection and target translation. Luciferase protein activity was evaluated 6 p.i. by bioluminescent imaging at the target anatomical site (tumor) as well as off-target organs. FIG. 12A shows target and off-target total flux measurements at 6 h p.i, and FIG. 12B shows total flux measurements up to 48 hours p.i. FIG. 12C shows the regional BLI value ratios at 6 h p.i.

FIGS. 13A-13E. Mice were injected intratumorally with preparations of two different mRNAs encoding for firefly luciferase and IL-12sc, respectively, in the indicated formulations to facilitate transfection and target translation. Luciferase protein activity was evaluated 6, 24 and 48 h p.i. by bioluminescent imaging at the target anatomical site (tumor) as well as off-target organs. IL-12 was determined by MSD ELISA in mouse serum withdrawn at 6 h p.i. FIGS. 13A and 13B show tumor total flux measurements at 6 h p.i. and kinetics out to 50 h p.i, respectively. FIGS. 13C and 13D show target/off-target BLI Ratios at 6 h p.i. and 24 h, respectively. FIG. 13E shows IL-12 serum levels.

FIG. 14. Erythropoietin (EPO) secretion by HepG2 cells transfected with in vitro-transcribed murine EPO mRNA formulated with phosphatidylserine PS-LNP24 and PS-LNP25. Murine EPO levels were measured in the culture medium by ELISA. F12 lipoplex (Kranz et al. 2016, Nature 534: 396) and TransIT were used as an internal and commercial control, respectively. Experiment was performed in triplicates.

FIG. 15. Plasma erythropoietin (EPO) levels in mice injected with in vitro-transcribed murine EPO-encoding mRNA formulated either with phosphatidylserine PS-LNP24, PS-LNP25, or TransIT a control. Mice were injected with either 5 μg mRNA by subcutaneous (s.c.) or 3 μg mRNA by intravenous (i.v.) delivery routes. Blood was collected at 6, 24, 48 and 72 hours following injection of the mRNA and murine EPO levels were measured in the plasma by ELISA as described (Mahiny and Karikó 2016, Methods in Mol Biol 1428: 297).

FIGS. 16A-16D. Mice were injected intratumorally with preparations of two different mRNAs encoding for firefly luciferase and IL-12sc, respectively, in the indicated formulations to facilitate transfection and target translation. Luciferase protein activity was evaluated 6, 24 and 48 h p.i. by bioluminescent imaging at the target anatomical site (tumor) as well as off-target organs. BLI Ratio indicates target-to-off target ratios at the given time-point. IL-12 was determined by MSD ELISA in mouse serum withdrawn at 6 h p.i. FIGS. 16A and 16B show tumor total flux measurements for tumor (16A) and target/off-target ratios (16B). FIG. 16C shows target and off-target total flux measurements. FIG. 16D shows IL-12 serum levels.

FIGS. 17A-D. Mice were injected intratumorally with preparations of two different mRNAs encoding for firefly luciferase and IL-12sc, respectively, in the indicated formulations to facilitate transfection and target translation. Luciferase protein activity was evaluated 6 and 24 h p.i. by bioluminescent imaging at the target anatomical site (tumor) as well as off-target organs. BLI Ratio indicates the extent of specific delivery to the tumor by means of formulation at 6 and 24 h p.i. FIG. 17A shows tumor and off-target BLI images at 6 h and 24 hours; FIG. 17B shows target and off-target BLI at 6 h; FIG. 17C shows target/off-target BLI Ratio at 6 h; FIG. 17D shows total flux measurements at 6 and 24 h p.i.

FIGS. 18A-18D. 4T1 tumor-bearing mice were injected intratumorally with preparations of two different mRNAs encoding for firefly luciferase and IL-12sc, respectively, in the indicated formulations to facilitate transfection and target translation. Luciferase protein activity was evaluated 6, 24 and 48 h p.i. by bioluminescent imaging at the target anatomical site (tumor) as well as off-target organs. Fold over spleen/liver indicates target-to-off target ratios at the given time-point. FIG. 18A shows images at 6, 24, and 48 hours; FIG. 18B shows target and off-target BLI at 6 h; FIG. 18C shows target/off-target kinetics at 6 and 24 hours; FIG. 18D shows total flux measurements at 6, 24, and 48 hours p.i.

FIGS. 19A-C. Mice were injected intratumorally with preparations of two different mRNAs encoding for firefly luciferase and IL-12sc, respectively, in the indicated formulations to facilitate transfection and target translation. Luciferase protein activity was evaluated 6, 28 and 49 h p.i. by bioluminescent imaging at the target anatomical site (tumor) as well as off-target organs. Fold over spleen/liver indicates target-to-off target ratios at the given time-point. FIG. 19A shows target and off-target BLI at 6 h; FIG. 19B shows target/off-target kinetics at 6, 28, and 50 hours; FIG. 19C shows target/off-target ratio kinetics for various particles at various time points.

FIGS. 20A-20H. CT26 tumor-bearing mice were injected intratumorally with mRNA encoding for Thy1.1 or control RNA complexed with the indicated formulations followed by organ harvesting 20 h p.i. and flow cytometric analysis for Thy 1.1 expression. FIG. 20A shows on-target Thy1.1 positive percentages; FIG. 20B shows off-target Thy1.1 positive percentages; FIGS. 20C-20E show results of flow cytometric analyses; FIG. 20F shows TIL subpopulations Thy1.1 positive percentages; FIG. 20G shows spleen subpopulations Thy1.1 positive percentages; FIG. 20H shows liver subpopulations Thy1.1 positive percentages.

FIGS. 21A and 21B. Mice were injected intratumorally with an mRNA encoding for firefly luciferase in the indicated formulations as in PS-PLX52 DODMA/DOPE/DOPS/polysorbate 20, 1.8/0.3/0.3/0.6 mM (Group A), PS-LPX52 DODMA/DOPE/DOPS/polysorbate 20, 1.8/0.3/0.3/0.3 mM (Group B) and PS-LPX52 DODMA/DOPE/DOPS/polysorbate 20, 1.8/0.3/0.3/0.15 mM (Group C) to facilitate transfection and target translation. Luciferase protein activity was evaluated 6 h, 24 h, 48 h p.i. by bioluminescent imaging at the target anatomical site (tumor) as well as off-target organs. BLI Ratio indicates the regional BLI value ratios at 6 h p.i. FIG. 21A shows images for tumor and off-target sites; FIG. 21B shows total flux for the three test groups at 6 h p.i.

FIGS. 22A-G. Mice bearing established CT26 tumors were treated intratumorally with a mixture of cytokine-encoding mRNAs or control mRNA complexed with the indicated formulations. Compared to naked RNA in Ringers solution, all formulations increased the complete response rate, with LNP49-3 curing 90% of treated mice. The sizes, indicated in Z-averages (Z.average), and the polydispersity indices (PIs) of the particles were measured by Photon Correlation Spectroscopy. FIGS. 22A and B show Ringer solution control for cytokine mRNA (A) and control mRNA (B); FIGS. 22 C and D show PS-LNP25 for cytokine mRNA (A) and control mRNA (B); FIGS. 22 E and F show PS-LNP49-3 for cytokine mRNA (A) and control mRNA (B); FIG. 22G shows the Z.average and polydispersity (PDI) for the tested particles.

FIGS. 23 A-G. Mice bearing established CT26 tumors were treated 4 times intratumorally with a mixture of cytokine-encoding mRNAs or control luc mRNA complexed with either PS-LPX52 (PS-LNP52 lipid blend) or in plain Ringers salt solution in two doses each. FIGS. 23A and B show individual tumor volumes for PS-LPX52 hi (A) and lo (B) treated animals; FIGS. 23 C and D show individual tumor volumes for Ringer hi (A) and lo (B) treated animals; FIG. 23 E shows individual tumor volumes for luc control treated animals; FIG. 23F shows mean tumor growth kinetics for the test and control groups; FIG. 23G shows the Z.average and polydispersity (PDI) for the tested particles.

FIGS. 24A and B. PSLPX2*It.IV was manufactured using a protocol compatible with clinical-scale manufacture and frozen and stored at −20° C. for up to 3 months, then injected into TC-1 tumor-bearing mice. Due to the optimized storage matrix and preparation protocol prior to injection, no effect of the storage duration was observed as a function of biodistribution or cytokine secretion over the course of the experiment. FIG. 24A shows images and BLI data; FIG. 24B shows a schematic of the experiment.

FIGS. 25A and B. Cryo-Transmission Electron Microscopy (Cryo-TEM) of PS-LNP1 (A) and PS-LNP2 (B). Lipid composition was DODMA/CHOL/DOPS/polysorbate 20 and DODMA/DOPE/DOPS/polysorbate 20, 57/30/10/3, 77/10/10/3, molar ratio for PS-LNP1 and PS-LNP2, respectively. They contained RNA at a positive/negative (DODMA lipid/RNA nucleotide) ratio of 3:1 and RNA concentration of 0.5 mg/mL.

FIG. 26. Cryo-Transmission Electron Microscopy (Cryo-TEM) of PS-LPX52. Lipid composition was DODMA/CHOL/DOPS/Tween20 and DODMA/DOPE/DOPS/Tween20, 60/10/10/20, molar ratio. It contained RNA at a positive/negative (DODMA lipid/RNA nucleotide) ratio of 3:1. Multilamellar structures, with individual species having a diameter 100 nm<2a<150 nm. Many particles are merged together, forming larger complexes. The insets show the Fourier transforms of the corresponding images with lamellar structures. The spacing obtained from the FT images is d=5.9-6.1 nm (pH=5.2±0.1).

FIG. 27. Buffer components of PS-LPX-A. (A, B) Comparison of physicochemical properties, particle size and polydispersity, of PS-LPX-A comprising citrate (A) or acetate (B) buffer, pH 4. (C) Protein expression from PS-LPX-A in vitro was measured and compared for PS-LPX-A with citrate buffer or acetate buffer. (D) Protein expression from PS-LPX-A was compared for PS-LPX-A supplemented with (i) sodium chloride, or (ii) citrate buffer and sodium chloride.

FIG. 28. N/P ratio of PS-LPX-A. (A) Protein expression from PS-LPX-A prepared at N/P ratios between 0.5 to 6. (B) Encapsulation efficiency (% EE) and RNA accessibility (%) was measured for PS-LPX-A prepared at N/P ratios between 0.5 to 6.

FIG. 29. PS-LPX-A mixing flow rates. (A) Size and polydispersity of PS-LPX-A particles formed at flow rates between 50 and 360 mL/min were measured. Numbers of particles having sizes of >10 μm and >25 μm for PS-LPX-A particles formed at flow rates between 50 and 360 mL/min were measured. (B) Protein expression from PS-LPX-A particles formed at flow rates between 50 and 388 mL/min was measured.

FIG. 30. Buffer conditions during PS-LPX-A particle formation. (A) Protein expression in vitro from PS-LPX-A produced in the presence of acetic acid concentrations between 2.5 mM and 30 mM at particle formation was measured and compared to benchmark (BM, 5 mM acetic acid). (B) Protein expression in vitro from PS-LPX-A manufactured with 5 mM or 20 mM acetic acid at particle formation was measured and compared to BM.

FIG. 31. Storage buffers for PS-LPX-A. (A, B) Particle size and the pH and zeta potential of the PS-LPX-A composition were measured following addition of 20 mM histidine storage buffer at pHs between 4.5 and 5.5. (C) Particle size of PS-LPX-A was measured over the indicated number of freeze-thaw cycles following addition of 20 mM histidine storage buffer at pHs between 4.5 and 5.5. (D) Protein expression from PS-LPX-A was measured following addition of 20 mM histidine storage buffer at pHs between 4.5 and 5.5, and compared to BM (pH 6.7).

FIG. 32. Refining lipid ratios in PS-LPX-A. Protein expression from PS-LPX-A prepared with the indicated % mol ratios of DODMA, DOPE and DOPS lipids was measured.

FIG. 33. Particle size of PS-LPX-A. (A) Particle size, polydispersity and number of particles having sizes >10 μm or >25 μm were measured for PS-LPX-A prepared at RNA concentrations at particle formation of between 0.11 and 0.66 mg/mL as indicated. (B) Protein expression from PS-LPX-A prepared with RNA concentrations between 0.4 and 0.6 mg/mL at particle formation was measured.

FIG. 34. Stability of 2× up-concentrated PS-LPX formulation. (A-B) Particle size and polydispersity (A) and number of particles having sizes >10 μm or >25 μm (B) were measured for 2λ, 3× and 4× PS-LPX formulations after the indicated storage conditions. (C-E) Particle size and polydispersity (C), number of particles having sizes >10 μm or >25 μm (D) and RNA concentration (E) were measured for 2× PS-LPX formulation after the indicated storage conditions.

### DETAILED DESCRIPTION OF THE INVENTION

Although the present disclosure is further described in more detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art. In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The practice of the present disclosure will employ, unless otherwise indicated, conventional chemistry, biochemistry, cell biology, immunology, and recombinant DNA techniques which are explained in the literature in the field.

Throughout this specification and the claims which follow, unless the context requires otherwise, the word “comprise”, and variations such as “comprises” and “comprising”, will be understood to imply the inclusion of a stated feature, element, member, integer or step or group of features, elements, members, integers or steps but not the exclusion of any other feature, element, member, integer or step or group of members, integers or steps. The term “consisting essentially of” limits the scope of a claim or disclosure to the specified features, elements, members, integers, or steps and those that do not materially affect the basic and novel characteristic(s) of the claim or disclosure. The term “consisting of” limits the scope of a claim or disclosure to the specified features, elements, members, integers, or steps. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by the context.

The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±10%, ±5%, ±4%, 3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%.

In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

The term “enhance” as used herein means the ability to cause an overall increase, or enhancement, for example, by at least about 5% or greater, about 10% or greater, about 15% or greater, about 20% or greater, about 25% or greater, about 30% or greater, about 40% or greater, about 50% or greater, about 75% or greater, or about 100% or greater in the level.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material such as surfactants, for example polysorbate 20.

The term “ionic strength” refers to the mathematical relationship between the number of different kinds of ionic species in a particular solution and their respective charges. Thus, ionic strength I is represented mathematically by the formula:

$$I = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in some embodiments relates to the presence of monovalent ions. Regarding the presence of divalent ions, in particular divalent cations, their concentration or effective concentration (presence of free ions) due to the presence of chelating agents is, in some embodiments, sufficiently low so as to prevent degradation of the RNA. In some embodiments, the concentration or effective concentration of divalent ions is below the catalytic level for hydrolysis of the phosphodiester bonds between RNA nucleotides. In some embodiments, the concentration of free divalent ions is 20 μM or less. In some embodiments, there are no or essentially no free divalent ions.

“Osmolality” refers to the concentration of a particular solute expressed as the number of osmoles of solute per kilogram of solvent.

The term “lyophilizing” or “lyophilization” refers to the freeze-drying of a substance by freezing it and then reducing the surrounding pressure (e.g., below 15 Pa, such as below 10 Pa, below 5 Pa, or 1 Pa or less) to allow the frozen medium in the substance to sublimate directly from the solid phase to the gas phase. Thus, the terms “lyophilizing” and “freeze-drying” are used herein interchangeably.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “reconstitute” relates to adding a solvent such as water to a dried product to return it to a liquid state such as its original liquid state.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C. In some embodiments, the temperature is from 15° C. to about 25° C. In some embodiments, the temperature is from 17° C. to about 25° C. In some embodiments, the temperature is about 15° C. In some embodiments, the temperature is about 16° C. In some embodiments, the temperature is about 17° C. In some embodiments, the temperature is about 18° C. In some embodiments, the temperature is about 19° C. In some embodiments, the temperature is about 20° C. In some embodiments, the temperature is about 21° C. In some embodiments, the temperature is about 22° C.

The term “EDTA” refers to ethylenediaminetetraacetic acid disodium salt. All concentrations are given with respect to the EDTA disodium salt.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

According to the present disclosure, the term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “polypeptide” refers to large peptides, in particular peptides having at least about 151 amino acids. The terms “peptide” and “polypeptide” are both protein molecules.

A “therapeutic protein” has a positive or advantageous effect on a condition or disease state of a subject when provided to the subject in a therapeutically effective amount. In some embodiments, a therapeutic protein has curative or palliative properties and may be administered to ameliorate, relieve, alleviate, reverse, delay onset of or lessen the severity of one or more symptoms of a disease. A therapeutic protein may have prophylactic properties and may be used to delay the onset of a disease or to lessen the severity of such disease or pathological condition. The term “therapeutic protein” includes entire peptides or polypeptides, and can also refer to therapeutically active fragments thereof. It can also include therapeutically active variants of a peptide or polypeptide. Examples of therapeutically active proteins include, but are not limited to, antigens for vaccination and immunostimulants such as cytokines. The terms “therapeutic protein” and “therapeutically active protein” are used interchangeably herein.

According to various embodiments of the present disclosure, a nucleic acid such as mRNA encoding a peptide or polypeptide is taken up by or introduced, i.e. transfected or transduced, into a cell which cell may be present in vitro or in a subject, resulting in expression of said peptide or polypeptide. The cell may, e.g., express the encoded peptide or polypeptide intracellularly (e.g. in the cytoplasm and/or in the nucleus), may secrete the encoded peptide or polypeptide, and/or may express it on the surface.

According to the present disclosure, terms such as “nucleic acid expressing” and “nucleic acid encoding” or similar terms are used interchangeably herein and with respect to a particular peptide or polypeptide mean that the nucleic acid, if present in the appropriate environment, e.g. within a cell, can be expressed to produce said peptide or polypeptide.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may be any portion from 0.1% to 99.9% (such as 0.1%, 0.5%, 1%, 5%, 10%, 50%, 90%, or 99%) of said composition.

“Fragment”, with reference to an amino acid sequence (peptide or polypeptide), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence comprises, e.g., at least 6, in particular at least 8, at least 12, at least 15, at least 20, at least 30, at least 50, or at least 100 consecutive amino acids from an amino acid sequence.

According to certain embodiments of the present disclosure, a part or fragment of a peptide or polypeptide has at least one functional property of the peptide or polypeptide from which it has been derived. Such functional properties may comprise a pharmacological activity, the interaction with other peptides or polypeptides, an enzymatic activity, the interaction with antibodies, and the selective binding of nucleic acids. E.g., a pharmacological active fragment of a peptide or polypeptide has at least one of the pharmacological activities of the peptide or polypeptide from which the fragment has been derived. A part or fragment of a peptide or polypeptide comprises, e.g., a sequence of at least 6, in particular at least 8, at least 10, at least 12, at least 15, at least 20, at least 30 or at least 50, consecutive amino acids of the peptide or polypeptide. A part or fragment of a peptide or polypeptide comprises, e.g., a sequence of up to 8, in particular up to 10, up to 12, up to 15, up to 20, up to 30 or up to 55, consecutive amino acids of the peptide or polypeptide.

“Variant,” as used herein and with reference to an amino acid sequence (peptide or polypeptide), is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference as compared to the parent amino acid sequence, e.g., from 1 to about 20 amino acid differences, such as from 1 to about 10 or from 1 to about 5 amino acid differences compared to the parent.

By “wild type” or “WT” or “native” herein is meant an amino acid sequence that is found in nature, including allelic variations. A wild type amino acid sequence, peptide or polypeptide has an amino acid sequence that has not been intentionally modified.

For the purposes of the present disclosure, “variants” of an amino acid sequence (peptide or polypeptide) may comprise amino acid insertion variants, amino acid addition variants, amino acid deletion variants and/or amino acid substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous peptides or polypeptides and/or to replacing amino acids with other ones having similar properties. In some embodiments, amino acid changes in peptide and polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

In some embodiments the degree of similarity, such as identity between a given amino acid sequence and an amino acid sequence which is a variant of said given amino acid sequence, will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the degree of similarity or identity is given for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given, e.g., for at least about 20, at least about 40, at least about 60, at least about 80, at least about 100, at least about 120, at least about 140, at least about 160, at least about 180, or about 200 amino acids, in some embodiments continuous amino acids. In some embodiments, the degree of similarity or identity is given for the entire length of the reference amino acid sequence. The alignment for determining sequence similarity, such as sequence identity, can be done with art known tools, such as using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and, e.g., at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or polypeptides having substitutions, additions, insertions or deletions, is described in detail in Molecular Cloning: A Laboratory Manual, 4^{th} Edition, M. R. Green and J. Sambrook eds., Cold Spring Harbor Laboratory Press, Cold Spring Harbor 2012, for example. Furthermore, the peptides, polypeptides and amino acid variants described herein may be readily prepared with the aid of known peptide synthesis techniques such as, for example, by solid phase synthesis and similar methods.

In some embodiments, a fragment or variant of an amino acid sequence (peptide or polypeptide) is a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., function of the functional fragment or functional variant may be at least 50%, at least 60%, at least 70%, at least 80%, or at least 90% of the parent molecule or sequence. However, in other embodiments, function of the functional fragment or functional variant may be enhanced compared to the parent molecule or sequence.

An amino acid sequence (peptide or polypeptide) “derived from” a designated amino acid sequence (peptide or polypeptide) refers to the origin of the first amino acid sequence. In some embodiments, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means removed (e.g., purified) from the natural state or from an artificial composition, such as a composition from a production process. For example, a nucleic acid, peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. Generally, nucleic acid encoding antigen is transiently transfected into cells. RNA can be transfected into cells to transiently express its coded protein.

According to the present disclosure, an analog of a peptide or polypeptide is a modified form of said peptide or polypeptide from which it has been derived and has at least one functional property of said peptide or polypeptide. E.g., a pharmacological active analog of a peptide or polypeptide has at least one of the pharmacological activities of the peptide or polypeptide from which the analog has been derived. Such modifications include any chemical modification and comprise single or multiple substitutions, deletions and/or additions of any molecules associated with the peptide or polypeptide, such as carbohydrates, lipids and/or peptides or polypeptides. In some embodiments, “analogs” of peptides or polypeptides include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said peptides and polypeptides.

“Activation” or “stimulation”, as used herein, refers to the state of a cell that has been sufficiently stimulated to induce detectable cellular proliferation, such as an immune effector cell such as T cell. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “expansion” refers to a process wherein a specific entity is multiplied. In some embodiments, the term is used in the context of an immunological response in which immune effector cells are stimulated by an antigen, proliferate, and the specific immune effector cell recognizing said antigen is amplified. In some embodiments, expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, such as a peptide or polypeptide, that reacts specifically with antibodies or T-lymphocytes (T-cells). The term “antigen” may comprise a molecule that comprises at least one epitope, such as a T cell epitope. In some embodiments, an antigen is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen.

According to the present disclosure, any suitable antigen may be used, which is a candidate for an immune response, wherein the immune response may be both a humoral as well as a cellular immune response. In the context of some embodiments of the present disclosure, the antigen is presented by a cell, such as by an antigen presenting cell, in the context of MHC molecules, which results in an immune response against the antigen. An antigen may be a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In some embodiments, the antigen is a tumor antigen, i.e., a part of a tumor cell, in particular those which primarily occur intracellularly or as surface antigens of tumor cells. In another embodiment, the antigen is a pathogen-associated antigen, i.e., an antigen derived from a pathogen, e.g., from a virus, bacterium, unicellular organism, or parasite, for example a viral antigen such as viral ribonucleoprotein or coat protein. In some embodiments, the antigen should be presented by MHC molecules which results in modulation, in particular activation of cells of the immune system, such as CD4+ and CD8+ lymphocytes, in particular via the modulation of the activity of a T-cell receptor.

The term “tumor antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, α1-fetoprotein, isoferritin, and fetal sulphoglycoprotein, α2-H-ferroprotein and γ-fetoprotein, as well as various virus tumor antigens. According to some embodiments of the present disclosure, a tumor antigen comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies, T cells or B cells, in particular when presented in the context of MHC molecules. An epitope of a protein may comprises a continuous or discontinuous portion of said protein and, e.g., may be between about 5 and about 100, between about 5 and about 50, between about 8 and about 30, or about 10 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, the epitope in the context of the present disclosure is a T cell epitope.

Terms such as “epitope”, “fragment of an antigen”, “immunogenic peptide” and “antigen peptide” are used interchangeably herein and, e.g., may relate to an incomplete representation of an antigen which is, e.g., capable of eliciting an immune response against the antigen or a cell expressing or comprising and presenting the antigen. In some embodiments, the terms relate to an immunogenic portion of an antigen. In some embodiments, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, between about 8 and about 30, or between about 8 and about 25 amino acids in length, for example, the epitope may be 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and polypeptide antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or polypeptide antigen can be any peptide or polypeptide that can induce or increase the ability of the immune system to develop antibodies and T cell responses to the peptide or polypeptide.

In some embodiments, vaccine antigen, i.e., an antigen whose inoculation into a subject induces an immune response, is recognized by an immune effector cell. In some embodiments, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen may be, e.g., presented or present on the surface of a cell, such as an antigen presenting cell. In some embodiments, an antigen is presented by a diseased cell (such as tumor cell or an infected cell). In some embodiments, an antigen receptor is a TCR which binds to an epitope of an antigen presented in the context of MHC. In some embodiments, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells release cytotoxic factors, e.g., performs and granzymes.

In some embodiments, an antigen receptor is an antibody or B cell receptor which binds to an epitope in an antigen. In some embodiments, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part may be, e.g., at least 4, at least 8, pat least 12, or at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. In some embodiments, the term refers to one or more extracellular loops or domains or a fragment thereof.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In some embodiments of the present disclosure, the RNA (in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In some embodiments, the target cell is a cell expressing an antigen and presenting said antigen with class I MHC.

“Antigen processing” refers to the degradation of an antigen into processing products which are fragments of said antigen (e.g., the degradation of a polypeptide into peptides) and the association of one or more of these fragments (e.g., via binding) with MHC molecules for presentation by cells, such as antigen-presenting cells to specific T-cells.

By “antigen-responsive CTL” is meant a CD8+ T-cell that is responsive to an antigen or a peptide derived from said antigen, which is presented with class I MHC on the surface of antigen presenting cells.

According to the disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and may refer to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4⁺ and CD8⁺ T-lymphocytes, e.g. CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, such as the induction of a cellular immune response, a humoral immune response, or both. The immune response may be protective/preventive/prophylactic and/or therapeutic. The immune response may be directed against any immunogen or antigen or antigen peptide, such as against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. In some embodiments, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CHi, CH2, CH3). The constant regions of the antibodies may mediate the binding of the immunoglobulin to host tissues or factors, including various cells of the immune system (e.g., effector cells) and the first component (C1q) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The term “immunoglobulin” relates to proteins of the immunoglobulin superfamily, such as to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the V_{L} (variable light chain) domain, C_{L} (constant light chain) domain, V_{H} (variable heavy chain) domain, and the C_{H} (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The terms “vaccination” and “immunization” describe the process of treating an individual for therapeutic or prophylactic reasons and relate to the procedure of administering one or more immunogen(s) or antigen(s) or derivatives thereof, in particular in the form of RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, such as MHC class I and/or MHC class II molecules. In some embodiments, the MHC molecules are MHC class I molecules.

In the context of the present disclosure, the term “transcription” relates to a process, wherein the genetic code in a DNA sequence is transcribed into RNA (especially mRNA). Subsequently, the RNA may be translated into peptide or polypeptide.

With respect to RNA, the term “expression” or “translation” relates to the process in the ribosomes of a cell by which a strand of mRNA directs the assembly of a sequence of amino acids to make a peptide or polypeptide.

The term “optional” or “optionally” as used herein means that the subsequently described event, circumstance or condition may or may not occur, and that the description includes instances where said event, circumstance, or condition occurs and instances in which it does not occur.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present invention includes all isomers such as geometrical isomers, optical isomers based on an asymmetrical carbon, stereoisomers, tautomers and the like which occur structurally and isomer mixtures and is not limited to the description of the formula.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (±). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimensional forms of (hetero)cyclic rings, such as chair, half-chair, boat, and twist-boat forms of cyclohexane.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is may be calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses m, (i=1, 2, 3, . . . , n), located at fixed distances s, from the center of mass, R_{g} is the square-root of the mass average of sᵢ² over all mass elements and can be calculated as follows:

$${R}_{g} = {( {\sum }_{i = 1}^{n} {m}_{i} \cdot {s}_{i}^{2} / {\sum }_{i = 1}^{n} {m}_{i} )}^{1 / 2}$$

The radius of gyration can be determined or calculated experimentally, e.g., by using light scattering. In particular, for small scattering vectors 4 the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; T is the temperature; η is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles contained in a sample or control composition as disclosed herein or the hydrodynamic radius of a particle peak obtained from subjecting such a sample or control composition to field-flow fractionation) is to measure the DLS signal of said particle or population of particles (such as DLS signal of particles contained in a sample or control composition as disclosed herein or the DLS signal of a particle peak obtained from subjecting such a sample or control composition to field-flow fractionation).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In certain embodiments, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation:

$$\frac{{R}_{θ}}{{K}^{*} c} = {M}_{w} P ( θ ) - 2 {A}_{2} c {M}_{w}^{2} {P}^{2} ( θ )$$

wherein c is the mass concentration of the particles in the solvent (g/mL); A₂ is the second virial coefficient (mol·mL/g²); P(θ) is a form factor relating to the dependence of scattered light intensity on angle; R_{θ} is the excess Rayleigh ratio (cm⁻¹); and K* is an optical constant that is equal to 4π²ηₒ (dn/dc)²λ₀⁻⁴N_{A}⁻¹, where ηₒ is the refractive index of the solvent at the incident radiation (vacuum) wavelength, λ₀ is the incident radiation (vacuum) wavelength (n), N_{A} is Avogadro's number (mol⁻¹), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term:

$$\sqrt{\frac{{R}_{θ}}{{K}^{*} c}}$$

wherein c, R_{θ} and K* are as defined above. Preferably, the Debye plot is calculated the following term:

$$\frac{{K}^{*} c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is preferably modified by one or more alkyl groups, more preferably one or more C₁₋₄ alkyl groups, even more preferably one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N⁶—C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, N⁶-methyl-adenine, 5-methyl-cytosine, 5-methyl-uracil, and N(1)-methyl-uracil.

Herein, the term “DNA” relates to a nucleic acid molecule which includes deoxyribonucleotide residues.

In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

The term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

mRNA

According to the present disclosure, the term “mRNA” means “messenger-RNA” and relates to a “transcript” which may be generated by using a DNA template and may encode a peptide or polypeptide. Typically, an mRNA comprises a 5′-UTR, a peptide/polypeptide coding region, and a 3′-UTR. In the context of the present disclosure, mRNA may be generated by in vitro transcription (IVT) from a DNA template. As set forth above, the in vitro transcription methodology is known to the skilled person, and a variety of in vitro transcription kits is commercially available.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide or polypeptide.

In some embodiments, the mRNA which preferably encodes a peptide or polypeptide has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

As established in the art, mRNA generally contains a 5′ untranslated region (5′-UTR), a peptide/polypeptide coding region and a 3′ untranslated region (3′-UTR). In some embodiments, the mRNA is produced by in vitro transcription or chemical synthesis. In some embodiments, the mRNA is produced by in vitro transcription using a DNA template. The in vitro transcription methodology is known to the skilled person; cf., e.g., Molecular Cloning: A Laboratory Manual, 4^{th} Edition, M. R. Green and J. Sambrook eds., Cold Spring Harbor Laboratory Press, Cold Spring Harbor 2012. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified mRNA, correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, mRNA is in vitro transcribed mRNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the mRNA is “replicon mRNA” or simply a “replicon”, in particular “self-replicating mRNA” or “self-amplifying mRNA”. In certain embodiments, the replicon or self-replicating mRNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see Josd et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the mRNA contains one or more modifications, e.g., in order to increase its stability and/or increase translation efficiency and/or decrease immunogenicity and/or decrease cytotoxicity. For example, in order to increase expression of the mRNA, it may be modified within the coding region, i.e., the sequence encoding the expressed peptide or polypeptide, preferably without altering the sequence of the expressed peptide or polypeptide. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides; and codon optimization (e.g., to alter, preferably increase, the GC content of the RNA).

In some embodiments, the mRNA comprises a 5′-cap structure. In some embodiments, the mRNA does not have uncapped 5′-triphosphates. In some embodiments, the mRNA may comprise a conventional 5′-cap and/or a 5′-cap analog. The term “conventional 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′ triphosphate linkage to the rest of the mRNA). The guanosine may be methylated at position N⁷ (resulting in the cap structure m⁷Gppp). The term “5′-cap analog” includes a 5′-cap which is based on a conventional 5′-cap but which has been modified at either the 2′- or 3′-position of the m⁷guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2′O}G(5′)ppSp(5′)G (referred to as beta-S-ARCA or β-S-ARCA)), as described in PCT/EP2019/056502. Providing an mRNA with a 5′-cap structure as described herein may be achieved by in vitro transcription of a DNA template in presence of a corresponding 5′-cap compound, wherein said 5′-cap structure is co-transcriptionally incorporated into the generated mRNA strand, or the mRNA may be generated, for example, by in vitro transcription, and the 5′-cap structure may be attached to the mRNA post-transcriptionally using capping enzymes, for example, capping enzymes of vaccinia virus.

In some embodiments, the mRNA comprises a 5′-cap structure selected from the group consisting of m₂^{7,2′O}G(5′)ppSp(5′)G (in particular its D1 diastereomer), m₂^{7,3′O}G(5′)ppp(5′)G, and m2^{7,2′-O}Gppp(m₁^{2′-O})ApG.

In some embodiments, the mRNA comprises a cap0, cap1, or cap2, preferably capI or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′.

According to the present disclosure, the term “cap2” means the structure “m⁷GpppNmNm”, wherein each Nm is independently any nucleoside bearing an OCH₃ moiety at position 2′.

The D1 diastereomer of beta-S-ARCA (3-S-ARCA) has the following structure:

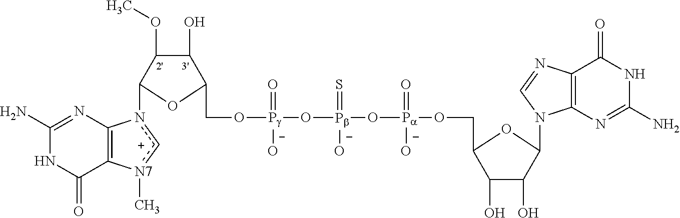

The “D1 diastereomer of beta-S-ARCA” or “beta-S-ARCA(D1)” is the diastereomer of beta-S-ARCA which elutes first on an HPLC column compared to the D2 diastereomer of beta-S-ARCA (beta-S-ARCA(D2)) and thus exhibits a shorter retention time. The HPLC preferably is an analytical HPLC. In some embodiments, a Supelcosil LC-18-T RP column, preferably of the format: 5 m, 4.6×250 mm is used for separation, whereby a flow rate of 1.3 ml/min can be applied. In some embodiments, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp(m₁₂′-O)ApG (also referred to as m2^{7,3′O}G(5′)ppp(5′)m^{2′-O}ApG) which is a building block of a cap1 has the following structure:

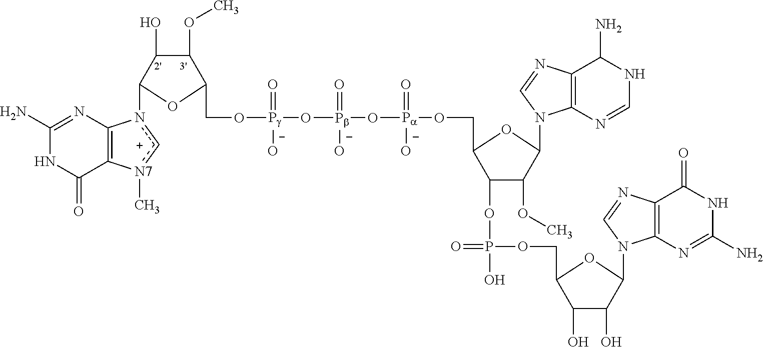

An exemplary cap0 mRNA comprising β-S-ARCA and mRNA has the following structure:

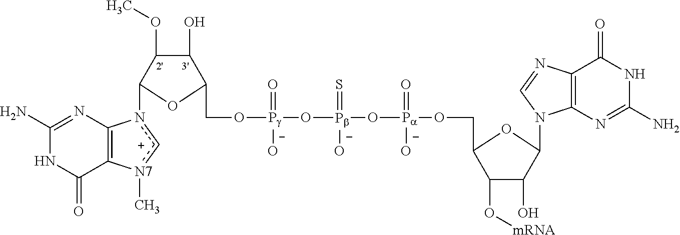

An exemplary cap0 mRNA comprising m₂^{7,2′O}G(5′)ppp(5′)G and mRNA has the following structure:

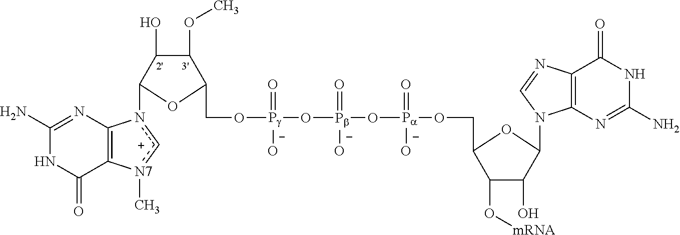

An exemplary cap1 mRNA comprising m₂^{7,2′O}Gppp(m₁^{2′-O})ApG and mRNA has the following structure:

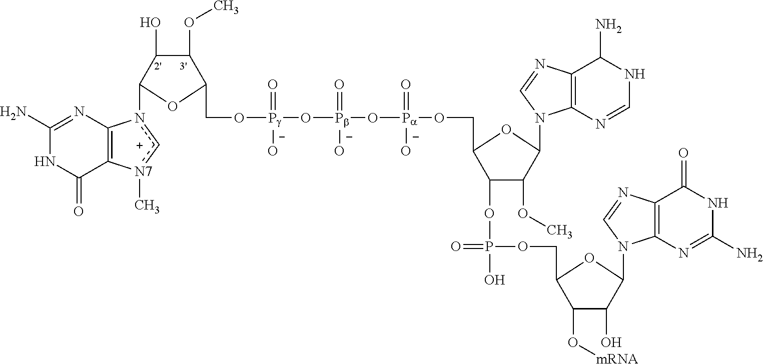

As used herein, the term “poly-A tail” or “poly-A sequence” refers to an uninterrupted or interrupted sequence of adenylate residues which is typically located at the 3′-end of an mRNA molecule. Poly-A tails or poly-A sequences are known to those of skill in the art and may follow the 3′-UTR in the mRNAs described herein. An uninterrupted poly-A tail is characterized by consecutive adenylate residues. In nature, an uninterrupted poly-A tail is typical. mRNAs disclosed herein can have a poly-A tail attached to the free 3′-end of the mRNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of mRNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an mRNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, mRNA used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does generally not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (e.g., mRNA) into which they are introduced. In certain embodiments, the 3′-UTR is derived from a globin gene or mRNA, such as a gene or mRNA of alpha2-globin, alpha1-globin, or beta-globin, e.g., beta-globin, e.g., human beta-globin. For example, the RNA (e.g., mRNA) may be modified by the replacement of the existing 3′-UTR with or the insertion of one or more, e.g., two copies of a 3′-UTR derived from a globin gene, such as alpha2-globin, alphal-globin, beta-globin, e.g., beta-globin, e.g., human beta-globin.

The mRNA may have modified ribonucleotides in order to increase its stability and/or decrease immunogenicity and/or decrease cytotoxicity. For example, in some embodiments, uridine in the mRNA described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (W), N1-methyl-pseudouridine (mly), 5-methyl-uridine (m5U), and combinations thereof.

In some embodiments, the modified nucleoside replacing (partially or completely, preferably completely) uridine in the mRNA may be any one or more of 3-methyl-uridine (m3U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (mcmo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (τm5U), 1-taurinomethyl-pseudouridine, 5-taurinomethyl-2-thio-uridine(τm5s2U), 1-taurinomethyl-4-thio-pseudouridine), 5-methyl-2-thio-uridine (m5s2U), 1-methyl-4-thio-pseudouridine (mls⁴ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp3U), 1-methyl-3-(3-amino-3-carboxypropyl)pseudouridine (acp3 W), 5-(isopentenylaminomethyl)uridine (imu5U), 5-(isopentenylaminomethyl)-2-thio-uridine (inm5s2U), α-thio-uridine, 2′-O-methyl-uridine (Um), 5,2′-O-dimethyl-uridine (m5Um), 2′-O-methyl-pseudouridine (Wm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (imu5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

An RNA (preferably mRNA) which is modified by pseudouridine (replacing partially or completely, preferably completely, uridine) is referred to herein as “Ψ-modified”, whereas the term “m1Ψ-modified” means that the RNA (preferably mRNA) contains N(1)-methylpseudouridine (replacing partially or completely, preferably completely, uridine). Furthermore, the term “m5U-modified” means that the RNA (preferably mRNA) contains 5-methyluridine (replacing partially or completely, preferably completely, uridine). Such Ψ- or m1Ψ- or m5U-modified RNAs usually exhibit decreased immunogenicity compared to their unmodified forms and, thus, are preferred in applications where the induction of an immune response is to be avoided or minimized.

The codons of the mRNA used in the present disclosure may further be optimized, e.g., to increase the GC content of the RNA and/or to replace codons which are rare in the cell (or subject) in which the peptide or polypeptide of interest is to be expressed by codons which are synonymous frequent codons in said cell (or subject). In some embodiments, the amino acid sequence encoded by the mRNA used in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present disclosure, coding regions may be codon-optimized for optimal expression in a subject to be treated using the mRNA described herein. Codon-optimization is based on the finding that the translation efficiency is also determined by a different frequency in the occurrence of tRNAs in cells. Thus, the sequence of mRNA may be modified such that codons for which frequently occurring tRNAs are available are inserted in place of “rare codons”.

In some embodiments, the guanosine/cytosine (G/C) content of the coding region of the mRNA described herein is increased compared to the G/C content of the corresponding coding sequence of the wild type RNA, wherein the amino acid sequence encoded by the mRNA is preferably not modified compared to the amino acid sequence encoded by the wild type RNA. This modification of the mRNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the mRNA, there are various possibilities for modification of the mRNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the mRNA described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (F) or N(1)-methylpseudouridine (m1Ψ) or 5-methyluridine (m5U) for uridine), and codon optimization, has a synergistic influence on the stability of RNA (preferably mRNA) and increase in translation efficiency. Thus, in some embodiments, the mRNA used in the present disclosure contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (T) or N(1)-methylpseudouridine (m1Ψ) or 5-methyluridine (m5U) for uridine), and (v) codon optimization.

Some aspects of the disclosure involve the targeted delivery of the mRNA disclosed herein to certain cells or tissues. In some embodiments, the disclosure involves targeting the lymphatic system, in particular secondary lymphoid organs, more specifically spleen. Targeting the lymphatic system, in particular secondary lymphoid organs, more specifically spleen is in particular preferred if the mRNA administered is mRNA encoding an antigen or epitope for inducing an immune response. In some embodiments, the target cell is a spleen cell. In some embodiments, the target cell is an antigen presenting cell such as a professional antigen presenting cell in the spleen. In some embodiments, the target cell is a dendritic cell in the spleen. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

Lipid-based mRNA delivery systems have an inherent preference to the liver. Liver accumulation is caused by the discontinuous nature of the hepatic vasculature or the lipid metabolism (liposomes and lipid or cholesterol conjugates). In some embodiments, the target organ is liver and the target tissue is liver tissue. The delivery to such target tissue is preferred, in particular, if presence of mRNA or of the encoded peptide or polypeptide in this organ or tissue is desired and/or if it is desired to express large amounts of the encoded peptide or polypeptide and/or if systemic presence of the encoded peptide or polypeptide, in particular in significant amounts, is desired or required.

In some embodiments, after administration of the mRNA particles described herein, at least a portion of the mRNA is delivered to a target cell or target organ. In some embodiments, at least a portion of the mRNA is delivered to the cytosol of the target cell. In some embodiments, the mRNA is mRNA encoding a peptide or polypeptide and the mRNA is translated by the target cell to produce the peptide or polypeptide. In some embodiments, the target cell is a cell in the liver. In some embodiments, the target cell is a muscle cell. In some embodiments, the target cell is an endothelial cell. In some embodiments the target cell is a tumor cell or a cell in the tumor microenvironment. In some embodiments, the target cell is a blood cell. In some embodiments, the target cell is a cell in the lymph nodes. In some embodiments, the target cell is a cell in the lung. In some embodiments, the target cell is a blood cell. In some embodiments, the target cell is a cell in the skin. In some embodiments, the target cell is a spleen cell. In some embodiments, the target cell is an antigen presenting cell such as a professional antigen presenting cell in the spleen. In some embodiments, the target cell is a dendritic cell in the spleen. In some embodiments, the target cell is a T cell. In some embodiments, the target cell is a B cell. In some embodiments, the target cell is a NK cell. In some embodiments, the target cell is a monocyte. Thus, RNA particles described herein may be used for delivering mRNA to such target cell. Accordingly, the present disclosure also relates to a method for delivering mRNA to a target cell in a subject comprising the administration of the mRNA particles described herein to the subject. In some embodiments, the mRNA is delivered to the cytosol of the target cell. In some embodiments, the mRNA is mRNA encoding a peptide or polypeptide and the RNA is translated by the target cell to produce the peptide or polypeptide.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

In some embodiments, mRNA used in the present disclosure comprises a nucleic acid sequence encoding one or more peptides or polypeptides, preferably a pharmaceutically active peptide or polypeptide.

In a preferred embodiment, mRNA used in the present disclosure comprises a nucleic acid sequence encoding a peptide or polypeptide, preferably a pharmaceutically active peptide or polypeptide, and is capable of expressing said peptide or polypeptide, in particular if transferred into a cell or subject. Thus, in some embodiments, the mRNA used in the present disclosure contains a coding region (open reading frame (ORF)) encoding a peptide or polypeptide, e.g., encoding a pharmaceutically active peptide or polypeptide. In this respect, an “open reading frame” or “ORF” is a continuous stretch of codons beginning with a start codon and ending with a stop codon. Such mRNA encoding a pharmaceutically active peptide or polypeptide is also referred to herein as “pharmaceutically active mRNA”.

According to the present disclosure, the term “pharmaceutically active peptide or polypeptide” means a peptide or polypeptide that can be used in the treatment of an individual where the expression of a peptide or polypeptide would be of benefit, e.g., in ameliorating the symptoms of a disease. Preferably, a pharmaceutically active peptide or polypeptide has curative or palliative properties and may be administered to ameliorate, relieve, alleviate, reverse, delay onset of or lessen the severity of one or more symptoms of a disease. In some embodiments, a pharmaceutically active peptide or polypeptide has a positive or advantageous effect on the condition or disease state of an individual when administered to the individual in a therapeutically effective amount. A pharmaceutically active peptide or polypeptide may have prophylactic properties and may be used to delay the onset of a disease or to lessen the severity of such disease. The term “pharmaceutically active peptide or polypeptide” includes entire peptides or polypeptides, and can also refer to pharmaceutically active fragments thereof. It can also include pharmaceutically active analogs of a peptide or polypeptide.

Specific examples of pharmaceutically active peptides and polypeptide include, but are not limited to, cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

The term “cytokines” relates to proteins which have a molecular weight of about 5 to 60 kDa and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. IFN-gamma is the sole type II interferon; all others are type I interferons. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFγ), interleukin 2 (IL-2), interleukin 4 (IL-4), interleukin 10 (IL-10), interleukin 11 (IL-11), interleukin 12 (IL-12), interleukin 15 (IL-15), and interleukin 21 (IL-21), as well as variants and derivatives thereof.

In some embodiments, a pharmaceutically active peptide or polypeptide comprises a replacement protein. In these embodiments, the present disclosure provides a method for treatment of a subject having a disorder requiring protein replacement (e.g., protein deficiency disorders) comprising administering to the subject RNA as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In some embodiments, a “hormone” is a peptide or polypeptide hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., P-selectin), and addressins.

Integrins are also involved in signal transduction. In particular, upon ligand binding, integrins modulate cell signaling pathways, e.g., pathways of transmembrane protein kinases such as receptor tyrosine kinases (RTK). Such regulation can lead to cellular growth, division, survival, or differentiation or to apoptosis. Particular examples of integrins include: α₁β₁, α₂β₁, α₃β₁, α₄β₁, α₅β₁, α₆β₁, α₇β₁, α_{L}β₂, α_{M}β₂, α_{IIb}β₃, α_{V}β₁, α_{V}β₃, α_{V}β₅, α_{V}β₆, α_{V}β₈, and α₆β₄.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-6, CD-3R, CD79a, CD79b), co-stimulatory or inhibitory molecules (e.g., CD28, CD80, CD86), and other.

The term “immunologically active compound” relates to any compound altering an immune response, e.g., by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants. Particular examples of immunologically active compounds include interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, and antigens, in particular tumor-associated antigens, pathogen-associated antigens (such as bacterial, parasitic, or viral antigens), allergens, and autoantigens. An immunologically active compound may be a vaccine antigen, i.e., an antigen whose inoculation into a subject induces an immune response.

The term “autoantigen” or “self-antigen” refers to an antigen which originates from within the body of a subject (i.e., the autoantigen can also be called “autologous antigen”) and which produces an abnormally vigorous immune response against this normal part of the body. Such vigorous immune reactions against autoantigens may be the cause of “autoimmune diseases”.

The term “allergen” refers to a kind of antigen which originates from outside the body of a subject (i.e., the allergen can also be called “heterologous antigen”) and which produces an abnormally vigorous immune response in which the immune system of the subject fights off a perceived threat that would otherwise be harmless to the subject. “Allergies” are the diseases caused by such vigorous immune reactions against allergens. An allergen usually is an antigen which is able to stimulate a type-I hypersensitivity reaction in atopic individuals through immunoglobulin E (IgE) responses. Particular examples of allergens include allergens derived from peanut proteins (e.g., Ara h 2.02), ovalbumin, grass pollen proteins (e.g., Phl p 5), and proteins of dust mites (e.g., Der p 2).

The term “growth factors” refers to molecules which are able to stimulate cellular growth, proliferation, healing, and/or cellular differentiation. Typically, growth factors act as signaling molecules between cells. The term “growth factors” include particular cytokines and hormones which bind to specific receptors on the surface of their target cells. Examples of growth factors include bone morphogenetic proteins (BMPs), fibroblast growth factors (FGFs), vascular endothelial growth factors (VEGFs), such as VEGFA, epidermal growth factor (EGF), insulin-like growth factor, ephrins, macrophage colony-stimulating factor, granulocyte colony-stimulating factor, granulocyte macrophage colony-stimulating factor, neuregulins, neurotrophins (e.g., brain-derived neurotrophic factor (BDNF), nerve growth factor (NGF)), placental growth factor (PGF), platelet-derived growth factor (PDGF), renalase (RNLS) (anti-apoptotic survival factor), T-cell growth factor (TCGF), thrombopoietin (TPO), transforming growth factors (transforming growth factor alpha (TGF-α), transforming growth factor beta (TGF-β)), and tumor necrosis factor-alpha (TNF-α). In some embodiments, a “growth factor” is a peptide or polypeptide growth factor.

The term “protease inhibitors” refers to molecules, in particular peptides or polypeptides, which inhibit the function of proteases. Protease inhibitors can be classified by the protease which is inhibited (e.g., aspartic protease inhibitors) or by their mechanism of action (e.g., suicide inhibitors, such as serpins). Particular examples of protease inhibitors include serpins, such as alpha 1-antitrypsin, aprotinin, and bestatin.

The term “enzymes” refers to macromolecular biological catalysts which accelerate chemical reactions. Like any catalyst, enzymes are not consumed in the reaction they catalyze and do not alter the equilibrium of said reaction. Unlike many other catalysts, enzymes are much more specific. In some embodiments, an enzyme is essential for homeostasis of a subject, e.g., any malfunction (in particular, decreased activity which may be caused by any of mutation, deletion or decreased production) of the enzyme results in a disease. Examples of enzymes include herpes simplex virus type 1 thymidine kinase (HSV1-TK), hexosaminidase, phenylalanine hydroxylase, pseudocholinesterase, and lactase.

The term “receptors” refers to protein molecules which receive signals (in particular chemical signals called ligands) from outside a cell. The binding of a signal (e.g., ligand) to a receptor causes some kind of response of the cell, e.g., the intracellular activation of a kinase. Receptors include transmembrane receptors (such as ion channel-linked (ionotropic) receptors, G protein-linked (metabotropic) receptors, and enzyme-linked receptors) and intracellular receptors (such as cytoplasmic receptors and nuclear receptors). Particular examples of receptors include steroid hormone receptors, growth factor receptors, and peptide receptors (i.e., receptors whose ligands are peptides), such as P-selectin glycoprotein ligand-1 (PSGL-1). The term “growth factor receptors” refers to receptors which bind to growth factors.

The term “apoptosis regulators” refers to molecules, in particular peptides or polypeptides, which modulate apoptosis, i.e., which either activate or inhibit apoptosis. Apoptosis regulators can be grouped into two broad classes: those which modulate mitochondrial function and those which regulate caspases. The first class includes proteins (e.g., BCL-2, BCL-xL) which act to preserve mitochondrial integrity by preventing loss of mitochondrial membrane potential and/or release of pro-apoptotic proteins such as cytochrome C into the cytosol. Also to this first class belong proapoptotic proteins (e.g., BAX, BAK, BIM) which promote release of cytochrome C. The second class includes proteins such as the inhibitors of apoptosis proteins (e.g., XIAP) or FLIP which block the activation of caspases.

The term “transcription factors” relates to proteins which regulate the rate of transcription of genetic information from DNA to messenger RNA, in particular by binding to a specific DNA sequence. Transcription factors may regulate cell division, cell growth, and cell death throughout life; cell migration and organization during embryonic development; and/or in response to signals from outside the cell, such as a hormone. Transcription factors contain at least one DNA-binding domain which binds to a specific DNA sequence, usually adjacent to the genes which are regulated by the transcription factors. Particular examples of transcription factors include MECP2, FOXP2, FOXP3, the STAT protein family, and the HOX protein family.

The term “tumor suppressor proteins” relates to molecules, in particular peptides or polypeptides, which protect a cell from one step on the path to cancer. Tumor-suppressor proteins (usually encoded by corresponding tumor-suppressor genes) exhibit a weakening or repressive effect on the regulation of the cell cycle and/or promote apoptosis. Their functions may be one or more of the following: repression of genes essential for the continuing of the cell cycle; coupling the cell cycle to DNA damage (as long as damaged DNA is present in a cell, no cell division should take place); initiation of apoptosis, if the damaged DNA cannot be repaired; metastasis suppression (e.g., preventing tumor cells from dispersing, blocking loss of contact inhibition, and inhibiting metastasis); and DNA repair. Particular examples of tumor-suppressor proteins include p53, phosphatase and tensin homolog (PTEN), SWI/SNF (SWItch/Sucrose Non-Fermentable), von Hippel-Lindau tumor suppressor (pVHL), adenomatous polyposis coli (APC), CD95, suppression of tumorigenicity 5 (ST5), suppression of tumorigenicity 5 (ST5), suppression of tumorigenicity 14 (ST14), and Yippee-like 3 (YPEL3).

The term “structural proteins” refers to proteins which confer stiffness and rigidity to otherwise-fluid biological components. Structural proteins are mostly fibrous (such as collagen and elastin) but may also be globular (such as actin and tubulin). Usually, globular proteins are soluble as monomers, but polymerize to form long, fibers which, for example, may make up the cytoskeleton. Other structural proteins are motor proteins (such as myosin, kinesin, and dynein) which are capable of generating mechanical forces, and surfactant proteins. Particular examples of structural proteins include collagen, surfactant protein A, surfactant protein B, surfactant protein C, surfactant protein D, elastin, tubulin, actin, and myosin.

The term “reprogramming factors” or “reprogramming transcription factors” relates to molecules, in particular peptides or polypeptides, which, when expressed in somatic cells optionally together with further agents such as further reprogramming factors, lead to reprogramming or de-differentiation of said somatic cells to cells having stem cell characteristics, in particular pluripotency. Particular examples of reprogramming factors include OCT4, SOX2, c-MYC, KLF4, LIN28, and NANOG.

The term “genomic engineering proteins” relates to proteins which are able to insert, delete or replace DNA in the genome of a subject. Particular examples of genomic engineering proteins include meganucleases, zinc finger nucleases (ZFNs), transcription activator-like effector nucleases (TALENs), and clustered regularly spaced short palindromic repeat-CRISPR-associated protein 9 (CRISPR-Cas9).

The term “blood proteins” relates to peptides or polypeptides which are present in blood plasma of a subject, in particular blood plasma of a healthy subject. Blood proteins have diverse functions such as transport (e.g., albumin, transferrin), enzymatic activity (e.g., thrombin or ceruloplasmin), blood clotting (e.g., fibrinogen), defense against pathogens (e.g., complement components and immunoglobulins), protease inhibitors (e.g., alpha 1-antitrypsin), etc. Particular examples of blood proteins include thrombin, serum albumin, Factor VII, Factor VIII, insulin, Factor IX, Factor X, tissue plasminogen activator, protein C, von Willebrand factor, antithrombin III, glucocerebrosidase, erythropoietin, granulocyte colony stimulating factor (G-CSF), modified Factor VIII, and anticoagulants.

Thus, in some embodiments, the pharmaceutically active peptide or polypeptide is (i) a cytokine, preferably selected from the group consisting of erythropoietin (EPO), interleukin 4 (IL-2), and interleukin 10 (IL-11), more preferably EPO; (ii) an adhesion molecule, in particular an integrin; (iii) an immunoglobulin, in particular an antibody; (iv) an immunologically active compound, in particular an antigen; (v) a hormone, in particular vasopressin, insulin or growth hormone; (vi) a growth factor, in particular VEGFA; (vii) a protease inhibitor, in particular alpha 1-antitrypsin; (viii) an enzyme, preferably selected from the group consisting of herpes simplex virus type 1 thymidine kinase (HSV1-TK), hexosaminidase, phenylalanine hydroxylase, pseudocholinesterase, pancreatic enzymes, and lactase; (ix) a receptor, in particular growth factor receptors; (x) an apoptosis regulator, in particular BAX; (xi) a transcription factor, in particular FOXP3; (xii) a tumor suppressor protein, in particular p53; (xiii) a structural protein, in particular surfactant protein B; (xiv) a reprogramming factor, e.g., selected from the group consisting of OCT4, SOX2, c-MYC, KLF4, LIN28 and NANOG; (xv) a genomic engineering protein, in particular clustered regularly spaced short palindromic repeat-CRISPR-associated protein 9 (CRISPR-Cas9); and (xvi) a blood protein, in particular fibrinogen.

In some embodiments, a pharmaceutically active peptide or polypeptide comprises one or more antigens or one or more epitopes, i.e., administration of the peptide or polypeptide to a subject elicits an immune response against the one or more antigens or one or more epitopes in a subject which may be therapeutic or partially or fully protective.

In some embodiments, the mRNA encodes at least one epitope.

In some embodiments, the epitope is derived from a tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUD 1N-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by mRNA described herein, e.g., as a single polypeptide wherein the epitopes are optionally separated by linkers. In some embodiments of the present disclosure, the mRNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include mRNA that encodes at least five epitopes (termed a “pentatope”) and mRNA that encodes at least ten epitopes (termed a “decatope”).

In some embodiments, the epitope is derived from a pathogen-associated antigen, in particular from a viral antigen. In some embodiments, the epitope is derived from a SARS-CoV-2 S protein, an immunogenic variant thereof, or an immunogenic fragment of the SARS-CoV-2 S protein or the immunogenic variant thereof. Thus, in some embodiments, the mRNA used in the present disclosure encodes an amino acid sequence comprising a SARS-CoV-2 S protein, an immunogenic variant thereof, or an immunogenic fragment of the SARS-CoV-2 S protein or the immunogenic variant thereof.

In some embodiments of the present disclosure the antigen (such as a tumor antigen or vaccine antigen) is preferably administered as single-stranded, 5′ capped mRNA that is translated into the respective protein upon entering cells of a subject being administered the RNA. Preferably, the RNA contains structural elements optimized for maximal efficacy of the RNA with respect to stability and translational efficiency (5′ cap, 5′ UTR, 3′ UTR, poly(A) sequence).

In some embodiments, beta-S-ARCA(D1) is utilized as specific capping structure at the 5′-end of the mRNA. In some embodiments, m₂^{7,3′-O}Gppp(m₁^{2′-O}) ApG is utilized as specific capping structure at the 5′-end of the mRNA. In some embodiments, the 5′-UTR sequence is derived from the human alpha-globin mRNA and optionally has an optimized ‘Kozak sequence’ to increase translational efficiency. In some embodiments, a combination of two sequence elements (FI element) derived from the “amino terminal enhancer of split” (AES) mRNA (called F) and the mitochondrial encoded 12S ribosomal RNA (called I) are placed between the coding sequence and the poly(A) sequence to assure higher maximum protein levels and prolonged persistence of the mRNA. In some embodiments, two re-iterated 3′-UTRs derived from the human beta-globin mRNA are placed between the coding sequence and the poly(A) sequence to assure higher maximum protein levels and prolonged persistence of the mRNA. In some embodiments, a poly(A) sequence measuring 110 nucleotides in length, consisting of a stretch of 30 adenosine residues, followed by a 10 nucleotide linker sequence and another 70 adenosine residues is used. This poly(A) sequence was designed to enhance RNA stability and translational efficiency.

In some embodiments, mRNA encoding an antigen (such as a tumor antigen or a vaccine antigen) is expressed in cells of the subject treated to provide the antigen. In some embodiments, the mRNA is transiently expressed in cells of the subject. In some embodiments, the mRNA is in vitro transcribed. In some embodiments, expression of the antigen is at the cell surface. In some embodiments, the antigen is expressed and presented in the context of MHC. In some embodiments, expression of the antigen is into the extracellular space, i.e., the antigen is secreted.

The antigen molecule or a procession product thereof, e.g., a fragment thereof, may bind to an antigen receptor such as a BCR or TCR carried by immune effector cells, or to antibodies.

A peptide and polypeptide antigen which is provided to a subject according to the present disclosure by administering mRNA encoding a peptide and polypeptide antigen, wherein the antigen is a vaccine antigen, preferably results in the induction of an immune response, e.g., a humoral and/or cellular immune response in the subject being provided the peptide or polypeptide antigen. Said immune response is preferably directed against a target antigen. Thus, a vaccine antigen may comprise the target antigen, a variant thereof, or a fragment thereof. In some embodiments, such fragment or variant is immunologically equivalent to the target antigen. In the context of the present disclosure, the term “fragment of an antigen” or “variant of an antigen” means an agent which results in the induction of an immune response which immune response targets the antigen, i.e. a target antigen. Thus, the vaccine antigen may correspond to or may comprise the target antigen, may correspond to or may comprise a fragment of the target antigen or may correspond to or may comprise an antigen which is homologous to the target antigen or a fragment thereof. Thus, according to the present disclosure, a vaccine antigen may comprise an immunogenic fragment of a target antigen or an amino acid sequence being homologous to an immunogenic fragment of a target antigen. An “immunogenic fragment of an antigen” according to the disclosure preferably relates to a fragment of an antigen which is capable of inducing an immune response against the target antigen. The vaccine antigen may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence.

In some embodiments, the mRNA used in the present disclosure is non-immunogenic. RNA encoding an immunostimulant may be administered according to the present disclosure to provide an adjuvant effect. The RNA encoding an immunostimulant may be standard RNA or non-immunogenic RNA.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA, which is also termed modified RNA (modRNA) herein, is rendered non-immunogenic by incorporating modified nucleosides suppressing RNA-mediated activation of innate immune receptors into the RNA and removing double-stranded RNA (dsRNA).

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmoSU), uridine 5-oxyacetic acid methyl ester (mcmoSU), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)uridine. In certain embodiments, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the removal of dsRNA (especially mRNA) from non-immunogenic RNA comprises a removal of dsRNA such that less than 10%, less than 5%, less than 4%, less than 3%, less than 2%, less than 1%, less than 0.5%, less than 0.3%, or less than 0.1% of the RNA in the non-immunogenic RNA composition is dsRNA. In some embodiments, the non-immunogenic RNA (especially mRNA) is free or essentially free of dsRNA. In some embodiments, the non-immunogenic RNA (especially mRNA) composition comprises a purified preparation of single-stranded nucleoside modified RNA. For example, in some embodiments, the purified preparation of single-stranded nucleoside modified RNA (especially mRNA) is substantially free of double stranded RNA (dsRNA). In some embodiments, the purified preparation is at least 90%, at least 91%, at least 92%, at least 93%, at least 94%, at least 95%, at least 96%, at least 97%, at least 98%, at least 99%, at least 99.5%, or at least 99.9% single stranded nucleoside modified RNA, relative to all other nucleic acid molecules (DNA, dsRNA, etc.).

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked”, “fused”, or “fusion” are used interchangeably. These terms refer to the joining together of two or more elements or components or domains.

### Particles

Provided herein are RNA particles comprising (i) RNA, (ii) at least one cationic or cationically ionizable lipid, and (iii) at least one phosphatidylserine. In some embodiments, RNA particles provided herein comprise (i) RNA, (ii) at least one cationic or cationically ionizable lipid, (iii) a first phospholipid which is a phosphatidylserine, and (iv) a second phospholipid. In some embodiments, the RNA particles further comprise one or more additional lipids. In some embodiments, the RNA particles further comprise a non-ionic amphiphilic organic compound, e.g., a surfactant. In some embodiments, the level of non-ionic amphiphilic organic compound (such as a polysorbate) present in a RNA particle (or composition comprising an RNA particle) is at least about 5 mol % of the total lipid present in the particle or composition, or at least about 0.15 mM. In some embodiments, the level of non-ionic amphiphilic organic compound (such as a polysorbate) present in a RNA particle (or composition comprising an RNA particle) is at least about 5 mol % of the total lipid present in the particle or composition, or at least about 0.15 mM.

Different types of RNA containing particles have been described previously to be suitable for delivery of RNA in particulate form (cf., e.g., Kaczmarek, J. C. et al., 2017, Genome Medicine 9, 60). For non-viral RNA delivery vehicles, nanoparticle encapsulation of RNA physically protects RNA from degradation and, depending on the specific chemistry, can aid in cellular uptake and endosomal escape.

Electrostatic interactions between positively charged molecules such as polymers and lipids and negatively charged nucleic acid are involved in particle formation. This results in complexation and spontaneous formation of nucleic acid particles.

In some embodiments, components and/or ratios of components in RNA particles result in increased expression of RNA contained within the particles upon, e.g., administration into a subject such as a mammal compared to RNA particles with different components and/or ratios of components. In some embodiments, components and/or ratios of components in RNA particles result in reduced off-target expression of RNA contained within the particles upon, e.g., local administration into a mammalian subject (e.g., intratumoral or peritumoral injection) compared to RNA particles with different components and/or ratios of components. In some embodiments, RNA particles and/or RNA particle-containing compositions provided herein comprise a non-ionic amphiphilic organic compound, wherein the non-ionic amphiphilic organic compound is present in an amount that is effective to reduce off-target expression of the RNA after local administration into a mammalian subject (e.g., intratumoral or peritumoral injection). In some embodiments, the off-target expression is expression of the RNA in an organ into which no RNA particles have been injected. In some embodiments, off-target expression is expression in the liver. In some embodiments, the subject is a human.

In the context of the present disclosure, the term “particle” relates to a structured entity formed by molecules or molecule complexes, in particular particle forming compounds. Preferably, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” includes lipoplex particles (LPXs), and lipid nanoparticles (LNPs). According to the present disclosure, the term “particle” includes nanoparticles.

An “RNA particle” can be used to deliver RNA to a target site of interest (e.g., cell, tissue, organ, and the like). An RNA particle may be formed from lipids comprising at least one cationic or cationically ionizable lipid or lipid-like material. Without intending to be bound by any theory, it is believed that the cationic or cationically ionizable lipid or lipid-like material combines together with the RNA to form aggregates, and this aggregation results in colloidally stable particles. RNA particles described herein include lipid nanoparticle (LNP)-based and lipoplex (LPX)-based formulations.

In general, a lipoplex (LPX) is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that lipids in the mixture as required are able to form lamellar (bilayer) phases in the applied aqueous environment.

In some embodiments, liposomes are spherical vesicles comprising unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of nucleic acids are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with RNAs. In some embodiments, formed lipoplexes possess distinct internal arrangements of molecules that arise due to the transformation from liposomal structure into compact RNA-lipoplexes. In some embodiments, these formulations are characterized by their poor encapsulation of the RNA and incomplete entrapment of the RNA.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged nucleic acid (especially mRNA) results in complexation and spontaneous formation of nucleic acid lipoplex particles. Positively charged liposomes may be generally synthesized using a cationic or cationically ionizable amphiphilic lipid, such as DODMA, and additional lipids, such as DOPE. In some embodiments, an RNA (especially mRNA) lipoplex particle is a nanoparticle.

In general, a lipid nanoparticle (LNP) is obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

In some embodiments, LNPs comprise or consist of a cationic/ionizable lipid and helper lipids such as phospholipids, cholesterol, and/or polyethylene glycol (PEG) lipids. In some embodiments, in the RNA LNPs described herein the mRNA is bound by ionizable lipid that occupies the central core of the LNP.

In some embodiments, PEG lipid forms the surface of the LNP, along with phospholipids. In some embodiments, the surface comprises a bilayer. In some embodiments, cholesterol and ionizable lipid in charged and uncharged forms can be distributed throughout the LNP.

In some embodiments, RNA (e.g., mRNA) may be noncovalently associated with a particle as described herein. In some embodiments, the RNA (especially mRNA) may be adhered to the outer surface of the particle (surface RNA (especially surface mRNA)) and/or may be contained in the particle (encapsulated RNA (especially encapsulated mRNA)).

In some embodiments, the particles described herein have a size (such as a diameter) in the range of about 10 to about 2000 n, such as at least about 15 nm (e.g., at least about 20 n, at least about 25 nm, at least about 30 nm, at least about 35 n, at least about 40 nm, at least about 45 n, at least about 50 nm, at least about 55 n, at least about 60 n, at least about 65 n, at least about 70 n, at least about 75 n, at least about 80 nm, at least about 85 nm, at least about 90 n, at least about 95 nm, or at least about 100 nm) and/or at most 1900 nm (e.g., at most about 1900 nm, at most about 1800 nm, at most about 1700 nm, at most about 1600 n, at most about 1500 nm, at most about 1400 nm, at most about 1300 nm, at most about 1200 n, at most about 1100 nm, at most about 1000 nm, at most about 950 n, at most about 900 n, at most about 850 nm, at most about 800 nm, at most about 750 nm, at most about 700 nm, at most about 650 nm, at most about 600 nm, at most about 550 n, or at most about 500 n), such as in the range of about 20 to about 1500 n, such as about 30 to about 1200 nm, about 40 to about 1100 nm, about 50 to about 1000 nm, about 60 to about 900 n, about 70 to 800 nm, about 80 to 700 nm, about 90 to 600 nm, or about 50 to 500 nm or about 100 to 500 nm, such as in the range of 10 to 1000 nm, 15 to 500 n, 20 to 450 n, 25 to 400 nm, 30 to 350 nm, 40 to 300 nm, 50 to 250 nm, 60 to 200 nm, or 70 to 150 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 n, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 n, from about 100 nm to about 200 nm, from about 150 nm to about 1000 n, from about 150 nm to about 800 nm, from about 150 nm to about 700 n, from about 150 nm to about 600 n, from about 150 nm to about 500 n, from about 150 nm to about 450 nm, from about 150 nm to about 400 nm, from about 150 nm to about 350 nm, from about 150 nm to about 300 n, from about 150 nm to about 250 nm, from about 150 nm to about 200 nm, from about 200 nm to about 1000 n, from about 200 nm to about 800 n, from about 200 nm to about 700 nm, from about 200 nm to about 600 nm, from about 200 nm to about 500 nm, from about 200 nm to about 450 n, from about 200 nm to about 400 nm, from about 200 nm to about 350 n, from about 200 nm to about 300 n, or from about 200 nm to about 250 n.

In some embodiments, the RNA particles described herein are nanoparticles. The term “nanoparticle” relates to a nano-sized particle comprising RNA (especially mRNA) as described herein and at least one cationic or cationically ionizable lipid, wherein all three external dimensions of the particle are in the nanoscale, i.e., at least about 1 nm and below about 1000 nm. Preferably, the size of a particle is its diameter.

RNA particles described herein (especially mRNA particles) may exhibit a polydispersity index (PDI) less than about 0.5, less than about 0.4, less than about 0.3, less than about 0.2, less than about 0.1, or less than about 0.05. By way of example, the RNA particles can exhibit a polydispersity index in a range of about 0.01 to about 0.4 or about 0.1 to about 0.3.

With respect to RNA lipid particles (especially mRNA particles), the N/P ratio gives the ratio of the nitrogen groups in the lipid to the number of phosphate groups in the RNA. It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, RNA is considered to be completely bound to nanoparticles.

RNA particles (especially mRNA particles) described herein can be prepared using a wide range of methods that may involve obtaining a colloid from at least one cationic or cationically ionizable lipid and mixing the colloid with RNA to obtain RNA particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA (especially mRNA) lipoplex particles described herein are obtainable by adding RNA (especially mRNA) to a colloidal liposome dispersion. Using the ethanol injection technique, such colloidal liposome dispersion is, in some embodiments, formed as follows: an ethanol solution comprising lipids, such as cationic or cationically ionizable lipids like DODMA and additional lipids, is injected into an aqueous solution under stirring. In some embodiments, the RNA (especially mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some embodiments, LNPs comprise four components: ionizable cationic lipids, neutral lipids such as phospholipids, a steroid such as cholesterol, and a polymer conjugated lipid. In some embodiments, LNPs may be prepared by mixing lipids dissolved in ethanol rapidly with RNA in an aqueous buffer. While RNA particles described herein may comprise polymer conjugated lipids such as PEG lipids, provided herein are also RNA particles which do not comprise polymer conjugated lipids such as PEG lipids.

In some embodiments, the LNPs comprising RNA and at least one cationic or cationically ionizable lipid described herein are prepared by (a) preparing an RNA solution containing water and a buffering system; (b) preparing an ethanolic solution comprising the cationic or cationically ionizable lipid and, if present, one or more additional lipids; and (c) mixing the RNA solution prepared under (a) with the ethanolic solution prepared under (b), thereby preparing the formulation comprising LNPs. After step (c) one or more steps selected from diluting and filtrating, such as tangential flow filtrating, can follow.

In some embodiments, the LNPs comprising RNA and at least one cationic or cationically ionizable lipid described herein are prepared by (a′) preparing liposomes or a colloidal preparation of the cationic or cationically ionizable lipid and, if present, one or more additional lipids in an aqueous phase; and (b′) preparing an RNA solution containing water and a buffering system; and (c′) mixing the liposomes or colloidal preparation prepared under (a′) with the RNA solution prepared under (b′). After step (c′) one or more steps selected from diluting and filtrating, such as tangential flow filtrating, can follow.

The present disclosure describes particles comprising RNA (especially mRNA) and at least one cationic or cationically ionizable lipid which associates with the RNA to form RNA particles and compositions comprising such particles. The RNA particles may comprise RNA which is complexed in different forms by non-covalent interactions to the particle. The particles described herein are not viral particles, in particular infectious viral particles, i.e., they are not able to virally infect cells.

Suitable cationic or cationically ionizable lipids are those that form RNA particles and are included by the term “particle forming components” or “particle forming agents”. The term “particle forming components” or “particle forming agents” relates to any components which associate with RNA to form RNA particles. Such components include any component which can be part of RNA particles.

In some embodiments, RNA particles (especially mRNA particles) comprise more than one type of RNA molecules, where the molecular parameters of the RNA molecules may be similar or different from each other, like with respect to molar mass or fundamental structural elements such as molecular architecture, capping, coding regions or other features,

In particulate formulation, it is possible that each RNA species is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, followed by a step of mixing of the individual particulate formulations. By the step of mixing, a formulation comprising a mixed population of RNA-containing particles is obtainable. Individual particulate populations may be together in one container, comprising a mixed population of individual particulate formulations. Alternatively, it is possible that all RNA species of the pharmaceutical composition are formulated together as a combined particulate formulation. Such formulations are obtainable by providing a combined formulation (typically combined solution) of all RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

### Cationically Ionizable Lipids

The RNA particles described herein comprise at least one cationic or cationically ionizable lipid as particle forming agent. Cationic or cationically ionizable lipids contemplated for use herein include any cationic or cationically ionizable lipids (including lipid-like materials) which are able to electrostatically bind nucleic acid. In some embodiments, cationic or cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In some embodiments, a cationic lipid has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., which is not permanently cationic. Thus, depending on the pH of the composition in which the cationically ionizable lipid is solved, the cationically ionizable lipid is either positively charged or neutral. For purposes of the present disclosure, cationically ionizable lipids are covered by the term “cationic lipid” unless contradicted by the circumstances.

In some embodiments, the cationic or cationically ionizable lipid comprises a head group which includes at least one nitrogen atom (N) which is positive charged or capable of being protonated, e.g., under physiological conditions.

Examples of cationic or cationically ionizable lipids include, but are not limited to N,N-dimethyl-2,3-dioleyloxypropylamine (DODMA), 1,2-dioleoyl-3-trimethylammonium propane (DOTAP); 1,2-di-O-octadecenyl-3-trimethylammonium propane (DOTMA), 3-(N-(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (PAE-DMRIE), N-(4-carboxybenzyl)-N,N-dimethyl-2,3-bis(oleoyloxy)propan-1-aminium (DOBAQ), 2-({8-[(3f)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N₁₂-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino)butanoate (DLin-MC3-DMA), and 4-((di((9Z,12Z)-octadeca-9,12-dien-1-yl)amino)oxy)-N,N-dimethyl-4-oxobutan-1-amine (DPL-14). In some embodiments, the cationically ionizable lipid is DODMA.

Further examples of cationically ionizable lipids include, but are not limited to, 3-(N-(N′,N′-dimethylaminoethane)-carbamoyl)cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-l9-yl-4-(dimethylamino)butanoate (DLin-MC3-DMA), 2-({8-[(33)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), di((Z)-non-2-en-1-yl)-9-((4-(dimethylaminobutanoyl)oxy)heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

DODMA is an ionizable cationic lipid with a tertiary amine headgroup. The structure of DODMA may be represented as follows:

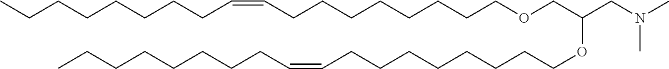 [SMILES: CCCCCCCC/C=C\CCCCCCCCOCC(CN(C)C)OCCCCCCCC/C=C\CCCCCCCC]

In some embodiments, the cationic or cationically ionizable lipid may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle. In some embodiments, the cationic or cationically ionizable lipid comprises about 60 mol % of the total lipid present in the particle.

### Additional Lipids

Particles described herein may also comprise lipids (including lipid-like materials) other than cationic or cationically ionizable lipids (also collectively referred to herein as cationic lipids), i.e., non-cationic lipids (including non-cationic or non-cationically ionizable lipids or lipid-like materials). Collectively, anionic and neutral lipids or lipid-like materials are referred to herein as non-cationic lipids. Optimizing the formulation of RNA particles by addition of other hydrophobic moieties, such as cholesterol and lipids, in addition to a cationic or cationically ionizable lipid may enhance particle stability and efficacy of RNA delivery.

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes alkyl, alkenyl, or alkynyl as defined herein. It should be appreciated that one or more of the hydrogen in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have six to thirty, typically six to twenty, often six to eighteen carbon atoms. Exemplary nonpolar alkyl groups include, but are not limited to, hexyl, decyl, dodecyl, tetradecyl, hexadecyl, octadecyl, and the like.

The term “alkenyl” refers to a linear or branched monovalent hydrocarbon moiety having at least one carbon carbon double bond in which the total carbon atoms may be six to thirty, typically six to twenty often six to eighteen.

The term “alkynyl” refers to a linear or branched monovalent hydrocarbon moiety having at least one carbon carbon triple bond in which the total carbon atoms may be six to thirty, typically six to twenty, often six to eighteen. Alkynyl groups can optionally have one or more carbon carbon double bonds.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids are generally cylindrical. The area occupied by the two alkyl chains is similar to the area occupied by the polar head group. Lipids have low solubility as monomers and tend to aggregate into planar bilayers that are water insoluble. Traditional surfactant monomers are generally cone shaped. The hydrophilic head groups tend to occupy more molecular space than the linear alkyl chains. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

In some embodiments, a particle or composition provided herein comprises a non-ionic amphiphilic organic compound. In some embodiments, the level of non-ionic amphiphilic organic compound present in a RNA particle (or composition comprising an RNA particle) is at least about 5 mol % of the total lipid present in the particle or composition, or at least about 0.15 mM. In some embodiments, the level of non-ionic amphiphilic organic compound present in a RNA particle (or composition comprising an RNA particle) is at least about 20 mol % of the total lipid present in the particle or composition, or at least about 0.6 mM.

Specific examples of amphiphilic compounds that may be included in an amphiphilic layer include, but are not limited to, phospholipids, aminolipids and sphingolipids.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

One or more additional lipids may be incorporated into particles described herein which may or may not affect the overall charge of the RNA particles. In some embodiments, the or more additional lipids are a non-cationic lipid or lipid-like material. The non-cationic lipid may comprise, e.g., one or more anionic lipids and/or neutral lipids. As used herein, an “anionic lipid” refers to any lipid that is negatively charged at a selected pH. As used herein, a “neutral lipid” refers to any of a number of lipid species that exist either in an uncharged or neutral zwitterionic form at a selected pH.

In some embodiments, the RNA particles (especially the particles comprising mRNA) described herein comprise a cationic or cationically ionizable lipid and one or more additional lipids.

Without wishing to be bound by theory, the amount of the cationic or cationically ionizable lipid compared to the amount of the one or more additional lipids may affect important RNA particle characteristics, such as charge, particle size, stability, tissue selectivity, and bioactivity of the RNA.

Accordingly, in some embodiments, the molar ratio of the cationic or cationically ionizable lipid to the one or more additional lipids is from about 10:0 to about 1:9, about 4:1 to about 1:2, about 4:1 to about 1:1, about 3:1 to about 1:1, or about 3:1 to about 2:1.

In some embodiments, the one or more additional lipids comprised in the RNA particles (especially in the particles comprising mRNA) described herein comprise one or more of the following: neutral lipids, steroids, and combinations thereof.

In some embodiments, the one or more additional lipids comprise a neutral lipid which is a phospholipid. In some embodiments, the phospholipid is selected from the group consisting of phosphatidylcholines, phosphatidylethanolamines, phosphatidylglycerols, phosphatidic acids, phosphatidylserines and sphingomyelins. Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidylethanolamine lipids with different hydrophobic chains. In some embodiments, the neutral lipid is selected from the group consisting of DSPC, DOPC, DMPC, DPPC, POPC, DOPE, DOPG, DPPG, POPE, DPPE, DMPE, DSPE, and SM. In some embodiments, the neutral lipid is selected from the group consisting of DSPC, DPPC, DMPC, DOPC, POPC, DOPE and SM. In some embodiments, the neutral lipid is DOPE.

In some embodiments, the additional lipid comprises one of the following: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Thus, in some embodiments, the RNA particles (especially the particles comprising mRNA) described herein comprise (1) a cationic or cationically ionizable lipid, and a phospholipid such as DOPE or (2) a cationic or cationically ionizable lipid and a phospholipid such as DOPE and cholesterol.

In some embodiments, the RNA particles (especially the particles comprising mRNA) described herein comprise (1) DODMA and DOPE or (2) DODMA, DOPE and cholesterol.

DOPE is a neutral phospholipid. The structure of DOPE may be represented as follows:

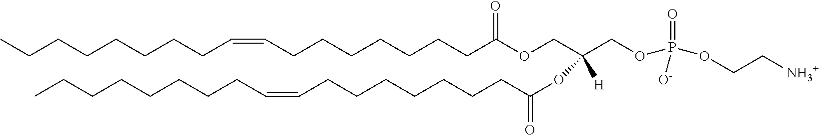 [SMILES: CCCCCCCC/C=C\CCCCCCCC(=O)OC[C@H](COP(=O)([O-])OCC[NH3+])OC(=O)CCCCCCC/C=C\CCCCCCCC]

The structure of cholesterol may be represented as follows:

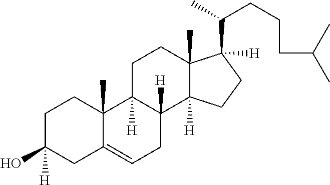

In some embodiments, particles described herein do not include a polymer conjugated lipid such as a pegylated lipid. The term “pegylated lipid” refers to a molecule comprising both a lipid portion and a polyethylene glycol portion. Pegylated lipids are known in the art.

In some embodiments, the additional lipid (e.g., one or more phospholipids and/or cholesterol) may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle. In some embodiments, the additional lipid (e.g., one or more phospholipids and/or cholesterol) comprises about 10 mol %, about 15 mol %, or about 20 mol % of the total lipid present in the particle.

In some embodiments, the additional lipid comprises a mixture of: (i) a phospholipid such as DOPE; and (ii) cholesterol or a derivative thereof. In some embodiments, the molar ratio of the phospholipid such as DOPE to the cholesterol or a derivative thereof is from about 9:0 to about 1:10, about 2:1 to about 1:4, about 1:1 to about 1:4, or about 1:1 to about 1:3.

### Phosphatidylserine

The RNA particles described herein comprise one or more phosphatidylserines. Such particles further comprise one or more cationic or cationically ionizable lipids and optionally one or more additional lipids.

The term “phosphatidylserine” relates to a phospholipid, more specifically a glycerophospholipid, which consists of two fatty acids attached in ester linkage to the first and second carbons of glycerol and serine attached through a phosphodiester linkage to the third carbon of the glycerol. The term “phosphatidylserine” as used herein includes phosphatidyl-L-serine.

In some embodiments, phosphatidylserine is a compound of formula (I):

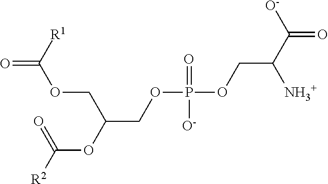

wherein R¹ and R² are each independently a C2 to C43 branched or unbranched acyclic alkyl or acyclic alkenyl group. In different embodiments, R¹ and R² are identical or different.

In some embodiments, R¹ and R² are each independently a C13 to C43 branched or unbranched acyclic alkyl or acyclic alkenyl group which together with their adjacent carbonyl group corresponds to a C14 to C44 saturated or unsaturated fatty acid residue, such as a C14 to C24 saturated or unsaturated fatty acid residue. In some embodiments, R¹ and R² are C13 to C23 branched or unbranched acyclic alkyl, or acyclic alkenyl groups which together with their adjacent carbonyl group are C14 to C24 saturated or unsaturated fatty acid residues,

In various embodiments, R¹ and R² together with their adjacent carbonyl group are saturated or unsaturated fatty acid residues, wherein the fatty acids from which the fatty acid residues are derived are selected from the group consisting of: caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid. In some embodiments, R¹ and R² together with their adjacent carbonyl group are both derived from oleic acid.

The term “phosphatidylserine” as used herein also includes lysophosphatidylserine which relates to a derivative of phosphatidylserine in which one or both acyl derivatives have been removed by hydrolysis.

In different embodiments, the phosphatidylserine comprises dioleoylphosphatidylserine (DOPS), 1,2-dioctanoyl-sn-glycero-3-phospho-L-serine (08:0 PS), 1,2-didecanoyl-sn-glycero-3-phospho-L-serine (10:0 PS), 1,2-dilauroyl-sn-glycero-3-phospho-L-serine (12:0 PS), dimyristoylphosphatidylserine (DMPS), dipalmitoylphosphatidylserine (DPPS), 1,2-diheptadecanoyl-sn-glycero-3-phospho-L-serine (17:0 PS), 1,2-distearoyl-sn-glycero-3-phospho-L-serine (18:0 PS), 1-palmitoyl-2-oleoyl-sn-glycero-3-phospho-L-serine (POPS), 1-stearoyl-2-oleoyl-sn-glycero-3-phospho-L-serine (SOPS), 1-palmitoyl-2-linoleoyl-sn-glycero-3-phospho-L-serine (PLenPS), 1-palmitoyl-2-arachidonoyl-sn-glycero-3-phospho-L-serine (16:0-20:4 PS), 1-palmitoyl-2-docosahexaenoyl-sn-glycero-3-phospho-L-serineor (16:0-22:6 PS) 1-stearoyl-2-linoleoyl-sn-glycero-3-phospho-L-serine (18:0-18:2 PS), 1-stearoyl-2-arachidonoyl-sn-glycero-3-phospho-L-serine (18:0-20:4 PS), 1-stearoyl-2-docosahexaenoyl-sn-glycero-3-phospho-L-serine (18:0-22:6 PS), 1,2-dilinoleoyl-sn-glycero-3-phospho-L-serine (18:2 PS), 1,2-diarachidonoyl-sn-glycero-3-phospho-L-serine (20:4 PS), 1,2-didocosahexaenoyl-sn-glycero-3-phospho-L-serine (22:6 PS), 1-tridecanoyl-sn-glycero-3-phospho-L-serine (13:0 Lyso PS), 1-(10Z-heptadecenoyl)-2-hydroxy-sn-glycero-3-[phospho-L-serine] (17:1 Lyso PS), 1-palmitoyl-2-hydroxy-sn-glycero-3-phospho-L-serine (16:0 Lyso PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), brain PS, brain lyso PS, soy PS, or a mixture thereof.

One particularly preferred phosphatidylserine is 1,2-dioleoyl-sn-glycero-3-phospho-L-serine (DOPS).

The structure of DOPS may be represented as follows:

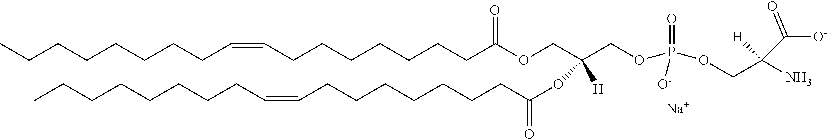 [SMILES: CCCCCCCC/C=C\CCCCCCCC(=O)OC[C@H](COP(=O)([O-])OC[C@H]([NH3+])C(=O)[O-])OC(=O)CCCCCCC/C=C\CCCCCCCC ; [Na+]]

In certain instances, the phosphatidylserine may comprise from about 0.5 mol % to about 80 mol %, from about 1 mol % to about 70 mol %, from about 2 mol % to about 70 mol %, from about 2 mol % to about 60 mol %, from about 2 mol % to about 50 mol %, from about 2 mol % to about 40 mol %, from about 2 mol % to about 30 mol %, from about 5 mol % to about 20 mol %, or from about 7.5 mol % to about 15 mol % of the total lipid present in the particle. In certain instances, the phosphatidylserine may comprise about 10 mol % of the total lipid present in the particle.

In some embodiments, the molar ratio of the cationic or cationically ionizable lipid to the phosphatidylserine is from about 10:1 to about 1:1, about 8:1 to about 1:1, about 6:1 to about 2:1, or about 4:1 to about 2:1.

### RNA-Lipid Particles

In some aspects, an RNA particle comprising:
(i) RNA,
(ii) at least one cationic or cationically ionizable lipid, and
(iii) at least one phosphatidylserine
   is provided.

In some embodiments, the RNA particle further comprises at least one additional lipid. In some embodiments, the additional lipid is selected from the group consisting of neutral lipids, steroids, and combinations thereof. In some embodiments, the additional lipid comprises a neutral lipid. In some embodiments, the additional lipid comprises a phospholipid. In some embodiments, the additional lipid comprises cholesterol or a cholesterol derivative. In some embodiments, the additional lipid comprises a mixture of a phospholipid and cholesterol or a cholesterol derivative. In some embodiments, the additional lipid comprises DOPE, cholesterol, or a mixture of DOPE and cholesterol.

In some embodiments, the RNA particle comprises a cationic or cationically ionizable lipid, a phosphatidylserine, and an additional lipid.

In some embodiments, the cationic or cationically ionizable lipid comprises at least about 10 mol % of the total lipid present in the particle. In some embodiments, the cationic or cationically ionizable lipid comprises at least about 15 mol % of the total lipid present in the particle. In some embodiments, the cationic or cationically ionizable lipid comprises at least about 20 mol % of the total lipid present in the particle. In some embodiments, the cationic or cationically ionizable lipid comprises at least about 25 mol % of the total lipid present in the particle. In some embodiments, the cationic or cationically ionizable lipid comprises at least about 30 mol % of the total lipid present in the particle. In some embodiments, the cationic or cationically ionizable lipid comprises at least about 35 mol % of the total lipid present in the particle. In some embodiments, the cationic or cationically ionizable lipid comprises at least about 40 mol % of the total lipid present in the particle. In some embodiments, the cationic or cationically ionizable lipid comprises at least about 45 mol % of the total lipid present in the particle. In some embodiments, the cationic or cationically ionizable lipid comprises at least about 50 mol % of the total lipid present in the particle. In some embodiments, the cationic or cationically ionizable lipid comprises at least about 55 mol % of the total lipid present in the particle. In some embodiments, the cationic or cationically ionizable lipid comprises at least about 60 mol % of the total lipid present in the particle.

In some embodiments, the cationic or cationically ionizable lipid comprises from about 10 mol % to about 95 mol % of the total lipid present in the particle. In some embodiments, the cationic or cationically ionizable lipid comprises from about 20 mol % to about 95 mol % of the total lipid present in the particle. In some embodiments, the cationic or cationically ionizable lipid comprises from about 20 mol % to about 90 mol % of the total lipid present in the particle. In some embodiments, the cationic or cationically ionizable lipid comprises from about 30 mol % to about 90 mol % of the total lipid present in the particle. In some embodiments, the cationic or cationically ionizable lipid comprises from about 40 mol % to about 90 mol % of the total lipid present in the particle. In some embodiments, the cationic or cationically ionizable lipid comprises from about 40 mol % to about 80 mol % of the total lipid present in the particle. In some embodiments, the cationic or cationically ionizable lipid comprises from about 50 mol % to about 80 mol % of the total lipid present in the particle. In some embodiments, the cationic or cationically ionizable lipid comprises from about 50 mol % to about 70 mol % of the total lipid present in the particle. In some embodiments, the cationic or cationically ionizable lipid comprises about 60 mol % of the total lipid present in the particle.

In some embodiments, the phosphatidylserine comprises from about 0.5 mol % to about 80 mol % of the total lipid present in the particle. In some embodiments, the phosphatidylserine comprises from about 1 mol % to about 70 mol % of the total lipid present in the particle. In some embodiments, the phosphatidylserine comprises from about 2 mol % to about 70 mol % of the total lipid present in the particle. In some embodiments, the phosphatidylserine comprises from about 2 mol % to about 60 mol % of the total lipid present in the particle. In some embodiments, the phosphatidylserine comprises from about 2 mol % to about 50 mol % of the total lipid present in the particle. In some embodiments, the phosphatidylserine comprises from about 2 mol % to about 40 mol % of the total lipid present in the particle. In some embodiments, the phosphatidylserine comprises from about 2 mol % to about 30 mol % of the total lipid present in the particle. In some embodiments, the phosphatidylserine comprises from about 5 mol % to about 30 mol % of the total lipid present in the particle. In some embodiments, the phosphatidylserine comprises from about 5 mol % to about 20 mol % of the total lipid present in the particle. In some embodiments, the phosphatidylserine comprises from about 5 mol % to about 15 mol % of the total lipid present in the particle. In some embodiments, the phosphatidylserine comprises from about 7.5 mol % to about 15 mol % of the total lipid present in the particle. In some embodiments, the phosphatidylserine comprises about 10 mol % of the total lipid present in the particle.

In some embodiments, the additional lipid comprises from about 0 mol % to about 90 mol % of the total lipid present in the particle. In some embodiments, the additional lipid comprises from about 0 mol % to about 80 mol % of the total lipid present in the particle. In some embodiments, the additional lipid comprises from about 2 mol % to about 80 mol % of the total lipid present in the particle. In some embodiments, the additional lipid comprises from about 5 mol % to about 80 mol % of the total lipid present in the particle. In some embodiments, the additional lipid comprises from about 5 mol % to about 70 mol % of the total lipid present in the particle. In some embodiments, the additional lipid comprises from about 5 mol % to about 60 mol % of the total lipid present in the particle. In some embodiments, the additional lipid comprises from about 5 mol % to about 50 mol % of the total lipid present in the particle. In some embodiments, the additional lipid comprises from about 5 mol % to about 40 mol % of the total lipid present in the particle. In some embodiments, the additional lipid comprises from about 5 mol % to about 30 mol % of the total lipid present in the particle. In some embodiments, the additional lipid comprises from about 5 mol % to about 20 mol % of the total lipid present in the particle. In some embodiments, the additional lipid comprises from about 5 mol % to about 15 mol % of the total lipid present in the particle. In some embodiments, the additional lipid comprises from about 7.5 mol % to about 15 mol % of the total lipid present in the particle. In some embodiments, the additional lipid comprises about 10 mol % of the total lipid present in the particle.

In some embodiments, the molar ratio of cationic or cationically ionizable lipid and additional lipid is 2-9.5:0.2-8. In some embodiments, the molar ratio of cationic or cationically ionizable lipid and additional lipid is 2-9:0.5-8. In some embodiments, the molar ratio of cationic or cationically ionizable lipid and additional lipid is 3-9:0.5-7. In some embodiments, the molar ratio of cationic or cationically ionizable lipid and additional lipid is 4-9:0.5-6. In some embodiments, the molar ratio of cationic or cationically ionizable lipid and additional lipid is 4-8:0.5-5. In some embodiments, the molar ratio of cationic or cationically ionizable lipid and additional lipid is 5-8:0.5-4. In some embodiments, the molar ratio of cationic or cationically ionizable lipid and additional lipid is 5-7:0.5-3. In some embodiments, the molar ratio of cationic or cationically ionizable lipid and additional lipid is 5-7:0.5-2. In some embodiments, the molar ratio of cationic or cationically ionizable lipid and additional lipid is 5-7:0.5-1.5. In some embodiments, the molar ratio of cationic or cationically ionizable lipid and additional lipid is about 6:about 1.

In some embodiments, the molar ratio of cationic or cationically ionizable lipid, phosphatidylserine, and additional lipid is 2-9.5:0.2-7:0.2-8. In some embodiments, the molar ratio of cationic or cationically ionizable lipid, phosphatidylserine, and additional lipid is 2-9:0.2-6:0.5-8. In some embodiments, the molar ratio of cationic or cationically ionizable lipid, phosphatidylserine, and additional lipid is 3-9:0.2-5:0.5-7. In some embodiments, the molar ratio of cationic or cationically ionizable lipid, phosphatidylserine, and additional lipid is 4-9:0.2-4:0.5-6. In some embodiments, the molar ratio of cationic or cationically ionizable lipid, phosphatidylserine, and additional lipid is 4-8:0.2-3:0.5-5. In some embodiments, the molar ratio of cationic or cationically ionizable lipid, phosphatidylserine, and additional lipid is 5-8:0.5-3:0.5-4. In some embodiments, the molar ratio of cationic or cationically ionizable lipid, phosphatidylserine, and additional lipid is 5-7:0.5-3:0.5-3. In some embodiments, the molar ratio of cationic or cationically ionizable lipid, phosphatidylserine, and additional lipid is 5-7:0.5-2:0.5-3. In some embodiments, the molar ratio of cationic or cationically ionizable lipid, phosphatidylserine, and additional lipid is 5-7:0.5-2:0.5-2. In some embodiments, the molar ratio of cationic or cationically ionizable lipid, phosphatidylserine, and additional lipid is 5-7:0.5-1.5:0.5-1.5. In some embodiments, the molar ratio of cationic or cationically ionizable lipid, phosphatidylserine, and additional lipid is about 6:about 1:about 1. In some embodiments, the molar ratio of cationic or cationically ionizable lipid, phosphatidylserine, and additional lipid is about 6:about 1:about 1.2-1.8. In some embodiments, the molar ratio of cationic or cationically ionizable lipid, phosphatidylserine, and additional lipid is about 6:about 1:about 1.2. In some embodiments, the molar ratio of cationic or cationically ionizable lipid, phosphatidylserine, and additional lipid is about 6:about 1:about 1.7. In some embodiments, the molar ratio of cationic or cationically ionizable lipid, phosphatidylserine, and additional lipid is about 6:about 1:about 1.8.

In some aspects, an RNA particle comprising:
(i) RNA,
(ii) at least one cationic or cationically ionizable lipid,
(iii) a first phospholipid which is a phosphatidylserine, and
(iv) a second phospholipid
   is provided.

In some embodiments, the cationic or cationically ionizable lipid comprises DODMA. In some embodiments, the phosphatidylserine comprises DOPS. In some embodiments, the second phospholipid comprises DOPE.

In some embodiments, the cationic or cationically ionizable lipid comprises at least about 10 mol % of the total lipid present in the particle. In some embodiments, the cationic or cationically ionizable lipid comprises at least about 15 mol % of the total lipid present in the particle. In some embodiments, the cationic or cationically ionizable lipid comprises at least about 20 mol % of the total lipid present in the particle. In some embodiments, the cationic or cationically ionizable lipid comprises at least about 25 mol % of the total lipid present in the particle. In some embodiments, the cationic or cationically ionizable lipid comprises at least about 30 mol % of the total lipid present in the particle. In some embodiments, the cationic or cationically ionizable lipid comprises at least about 35 mol % of the total lipid present in the particle. In some embodiments, the cationic or cationically ionizable lipid comprises at least about 40 mol % of the total lipid present in the particle. In some embodiments, the cationic or cationically ionizable lipid comprises at least about 45 mol % of the total lipid present in the particle. In some embodiments, the cationic or cationically ionizable lipid comprises at least about 50 mol % of the total lipid present in the particle. In some embodiments, the cationic or cationically ionizable lipid comprises at least about 55 mol % of the total lipid present in the particle. In some embodiments, the cationic or cationically ionizable lipid comprises at least about 60 mol % of the total lipid present in the particle.

In some embodiments, the cationic or cationically ionizable lipid comprises from about 10 mol % to about 95 mol % of the total lipid present in the particle. In some embodiments, the cationic or cationically ionizable lipid comprises from about 20 mol % to about 95 mol % of the total lipid present in the particle. In some embodiments, the cationic or cationically ionizable lipid comprises from about 20 mol % to about 90 mol % of the total lipid present in the particle. In some embodiments, the cationic or cationically ionizable lipid comprises from about 30 mol % to about 90 mol % of the total lipid present in the particle. In some embodiments, the cationic or cationically ionizable lipid comprises from about 40 mol % to about 90 mol % of the total lipid present in the particle. In some embodiments, the cationic or cationically ionizable lipid comprises from about 40 mol % to about 80 mol % of the total lipid present in the particle. In some embodiments, the cationic or cationically ionizable lipid comprises from about 50 mol % to about 80 mol % of the total lipid present in the particle. In some embodiments, the cationic or cationically ionizable lipid comprises from about 50 mol % to about 70 mol % of the total lipid present in the particle. In some embodiments, the cationic or cationically ionizable lipid comprises about 60 mol % of the total lipid present in the particle.

In some embodiments, the phosphatidylserine comprises from about 0.5 mol % to about 80 mol % of the total lipid present in the particle. In some embodiments, the phosphatidylserine comprises from about 1 mol % to about 70 mol % of the total lipid present in the particle. In some embodiments, the phosphatidylserine comprises from about 2 mol % to about 70 mol % of the total lipid present in the particle. In some embodiments, the phosphatidylserine comprises from about 2 mol % to about 60 mol % of the total lipid present in the particle. In some embodiments, the phosphatidylserine comprises from about 2 mol % to about 50 mol % of the total lipid present in the particle. In some embodiments, the phosphatidylserine comprises from about 2 mol % to about 40 mol % of the total lipid present in the particle. In some embodiments, the phosphatidylserine comprises from about 2 mol % to about 30 mol % of the total lipid present in the particle. In some embodiments, the phosphatidylserine comprises from about 5 mol % to about 30 mol % of the total lipid present in the particle. In some embodiments, the phosphatidylserine comprises from about 5 mol % to about 20 mol % of the total lipid present in the particle. In some embodiments, the phosphatidylserine comprises from about 5 mol % to about 15 mol % of the total lipid present in the particle. In some embodiments, the phosphatidylserine comprises from about 7.5 mol % to about 15 mol % of the total lipid present in the particle. In some embodiments, the phosphatidylserine comprises about 10 mol % of the total lipid present in the particle.

In some embodiments, the second phospholipid comprises from about 0.5 mol % to about 90 mol % of the total lipid present in the particle. In some embodiments, the second phospholipid comprises from about 1 mol % to about 80 mol % of the total lipid present in the particle. In some embodiments, the second phospholipid comprises from about 2 mol % to about 80 mol % of the total lipid present in the particle. In some embodiments, the second phospholipid comprises from about 5 mol % to about 80 mol % of the total lipid present in the particle. In some embodiments, the second phospholipid comprises from about 5 mol % to about 70 mol % of the total lipid present in the particle. In some embodiments, the second phospholipid comprises from about 5 mol % to about 60 mol % of the total lipid present in the particle. In some embodiments, the second phospholipid comprises from about 5 mol % to about 50 mol % of the total lipid present in the particle. In some embodiments, the second phospholipid comprises from about 5 mol % to about 40 mol % of the total lipid present in the particle. In some embodiments, the second phospholipid comprises from about 5 mol % to about 30 mol % of the total lipid present in the particle. In some embodiments, the second phospholipid comprises from about 5 mol % to about 20 mol % of the total lipid present in the particle. In some embodiments, the second phospholipid comprises from about 5 mol % to about 15 mol % of the total lipid present in the particle. In some embodiments, the second phospholipid comprises from about 7.5 mol % to about 15 mol % of the total lipid present in the particle. In some embodiments, the second phospholipid comprises about 10 mol % of the total lipid present in the particle.

In some embodiments, the molar ratio of cationic or cationically ionizable lipid and second phospholipid is 2-9.5:0.2-8. In some embodiments, the molar ratio of cationic or cationically ionizable lipid and second phospholipid is 2-9:0.5-8. In some embodiments, the molar ratio of cationic or cationically ionizable lipid and second phospholipid is 3-9:0.5-7. In some embodiments, the molar ratio of cationic or cationically ionizable lipid and second phospholipid is 4-9:0.5-6. In some embodiments, the molar ratio of cationic or cationically ionizable lipid and second phospholipid is 4-8:0.5-5. In some embodiments, the molar ratio of cationic or cationically ionizable lipid and second phospholipid is 5-8:0.5-4. In some embodiments, the molar ratio of cationic or cationically ionizable lipid and second phospholipid is 5-7:0.5-3. In some embodiments, the molar ratio of cationic or cationically ionizable lipid and second phospholipid is 5-7:0.5-2. In some embodiments, the molar ratio of cationic or cationically ionizable lipid and second phospholipid is 5-7:0.5-1.5. In some embodiments, the molar ratio of cationic or cationically ionizable lipid and second phospholipid is about 6:about 1.

In some embodiments, the molar ratio of cationic or cationically ionizable lipid, phosphatidylserine, and second phospholipid is 2-9.5:0.2-7:0.2-8. In some embodiments, the molar ratio of cationic or cationically ionizable lipid, phosphatidylserine, and second phospholipid is 2-9:0.2-6:0.5-8. In some embodiments, the molar ratio of cationic or cationically ionizable lipid, phosphatidylserine, and second phospholipid is 3-9:0.2-5:0.5-7. In some embodiments, the molar ratio of cationic or cationically ionizable lipid, phosphatidylserine, and second phospholipid is 4-9:0.2-4:0.5-6. In some embodiments, the molar ratio of cationic or cationically ionizable lipid, phosphatidylserine, and second phospholipid is 4-8:0.2-3:0.5-5. In some embodiments, the molar ratio of cationic or cationically ionizable lipid, phosphatidylserine, and second phospholipid is 5-8:0.5-3:0.5-4. In some embodiments, the molar ratio of cationic or cationically ionizable lipid, phosphatidylserine, and second phospholipid is 5-7:0.5-3:0.5-3. In some embodiments, the molar ratio of cationic or cationically ionizable lipid, phosphatidylserine, and second phospholipid is 5-7:0.5-2:0.5-3. In some embodiments, the molar ratio of cationic or cationically ionizable lipid, phosphatidylserine, and second phospholipid is 5-7:0.5-2:0.5-2. In some embodiments, the molar ratio of cationic or cationically ionizable lipid, phosphatidylserine, and second phospholipid is 5-7:0.5-1.5:0.5-1.5. In some embodiments, the molar ratio of cationic or cationically ionizable lipid, phosphatidylserine, and second phospholipid is about 6:about 1:about 1. In some embodiments, the molar ratio of cationic or cationically ionizable lipid, phosphatidylserine, and second phospholipid is about 6:about 1:about 1.2-1.8. In some embodiments, the molar ratio of cationic or cationically ionizable lipid, phosphatidylserine, and second phospholipid is about 6:about 1:about 1.2. In some embodiments, the molar ratio of cationic or cationically ionizable lipid, phosphatidylserine, and second phospholipid is about 6:about 1:about 1.7. In some embodiments, the molar ratio of cationic or cationically ionizable lipid, phosphatidylserine, and second phospholipid is about 6:about 1:about 1.8.

In some aspects, an RNA particle comprising: (i) RNA, (ii) N,N-dimethyl-2,3-dioleyloxypropylamine (DODMA) and (iii) dioleoylphosphatidylserine (DOPS) is provided.

In some embodiments, the RNA particle further comprises dioleoylphosphatidylethanolamine (DOPE).

In some embodiments, the DODMA comprises at least about 10 mol % of the total lipid present in the particle. In some embodiments, the DODMA comprises at least about 15 mol % of the total lipid present in the particle. In some embodiments, the DODMA comprises at least about 20 mol % of the total lipid present in the particle. In some embodiments, the DODMA comprises at least about 25 mol % of the total lipid present in the particle. In some embodiments, the DODMA comprises at least about 30 mol % of the total lipid present in the particle. In some embodiments, the DODMA comprises at least about 35 mol % of the total lipid present in the particle. In some embodiments, the DODMA comprises at least about 40 mol % of the total lipid present in the particle. In some embodiments, the DODMA comprises at least about 45 mol % of the total lipid present in the particle. In some embodiments, the DODMA comprises at least about 50 mol % of the total lipid present in the particle. In some embodiments, the DODMA comprises at least about 55 mol % of the total lipid present in the particle. In some embodiments, the DODMA comprises at least about 60 mol % of the total lipid present in the particle.

In some embodiments, the DODMA comprises from about 10 mol % to about 95 mol % of the total lipid present in the particle. In some embodiments, the DODMA comprises from about 20 mol % to about 95 mol % of the total lipid present in the particle. In some embodiments, the DODMA comprises from about 20 mol % to about 90 mol % of the total lipid present in the particle. In some embodiments, the DODMA comprises from about 30 mol % to about 90 mol % of the total lipid present in the particle. In some embodiments, the DODMA comprises from about 40 mol % to about 90 mol % of the total lipid present in the particle. In some embodiments, the DODMA comprises from about 40 mol % to about 80 mol % of the total lipid present in the particle. In some embodiments, the DODMA comprises from about 50 mol % to about 80 mol % of the total lipid present in the particle. In some embodiments, the DODMA comprises from about 50 mol % to about 70 mol % of the total lipid present in the particle. In some embodiments, the DODMA comprises about 60 mol % of the total lipid present in the particle.

In some embodiments, the DOPS comprises from about 0.5 mol % to about 80 mol % of the total lipid present in the particle. In some embodiments, the DOPS comprises from about 1 mol % to about 70 mol % of the total lipid present in the particle. In some embodiments, the DOPS comprises from about 2 mol % to about 70 mol % of the total lipid present in the particle. In some embodiments, the DOPS comprises from about 2 mol % to about 60 mol % of the total lipid present in the particle. In some embodiments, the DOPS comprises from about 2 mol % to about 50 mol % of the total lipid present in the particle. In some embodiments, the DOPS comprises from about 2 mol % to about 40 mol % of the total lipid present in the particle. In some embodiments, the DOPS comprises from about 2 mol % to about 30 mol % of the total lipid present in the particle. In some embodiments, the DOPS comprises from about 5 mol % to about 30 mol % of the total lipid present in the particle. In some embodiments, the DOPS comprises from about 5 mol % to about 20 mol % of the total lipid present in the particle. In some embodiments, the DOPS comprises from about 5 mol % to about 15 mol % of the total lipid present in the particle. In some embodiments, the DOPS comprises from about 7.5 mol % to about 15 mol % of the total lipid present in the particle. In some embodiments, the DOPS comprises about 10 mol % of the total lipid present in the particle.

In some embodiments, the DOPE comprises from about 0 mol % to about 90 mol % of the total lipid present in the particle. In some embodiments, the DOPE comprises from about 0 mol % to about 80 mol % of the total lipid present in the particle. In some embodiments, the DOPE comprises from about 2 mol % to about 80 mol % of the total lipid present in the particle. In some embodiments, the DOPE comprises from about 5 mol % to about 80 mol % of the total lipid present in the particle. In some embodiments, the DOPE comprises from about 5 mol % to about 70 mol % of the total lipid present in the particle. In some embodiments, the DOPE comprises from about 5 mol % to about 60 mol % of the total lipid present in the particle. In some embodiments, the DOPE comprises from about 5 mol % to about 50 mol % of the total lipid present in the particle. In some embodiments, the DOPE comprises from about 5 mol % to about 40 mol % of the total lipid present in the particle. In some embodiments, the DOPE comprises from about 5 mol % to about 30 mol % of the total lipid present in the particle. In some embodiments, the DOPE comprises from about 5 mol % to about 20 mol % of the total lipid present in the particle. In some embodiments, the DOPE comprises from about 5 mol % to about 15 mol % of the total lipid present in the particle. In some embodiments, the DOPE comprises from about 7.5 mol % to about 15 mol % of the total lipid present in the particle. In some embodiments, the DOPE comprises about 10 mol % of the total lipid present in the particle.

In some embodiments, the molar ratio of DODMA and DOPE is 2-9.5:0.2-8. In some embodiments, the molar ratio of DODMA and DOPE is 2-9:0.5-8. In some embodiments, the molar ratio of DODMA and DOPE is 3-9:0.5-7. In some embodiments, the molar ratio of DODMA and DOPE is 4-9:0.5-6. In some embodiments, the molar ratio of DODMA and DOPE is 4-8:0.5-5. In some embodiments, the molar ratio of DODMA and DOPE is 5-8:0.5-4. In some embodiments, the molar ratio of DODMA and DOPE is 5-7:0.5-3. In some embodiments, the molar ratio of DODMA and DOPE is 5-7:0.5-2. In some embodiments, the molar ratio of DODMA and DOPE is 5-7:0.5-1.5. In some embodiments, the molar ratio of DODMA and DOPE is about 6:about 1.

In some embodiments, the molar ratio of DODMA, DOPS, and DOPE is 2-9.5:0.2-7:0.2-8. In some embodiments, the molar ratio of DODMA, DOPS, and DOPE is 2-9:0.2-6:0.5-8. In some embodiments, the molar ratio of DODMA, DOPS, and DOPE is 3-9:0.2-5:0.5-7. In some embodiments, the molar ratio of DODMA, DOPS, and DOPE is 4-9:0.2-4:0.5-6. In some embodiments, the molar ratio of DODMA, DOPS, and DOPE is 4-8:0.2-3:0.5-5. In some embodiments, the molar ratio of DODMA, DOPS, and DOPE is 5-8:0.5-3:0.5-4. In some embodiments, the molar ratio of DODMA, DOPS, and DOPE is 5-7:0.5-3:0.5-3. In some embodiments, the molar ratio of DODMA, DOPS, and DOPE is 5-7:0.5-2:0.5-3. In some embodiments, the molar ratio of DODMA, DOPS, and DOPE is 5-7:0.5-2:0.5-2. In some embodiments, the molar ratio of DODMA, DOPS, and DOPE is 5-7:0.5-1.5:0.5-1.5. In some embodiments, the molar ratio of DODMA, DOPS, and DOPE is about 6:about 1:about 1. In some embodiments, the molar ratio of DODMA, DOPS, and DOPE is about 6:about 1:about 1.2-1.8. In some embodiments, the molar ratio of DODMA, DOPS, and DOPE is about 6:about 1:about 1.2. In some embodiments, the molar ratio of DODMA, DOPS, and DOPE is about 6:about 1:about 1.7. In some embodiments, the molar ratio of DODMA, DOPS, and DOPE is about 6:about 1:about 1.8.

In some aspects, an RNA particle comprising RNA, a cationic or cationically ionizable lipid, DOPS, and DOPE is provided.

In some embodiments, the cationic or cationically ionizable lipid comprises at least about 10 mol % of the total lipid present in the particle. In some embodiments, the cationic or cationically ionizable lipid comprises at least about 15 mol % of the total lipid present in the particle. In some embodiments, the cationic or cationically ionizable lipid comprises at least about 20 mol % of the total lipid present in the particle. In some embodiments, the cationic or cationically ionizable lipid comprises at least about 25 mol % of the total lipid present in the particle. In some embodiments, the cationic or cationically ionizable lipid comprises at least about 30 mol % of the total lipid present in the particle. In some embodiments, the cationic or cationically ionizable lipid comprises at least about 35 mol % of the total lipid present in the particle. In some embodiments, the cationic or cationically ionizable lipid comprises at least about 40 mol % of the total lipid present in the particle. In some embodiments, the cationic or cationically ionizable lipid comprises at least about 45 mol % of the total lipid present in the particle. In some embodiments, the cationic or cationically ionizable lipid comprises at least about 50 mol % of the total lipid present in the particle. In some embodiments, the cationic or cationically ionizable lipid comprises at least about 55 mol % of the total lipid present in the particle. In some embodiments, the cationic or cationically ionizable lipid comprises at least about 60 mol % of the total lipid present in the particle.

In some embodiments, the cationic or cationically ionizable lipid comprises from about 10 mol % to about 95 mol % of the total lipid present in the particle. In some embodiments, the cationic or cationically ionizable lipid comprises from about 20 mol % to about 95 mol % of the total lipid present in the particle. In some embodiments, the cationic or cationically ionizable lipid comprises from about 20 mol % to about 90 mol % of the total lipid present in the particle. In some embodiments, the cationic or cationically ionizable lipid comprises from about 30 mol % to about 90 mol % of the total lipid present in the particle. In some embodiments, the cationic or cationically ionizable lipid comprises from about 40 mol % to about 90 mol % of the total lipid present in the particle. In some embodiments, the cationic or cationically ionizable lipid comprises from about 40 mol % to about 80 mol % of the total lipid present in the particle. In some embodiments, the cationic or cationically ionizable lipid comprises from about 50 mol % to about 80 mol % of the total lipid present in the particle. In some embodiments, the cationic or cationically ionizable lipid comprises from about 50 mol % to about 70 mol % of the total lipid present in the particle. In some embodiments, the cationic or cationically ionizable lipid comprises about 60 mol % of the total lipid present in the particle.

In some embodiments, the DOPS comprises from about 0.5 mol % to about 80 mol % of the total lipid present in the particle. In some embodiments, the DOPS comprises from about 1 mol % to about 70 mol % of the total lipid present in the particle. In some embodiments, the DOPS comprises from about 2 mol % to about 70 mol % of the total lipid present in the particle. In some embodiments, the DOPS comprises from about 2 mol % to about 60 mol % of the total lipid present in the particle. In some embodiments, the DOPS comprises from about 2 mol % to about 50 mol % of the total lipid present in the particle. In some embodiments, the DOPS comprises from about 2 mol % to about 40 mol % of the total lipid present in the particle. In some embodiments, the DOPS comprises from about 2 mol % to about 30 mol % of the total lipid present in the particle. In some embodiments, the DOPS comprises from about 5 mol % to about 30 mol % of the total lipid present in the particle. In some embodiments, the DOPS comprises from about 5 mol % to about 20 mol % of the total lipid present in the particle. In some embodiments, the DOPS comprises from about 5 mol % to about 15 mol % of the total lipid present in the particle. In some embodiments, the DOPS comprises from about 7.5 mol % to about 15 mol % of the total lipid present in the particle. In some embodiments, the DOPS comprises about 10 mol % of the total lipid present in the particle.

In some embodiments, the DOPE comprises from about 0.5 mol % to about 90 mol % of the total lipid present in the particle. In some embodiments, the DOPE comprises from about 1 mol % to about 80 mol % of the total lipid present in the particle. In some embodiments, the DOPE comprises from about 2 mol % to about 80 mol % of the total lipid present in the particle. In some embodiments, the DOPE comprises from about 5 mol % to about 80 mol % of the total lipid present in the particle. In some embodiments, the DOPE comprises from about 5 mol % to about 70 mol % of the total lipid present in the particle. In some embodiments, the DOPE comprises from about 5 mol % to about 60 mol % of the total lipid present in the particle. In some embodiments, the DOPE comprises from about 5 mol % to about 50 mol % of the total lipid present in the particle. In some embodiments, the DOPE comprises from about 5 mol % to about 40 mol % of the total lipid present in the particle. In some embodiments, the DOPE comprises from about 5 mol % to about 30 mol % of the total lipid present in the particle. In some embodiments, the DOPE comprises from about 5 mol % to about 20 mol % of the total lipid present in the particle. In some embodiments, the DOPE comprises from about 5 mol % to about 15 mol % of the total lipid present in the particle. In some embodiments, the DOPE comprises from about 7.5 mol % to about 15 mol % of the total lipid present in the particle. In some embodiments, the DOPE comprises about 10 mol % of the total lipid present in the particle.

In some embodiments, the molar ratio of cationic or cationically ionizable lipid and DOPE is 2-9.5:0.2-8. In some embodiments, the molar ratio of cationic or cationically ionizable lipid and DOPE is 2-9:0.5-8. In some embodiments, the molar ratio of cationic or cationically ionizable lipid and DOPE is 3-9:0.5-7. In some embodiments, the molar ratio of cationic or cationically ionizable lipid and DOPE is 4-9:0.5-6. In some embodiments, the molar ratio of cationic or cationically ionizable lipid and DOPE is 4-8:0.5-5. In some embodiments, the molar ratio of cationic or cationically ionizable lipid and DOPE is 5-8:0.5-4. In some embodiments, the molar ratio of cationic or cationically ionizable lipid and DOPE is 5-7:0.5-3. In some embodiments, the molar ratio of cationic or cationically ionizable lipid and DOPE is 5-7:0.5-2. In some embodiments, the molar ratio of cationic or cationically ionizable lipid and DOPE is 5-7:0.5-1.5. In some embodiments, the molar ratio of cationic or cationically ionizable lipid and DOPE is about 6:about 1.

In some embodiments, the molar ratio of cationic or cationically ionizable lipid, DOPS, and DOPE is 2-9.5:0.2-7:0.2-8. In some embodiments, the molar ratio of cationic or cationically ionizable lipid, DOPS, and DOPE is 2-9:0.2-6:0.5-8. In some embodiments, the molar ratio of cationic or cationically ionizable lipid, DOPS, and DOPE is 3-9:0.2-5:0.5-7. In some embodiments, the molar ratio of cationic or cationically ionizable lipid, DOPS, and DOPE is 4-9:0.2-4:0.5-6. In some embodiments, the molar ratio of cationic or cationically ionizable lipid, DOPS, and DOPE is 4-8:0.2-3:0.5-5. In some embodiments, the molar ratio of cationic or cationically ionizable lipid, DOPS, and DOPE is 5-8:0.5-3:0.5-4. In some embodiments, the molar ratio of cationic or cationically ionizable lipid, DOPS, and DOPE is 5-7:0.5-3:0.5-3. In some embodiments, the molar ratio of cationic or cationically ionizable lipid, DOPS, and DOPE is 5-7:0.5-2:0.5-3. In some embodiments, the molar ratio of cationic or cationically ionizable lipid, DOPS, and DOPE is 5-7:0.5-2:0.5-2. In some embodiments, the molar ratio of cationic or cationically ionizable lipid, DOPS, and DOPE is 5-7:0.5-1.5:0.5-1.5. In some embodiments, the molar ratio of cationic or cationically ionizable lipid, DOPS, and DOPE is about 6:about 1:about 1. In some embodiments, the molar ratio of cationic or cationically ionizable lipid, DOPS, and DOPE is about 6:about 1:about 1.2-1.8. In some embodiments, the molar ratio of cationic or cationically ionizable lipid, DOPS, and DOPE is about 6:about 1:about 1.2. In some embodiments, the molar ratio of cationic or cationically ionizable lipid, DOPS, and DOPE is about 6:about 1:about 1.7. In some embodiments, the molar ratio of cationic or cationically ionizable lipid, DOPS, and DOPE is about 6:about 1:about 1.8.

In some aspects, an RNA particle comprising RNA, DODMA, DOPS, and DOPE is provided.

In some embodiments, the DODMA comprises at least about 10 mol % of the total lipid present in the particle. In some embodiments, the DODMA comprises at least about 15 mol % of the total lipid present in the particle. In some embodiments, the DODMA comprises at least about 20 mol % of the total lipid present in the particle. In some embodiments, the DODMA comprises at least about 25 mol % of the total lipid present in the particle. In some embodiments, the DODMA comprises at least about 30 mol % of the total lipid present in the particle. In some embodiments, the DODMA comprises at least about 35 mol % of the total lipid present in the particle. In some embodiments, the DODMA comprises at least about 40 mol % of the total lipid present in the particle. In some embodiments, the DODMA comprises at least about 45 mol % of the total lipid present in the particle. In some embodiments, the DODMA comprises at least about 50 mol % of the total lipid present in the particle. In some embodiments, the DODMA comprises at least about 55 mol % of the total lipid present in the particle. In some embodiments, the DODMA comprises at least about 60 mol % of the total lipid present in the particle.

In some embodiments, the DODMA comprises from about 10 mol % to about 95 mol % of the total lipid present in the particle. In some embodiments, the DODMA comprises from about 20 mol % to about 95 mol % of the total lipid present in the particle. In some embodiments, the DODMA comprises from about 20 mol % to about 90 mol % of the total lipid present in the particle. In some embodiments, the DODMA comprises from about 30 mol % to about 90 mol % of the total lipid present in the particle. In some embodiments, the DODMA comprises from about 40 mol % to about 90 mol % of the total lipid present in the particle. In some embodiments, the DODMA comprises from about 40 mol % to about 80 mol % of the total lipid present in the particle. In some embodiments, the DODMA comprises from about 50 mol % to about 80 mol % of the total lipid present in the particle. In some embodiments, the DODMA comprises from about 50 mol % to about 70 mol % of the total lipid present in the particle. In some embodiments, the DODMA comprises about 60 mol % of the total lipid present in the particle.

In some embodiments, the DOPS comprises from about 0.5 mol % to about 80 mol % of the total lipid present in the particle. In some embodiments, the DOPS comprises from about 1 mol % to about 70 mol % of the total lipid present in the particle. In some embodiments, the DOPS comprises from about 2 mol % to about 70 mol % of the total lipid present in the particle. In some embodiments, the DOPS comprises from about 2 mol % to about 60 mol % of the total lipid present in the particle. In some embodiments, the DOPS comprises from about 2 mol % to about 50 mol % of the total lipid present in the particle. In some embodiments, the DOPS comprises from about 2 mol % to about 40 mol % of the total lipid present in the particle. In some embodiments, the DOPS comprises from about 2 mol % to about 30 mol % of the total lipid present in the particle. In some embodiments, the DOPS comprises from about 5 mol % to about 30 mol % of the total lipid present in the particle. In some embodiments, the DOPS comprises from about 5 mol % to about 20 mol % of the total lipid present in the particle. In some embodiments, the DOPS comprises from about 5 mol % to about 15 mol % of the total lipid present in the particle. In some embodiments, the DOPS comprises from about 7.5 mol % to about 15 mol % of the total lipid present in the particle. In some embodiments, the DOPS comprises about 10 mol % of the total lipid present in the particle.

In some embodiments, the DOPE comprises from about 0.5 mol % to about 90 mol % of the total lipid present in the particle. In some embodiments, the DOPE comprises from about 1 mol % to about 80 mol % of the total lipid present in the particle. In some embodiments, the DOPE comprises from about 2 mol % to about 80 mol % of the total lipid present in the particle. In some embodiments, the DOPE comprises from about 5 mol % to about 80 mol % of the total lipid present in the particle. In some embodiments, the DOPE comprises from about 5 mol % to about 70 mol % of the total lipid present in the particle. In some embodiments, the DOPE comprises from about 5 mol % to about 60 mol % of the total lipid present in the particle. In some embodiments, the DOPE comprises from about 5 mol % to about 50 mol % of the total lipid present in the particle. In some embodiments, the DOPE comprises from about 5 mol % to about 40 mol % of the total lipid present in the particle. In some embodiments, the DOPE comprises from about 5 mol % to about 30 mol % of the total lipid present in the particle. In some embodiments, the DOPE comprises from about 5 mol % to about 20 mol % of the total lipid present in the particle. In some embodiments, the DOPE comprises from about 5 mol % to about 15 mol % of the total lipid present in the particle. In some embodiments, the DOPE comprises from about 7.5 mol % to about 15 mol % of the total lipid present in the particle. In some embodiments, the DOPE comprises about 10 mol % of the total lipid present in the particle.

In some embodiments, the molar ratio of DODMA and DOPE is 2-9.5:0.2-8. In some embodiments, the molar ratio of DODMA and DOPE is 2-9:0.5-8. In some embodiments, the molar ratio of DODMA and DOPE is 3-9:0.5-7. In some embodiments, the molar ratio of DODMA and DOPE is 4-9:0.5-6. In some embodiments, the molar ratio of DODMA and DOPE is 4-8:0.5-5. In some embodiments, the molar ratio of DODMA and DOPE is 5-8:0.5-4. In some embodiments, the molar ratio of DODMA and DOPE is 5-7:0.5-3. In some embodiments, the molar ratio of DODMA and DOPE is 5-7:0.5-2. In some embodiments, the molar ratio of DODMA and DOPE is 5-7:0.5-1.5. In some embodiments, the molar ratio of DODMA and DOPE is about 6:about 1.

In some embodiments, the molar ratio of DODMA, DOPS, and DOPE is 2-9.5:0.2-7:0.2-8. In some embodiments, the molar ratio of DODMA, DOPS, and DOPE is 2-9:0.2-6:0.5-8. In some embodiments, the molar ratio of DODMA, DOPS, and DOPE is 3-9:0.2-5:0.5-7. In some embodiments, the molar ratio of DODMA, DOPS, and DOPE is 4-9:0.2-4:0.5-6. In some embodiments, the molar ratio of DODMA, DOPS, and DOPE is 4-8:0.2-3:0.5-5. In some embodiments, the molar ratio of DODMA, DOPS, and DOPE is 5-8:0.5-3:0.5-4. In some embodiments, the molar ratio of DODMA, DOPS, and DOPE is 5-7:0.5-3:0.5-3. In some embodiments, the molar ratio of DODMA, DOPS, and DOPE is 5-7:0.5-2:0.5-3. In some embodiments, the molar ratio of DODMA, DOPS, and DOPE is 5-7:0.5-2:0.5-2. In some embodiments, the molar ratio of DODMA, DOPS, and DOPE is 5-7:0.5-1.5:0.5-1.5. In some embodiments, the molar ratio of DODMA, DOPS, and DOPE is about 6:about 1:about 1. In some embodiments, the molar ratio of DODMA, DOPS, and DOPE is about 6:about 1:about 1.2-1.8. In some embodiments, the molar ratio of DODMA, DOPS, and DOPE is about 6:about 1:about 1.2. In some embodiments, the molar ratio of DODMA, DOPS, and DOPE is about 6:about 1:about 1.7. In some embodiments, the molar ratio of DODMA, DOPS, and DOPE is about 6:about 1:about 1.8.

In some embodiments, the RNA particle described herein comprises a non-ionic amphiphilic organic compound. In some embodiments, the non-ionic amphiphilic organic compound is a surfactant. In some embodiments, the non-ionic amphiphilic organic compound is a poly(ethylene glycol) (PEG) surfactant. In some embodiments, the non-ionic amphiphilic organic compound comprises a poly(ethylene glycol) (PEG) chain linked to a single hydrophobic chain. In some embodiments, the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof. In some embodiments, the level of non-ionic amphiphilic organic compound present in a RNA particle (or composition comprising an RNA particle) is at least about 5 mol % of the total lipid present in the particle or composition, or at least about 0.15 mM. In some embodiments, the level of non-ionic amphiphilic organic compound present in a RNA particle (or composition comprising an RNA particle) is at least about 20 mol % of the total lipid present in the particle or composition, or at least about 0.6 mM. In some embodiments, the non-ionic amphiphilic organic compound comprises a polyoxyethylene sorbitan fatty acid ester. In some embodiments, the level of polyoxyethylene sorbitan fatty acid ester present in a RNA particle (or composition comprising an RNA particle) is at least about 5 mol % of the total lipid present in the particle or composition, or at least about 0.15 mM. In some embodiments, the level of polyoxyethylene sorbitan fatty acid ester present in a RNA particle (or composition comprising an RNA particle) is at least about 20 mol % of the total lipid present in the particle or composition, or at least about 0.6 mM. In some embodiments, the non-ionic amphiphilic organic compound comprises a polysorbate. In some embodiments, the level of polysorbate present in a RNA particle (or composition comprising an RNA particle) is at least about 5 mol % of the total lipid present in the particle or composition, or at least about 0.3 mM. In some embodiments, the level of polysorbate present in a RNA particle (or composition comprising an RNA particle) is at least about 20 mol % of the total lipid present in the particle or composition, or at least about 0.6 mM. In some embodiments, the non-ionic amphiphilic organic compound comprises polysorbate 20. In some embodiments, the level of polysorbate 20 present in a RNA particle (or composition comprising an RNA particle) is at least about 5 mol % of the total lipid present in the particle or composition, or at least about 0.3 mM. In some embodiments, the level of polysorbate 20 present in a RNA particle (or composition comprising an RNA particle) is at least about 20 mol % of the total lipid present in the particle or composition, or at least about 0.6 mM.

In some embodiments, a composition comprises a non-ionic amphiphilic organic compound at a level of at least about 0.15 mM. In some embodiments, a composition comprises a non-ionic amphiphilic organic compound at a level of at least about 0.2 mM. In some embodiments, a composition comprises a non-ionic amphiphilic organic compound at a level of at least about 0.25 mM. In some embodiments, a composition comprises a non-ionic amphiphilic organic compound at a level of at least about 0.3 mM. In some embodiments, a composition comprises a non-ionic amphiphilic organic compound at a level of at least about 0.35 mM. In some embodiments, a composition comprises a non-ionic amphiphilic organic compound at a level of at least about 0.4 mM. In some embodiments, a composition comprises a non-ionic amphiphilic organic compound at a level of at least about 0.45 mM. In some embodiments, a composition comprises a non-ionic amphiphilic organic compound at a level of at least about 0.5 mM. In some embodiments, a composition comprises a non-ionic amphiphilic organic compound at a level of at least about 0.55 mM. In some embodiments, a composition comprises a non-ionic amphiphilic organic compound at a level of at least about 0.6 mM. In some embodiments, a composition comprises a non-ionic amphiphilic organic compound at a level of at least about 0.7 mM. In some embodiments, a composition comprises a non-ionic amphiphilic organic compound at a level of at least about 0.8 mM. In some embodiments, a composition comprises a non-ionic amphiphilic organic compound at a level of at least about 0.9 mM. In some embodiments, a composition comprises a non-ionic amphiphilic organic compound at a level of at least about 1.0 mM. In some embodiments, a composition comprises a non-ionic amphiphilic organic compound at a level of at least about 1.1 mM. In some embodiments, a composition comprises a non-ionic amphiphilic organic compound at a level of at least about 1.2 mM. In some embodiments, a composition comprises a non-ionic amphiphilic organic compound at a level of at least about 1.3 mM. In some embodiments, a composition comprises a non-ionic amphiphilic organic compound at a level of at least about 1.4 mM. In some embodiments, a composition comprises a non-ionic amphiphilic organic compound at a level of at least about 1.5 mM. In some embodiments, a composition comprises a non-ionic amphiphilic organic compound at a level of at least about 2.0 mM. In some embodiments, a composition comprises a non-ionic amphiphilic organic compound at a level of at least about 2.5 mM. In some embodiments, a composition comprises a non-ionic amphiphilic organic compound at a level of at least about 3.0 mM. In some embodiments, a composition comprises a non-ionic amphiphilic organic compound at a level of at least about 4.0 mM. In some embodiments, a composition comprises a non-ionic amphiphilic organic compound at a level of at least about 5.0 mM.

In some embodiments, a composition comprises a non-ionic amphiphilic organic compound at a level of from about 0.6 mM to about 0.75 mM. In some embodiments, a composition comprises a non-ionic amphiphilic organic compound at a level of from about 0.75 mM to about 1.0 mM. In some embodiments, a composition comprises a non-ionic amphiphilic organic compound at a level of from about 1.0 mM to about 1.25 mM. In some embodiments, a composition comprises a non-ionic amphiphilic organic compound at a level of from about 1.25 mM to about 1.5 mM. In some embodiments, a composition comprises a non-ionic amphiphilic organic compound at a level of from about 1.75 mM to about 2.0 mM. In some embodiments, a composition comprises a non-ionic amphiphilic organic compound at a level of from about 2.0 mM to about 2.25 mM. In some embodiments, a composition comprises a non-ionic amphiphilic organic compound at a level of from about 2.25 mM to about 2.5 mM. In some embodiments, a composition comprises a non-ionic amphiphilic organic compound at a level of from about 2.5 mM to about 2.75 mM. In some embodiments, a composition comprises a non-ionic amphiphilic organic compound at a level of from about 2.75 mM to about 3.0 mM. In some embodiments, a composition comprises a non-ionic amphiphilic organic compound at a level of from about 3.0 mM to about 3.5 mM. In some embodiments, a composition comprises a non-ionic amphiphilic organic compound at a level of from about 4.0 mM to about 4.5 mM. In some embodiments, a composition comprises a non-ionic amphiphilic organic compound at a level of from about 4.5 mM to about 5.0 mM.

In some embodiments, the RNA particle described herein does not comprise cholesterol. In some embodiments, the RNA particle described herein does not comprise a steroid.

In some embodiments, the RNA particle described herein is a lipid nanoparticle (LNP) or a lipoplex particle (LPX).

In some embodiments, the RNA particle described herein in addition to RNA comprises: (i) a cationic or cationically ionizable lipid, (ii) a non-cationic lipid, in particular neutral lipid, (e.g., one or more phospholipids and/or cholesterol), and (iii) a phosphatidylserine.

In some embodiments, the RNA particle described herein in addition to RNA comprises:
(i) a cationic lipid or cationically ionizable lipid which may comprise from about 10 mol % to about 95 mol %, from about 20 mol % to about 95 mol %, from about 20 mol % to about 90 mol %, from about 30 mol % to about 90 mol %, from about 40 mol % to about 90 mol %, or from about 40 mol % to about 80 mol % of the total lipid present in the particle,
(ii) a non-cationic lipid, in particular neutral lipid, (e.g., one or more phospholipids and/or cholesterol) which may comprise from about 0 mol % to about 90 mol %, from about 0 mol % to about 80 mol %, from about 2 mol % to about 80 mol %, from about 5 mol % to about 80 mol %, from about 5 mol % to about 60 mol %, from about 5 mol % to about 50 mol %, from about 7.5 mol % to about 50 mol %, or from about 10 mol % to about 40 mol % of the total lipid present in the particle, and
(iii) a phosphatidylserine which may comprise from about 0.5 mol % to about 80 mol %, from about 1 mol % to about 70 mol %, from about 2 mol % to about 70 mol %, from about 2 mol % to about 60 mol %, from about 2 mol % to about 50 mol %, from about 2 mol % to about 40 mol %, from about 2 mol % to about 30 mol %, from about 5 mol % to about 20 mol %, or from about 7.5 mol % to about 15 mol % of the total lipid present in the particle.

In some embodiments, the RNA particle described herein in addition to RNA comprises: (i) a cationic or cationically ionizable lipid, (ii) a non-cationic lipid, in particular neutral lipid, (e.g., one or more phospholipids and/or cholesterol), and (iii) a phosphatidylserine, wherein the molar ratio of (i) to (ii) to (iii) is 5.5-6.5:0.9-1.1:0.9-1.1, such as about 6:1:1.

In some embodiments, the RNA component in the RNA particles described herein is mRNA or saRNA. In some embodiments, the RNA component in the RNA particles described herein is mRNA. In some embodiments, the RNA is coding RNA.

In some embodiments, the RNA is of unimolecular or multimolecular species. In some embodiments, the RNA comprises a mixture of mRNAs that encode different peptides or polypeptides.

In some embodiments:
i. each RNA is in a 1:1:1:1 (w/w/w/w) ratio; and/or
ii. each RNA comprises a modified nucleoside in place of each uridine; and/or
iii. each RNA comprises a modified nucleoside in place of each uridine, wherein the modified nucleoside is independently selected from pseudouridine (W), N1-methyl-pseudouridine (mly), and 5-methyl-uridine (m5U); and/or
iv. each RNA comprises the 5′ cap m₂^{7,3′-O}Gppp(m₁^{2′-O})ApG or 3′-O-Me-m⁷G(5′)ppp(5′)G; and/or
v. each RNA comprises a 5′ UTR comprising a nucleotide sequence selected from the group consisting of SEQ ID NOs: 2 and 4, or a nucleotide sequence having at least 99%, 98%, 97%, 96%, 95%, 90%, 85%, or 80% identity to a nucleotide sequence selected from the group consisting of SEQ ID NOs: 2 and 4; and/or
vi. each RNA comprises a 3′ UTR comprising the nucleotide sequence of SEQ ID NO: 6, or a nucleotide sequence having at least 99%, 98%, 97%, 96%, 95%, 90%, 85%, or 80% identity to the nucleotide sequence of SEQ ID NO: 6; and/or
vii. each RNA comprises a poly-A tail, wherein the poly-A tail comprises at least 100 nucleotides, and wherein the poly-A tail comprises or consists of the poly-A tail shown in SEQ ID NO: 27.

In some embodiments, the RNA encodes one or more peptides or polypeptides selected from the group consisting of cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

In some embodiments, the RNA comprises RNA encoding an IL-12sc protein, RNA encoding an IL-15 sushi protein, RNA encoding an IFNα protein, and RNA encoding a GM-CSF protein.

In some embodiments,
i. the RNA encoding IL-12sc protein comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
ii. the RNA encoding an IL-15 sushi protein comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
iii. the RNA encoding an IFNα protein comprises the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
iv. the RNA encoding a GM-CSF protein comprises the nucleotide sequence of SEQ ID NO: 10, or a nucleotide sequence having at least 99%, 98%, 97%, 96%, 95%, 90%, 85%, or 80% identity to the nucleotide sequence of SEQ ID NO: 10.

In some embodiments,
i. the IL-12sc protein comprises the amino acid sequence of SEQ ID NO: 11, or an amino acid sequence having at least 99%, 98%, 97%, 96%, 95%, 90%, 85%, or 80% identity to the amino acid sequence of SEQ ID NO: 11; and/or
ii. the IL-15 sushi protein comprises the amino acid sequence of SEQ ID NO: 21, or an amino acid sequence having at least 99%, 98%, 97%, 96%, 95%, 90%, 85%, or 80% identity to the amino acid sequence of SEQ ID NO: 21; and/or
iii. the IFNα protein comprises the amino acid sequence of SEQ ID NO: 16, or an amino acid sequence having at least 99%, 98%, 97%, 96%, 95%, 90%, 85%, or 80% identity to the amino acid sequence of SEQ ID NO: 16; and/or
iv. the GM-CSF protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

mRNA particles (e.g., LNPs and LPXs) described herein have an average diameter that in some embodiments ranges from about 50 nm to about 1000 n, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 n, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 n, from about 200 nm to about 300 nm, or from about 200 nm to about 250 nm.

RNA particles described herein, e.g. prepared by the methods described herein, exhibit a polydispersity index less than about 0.5, less than about 0.4, less than about 0.3, less than about 0.2, less than about 0.1 or about 0.05 or less. By way of example, the mRNA LNPs can exhibit a polydispersity index in a range of about 0.05 to about 0.2, such as about 0.05 to about 0.1.

In some embodiments of the present disclosure, the mRNA in the mRNA particles (e.g., LNPs and LPXs) described herein is at a concentration from about 0.002 mg/mL to about 5 mg/mL, from about 0.002 mg/mL to about 2 mg/mL, from about 0.005 mg/mL to about 2 mg/mL, from about 0.01 mg/mL to about 1 mg/mL, from about 0.05 mg/mL to about 0.5 mg/mL or from about 0.1 mg/mL to about 0.5 mg/mL. In specific embodiments, the mRNA is at a concentration from about 0.005 mg/mL to about 0.1 mg/mL, from about 0.005 mg/mL to about 0.09 mg/mL, from about 0.005 mg/mL to about 0.08 mg/mL, from about 0.005 mg/mL to about 0.07 mg/mL, from about 0.005 mg/mL to about 0.06 mg/mL, or from about 0.005 mg/mL to about 0.05 mg/mL.

In some embodiments, the N/P value ranges from 2 to 20, 2 to 12, 3 to 10, 3 to 8, or 3 to 7. In some embodiments, the N/P value is about 3, 4 or 5. In some embodiments, the N/P value is from about 3 to about 5. In some embodiments, the N/P value is about 3. In some embodiments, the N/P value is about 4. In some embodiments, the N/P value is about 5.

RNA-lipid particles or simply RNA particles described herein are typically formed from a cationic or cationically ionizable lipid such as DODMA, and one or more non-cationic lipids such as phospholipids (e.g., DOPE), cholesterol or a mixture thereof, and a phosphatidylserine such as DOPS. In some embodiments, the RNA particles are formed from DODMA, DOPE and/or cholesterol, and a phosphatidylserine such as DOPS.

RNA particles described herein can be prepared using different techniques. For example, RNA can be incubated with an ethanolic solution of lipids (comprising cationic or cationically ionizable lipid, optionally additional lipid, and phosphatidylserine) or with liposomes (comprising cationic or cationically ionizable lipid, optionally additional lipid, and phosphatidylserine) which liposomes may be obtained by the ethanol injection method. The RNA may be present in an aqueous solution upon mixing. By acidification of the ethanol, e.g., by using hydrochloric acid or a mixture of acetic acid and hydrochloric acid, it is possible to substantially improve the solubility of the phosphatidylserine.

In some embodiments, RNA particle formation (e.g., by mixing an ethanolic solution of lipids and an aqueous solution of RNA) can be controlled (particularly in terms of resulting particle size) by adding lipids grafted with hydrophilic moieties, e.g., polyethylenglycol, polypeptides or similar moieties. Thus, one or more of the particle-forming lipids or lipid-like materials described herein may comprise one or more of such hydrophilic moieties. Alternatively, particle formation can be controlled by using non-ionic amphiphilic organic compounds, e.g., surfactants like polysorbates (TWEENS), for example, polysorbate 20 (also referred to herein as Tween 20 and Tween20), D-α-tocopherol polyethylene glycol succinate (TPGS), solutols, Myrjs, Brijs in either phase, i.e., the RNA phase and/or the lipid or liposome phase, such as the RNA phase. In different embodiment, the non-ionic amphiphilic organic compound may comprise from about 0.1 mol % to about 50 mol %, from about 1 mol % to about 50 mol %, from about 1 mol % to about 40 mol %, from about 1 mol % to about 30 mol %, from about 1 mol % to about 20 mol %, from about 1 mol % to about 15 mol %, from about 1 mol % to about 10 mol %, from about 1 mol % to about 7.5 mol %, or from about 2 mol % to about 5 mol % of the total lipid (including lipid-like material, e.g., non-ionic amphiphilic organic compound, e.g., surfactant) present in the mixture.

For example, particles may be obtained by preparing an aqueous solution containing RNA, a surfactant such as polysorbate 20 (Tween 20) and a buffer (e.g., citrate buffer, 100 mM, pH 4) and a lipid solution in acidified ethanol, and mixing the solutions. The RNA particle solution may be dialysed, e.g., against buffer with a pH>5.5.

Alternatively, particles may be obtained by preparing a lipid solution in acidified ethanol and injecting the lipid solution into water, such as under stirring of the water (e.g., at a stirring speed of 250 rpm) so as to form liposomes. The liposome solution may be dialysed, e.g., against an acidic aqueous solution. Liposomes may be mixed with an aqueous solution containing RNA, a surfactant such as Tween 20 and a buffer (e.g., citrate buffer, 100 mM, pH 4) so as to form RNA particles.

In some embodiments, an RNA particle described herein is obtainable by a method comprising the steps:
(i) preparing an acidified lipid solution in an organic solvent;
(ii) adding the acidified lipid solution to an aqueous phase to produce a liposome colloid; and
(iii) adding the liposome colloid to an aqueous solution comprising RNA and a non-ionic amphiphilic organic compound to produce the RNA lipid particle. In some embodiments, the particle is a lipoplex particle (LPX).

In some embodiments, an RNA particle described herein is obtainable by a method comprising the steps:
(i) preparing an acidified lipid solution in an organic solvent; and
(ii) adding the acidified lipid solution to an aqueous solution comprising RNA and a non-ionic amphiphilic organic compound to produce the RNA lipid particle. In some embodiments, the particle is a lipid nanoparticle (LNP).

In some embodiments, the organic solvent comprises an alcohol. In some embodiments, the alcohol is ethanol.

In some embodiments, the non-ionic amphiphilic organic compound comprises at least about 0.1 mol % of the total lipid. In some embodiments, the non-ionic amphiphilic organic compound comprises at least about 0.5 mol % of the total lipid. In some embodiments, the non-ionic amphiphilic organic compound comprises at least about 1 mol % of the total lipid. In some embodiments, the non-ionic amphiphilic organic compound comprises at least about 2 mol % of the total lipid. In some embodiments, the non-ionic amphiphilic organic compound comprises at least about 3 mol % of the total lipid. In some embodiments, the non-ionic amphiphilic organic compound comprises at least about 4 mol % of the total lipid. In some embodiments, the non-ionic amphiphilic organic compound comprises at least about 5 mol % of the total lipid. In some embodiments, the non-ionic amphiphilic organic compound comprises at least about 6 mol % of the total lipid. In some embodiments, the non-ionic amphiphilic organic compound comprises at least about 7 mol % of the total lipid. In some embodiments, the non-ionic amphiphilic organic compound comprises at least about 8 mol % of the total lipid. In some embodiments, the non-ionic amphiphilic organic compound comprises at least about 9 mol % of the total lipid. In some embodiments, the non-ionic amphiphilic organic compound comprises at least about 10 mol % of the total lipid. In some embodiments, the non-ionic amphiphilic organic compound comprises at least about 15 mol % of the total lipid. In some embodiments, the non-ionic amphiphilic organic compound comprises at least about 20 mol % of the total lipid.

In some embodiments, the non-ionic amphiphilic organic compound comprises from about 0.1 mol % to about 40 mol % of the total lipid. In some embodiments, the non-ionic amphiphilic organic compound comprises from about 0.1 mol % to about 35 mol % of the total lipid. In some embodiments, the non-ionic amphiphilic organic compound comprises from about 0.1 mol % to about 30 mol % of the total lipid. In some embodiments, the non-ionic amphiphilic organic compound comprises from about 0.5 mol % to about 30 mol % of the total lipid. In some embodiments, the non-ionic amphiphilic organic compound comprises from about 1 mol % to about 30 mol % of the total lipid. In some embodiments, the non-ionic amphiphilic organic compound comprises from about 5 mol % to about 30 mol % of the total lipid. In some embodiments, the non-ionic amphiphilic organic compound comprises from about 5 mol % to about 25 mol % of the total lipid. In some embodiments, the non-ionic amphiphilic organic compound comprises from about 10 mol % to about 25 mol % of the total lipid. In some embodiments, the non-ionic amphiphilic organic compound comprises from about 15 mol % to about 25 mol % of the total lipid. In some embodiments, the non-ionic amphiphilic organic compound comprises about 20 mol % of the total lipid.

### Compositions Comprising RNA Particles

In some aspects, the invention provides a composition comprising one or more of the RNA particles described herein. If the composition comprises more than one particle described herein the composition of the particles may be identical or different, e.g., with respect to lipid or lipid-like material and/or RNA.

In some embodiments, a composition is an aqueous composition. In some embodiments, a composition is a pharmaceutical composition.

In some embodiments, a population of one or more RNA particles comprises more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles. In some embodiments, the RNA particles are present (e.g. in a composition) or used (e.g., in a treatment method or process for preparing a medicament) in a pharmaceutically effective amount. In some embodiments, a population of more than one RNA particles comprises a population of RNA particles that comprise the same lipid or lipid-like material and/or RNA. In some embodiments, a population of more than one RNA particles comprises a population of RNA particles that comprise the same lipid or lipid-like material and/or RNA in the same amount(s). In some embodiments, a population of more than one RNA particles comprises a population of RNA particles that are identical with respect to lipid or lipid-like material and/or RNA.

The term “plurality of RNA particles” refers to a population of RNA particles that comprises more than one species within the genus of a given embodiment or claim; e.g., at least 2, at least 3, at least 4, at least 5, at least 6, at least 7, at least 8, at least 9, or at least 10 species, and, e.g., up to 100, up to 50, up to 30, up to 20, or up to 15 species. In some embodiments, a plurality of RNA particles comprises at least 2, at least 3, at least 4, at least 5, at least 6, at least 7, at least 8, at least 9, at least 10 species, at least 50 species, or at least 100 species.

In some embodiments, the composition described herein is a liquid or a solid, a solid refers to a frozen form or a lyophilized form or a spray-dried form. In a preferred embodiment, the composition is a liquid.

According to the present disclosure, the compositions described herein may comprise salts such as sodium chloride. Without wishing to be bound by theory, sodium chloride functions as an ionic osmolality agent for preconditioning RNA prior to mixing with lipids. In some embodiments, the compositions described herein may comprise alternative organic or inorganic salts. Alternative salts include, without limitation, potassium chloride, dipotassium phosphate, monopotassium phosphate, potassium acetate, potassium bicarbonate, potassium sulfate, disodium phosphate, monosodium phosphate, sodium acetate, sodium bicarbonate, sodium sulfate, lithium chloride, magnesium chloride, magnesium phosphate, calcium chloride, and sodium salts of ethylenediaminetetraacetic acid (EDTA).

Generally, compositions for storing RNA particles such as for freezing RNA particles comprise low sodium chloride concentrations, or comprises a low ionic strength. In some embodiments, the sodium chloride is at a concentration from 0 mM to about 50 mM, from 0 mM to about 40 mM, or from about 10 mM to about 50 mM.

According to the present disclosure, the RNA particle compositions described herein have a pH suitable for the stability of the RNA particles and, in particular, for the stability of the RNA. Without wishing to be bound by theory, the use of a buffer system maintains the pH of the particle compositions described herein during manufacturing, storage and use of the compositions. In some embodiments of the present disclosure, the buffer system may comprise a solvent (in particular, water, such as deionized water, in particular water for injection) and a buffering substance. The buffering substance may be selected from 2-[4-(2-hydroxyethyl)piperazin-1-yl]ethanesulfonic acid (HEPES), 2-amino-2-(hydroxymethyl)propane-1,3-diol (Tris), acetate, and histidine. A preferred buffering substance is HEPES.

Compositions described herein may also comprise a cyroprotectant and/or a surfactant as stabilizer to avoid substantial loss of the product quality and, in particular, substantial loss of mRNA activity during storage, freezing, and/or lyophilization, for example to reduce or prevent aggregation, particle collapse, mRNA degradation and/or other types of damage.

In an embodiment, the cryoprotectant is a carbohydrate. The term “carbohydrate”, as used herein, refers to and encompasses monosaccharides, disaccharides, trisaccharides, oligosaccharides and polysaccharides.

In an embodiment, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In an embodiment, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like.

The term “trisaccharide” means three sugars linked together to form one molecule. Examples of a trisaccharides include raffinose and melezitose.

In an embodiment, the cryoprotectant is an oligosaccharide. The term “oligosaccharide”, as used herein refers to a compound or a chemical moiety formed by 3 to about 15, such as 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In an embodiment, the cryoprotectant is a cyclic oligosaccharide. The term “cyclic oligosaccharide”, as used herein refers to a compound or a chemical moiety formed by 3 to about 15, such as 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as α cyclodextrin, β cyclodextrin, or γ cyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, RNA particle compositions may include sucrose. Without wishing to be bound by theory, sucrose functions to promote cryoprotection of the compositions, thereby preventing RNA (especially mRNA) particle aggregation and maintaining chemical and physical stability of the composition. In some embodiments, RNA particle compositions may include alternative cryoprotectants to sucrose. Alternative stabilizers include, without limitation, trehalose and glucose. In a specific embodiment, an alternative stabilizer to sucrose is trehalose or a mixture of sucrose and trehalose.

A preferred cryoprotectant is selected from the group consisting of sucrose, trehalose, glucose, and a combination thereof, such as a combination of sucrose and trehalose. In a preferred embodiment, the cryoprotectant is sucrose.

In some embodiments, the RNA particles and/or RNA particle compositions described herein comprise a non-ionic amphiphilic organic compound. Examples of such non-ionic amphiphilic organic compounds are described herein. In some embodiments, presence of the non-ionic amphiphilic organic compound stabilizes particles and prevents off-target effects.

Any suitable non-ionic amphiphilic organic compound or compounds which will not adversely affect the RNA particles or their constituents may be used, either low molar mass or polymeric. These compounds may be surfactants.

In some embodiments, the non-ionic amphiphilic organic compound comprises a surfactant. In some embodiments, the non-ionic amphiphilic organic compound comprises a poly(ethylene glycol) (PEG) surfactant. In some embodiments, the non-ionic amphiphilic organic compound comprises a poly(ethylene glycol) (PEG) chain linked to a single hydrophobic chain. In some embodiments, the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof.

In some embodiments, the non-ionic amphiphilic organic compound comprises a polyoxyethylene sorbitan fatty acid ester. In some embodiments, the non-ionic amphiphilic organic compound comprises a polysorbate. In some embodiments, the non-ionic amphiphilic organic compound comprises polysorbate 20.

The non-ionic amphiphilic organic compound may, in particular, be selected from
polyoxyethylene fatty acid esters that include polyoxyethylene stearic acid esters, such as the MYRJ series. Particular compounds in the MYRJ series are, e.g., MYRJ 53 and PEG-40-stearate available as MYRJ52;
sorbitan derivatives that include polyoxyethylene sorbitan fatty acid esters (polysorbates) (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens);
polyoxyethylene-polyoxypropylene co-polymers and block co-polymers or poloxamers, e.g., Pluronic F127 or Pluronic F68;
polyoxyethylene alkyl ethers, e.g., such as polyoxyethylene glycol ethers of C12-C18 alcohols, e.g., polyoxyl 10- or 20-cetyl ether or polyoxyl 23-lauryl ether, or 20-oleyl ether, or polyoxyl 10-, 20- or 100-stearyl ether, as known and commercially available as the BRIJ series. Particularly useful products from the BRIJ series are BRIJ58; BRIJ76; BRIJ78; BRIJ35, i.e. polyoxyl 23 lauryl ether, and BRIJ 98, i.e., polyoxyl 20 oleyl ether;
water-soluble tocopheryl PEG succinic acid esters, e.g, TPGS;
polyoxyethylene mono esters of a saturated C10 to C22, such as C18 substituted e.g. hydroxy fatty acid; e.g. 12 hydroxy stearic acid PEG ester, e.g. of PEG about e.g. 600-900 e.g. 660 Daltons MW, e.g. SOLUTOL.

Embodiments of non-ionic amphiphilic organic compound (e.g., surfactants) include polysorbates (TWEENS) such as polysorbate 20 (Tween 20), polysorbate 40, polysorbate 60, polysorbate 80, D-α-tocopherol polyethylene glycol succinate (TPGS), solutols, Myrjs, Brijs. In some embodiments, the surfactant is polysorbate 20.

The term “polysorbate 20” or “polyoxyethylene (20) sorbitan monolaurate” refers to a non-ionic polysorbate-type surfactant formed by the ethoxylation of sorbitan for the addition of lauric acid. The ethoxylation process provides the molecule with 20 repeating units of polyethylene glycol. In practice, these are divided into 4 different chains, which leads to a commercial product that contains a range of chemical entities. (CAS number 9005-64-5). In some embodiments, polysorbate 20 is a mixture of laurate partial esters of sorbitol and sorbitol anhydrides condensed with approximately 20 mole of ethylene oxide for each mole of sorbitol and its mono- and dianhydrides.

The structure of polysorbate 20 may be represented as follows:

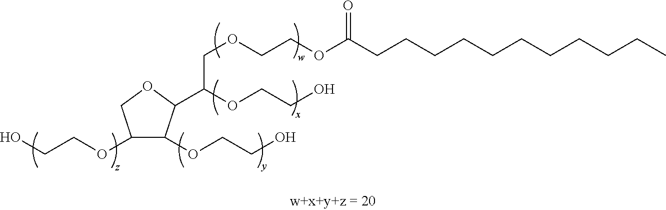

In some embodiments, the non-ionic amphiphilic organic compound comprises at least about 0.1 mol % of the total lipid present in the composition. In some embodiments, the non-ionic amphiphilic organic compound comprises at least about 0.5 mol % of the total lipid present in the composition. In some embodiments, the non-ionic amphiphilic organic compound comprises at least about 1 mol % of the total lipid present in the composition. In some embodiments, the non-ionic amphiphilic organic compound comprises at least about 2 mol % of the total lipid present in the composition. In some embodiments, the non-ionic amphiphilic organic compound comprises at least about 3 mol % of the total lipid present in the composition. In some embodiments, the non-ionic amphiphilic organic compound comprises at least about 4 mol % of the total lipid present in the composition. In some embodiments, the non-ionic amphiphilic organic compound comprises at least about 5 mol % of the total lipid present in the composition. In some embodiments, the non-ionic amphiphilic organic compound comprises at least about 6 mol % of the total lipid present in the composition. In some embodiments, the non-ionic amphiphilic organic compound comprises at least about 7 mol % of the total lipid present in the composition. In some embodiments, the non-ionic amphiphilic organic compound comprises at least about 8 mol % of the total lipid present in the composition. In some embodiments, the non-ionic amphiphilic organic compound comprises at least about 9 mol % of the total lipid present in the composition. In some embodiments, the non-ionic amphiphilic organic compound comprises at least about 10 mol % of the total lipid present in the composition. In some embodiments, the non-ionic amphiphilic organic compound comprises at least about 15 mol % of the total lipid present in the composition. In some embodiments, the non-ionic amphiphilic organic compound comprises at least about 20 mol % of the total lipid present in the composition.

In some embodiments, the non-ionic amphiphilic organic compound comprises from about 0.1 mol % to about 40 mol % of the total lipid present in the composition. In some embodiments, the non-ionic amphiphilic organic compound comprises from about 0.1 mol % to about 35 mol % of the total lipid present in the composition. In some embodiments, the non-ionic amphiphilic organic compound comprises from about 0.1 mol % to about 30 mol % of the total lipid present in the composition. In some embodiments, the non-ionic amphiphilic organic compound comprises from about 0.5 mol % to about 30 mol % of the total lipid present in the composition. In some embodiments, the non-ionic amphiphilic organic compound comprises from about 1 mol % to about 30 mol % of the total lipid present in the composition. In some embodiments, the non-ionic amphiphilic organic compound comprises from about 5 mol % to about 30 mol % of the total lipid present in the composition. In some embodiments, the non-ionic amphiphilic organic compound comprises from about 5 mol % to about 25 mol % of the total lipid present in the composition. In some embodiments, the non-ionic amphiphilic organic compound comprises from about 10 mol % to about 25 mol % of the total lipid present in the composition. In some embodiments, the non-ionic amphiphilic organic compound comprises from about 15 mol % to about 25 mol % of the total lipid present in the composition. In some embodiments, the non-ionic amphiphilic organic compound comprises about 20 mol % of the total lipid present in the composition.

In some embodiments, a composition comprises a non-ionic amphiphilic organic compound at a level of at least about 0.15 mM. In some embodiments, a composition comprises a non-ionic amphiphilic organic compound at a level of at least about 0.2 mM. In some embodiments, a composition comprises a non-ionic amphiphilic organic compound at a level of at least about 0.25 mM. In some embodiments, a composition comprises a non-ionic amphiphilic organic compound at a level of at least about 0.3 mM. In some embodiments, a composition comprises a non-ionic amphiphilic organic compound at a level of at least about 0.35 mM. In some embodiments, a composition comprises a non-ionic amphiphilic organic compound at a level of at least about 0.4 mM. In some embodiments, a composition comprises a non-ionic amphiphilic organic compound at a level of at least about 0.45 mM. In some embodiments, a composition comprises a non-ionic amphiphilic organic compound at a level of at least about 0.5 mM. In some embodiments, a composition comprises a non-ionic amphiphilic organic compound at a level of at least about 0.55 mM. In some embodiments, a composition comprises a non-ionic amphiphilic organic compound at a level of at least about 0.6 mM. In some embodiments, a composition comprises a non-ionic amphiphilic organic compound at a level of at least about 0.7 mM. In some embodiments, a composition comprises a non-ionic amphiphilic organic compound at a level of at least about 0.8 mM. In some embodiments, a composition comprises a non-ionic amphiphilic organic compound at a level of at least about 0.9 mM. In some embodiments, a composition comprises a non-ionic amphiphilic organic compound at a level of at least about 1.0 mM. In some embodiments, a composition comprises a non-ionic amphiphilic organic compound at a level of at least about 1.1 mM. In some embodiments, a composition comprises a non-ionic amphiphilic organic compound at a level of at least about 1.2 mM. In some embodiments, a composition comprises a non-ionic amphiphilic organic compound at a level of at least about 1.3 mM. In some embodiments, a composition comprises a non-ionic amphiphilic organic compound at a level of at least about 1.4 mM. In some embodiments, a composition comprises a non-ionic amphiphilic organic compound at a level of at least about 1.5 mM. In some embodiments, a composition comprises a non-ionic amphiphilic organic compound at a level of at least about 2.0 mM.

In some embodiments, a composition comprises a non-ionic amphiphilic organic compound at a level of at least about 2.5 mM. In some embodiments, a composition comprises a non-ionic amphiphilic organic compound at a level of at least about 3.0 mM. In some embodiments, a composition comprises a non-ionic amphiphilic organic compound at a level of at least about 4.0 mM. In some embodiments, a composition comprises a non-ionic amphiphilic organic compound at a level of at least about 5.0 mM. In some embodiments, a composition comprises a non-ionic amphiphilic organic compound at a level of from about 0.15 mM to about 0.3 mM. In some embodiments, a composition comprises a non-ionic amphiphilic organic compound at a level of from about 0.3 mM to about 0.6 mM. In some embodiments, a composition comprises a non-ionic amphiphilic organic compound at a level of from about 0.6 mM to about 0.75 mM. In some embodiments, a composition comprises a non-ionic amphiphilic organic compound at a level of from about 0.75 mM to about 1.0 mM. In some embodiments, a composition comprises a non-ionic amphiphilic organic compound at a level of from about 1.0 mM to about 1.25 mM. In some embodiments, a composition comprises a non-ionic amphiphilic organic compound at a level of from about 1.25 mM to about 1.5 mM. In some embodiments, a composition comprises a non-ionic amphiphilic organic compound at a level of from about 1.75 mM to about 2.0 mM. In some embodiments, a composition comprises a non-ionic amphiphilic organic compound at a level of from about 2.0 mM to about 2.25 mM. In some embodiments, a composition comprises a non-ionic amphiphilic organic compound at a level of from about 2.25 mM to about 2.5 mM. In some embodiments, a composition comprises a non-ionic amphiphilic organic compound at a level of from about 2.5 mM to about 2.75 mM. In some embodiments, a composition comprises a non-ionic amphiphilic organic compound at a level of from about 2.75 mM to about 3.0 mM. In some embodiments, a composition comprises a non-ionic amphiphilic organic compound at a level of from about 3.0 mM to about 3.5 mM. In some embodiments, a composition comprises a non-ionic amphiphilic organic compound at a level of from about 4.0 mM to about 4.5 mM. In some embodiments, a composition comprises a non-ionic amphiphilic organic compound at a level of from about 4.5 mM to about 5.0 mM.

In some embodiments, the molar ratio of cationic or cationically ionizable lipid, phosphatidylserine, additional lipid, and non-ionic amphiphilic organic compound (e.g., the molar ratio of DODMA, DOPS, DOPE, and polysorbate 20) is 2-9.5:0.2-7:0.2-8:0.05-4. In some embodiments, the molar ratio of cationic or cationically ionizable lipid, phosphatidylserine, additional lipid, and non-ionic amphiphilic organic compound is 2-9:0.2-6:0.5-8:0.1-4. In some embodiments, the molar ratio of cationic or cationically ionizable lipid, phosphatidylserine, additional lipid, and non-ionic amphiphilic organic compound is 3-9:0.2-5:0.5-7:0.2-4. In some embodiments, the molar ratio of cationic or cationically ionizable lipid, phosphatidylserine, additional lipid, and non-ionic amphiphilic organic compound is 4-9:0.2-4:0.5-6:0.3-4. In some embodiments, the molar ratio of cationic or cationically ionizable lipid, phosphatidylserine, additional lipid, and non-ionic amphiphilic organic compound is 4-8:0.2-3:0.5-5:0.4-4. In some embodiments, the molar ratio of cationic or cationically ionizable lipid, phosphatidylserine, additional lipid, and non-ionic amphiphilic organic compound is 5-8:0.5-3:0.5-4:0.5-4. In some embodiments, the molar ratio of cationic or cationically ionizable lipid, phosphatidylserine, additional lipid, and non-ionic amphiphilic organic compound is 5-7:0.5-3:0.5-3:0.5-3. In some embodiments, the molar ratio of cationic or cationically ionizable lipid, phosphatidylserine, additional lipid, and non-ionic amphiphilic organic compound is 5-7:0.5-2:0.5-3:0.75-3. In some embodiments, the molar ratio of cationic or cationically ionizable lipid, phosphatidylserine, additional lipid, and non-ionic amphiphilic organic compound is 5-7:0.5-2:0.5-2:1-3. In some embodiments, the molar ratio of cationic or cationically ionizable lipid, phosphatidylserine, additional lipid, and non-ionic amphiphilic organic compound is 5-7:0.5-1.5:0.5-1.5:1.5-2.5. In some embodiments, the molar ratio of cationic or cationically ionizable lipid, phosphatidylserine, additional lipid, and non-ionic amphiphilic organic compound is 5.5-6.5:0.9-1.1:0.9-1.1:1.5-2.5. In some embodiments, the molar ratio of cationic or cationically ionizable lipid, phosphatidylserine, additional lipid, and non-ionic amphiphilic organic compound is about 6:about 1:about 1:about 2. In some embodiments, the molar ratio of cationic or cationically ionizable lipid, phosphatidylserine, additional lipid, and non-ionic amphiphilic organic compound is about 6:about 1:about 1.2-1.8:about 2. In some embodiments, the molar ratio of cationic or cationically ionizable lipid, phosphatidylserine, additional lipid, and non-ionic amphiphilic organic compound is about 6:about 1:about 1.2:about 2. In some embodiments, the molar ratio of cationic or cationically ionizable lipid, phosphatidylserine, additional lipid, and non-ionic amphiphilic organic compound is about 6:about 1:about 1.7:about 2. In some embodiments, the molar ratio of cationic or cationically ionizable lipid, phosphatidylserine, additional lipid, and non-ionic amphiphilic organic compound is about 6:about 1:about 1.8:about 2.

In some embodiments, a composition comprises a non-ionic amphiphilic organic compound (e.g., polysorbate 20) at a level of at least about 0.1 mM. In some embodiments, a composition comprises a non-ionic amphiphilic organic compound at a level of at least about 0.2 mM. In some embodiments, a composition comprises a non-ionic amphiphilic organic compound at a level of at least about 0.3 mM.

In some embodiments, a composition comprises a non-ionic amphiphilic organic compound at a level of at least about 0.4 mM. In some embodiments, a composition comprises a non-ionic amphiphilic organic compound at a level of at least about 0.5 mM. In some embodiments, a composition comprises a non-ionic amphiphilic organic compound at a level of at least about 0.6 mM.

In some embodiments of the present disclosure contemplate the use of a chelating agent in an RNA composition described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure.

Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In some embodiments, the chelating agent is EDTA or a salt of EDTA. In an exemplary embodiment, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In an alternative embodiment, the RNA particle compositions described herein do not comprise a chelating agent.

### Pharmaceutical Compositions

The RNA particle compositions described herein are useful as or for preparing pharmaceutical compositions or medicaments for therapeutic or prophylactic treatments.

The RNA particles described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease by administration of said pharmaceutical composition to a subject. In the context of the present disclosure, the pharmaceutical composition comprises one or more RNA particles, or a plurality of RNA particles, as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The chemokines may be IL-1, IL-2, IL-3, IL-4, IL-5, IL-6, IL-7, IL-8, IL-9, IL-10, IL-12, INFα, INF-γ, GM-CSF, LT-a. Further known adjuvants are aluminum hydroxide, Freund's adjuvant or oil such as Montanide® ISA51. Other suitable adjuvants for use in the present disclosure include lipopeptides, such as Pam3Cys, as well as lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In some embodiments relating to the treatment of a particular disease, the desired reaction may relate to inhibition of the course of the disease. This comprises slowing down the progress of the disease and, in some embodiments, interrupting or reversing the progress of the disease. The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In some embodiments, the pharmaceutical compositions described herein may be administered intravenously, intraarterially, subcutaneously, intradermally, dermally, intranodally, intramuscularly, intratumorally, or peritumorally. In some embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In some embodiments, the pharmaceutical compositions are formulated for systemic administration. In some embodiments, the systemic administration is by intravenous administration. In some embodiments, pharmaceutical compositions comprising RNA particles described herein are formulated for intratumoral administration/are administered intratumorally. In some embodiments, pharmaceutical compositions comprising RNA particles described herein are formulated for peritumoral administration/are administered peritumorally. In some embodiments, the RNA may encode one or more cytokines or cytokine fusions. In some embodiments, the one or more cytokines or cytokine fusions modulate tumor microenvironment. In some embodiments, the one or more cytokines or cytokine fusions have antitumoral activity (e.g., increase an immune response to a tumor).

### Use of Pharmaceutical Compositions

RNA particles described herein may be used in the therapeutic or prophylactic treatment of various diseases, in particular diseases in which provision of a peptide or polypeptide to a subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease (e.g., cancer, infectious diseases) but may or may not have the disease, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In some embodiments of the present disclosure, the “individual” or “subject” is a “patient”.

The term “patient” means an individual or subject for treatment, in particular a diseased individual or subject.

Some embodiments relate to a method for delivering RNA to cells of a subject, the method comprising administering to a subject an RNA particle described herein. Some embodiments relate to a method for delivering RNA to cells of a subject, the method comprising administering to a subject one or more RNA particles, a plurality of RNA particles or a composition described herein.

Some embodiments relate to a method for delivering a therapeutic or prophylactic peptide or polypeptide to a subject, the method comprising administering to a subject an RNA particle described herein, wherein the RNA encodes a therapeutic peptide or polypeptide. Some embodiments relate to a method for delivering a therapeutic or prophylactic peptide or polypeptide to a subject, the method comprising administering to a subject one or more RNA particles, a plurality of RNA particles or a composition described herein, wherein the RNA encodes one or more therapeutic peptides or polypeptides.

Some embodiments relate to a method for treating or preventing a disease in a subject, the method comprising administering to a subject an RNA particle described herein, wherein delivering the RNA to cells of the subject is beneficial in treating or preventing the disease. Some embodiments relate to a method for treating or preventing a disease in a subject, the method comprising administering to a subject one or more RNA particles, a plurality of RNA particles or a composition described herein, wherein delivering the RNA to cells of the subject is beneficial in treating or preventing the disease.

Some embodiments relate to a method for treating or preventing a disease in a subject, the method comprising administering to a subject an RNA particle described herein, wherein the RNA encodes a therapeutic peptide or polypeptide and wherein delivering the therapeutic peptide or polypeptide to the subject is beneficial in treating or preventing the disease. Some embodiments relate to a method for treating or preventing a disease in a subject, the method comprising administering to a subject one or more RNA particles, a plurality of RNA particles or a composition described herein, wherein the RNA encodes a therapeutic peptide or polypeptide and wherein delivering the therapeutic peptide or polypeptide to the subject is beneficial in treating or preventing the disease.

In some embodiments, the one or more RNA particles or the plurality of RNA particles is administered in a pharmaceutically effective amount.

In some embodiments, the composition is administered in a pharmaceutically effective amount.

In some embodiments, administration is by intravenous, intratumoral, or peritumoral injection.

In some embodiments, the subject is a mammal. In some embodiments, the mammal is a human.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments of the disclosure, the aim is to provide one or more cytokines or cytokine fusions which modulate tumor microenvironment to a subject, in particular a subject in need thereof.

In some embodiments of the disclosure, the aim is to provide one or more cytokines or cytokine fusions which have antitumoral activity to a subject, in particular a subject in need thereof.

Some embodiments relate to a method of treating a subject having cancer, e.g., a solid tumor cancer, comprising administering an effective amount of RNA particles (e.g., a pharmaceutical composition comprising RNA particles), wherein the RNA in the particles comprises or consists of RNA encoding an IL-12sc protein, RNA encoding an IL-15 sushi protein, RNA encoding an IFNα protein, and RNA encoding a GM-CSF protein. Particles and particle compositions for use in treating a subject having cancer are contemplated, wherein the RNA in the particles comprises or consists of RNA encoding an IL-12sc protein, RNA encoding an IL-15 sushi protein, RNA encoding an IFNα protein, and RNA encoding a GM-CSF protein. Particles and particle compositions for administering such RNAs are described herein.

In some embodiments, the subject has an advanced-stage, unresectable, or metastatic solid tumor cancer. In some embodiments, the subject is human. In some embodiments, the subject has a metastatic solid tumor. In some embodiments, the subject has an unresectable solid tumor. In some embodiments, the solid tumor cancer is an epithelial tumor, prostate tumor, ovarian tumor, renal cell tumor, gastrointestinal tract tumor, hepatic tumor, colorectal tumor, tumor with vasculature, mesothelioma tumor, pancreatic tumor, breast tumor, sarcoma tumor, lung tumor, colon tumor, melanoma tumor, small cell lung tumor, non-small cell lung cancer, neuroblastoma tumor, testicular tumor, carcinoma tumor, adenocarcinoma tumor, seminoma tumor, retinoblastoma, cutaneous squamous cell carcinoma (CSCC), squamous cell carcinoma for the head and neck (HNSCC), head and neck cancer, osteosarcoma tumor, kidney tumor, thyroid tumor, anaplastic thyroid cancer (ATC), liver tumor, colon tumor, or other solid tumors amenable to intratumoral injection. In some embodiments, the solid tumor cancer is lymphoma. In some embodiments, the lymphoma is Non-Hodgkin lymphoma. In some embodiments, the solid tumor cancer is Hodgkin lymphoma. In some embodiments, the solid tumor cancer is melanoma. In some embodiments, the solid tumor cancer is melanoma, and wherein the melanoma is uveal melanoma or mucosal melanoma. In some embodiments, the solid tumor cancer is melanoma comprising superficial, subcutaneous and/or lymph node metastases amenable for intratumoral injection. In some embodiments, the solid tumor cancer is HNSCC and/or mucosal melanoma with only mucosal sites. In some embodiments, the solid tumor cancer is not melanoma. In some embodiments, the subject has more than one solid tumor. In some embodiments, the solid tumor cancer is stage III, subsets of stage III, stage IV, or subsets of stage IV. In some embodiments, the solid tumor cancer is advanced-stage and unresectable. In some embodiments, the solid tumor cancer has spread from its origin to another site in the subject. In some embodiments, the solid tumor cancer has one or more cutaneous or subcutaneous lesions, optionally wherein the cancer is not a skin cancer. In some embodiments, the solid tumor cancer is stage IIIB, stage IIIC, or stage IV melanoma. In some embodiments, the solid tumor cancer is not melanoma, non-small cell lung cancer, kidney cancer, head and neck cancer, breast cancer, or CSCC. In some embodiments, the solid tumor cancer comprises superficial or subcutaneous lesions and/or metastases.

Some embodiments relate to a method for treating an advanced-stage melanoma comprising administering to a subject having an advanced-stage melanoma an effective amount of RNA particles, wherein the RNA in the RNA particles comprises or consists of RNA encoding an IL-12sc protein, RNA encoding an IL-15 sushi protein, RNA encoding an IFNα protein, and RNA encoding a GM-CSF protein. Particles and particle compositions for use in treating an advanced-stage melanoma are contemplated, wherein the RNA in the particles comprises or consists of RNA encoding an IL-12sc protein, RNA encoding an IL-15 sushi protein, RNA encoding an IFNα protein, and RNA encoding a GM-CSF protein.

In some embodiments, the melanoma comprises a tumor that is suitable for direct intratumoral or peritumoral injection. Particles and particle compositions for administering such RNAs are described herein.

In some embodiments, treating the solid tumor cancer comprises reducing the size of a tumor or preventing cancer metastasis in a subject.

In some embodiments, the RNA particle(s) are administered by intratumoral injection. In some embodiments, the intratumoral injection comprises injection into a single solid tumor. In some embodiments, the RNA particle(s) are administered by peritumoral injection.

As used herein, and as is generally known in the art, a “solid tumor” is a malignant mass of tissue. A “solid tumor cancer” is a cancer that comprises a solid tumor. Exemplary solid tumor cancers are sarcomas, carcinomas, and lymphomas. With respect to lymphoma, a solid tumor may be a mass of lymphoma cells within a lymph node.

As used herein, an “advanced-stage solid tumor cancer” or “advanced solid tumor cancer” comprises a solid tumor cancer whose stage is identified as stage III, subsets of stage III, stage IV, or subsets of stage IV, assessed by a known system, e.g., the tumor, node, and metastasis (TNM) staging system developed by the American Joint Committee on Cancer (AJCC) (see AJCC Cancer Staging Manual, 8^{th} Edition). In some embodiments, the TNM staging system is used for solid tumor cancers other than melanoma. In some embodiments, the cancer is melanoma or advanced melanoma, which comprises stage IIIB, stage IIIC, or stage IV as assessed by the AJCC melanoma staging (edition 8, 2018). Non-limiting descriptions relating to AJCC melanoma staging are provided in Gershenwald J E, Scolyer R A, Hess K R, et al. Melanoma of the skin. In: Amin M B, ed. AJCC Cancer Staging Manual. 8th ed. Chicago, IL:AJCC-Springer; 2017:563-585, the entire contents of which are incorporated herein by reference. In some embodiments, the cancer is a sarcoma, carcinoma, or melanoma, including, for example, breast carcinoma, squamous cell carcinoma (SCC), basal cell carcinoma (BCC), Merkle cell carcinoma (MCC), cutaneous squamous cell carcinoma (CSCC), and squamous cell carcinoma of the head and neck (HNSCC), each of which may be advanced. Similar staging systems exist for all major cancers and are generally based on the clinical and/or pathological details of the tumor and how these factors have been shown to impact survival.

“Tumor” may also be referred to herein as “neoplasm”. For instance, the terms “solid tumor” and “solid neoplasm” are interchangeable.

An “unresectable” (e.g., advanced-stage unresectable) cancer typically cannot be removed with surgery.

A “superficial” (also sometimes referred to as “cutaneous”) lesion or metastasis is a lesion or metastasis that is within the skin or is at the surface of skin. In some embodiments, a superficial lesion or metastasis is within the cutis. In some embodiments, a superficial lesion or metastasis is within the dermis. In some embodiments, a superficial lesion or metastasis is within the epidermis.

A “subcutaneous” lesion or metastasis is under the skin. In some embodiments, a subcutaneous lesion or metastasis is with the subcutis.

In some embodiments, and in the context of a solid tumor cancer, a “tumor lesion” or “lesion” is a solid tumor, e.g., a primary solid tumor or a solid tumor that has arisen from a metastasis from another solid tumor.

A mixture of RNAs (e.g., a mixture of RNAs comprising RNA encoding an IL-12sc protein, RNA encoding an IL-15 sushi protein, RNA encoding an IFNα protein, and RNA encoding a GM-CSF protein) which may be present in the particles and/or particle compositions described herein may comprise the RNAs in about the same amount.

The phrase in “about the same amount” or in “about equal amounts” or the like is meant to encompass situations in which each RNA is in an exact 1:1:1:1 (w/w/w/w) ratio, as well as situations where the RNAs vary by no more than 10% from the stated amount for each RNA. In some embodiments, the RNAs vary by no more than 9%, 8%, 7%, 6%, 5%, 4%, 3%, 2%, 1% or less than 1% from the stated amount for each RNA. In some embodiments, the RNAs vary by no more than 0.9%, 0.8%, 0.7%, 0.6%, 0.5%, 0.4%, 0.3%, 0.2%, or 0.1% from the stated amount for each RNA.

The term “intratumorally,” or “intratumoral” or the like as used herein, means into the tumor.

As used herein, “lymphoma” is a solid tumor cancer derived from lymphocytes. Lymphoma includes Hodgkin and Non-Hodgkin lymphoma. Lymphoma forms solid tumors/neoplasms within lymph nodes, and can also be found in non-lymph node tissues when metastasized.

In some embodiments, (i) the RNA encoding an IL-12sc protein comprises the nucleotide sequence of SEQ ID NO: 7, 14 or 15, or a nucleotide sequence having at least 99%, 98%, 97%, 96%, 95%, 90%, 85%, or 80% identity to the nucleotide sequence of SEQ ID NO: 7, 14 or 15 and/or (ii) the IL-12sc protein comprises the amino acid sequence of SEQ ID NO: 11, or an amino acid sequence having at least 99%, 98%, 97%, 96%, 95%, 90%, 85%, or 80% identity to the amino acid sequence of SEQ ID NO: 11. In some embodiments, the RNA encoding an IL-12sc protein comprises SEQ ID NO: 7.

In some embodiments, (i) the RNA encoding an IL-15 sushi protein comprises the nucleotide sequence of SEQ ID NO: 8 or 23, or a nucleotide sequence having at least 99%, 98%, 97%, 96%, 95%, 90%, 85%, or 80% identity to the nucleotide sequence of SEQ ID NO: 8 or 23 and/or (ii) the IL-15 sushi protein comprises the amino acid sequence of SEQ ID NO: 21, or an amino acid sequence having at least 99%, 98%, 97%, 96%, 95%, 90%, 85%, or 80% identity to the amino acid sequence of SEQ ID NO: 21. In some embodiments, the RNA encoding an IL-15 sushi protein comprises SEQ ID NO: 8.

In some embodiments, (i) the RNA encoding an IFNα protein comprises the nucleotide sequence of SEQ ID NO: 9, 19 or 20, or a nucleotide sequence having at least 99%, 98%, 97%, 96%, 95%, 90%, 85%, or 80% identity to the nucleotide sequence of SEQ ID NO: 9, 19 or 20 and/or (ii) the IFNα protein comprises the amino acid sequence of SEQ ID NO: 16, or an amino acid sequence having at least 99%, 98%, 97%, 96%, 95%, 90%, 85%, or 80% identity to the amino acid sequence of SEQ ID NO: 16. In some embodiments, the RNA encoding an IFNα protein comprises SEQ ID NO: 9.

In some embodiments, (i) the RNA encoding a GM-CSF protein comprises the nucleotide sequence of SEQ ID NO: 10 or 26, or a nucleotide sequence having at least 99%, 98%, 97%, 96%, 95%, 90%, 85%, or 80% identity to the nucleotide sequence of SEQ ID NO: 10 or 26 and/or (ii) the GM-CSF protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24. In some embodiments, the RNA encoding an GM-CSF protein comprises SEQ ID NO: 10.

In some embodiments, the RNA encoding an IL-12sc protein comprises the nucleotide sequence of SEQ ID NO: 7.

In some embodiments, the RNA encoding an IL-15 sushi protein comprises the nucleotide sequence of SEQ ID NO: 8.

In some embodiments, the RNA encoding an IFNα protein comprises the nucleotide sequence of SEQ ID NO: 9.

In some embodiments, the RNA encoding a GM-CSF protein comprises the nucleotide sequence of SEQ ID NO: 10.

In some embodiments, the RNA encoding an IL-12sc protein comprises the nucleotide sequence of SEQ ID NO: 7, the RNA encoding an IL-15 sushi protein comprises the nucleotide sequence of SEQ ID NO: 8; the RNA encoding an IFNα protein comprises the nucleotide sequence of SEQ ID NO: 9; and the RNA encoding a GM-CSF protein comprises the nucleotide sequence of SEQ ID NO: 10.

In some embodiments, the RNA encoding an IL-12sc protein comprises the nucleotide sequence of SEQ ID NO: 7, the RNA encoding an IL-15 sushi protein comprises the nucleotide sequence of SEQ ID NO: 8; the RNA encoding an IFNα protein comprises the nucleotide sequence of SEQ ID NO: 9; the RNA encoding a GM-CSF protein comprises the nucleotide sequence of SEQ ID NO: 10 and each RNA comprises a 5′ cap comprising m₂^{7,3′-O}Gppp(m₁^{2′-O})ApG or 3′-O-Me-m⁷G(5′)ppp(5′)G. In some embodiments, the 5′ cap comprises m₂^{7,3′-O}Gppp(m₁^{2′-O})ApG.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

DESCRIPTION OF THE SEQUENCES

| | | |
|---|---|---|
| SEQ | | |
| ID | | |
| NO: | Description | SEQUENCE |

5′ UTR

| | | |
|---|---|---|
| 1 | 5′ UTR | GGAATAAACTAGTCTCAACACAACATATACAAAACAAACGAATCTCAAGCAATCAAGCA |
| | (DNA) | TTCTACTTCTATTGCAGCAATTTAAATCATTTCTTTTAAAGCAAAAGCAATTTTCTGAA |
| | | AATTTTCACCATTTACGAACGATAGCC |
| 2 | 5′ UTR | GGAAUAAACUAGUCUCAACACAACAUAUACAAAACAAACGAAUCUCAAGCAAUCAAGCA |
| | (RNA) | UUCUACUUCUAUUGCAGCAAUUUAAAUCAUUUCUUUUAAAGCAAAAGCAAUUUUCUGAA |
| | | AAUUUUCACCAUUUACGAACGAUAGCC |
| 3 | Mod 5′ UTR | AGACGAACTAGTATTCTTCTGGTCCCCACAGACTCAGAGAGAACCCGCCACC |
| | (DNA) | |
| 4 | Mod 5′ UTR | AGACGAACUAGUAUUCUUCUGGUCCCCACAGACUCAGAGAGAACCCGCCACC |
| | (RNA) | |

3′ UTR

| | | |
|---|---|---|
| 5 | 3′ UTR | CTCGAGCTGGTACTGCATGCACGCAATGCTAGCTGCCCCTTTCCCGTCCTGGGTACCCC |
| | (DNA) | GAGTCTCCCCCGACCTCGGGTCCCAGGTATGCTCCCACCTCCACCTGCCCCACTCACCA |
| | | CCTCTGCTAGTTCCAGACACCTCCCAAGCACGCAGCAATGCAGCTCAAAACGCTTAGCC |
| | | TAGCCACACCCCCACGGGAAACAGCAGTGATTAACCTTTAGCAATAAACGAAAGTTTAA |
| | | CTAAGCTATACTAACCCCAGGGTTGGTCAATTTCGTGCCAGCCACACCGAGACCTGGTC |
| | | CAGAGTCGCTAGCCGCGTCGCT |
| 6 | 3′ UTR | CUCGAGCUGGUACUGCAUGCACGCAAUGCUAGCUGCCCCUUUCCCGUCCUGGGUACCCC |
| | (RNA) | GAGUCUCCCCCGACCUCGGGUCCCAGGUAUGCUCCCACCUCCACCUGCCCCACUCACCA |
| | | CCUCUGCUAGUUCCAGACACCUCCCAAGCACGCAGCAAUGCAGCUCAAAACGCUUAGCC |
| | | UAGCCACACCCCCACGGGAAACAGCAGUGAUUAACCUUUAGCAAUAAACGAAAGUUUAA |
| | | CUAAGCUAUACUAACCCCAGGGUUGGUCAAUUUCGUGCCAGCCACACCGAGACCUGGUC |
| | | CAGAGUCGCUAGCCGCGUCGCU |
| 7 | mRNA | AGACGAACYA GYAYYCYYCY GGYCCCCACA GACYCAGAGA GAACCCGCCA |
| | encoding | CCAYGYGYCA CCAGCAGCYG GYGAYCYCAY GGYYCYCCCY GGYAYYYCYG |
| | IL-12sc | GCAYCYCCYC YYGYCGCAAY CYGGGAACYG AAGAAAGACG YGYAYGYCGY |
| | Sequence | YGAGCYCGAC YGGYAYCCGG AYGCGCCYGG CGAGAYGGYG GYGCYGACCY |
| | annotation | GYGACACCCC AGAGGAGGAY GGGAYCACYY GGACCCYYGA YCAAYCCYCC |
| | CAPS: 5′ UTR | GAAGYGCYCG GGYCYGGCAA GACYCYGACC AYACAAGYGA AAGAGYYYGG |
| | CAPS: coding | CGAYGCCGGG CAGYACACYY GCCAYAAGGG CGGAGAAGYY CYGYCCCACY |
| | sequence | CACYGCYGCY GCYGCACAAG AAAGAGGACG GAAYYYGGAG YACCGAYAYC |
| | CAPS: 3′ UTR | CYGAAAGAYC AGAAAGAGCC CAAGAACAAA ACCYYCYYGC GGYGCGAAGC |
| | CAPS: poly A | CAAGAACYAC YCAGGGAGAY YYACYYGYYG GYGGCYGACG ACGAYCAGCA |
| | ′Y′: | CCGAYCYGAC YYYCYCCGYG AAAYCAAGYA GGGGAYCAYC YGACCCYCAA |
| | N1- | GGAGYCACAY GYGGAGCGGC YACYCYGAGC GCYGAACGCG YAAGAGGGGA |
| | methyl- | CAAYAAGGAG YACGAGYAYA GCGYYGAGYG CCAAGAGGAY AGCGCAYGCC |
| | pseudouridine | CCGCCGCCGA AGAAYCAYYG CCCAYYGAAG YGAYGGYGGA YGCYGYACAC |
| | | AAGCYGAAGY AYGAGAACYA CACAAGCYCC YYCYYCAYCC GYGACAYCAY |
| | | CAAACCAGAY CCYCCYAAGA ACCYCCAGCY YAAACCYCYG AAGAACYCYA |
| | | GACAGGYGGA AGYGYCYYGG GAGYAYCCCG ACACCYGGYC YACACCACAY |
| | | YCCYACYYCA GYCYCACAYY CYGCGYYCAG GYACAGGGCA AGYCCAAAAG |
| | | GGAGAAGAAG GAYCGGGYCY YYACAGAYAA AACAAGYGCC ACCGYYAYAY |
| | | GCCGGAAGAA YGCCYCYAYY YCYGYGCGYG CGCAGGACAG AYACYAYAGC |
| | | AGCYCYYGGA GYGAAYGGGC CAGYGYCCCA YGYYCAGGGY CAYCCGGYGG |
| | | YGGCGGCAGC CCCGGAGGCG GYAGCYCCAG AAAYCYCCCY GYGGCYACAC |
| | | CYGAYCCAGG CAYGYYYCCC YGYYYGCACC AYAGCCAAAA CCYCCYGAGA |
| | | GCAGYCAGCA ACAYGCYCCA GAAAGCYAGA CAAACACYGG AAYYCYACCC |
| | | AYGCACCYCC GAGGAAAYAG AYCACGAGGA YAYCACYAAG GACAAAACAA |
| | | GCACYGYCGA AGCAYGCCYY CCCYYGGAAC YGACAAAGAA CGAGAGYYGC |
| | | CYYAAYYCAA GAGAAACAYC YYYCAYYACA AACGGYAGCY GCYYGGCAAG |
| | | CAGAAAAACA YCYYYYAYGA YGGCCCYYYG YCYGAGCAGY AYYYAYGAGG |
| | | AYCYCAAAAY GYACCAGGYG GAGYYYAAGA CCAYGAAYGC CAAGCYGCYG |
| | | AYGGACCCAA AGAGACAGAY YYYCCYCGAY CAGAAYAYGC YGGCYGYGAY |
| | | YGAYGAACYG AYGCAGGCCY YGAAYYYCAA CAGCGAAACC GYYCCCCAGA |
| | | AAAGCAGYCY YGAAGAACCY GACYYYYAYA AGACCAAGAY CAAACYGYGY |
| | | AYYCYCCYGC AYGCCYYYAG AAYCAGAGCA GYCACYAYAG AYAGAGYGAY |
| | | GYCCYACCYG AAYGCYYCCY GAYGACYCGA GCYGGYACYG CAYGCACGCA |
| | | AYGCYAGCYG CCCCYYYCCC GYCCYGGGYA CCCCGAGYCY CCCCCGACCY |
| | | CGGGYCCCAG GYAYGCYCCC ACCYCCACCY GCCCCACYCA CCACCYCYGC |
| | | YAGYYCCAGA CACCYCCCAA GCACGCAGCA AYGCAGCYCA AAACGCYYAG |
| | | CCYAGCCACA CCCCCACGGG AAACAGCAGY GAYYAACCYY YAGCAAYAAA |
| | | CGAAAGYYYA ACYAAGCYAY ACYAACCCCA GGGYYGGYCA AYYYCGYGCC |
| | | AGCCACACCG AGACCYGGYC CAGAGYCGCY AGCCGCGYCG CYAAAAAAAA |
| | | AAAAAAAAAA AAAAAAAAAA AAGCAYAYGA CYAAAAAAAA AAAAAAAAAA |
| | | AAAAAAAAAA AAAAAAAAAA AAAAAAAAAA AAAAAAAAAA AAAAAAAAAA AA |
| 8 | mRNA | AGACGAACYA GYAYYCYYCY GGYCCCCACA GACYCAGAGA GAACCCGCCA |
| | encoding | CCAYGGCCCC GCGGCGGGCG CGCGGCYGCC GGACCCYCGG YCYCCCGGCG |
| | IL-15 sushi | CYGCYACYGC YGCYGCYGCY CCGGCCGCCG GCGACGCGGG GCAYCACGYG |
| | Sequence | CCCYCCCCCC AYGYCCGYGG AACACGCAGA CAYCYGGGYC AAGAGCYACA |
| | annotation | GCYYGYACYC CAGGGAGCGG YACAYYYGYA ACYCYGGYYY CAAGCGYAAA |
| | CAPS: 5′ UTR | GCCGGCACGY CCAGCCYGAC GGAGYGCGYG YYGAACAAGG CCACGAAYGY |
| | CAPS: coding | CGCCCACYGG ACAACCCCCA GYCYCAAAYG CAYYAGAGAC CCYGCCCYGG |
| | sequence | YYCACCAAAG GCCAGCGCCA CCCGGGGGAG GAYCYGGCGG CGGYGGGYCY |
| | CAPS: 3′ UTR | GGCGGGGGAY CYGGCGGAGG AGGAAGCYYA CAGAACYGGG YGAAYGYAAY |
| | CAPS: poly A | AAGYGAYYYG AAAAAAAYYG AAGAYCYYAY YCAAYCYAYG CAYAYYGAYG |
| | ′Y′: | CYACYYYAYA YACGGAAAGY GAYGYYCACC CCAGYYGCAA AGYAACAGCA |
| | N1- | AYGAAGYGCY YYCYCYYGGA GYYACAAGYY AYYYCACYYG AGYCCGGAGA |
| | methyl- | YGCAAGYAYY CAYGAYACAG YAGAAAAYCY GAYCAYCCYA GCAAACAACA |
| | pseudouridine | GYYYGYCYYC YAAYGGGAAY GYAACAGAAY CYGGAYGCAA AGAAYGYGAG |
| | | GAACYGGAGG AAAAAAAYAY YAAAGAAYYY YYGCAGAGYY YYGYACAYAY |
| | | YGYCCAAAYG YYCAYCAACA CYYCYYGAYG ACYCGAGCYG GYACYGCAYG |
| | | CACGCAAYGC YAGCYGCCCC YYYCCCGYCC YGGGYACCCC GAGYCYCCCC |
| | | CGACCYCGGG YCCCAGGYAY GCYCCCACCY CCACCYGCCC CACYCACCAC |
| | | CYCYGCYAGY YCCAGACACC YCCCAAGCAC GCAGCAAYGC AGCYCAAAAC |
| | | GCYYAGCCYA GCCACACCCC CACGGGAAAC AGCAGYGAYY AACCYYYAGC |
| | | AAYAAACGAA AGYYYAACYA AGCYAYACYA ACCCCAGGGY YGGYCAAYYY |
| | | CGYGCCAGCC ACACCGAGAC CYGGYCCAGA GYCGCYAGCC GCGYCGCYAA |
| | | AAAAAAAAAA AAAAAAAAAA AAAAAAAAGC AYAYGACYAA AAAAAAAAAA |
| | | AAAAAAAAAA AAAAAAAAAA AAAAAAAAAA AAAAAAAAAA AAAAAAAAAA |
| | | AAAAAAAA |
| 9 | mRNA | AGACGAACYA GYAYYCYYCY GGYCCCCACA GACYCAGAGA GAACCCGCCA |
| | encoding | CCAYGGCCCY GACYYYYGCC CYYCYCGYGG CYYYGYYGGY GCYGAGYYGC |
| | IFNa2b | AAAYCYYCCY GYAGYGYCGG AYGYGAYCYG CCYCAAACCC ACAGYCYGGG |
| | Sequence | AYCYAGGAGA ACACYGAYGC YGYYGGCACA GAYGAGGAGA AYYAGCCYCY |
| | annotation | YYYCCYGCCY GAAGGAYAGA CAYGACYYCG GCYYYCCCCA AGAGGAGYYY |
| | CAPS: 5′ UTR | GGCAAYCAGY YCCAGAAAGC GGAAACGAYY CCCGYYCYGC ACGAGAYGAY |
| | CAPS: coding | CCAGCAGAYC YYCAACCYCY YYYCAACCAA AGACAGCYCA GCAGCCYGGG |
| | sequence | AYGAGACACY GCYGGACAAA YYCYACACAG AACYGYAYCA GCAGCYYAAC |
| | CAPS: 3′ UTR | GAYCYGGAGG CAYGCGYGAY CCAAGGGGYY GGYGYGACYG AAACYCCGCY |
| | CAPS: poly A | YAYGAAGGAG GACYCCAYYC YGGCYGYACG GAAGYACYYC CAGAGAAYAA |
| | ′Y′: | CCCYCYAYCY GAAGGAGAAG AAGYACYCAC CAYGYGCYYG GGAAGYCGYG |
| | N1- | AGAGCCGAAA YCAYGAGAYC CYYCAGCCYY AGCACCAAYC YCCAGGAAYC |
| | methyl- | YCYGAGAAGC AAAGAGYGAY GACYCGAGCY GGYACYGCAY GCACGCAAYG |
| | pseudouridine | CYAGCYGCCC CYYYCCCGYC CYGGGYACCC CGAGYCYCCC CCGACCYCGG |
| | | GYCCCAGGYA YGCYCCCACC |
| | | YCCACCYGCC CCACYCACCA CCYCYGCYAG YYCCAGACAC CYCCCAAGCA |
| | | CGCAGCAAYG CAGCYCAAAA CGCYYAGCCY AGCCACACCC CCACGGGAAA |
| | | CAGCAGYGAY YAACCYYYAG CAAYAAACGA AAGYYYAACY AAGCYAYACY |
| | | AACCCCAGGG YYGGYCAAYY YCGYGCCAGC CACACCGAGA CCYGGYCCAG |
| | | AGYCGCYAGC CGCGYCGCYA AAAAAAAAAA AAAAAAAAAA AAAAAAAAAG |
| | | CAYAYGACYA AAAAAAAAAA AAAAAAAAAA AAAAAAAAAA AAAAAAAAAA |
| | | AAAAAAAAAA AAAAAAAAAA AAAAAAAAA |
| 10 | mRNA | AGACGAACYA GYAYYCYYCY GGYCCCCACA GACYCAGAGA GAACCCGCCA |
| | encoding | CCAYGYGGCY CCAGAGCCYG CYGCYCYYGG GCACYGYGGC CYGCYCCAYC |
| | GM-CSF | YCYGCACCCG CCCGCYCGCC CAGCCCCAGC ACGCAGCCCY GGGAGCAYGY |
| | Sequence | GAAYGCCAYC CAGGAGGCCC GGCGYCYGCY GAACCYGAGY AGAGACACYG |
| | annotation | CYGCYGAGAY GAAYGAAACA GYAGAAGYCA YCYCAGAAAY GYYYGACCYC |
| | CAPS: 5′ UTR | CAGGAGCCGA CCYGCCYACA GACCCGCCYG GAGCYGYACA AGCAGGGCCY |
| | CAPS: coding | GCGGGGCAGC CYCACCAAGC YCAAGGGCCC CYYGACCAYG AYGGCCAGCC |
| | sequence | ACYACAAGCA GCACYGCCCY CCAACCCCGG AAACYYCCYG YGCAACCCAG |
| | CAPS: 3′ UTR | AYYAYCACCY YYGAAAGYYY CAAAGAGAAC CYGAAGGACY YYCYGCYYGY |
| | CAPS: poly A | CAYCCCCYYY GACYGCYGGG AGCCAGYCCA GGAGYGAYGA CYCGAGCYGG |
| | ′Y′: | YACYGCAYGC ACGCAAYGCY AGCYGCCCCY YYCCCGYCCY GGGYACCCCG |
| | N1- | AGYCYCCCCC GACCYCGGGY CCCAGGYAYG CYCCCACCYC CACCYGCCCC |
| | methyl- | ACYCACCACC YCYGCYAGYY CCAGACACCY CCCAAGCACG CAGCAAYGCA |
| | pseudouridine | GCYCAAAACG CYYAGCCYAG CCACACCCCC ACGGGAAACA GCAGYGAYYA |
| | | ACCYYYAGCA AYAAACGAAA |
| | | GYYYAACYAA GCYAYACYAA CCCCAGGGYY GGYCAAYYYC GYGCCAGCCA |
| | | CACCGAGACC YGGYCCAGAG YCGCYAGCCG CGYCGCYAAA AAAAAAAAAA |
| | | AAAAAAAAAA AAAAAAAGCA YAYGACYAAA AAAAAAAAAA AAAAAAAAAA |
| | | AAAAAAAAAA AAAAAAAAAA AAAAAAAAAA AAAAAAAAAA AAAAAAA |

IL-12sc

| | | |
|---|---|---|
| 11 | Human IL- | MCHQQLVISWFSLVFLASPLVAIWELKKDVYVVELDWYPDAPGEMVVLTCDTPEEDGIT |
| | 12sc (amino | WTLDQSSEVLGSGKTLTIQVKEFGDAGQYTCHKGGEVLSHSLLLLHKKEDGIWSTDILK |
| | acid) | DQKEPKNKTFLRCEAKNYSGRFTCWWLTTISTDLTFSVKSSRGSSDPQGVTCGAATLSA |
| | | ERVRGDNKEYEYSVECQEDSACPAAEESLPIEVMVDAVHKLKYENYTSSFFIRDIIKPD |
| | | PPKNLQLKPLKNSRQVEVSWEYPDTWSTPHSYFSLTFCVQVQGKSKREKKDRVFTDKTS |
| | | ATVICRKNASISVRAQDRYYSSSWSEWASVPCSGSSGGGGSPGGGSSRNLPVATPDPGM |
| | | FPCLHHSQNLLRAVSNMLQKARQTLEFYPCTSEEIDHEDITKDKTSTVEACLPLELTKN |
| | | ESCLNSRETSFITNGSCLASRKTSEMMALCLSSIYEDLKMYQVEFKTMNAKLLMDPKRQ |
| | | IFLDQNMLAVIDELMQALNENSETVPQKSSLEEPDFYKTKIKLCILLHAFRIRAVTIDR |
| | | VMSYLNAS |
| 12 | Human non- | ATGTGTCACCAGCAGTTGGTCATCTCTTGGTTTTCCCTGGTTTTTCTGGCATCTCCCCT |
| | optimized | CGTGGCCATATGGGAACTGAAGAAAGATGTTTATGTCGTAGAATTGGATTGGTATCCGG |
| | IL-12sc | ATGCCCCTGGAGAAATGGTGGTCCTCACCTGTGACACCCCTGAAGAAGATGGTATCACC |
| | (CDS DNA) | TGGACCTTGGACCAGAGCAGTGAGGTCTTAGGCTCTGGCAAAACCCTGACCATCCAAGT |
| | Sequence | CAAAGAGTTTGGAGATGCTGGCCAGTACACCTGTCACAAAGGAGGCGAGGTTCTAAGCC |
| | annotation | ATTCGCTCCTGCTGCTTCACAAAAAGGAAGATGGAATTTGGTCCACTGATATTTTAAAG |
| | CAPS: p40 | GACCAGAAAGAACCCAAAAATAAGACCTTTCTAAGATGCGAGGCCAAGAATTATTCTGG |
| | domain; | ACGTTTCACCTGCTGGTGGCTGACGACAATCAGTACTGATTTGACATTCAGTGTCAAAA |
| | CAPS: | GCAGCAGAGGGTCTTCTGACCCCCAAGGGGTGACGTGCGGAGCTGCTACACTCTCTGCA |
| | linker; | GAGAGAGTCAGAGGGGACAACAAGGAGTATGAGTACTCAGTGGAGTGCCAGGAGGACAG |
| | CAPS: p35. | TGCCTGCCCAGCTGCTGAGGAGAGTCTGCCCATTGAGGTCATGGTGGATGCCGTTCACA |
| | | AGCTCAAGTATGAAAACTACACCAGCAGCTTCTTCATCAGGGACATCATCAAACCTGAC |
| | | CCACCCAAGAACTTGCAGCTGAAGCCATTAAAGAATTCTCGGCAGGTGGAGGTCAGCTG |
| | | GGAGTACCCTGACACCTGGAGTACTCCACATTCCTACTTCTCCCTGACATTCTGCGTTC |
| | | AGGTCCAGGGCAAGAGCAAGAGAGAAAAGAAAGATAGAGTCTTCACGGACAAGACCTCA |
| | | GCCACGGTCATCTGCCGCAAAAATGCCAGCATTAGCGTGCGGGCCCAGGACCGCTACTA |
| | | TAGCTCATCTTGGAGCGAATGGGCATCTGTGCCCTGCAGTGGCTCTAGCGGAGGGGGAG |
| | | GCTCTCCTGGCGGGGGATCTAGCAGAAACCTCCCCGTGGCCACTCCAGACCCAGGAATG |
| | | TTCCCATGCCTTCACCACTCCCAAAACCTGCTGAGGGCCGTCAGCAACATGCTCCAGAA |
| | | GGCCAGACAAACTCTAGAATTTTACCCTTGCACTTCTGAGGAAATTGATCATGAAGATA |
| | | TCACAAAAGATAAAACCAGCACAGTGGAGGCCTGTTTACCATTGGAATTAACCAAGAAT |
| | | GAGAGTTGCCTAAATTCCAGAGAGACCTCTTTCATAACTAATGGGAGTTGCCTGGCCTC |
| | | CAGAAAGACCTCTTTTATGATGGCCCTGTGCCTTAGTAGTATTTATGAAGACTTGAAGA |
| | | TGTACCAGGTGGAGTTCAAGACCATGAATGCAAAGCTTCTGATGGATCCTAAGAGGCAG |
| | | ATCTTTCTAGATCAAAACATGCTGGCAGTTATTGATGAGCTGATGCAGGCCCTGAATTT |
| | | CAACAGTGAGACTGTGCCACAAAAATCCTCCCTTGAAGAACCGGATTTTTATAAAACTA |
| | | AAATCAAGCTCTGCATACTTCTTCATGCTTTCAGAATTCGGGCAGTGACTATTGATAGA |
| | | GTGATGAGCTATCTGAATGCTTCCTGATGA |
| 13 | Human | ATGTGTCACCAGCAGCTGGTGATCTCATGGTTCTCCCTGGTATTTCTGGCATCTCCTCT |
| | optimized | TGTCGCAATCTGGGAACTGAAGAAAGACGTGTATGTCGTTGAGCTCGACTGGTATCCGG |
| | IL-12sc | ATGCGCCTGGCGAGATGGTGGTGCTGACCTGTGACACCCCAGAGGAGGATGGGATCACT |
| | (CDS DNA) | TGGACCCTTGATCAATCCTCCGAAGTGCTCGGGTCTGGCAAGACTCTGACCATACAAGT |
| | Sequence | GAAAGAGTTTGGCGATGCCGGGCAGTACACTTGCCATAAGGGCGGAGAAGTTCTGTCCC |
| | annotation | ACTCACTGCTGCTGCTGCACAAGAAAGAGGACGGAATTTGGAGTACCGATATCCTGAAA |
| | CAPS: p40 | GATCAGAAAGAGCCCAAGAACAAAACCTTCTTGCGGTGCGAAGCCAAGAACTACTCAGG |
| | domain; | GAGATTTACTTGTTGGTGGCTGACGACGATCAGCACCGATCTGACTTTCTCCGTGAAAT |
| | CAPS: | CAAGTAGGGGATCATCTGACCCTCAAGGAGTCACATGTGGAGCGGCTACTCTGAGCGCT |
| | linker; | GAACGCGTAAGAGGGGACAATAAGGAGTACGAGTATAGCGTTGAGTGCCAAGAGGATAG |
| | CAPS: p35. | CGCATGCCCCGCCGCCGAAGAATCATTGCCCATTGAAGTGATGGTGGATGCTGTACACA |
| | | AGCTGAAGTATGAGAACTACACAAGCTCCTTCTTCATCCGTGACATCATCAAACCAGAT |
| | | CCTCCTAAGAACCTCCAGCTTAAACCTCTGAAGAACTCTAGACAGGTGGAAGTGTCTTG |
| | | GGAGTATCCCGACACCTGGTCTACACCACATTCCTACTTCAGTCTCACATTCTGCGTTC |
| | | AGGTACAGGGCAAGTCCAAAAGGGAGAAGAAGGATCGGGTCTTTACAGATAAAACAAGT |
| | | GCCACCGTTATATGCCGGAAGAATGCCTCTATTTCTGTGCGTGCGCAGGACAGATACTA |
| | | TAGCAGCTCTTGGAGTGAATGGGCCAGTGTCCCATGTTCAGGGTCATCCGGTGGTGGCG |
| | | GCAGCCCCGGAGGCGGTAGCTCCAGAAATCTCCCTGTGGCTACACCTGATCCAGGCATG |
| | | TTTCCCTGTTTGCACCATAGCCAAAACCTCCTGAGAGCAGTCAGCAACATGCTCCAGAA |
| | | AGCTAGACAAACACTGGAATTCTACCCATGCACCTCCGAGGAAATAGATCACGAGGATA |
| | | TCACTAAGGACAAAACAAGCACTGTCGAAGCATGCCTTCCCTTGGAACTGACAAAGAAC |
| | | GAGAGTTGCCTTAATTCAAGAGAAACATCTTTCATTACAAACGGTAGCTGCTTGGCAAG |
| | | CAGAAAAACATCTTTTATGATGGCCCTTTGTCTGAGCAGTATTTATGAGGATCTCAAAA |
| | | TGTACCAGGTGGAGTTTAAGACCATGAATGCCAAGCTGCTGATGGACCCAAAGAGACAG |
| | | ATTTTCCTCGATCAGAATATGCTGGCTGTGATTGATGAACTGATGCAGGCCTTGAATTT |
| | | CAACAGCGAAACCGTTCCCCAGAAAAGCAGTCTTGAAGAACCTGACTTTTATAAGACCA |
| | | AGATCAAACTGTGTATTCTCCTGCATGCCTTTAGAATCAGAGCAGTCACTATAGATAGA |
| | | GTGATGTCCTACCTGAATGCTTCCTGATGA |
| 14 | Human non- | AUGUGUCACCAGCAGUUGGUCAUCUCUUGGUUUUCCCUGGUUUUUCUGGCAUCUCCCCU |
| | optimized | CGUGGCCAUAUGGGAACUGAAGAAAGAUGUUUAUGUCGUAGAAUUGGAUUGGUAUCCGG |
| | IL-12sc | AUGCCCCUGGAGAAAUGGUGGUCCUCACCUGUGACACCCCUGAAGAAGAUGGUAUCACC |
| | (RNA | UGGACCUUGGACCAGAGCAGUGAGGUCUUAGGCUCUGGCAAAACCCUGACCAUCCAAGU |
| | encoding CDS) | CAAAGAGUUUGGAGAUGCUGGCCAGUACACCUGUCACAAAGGAGGCGAGGUUCUAAGCC |
| | | AUUCGCUCCUGCUGCUUCACAAAAAGGAAGAUGGAAUUUGGUCCACUGAUAUUUUAAAG |
| | | GACCAGAAAGAACCCAAAAAUAAGACCUUUCUAAGAUGCGAGGCCAAGAAUUAUUCUGG |
| | | ACGUUUCACCUGCUGGUGGCUGACGACAAUCAGUACUGAUUUGACAUUCAGUGUCAAAA |
| | | GCAGCAGAGGGUCUUCUGACCCCCAAGGGGUGACGUGCGGAGCUGCUACACUCUCUGCA |
| | | GAGAGAGUCAGAGGGGACAACAAGGAGUAUGAGUACUCAGUGGAGUGCCAGGAGGACAG |
| | | UGCCUGCCCAGCUGCUGAGGAGAGUCUGCCCAUUGAGGUCAUGGUGGAUGCCGUUCACA |
| | | AGCUCAAGUAUGAAAACUACACCAGCAGCUUCUUCAUCAGGGACAUCAUCAAACCUGAC |
| | | CCACCCAAGAACUUGCAGCUGAAGCCAUUAAAGAAUUCUCGGCAGGUGGAGGUCAGCUG |
| | | GGAGUACCCUGACACCUGGAGUACUCCACAUUCCUACUUCUCCCUGACAUUCUGCGUUC |
| | | AGGUCCAGGGCAAGAGCAAGAGAGAAAAGAAAGAUAGAGUCUUCACGGACAAGACCUCA |
| | | GCCACGGUCAUCUGCCGCAAAAAUGCCAGCAUUAGCGUGCGGGCCCAGGACCGCUACUA |
| | | UAGCUCAUCUUGGAGCGAAUGGGCAUCUGUGCCCUGCAGUGGCUCUAGCGGAGGGGGAG |
| | | GCUCUCCUGGCGGGGGAUCUAGCAGAAACCUCCCCGUGGCCACUCCAGACCCAGGAAUG |
| | | UUCCCAUGCCUUCACCACUCCCAAAACCUGCUGAGGGCCGUCAGCAACAUGCUCCAGAA |
| | | GGCCAGACAAACUCUAGAAUUUUACCCUUGCACUUCUGAGGAAAUUGAUCAUGAAGAUA |
| | | UCACAAAAGAUAAAACCAGCACAGUGGAGGCCUGUUUACCAUUGGAAUUAACCAAGAAU |
| | | GAGAGUUGCCUAAAUUCCAGAGAGACCUCUUUCAUAACUAAUGGGAGUUGCCUGGCCUC |
| | | CAGAAAGACCUCUUUUAUGAUGGCCCUGUGCCUUAGUAGUAUUUAUGAAGACUUGAAGA |
| | | UGUACCAGGUGGAGUUCAAGACCAUGAAUGCAAAGCUUCUGAUGGAUCCUAAGAGGCAG |
| | | AUCUUUCUAGAUCAAAACAUGCUGGCAGUUAUUGAUGAGCUGAUGCAGGCCCUGAAUUU |
| | | CAACAGUGAGACUGUGCCACAAAAAUCCUCCCUUGAAGAACCGGAUUUUUAUAAAACUA |
| | | AAAUCAAGCUCUGCAUACUUCUUCAUGCUUUCAGAAUUCGGGCAGUGACUAUUGAUAGA |
| | | GUGAUGAGCUAUCUGAAUGCUUCCUGAUGA |
| 15 | Human | AUGUGUCACCAGCAGCUGGUGAUCUCAUGGUUCUCCCUGGUAUUUCUGGCAUCUCCUCU |
| | optimized | UGUCGCAAUCUGGGAACUGAAGAAAGACGUGUAUGUCGUUGAGCUCGACUGGUAUCCGG |
| | IL-12sc | AUGCGCCUGGCGAGAUGGUGGUGCUGACCUGUGACACCCCAGAGGAGGAUGGGAUCACU |
| | (RNA | UGGACCCUUGAUCAAUCCUCCGAAGUGCUCGGGUCUGGCAAGACUCUGACCAUACAAGU |
| | encoding CDS) | GAAAGAGUUUGGCGAUGCCGGGCAGUACACUUGCCAUAAGGGCGGAGAAGUUCUGUCCC |
| | | ACUCACUGCUGCUGCUGCACAAGAAAGAGGACGGAAUUUGGAGUACCGAUAUCCUGAAA |
| | | GAUCAGAAAGAGCCCAAGAACAAAACCUUCUUGCGGUGCGAAGCCAAGAACUACUCAGG |
| | | GAGAUUUACUUGUUGGUGGCUGACGACGAUCAGCACCGAUCUGACUUUCUCCGUGAAAU |
| | | CAAGUAGGGGAUCAUCUGACCCUCAAGGAGUCACAUGUGGAGCGGCUACUCUGAGCGCU |
| | | GAACGCGUAAGAGGGGACAAUAAGGAGUACGAGUAUAGCGUUGAGUGCCAAGAGGAUAG |
| | | CGCAUGCCCCGCCGCCGAAGAAUCAUUGCCCAUUGAAGUGAUGGUGGAUGCUGUACACA |
| | | AGCUGAAGUAUGAGAACUACACAAGCUCCUUCUUCAUCCGUGACAUCAUCAAACCAGAU |
| | | CCUCCUAAGAACCUCCAGCUUAAACCUCUGAAGAACUCUAGACAGGUGGAAGUGUCUUG |
| | | GGAGUAUCCCGACACCUGGUCUACACCACAUUCCUACUUCAGUCUCACAUUCUGCGUUC |
| | | AGGUACAGGGCAAGUCCAAAAGGGAGAAGAAGGAUCGGGUCUUUACAGAUAAAACAAGU |
| | | GCCACCGUUAUAUGCCGGAAGAAUGCCUCUAUUUCUGUGCGUGCGCAGGACAGAUACUA |
| | | UAGCAGCUCUUGGAGUGAAUGGGCCAGUGUCCCAUGUUCAGGGUCAUCCGGUGGUGGCG |
| | | GCAGCCCCGGAGGCGGUAGCUCCAGAAAUCUCCCUGUGGCUACACCUGAUCCAGGCAUG |
| | | UUUCCCUGUUUGCACCAUAGCCAAAACCUCCUGAGAGCAGUCAGCAACAUGCUCCAGAA |
| | | AGCUAGACAAACACUGGAAUUCUACCCAUGCACCUCCGAGGAAAUAGAUCACGAGGAUA |
| | | UCACUAAGGACAAAACAAGCACUGUCGAAGCAUGCCUUCCCUUGGAACUGACAAAGAAC |
| | | GAGAGUUGCCUUAAUUCAAGAGAAACAUCUUUCAUUACAAACGGUAGCUGCUUGGCAAG |
| | | CAGAAAAACAUCUUUUAUGAUGGCCCUUUGUCUGAGCAGUAUUUAUGAGGAUCUCAAAA |
| | | UGUACCAGGUGGAGUUUAAGACCAUGAAUGCCAAGCUGCUGAUGGACCCAAAGAGACAG |
| | | AUUUUCCUCGAUCAGAAUAUGCUGGCUGUGAUUGAUGAACUGAUGCAGGCCUUGAAUUU |
| | | CAACAGCGAAACCGUUCCCCAGAAAAGCAGUCUUGAAGAACCUGACUUUUAUAAGACCA |
| | | AGAUCAAACUGUGUAUUCUCCUGCAUGCCUUUAGAAUCAGAGCAGUCACUAUAGAUAGA |
| | | GUGAUGUCCUACCUGAAUGCUUCCUGAUGA |

IFNalpha2b (IFNα2b)

| | | |
|---|---|---|
| 16 | Human | MALTFALLVALLVLSCKSSCSVGCDLPQTHSLGSRRTLMLLAOMRRISLFSCLKDRHDE |
| | IFNα2b | GFPQEEFGNQFQKAETIPVLHEMIQQIFNLFSTKDSSAAWDETLLDKFYTELYQQLNDL |
| | (amino acid) | EACVIQGVGVTETPLMKEDSILAVRKYFQRITLYLKEKKYSPCAWEVVRAEIMRSFSLS |
| | | TNLQESLRSKE |
| 17 | Human non- | ATGGCCTTGACCTTTGCTTTACTGGTGGCCCTCCTGGTGCTCAGCTGCAAGTCAAGCTG |
| | optimized | CTCTGTGGGCTGTGATCTGCCTCAAACCCACAGCCTGGGTAGCAGGAGGACCTTGATGC |
| | IFNα2b (CDS | TCCTGGCACAGATGAGGAGAATCTCTCTTTTCTCCTGCTTGAAGGACAGACATGACTTT |
| | DNA) | GGATTTCCCCAGGAGGAGTTTGGCAACCAGTTCCAAAAGGCTGAAACCATCCCTGTCCT |
| | | CCATGAGATGATCCAGCAGATCTTCAACCTTTTCAGCACAAAGGACTCATCTGCTGCTT |
| | | GGGATGAGACCCTCCTAGACAAATTCTACACTGAACTCTACCAGCAGCTGAATGACCTG |
| | | GAAGCCTGTGTGATACAGGGGGTGGGGGTGACAGAGACTCCCCTGATGAAGGAGGACTC |
| | | CATTCTGGCTGTGAGGAAATACTTCCAAAGAATCACTCTCTATCTGAAAGAGAAGAAAT |
| | | ACAGCCCTTGTGCCTGGGAGGTTGTCAGAGCAGAAATCATGAGATCTTTTTCTTTGTCA |
| | | ACAAACTTGCAAGAAAGTTTAAGAAGTAAGGAATGATGA |
| 18 | Human | ATGGCCCTGACTTTTGCCCTTCTCGTGGCTTTGTTGGTGCTGAGTTGCAAATCTTCCTG |
| | optimized | TAGTGTCGGATGTGATCTGCCTCAAACCCACAGTCTGGGATCTAGGAGAACACTGATGC |
| | IFNα2b (CDS | TGTTGGCACAGATGAGGAGAATTAGCCTCTTTTCCTGCCTGAAGGATAGACATGACTTC |
| | DNA) | GGCTTTCCCCAAGAGGAGTTTGGCAATCAGTTCCAGAAAGCGGAAACGATTCCCGTTCT |
| | | GCACGAGATGATCCAGCAGATCTTCAACCTCTTTTCAACCAAAGACAGCTCAGCAGCCT |
| | | GGGATGAGACACTGCTGGACAAATTCTACACAGAACTGTATCAGCAGCTTAACGATCTG |
| | | GAGGCATGCGTGATCCAAGGGGTTGGTGTGACTGAAACTCCGCTTATGAAGGAGGACTC |
| | | CATTCTGGCTGTACGGAAGTACTTCCAGAGAATAACCCTCTATCTGAAGGAGAAGAAGT |
| | | ACTCACCATGTGCTTGGGAAGTCGTGAGAGCCGAAATCATGAGATCCTTCAGCCTTAGC |
| | | ACCAATCTCCAGGAATCTCTGAGAAGCAAAGAGTGATGA |
| 19 | Human non- | AUGGCCUUGACCUUUGCUUUACUGGUGGCCCUCCUGGUGCUCAGCUGCAAGUCAAGCUG |
| | optimized | CUCUGUGGGCUGUGAUCUGCCUCAAACCCACAGCCUGGGUAGCAGGAGGACCUUGAUGC |
| | IFNα2b (RNA | UCCUGGCACAGAUGAGGAGAAUCUCUCUUUUCUCCUGCUUGAAGGACAGACAUGACUUU |
| | encoding | GGAUUUCCCCAGGAGGAGUUUGGCAACCAGUUCCAAAAGGCUGAAACCAUCCCUGUCCU |
| | CDS) | CCAUGAGAUGAUCCAGCAGAUCUUCAACCUUUUCAGCACAAAGGACUCAUCUGCUGCUU |
| | | GGGAUGAGACCCUCCUAGACAAAUUCUACACUGAACUCUACCAGCAGCUGAAUGACCUG |
| | | GAAGCCUGUGUGAUACAGGGGGUGGGGGUGACAGAGACUCCCCUGAUGAAGGAGGACUC |
| | | CAUUCUGGCUGUGAGGAAAUACUUCCAAAGAAUCACUCUCUAUCUGAAAGAGAAGAAAU |
| | | ACAGCCCUUGUGCCUGGGAGGUUGUCAGAGCAGAAAUCAUGAGAUCUUUUUCUUUGUCA |
| | | ACAAACUUGCAAGAAAGUUUAAGAAGUAAGGAAUGAUGA |
| 20 | Human | AUGGCCCUGACUUUUGCCCUUCUCGUGGCUUUGUUGGUGCUGAGUUGCAAAUCUUCCUG |
| | optimized | UAGUGUCGGAUGUGAUCUGCCUCAAACCCACAGUCUGGGAUCUAGGAGAACACUGAUGC |
| | IFNα2b (RNA | UGUUGGCACAGAUGAGGAGAAUUAGCCUCUUUUCCUGCCUGAAGGAUAGACAUGACUUC |
| | encoding | GGCUUUCCCCAAGAGGAGUUUGGCAAUCAGUUCCAGAAAGCGGAAACGAUUCCCGUUCU |
| | CDS) | GCACGAGAUGAUCCAGCAGAUCUUCAACCUCUUUUCAACCAAAGACAGCUCAGCAGCCU |
| | | GGGAUGAGACACUGCUGGACAAAUUCUACACAGAACUGUAUCAGCAGCUUAACGAUCUG |
| | | GAGGCAUGCGUGAUCCAAGGGGUUGGUGUGACUGAAACUCCGCUUAUGAAGGAGGACUC |
| | | CAUUCUGGCUGUACGGAAGUACUUCCAGAGAAUAACCCUCUAUCUGAAGGAGAAGAAGU |
| | | ACUCACCAUGUGCUUGGGAAGUCGUGAGAGCCGAAAUCAUGAGAUCCUUCAGCCUUAGC |
| | | ACCAAUCUCCAGGAAUCUCUGAGAAGCAAAGAGUGAUGA |

IL-15 sushi

| | | |
|---|---|---|
| 21 | Human IL-15 | MAPRRARGCRTLGLPALLLLLLLRPPATRGITCPPPMSVEHADIWVKSYSLYSRERYIC |
| | sushi | NSGFKRKAGTSSLTECVLNKATNVAHWTTPSLKCIRDPALVHQRPAPPGGGSGGGGSGG |
| | (amino | GSGGGGSLONWVNVISDLKKIEDLIQSMHIDATLYTESDVHPSCKVTAMKCFLLELQVI |
| | acid) | SLESGDASIHDTVENLIILANNSLSSNGNVTESGCKECEELEEKNIKEFLQSFVHIVQM |
| | | FINTS |
| 22 | Human IL-15 | ATGGCCCCGCGGCGGGCGCGCGGCTGCCGGACCCTCGGTCTCCCGGCGCTGCTACTGCT |
| | sushi (CDS | GCTGCTGCTCCGGCCGCCGGCGACGCGGGGCATCACGTGCCCTCCCCCCATGTCCGTGG |
| | DNA) | AACACGCAGACATCTGGGTCAAGAGCTACAGCTTGTACTCCAGGGAGCGGTACATTTGT |
| | Sequence | AACTCTGGTTTCAAGCGTAAAGCCGGCACGTCCAGCCTGACGGAGTGCGTGTTGAACAA |
| | annotations | GGCCACGAATGTCGCCCACTGGACAACCCCCAGTCTCAAATGCATTAGAGACCCTGCCC |
| | CAPS: IL-15 | TGGTTCACCAAAGGCCAGCGCCACCCGGGGGAGGATCTGGCGGCGGTGGGTCTGGCGGG |
| | sushi; | GGATCTGGCGGAGGAGGAAGCTTACAGAACTGGGTGAATGTAATAAGTGATTTGAAAAA |
| | CAPS: | AATTGAAGATCTTATTCAATCTATGCATATTGATGCTACTTTATATACGGAAAGTGATG |
| | linker; | TTCACCCCAGTTGCAAAGTAACAGCAATGAAGTGCTTTCTCTTGGAGTTACAAGTTATT |
| | CAPS: | TCACTTGAGTCCGGAGATGCAAGTATTCATGATACAGTAGAAAATCTGATCATCCTAGC |
| | mature IL-15 | AAACAACAGTTTGTCTTCTAATGGGAATGTAACAGAATCTGGATGCAAAGAATGTGAGG |
| | | AACTGGAGGAAAAAAATATTAAAGAATTTTTGCAGAGTTTTGTACATATTGTCCAAATG |
| | | TTCATCAACACTTCTTGATGA |
| 23 | Human IL-15 | AUGGCCCCGCGGCGGGCGCGCGGCUGCCGGACCCUCGGUCUCCCGGCGCUGCUACUGCU |
| | sushi (RNA | GCUGCUGCUCCGGCCGCCGGCGACGCGGGGCAUCACGUGCCCUCCCCCCAUGUCCGUGG |
| | encoding CDS) | AACACGCAGACAUCUGGGUCAAGAGCUACAGCUUGUACUCCAGGGAGCGGUACAUUUGU |
| | | AACUCUGGUUUCAAGCGUAAAGCCGGCACGUCCAGCCUGACGGAGUGCGUGUUGAACAA |
| | | GGCCACGAAUGUCGCCCACUGGACAACCCCCAGUCUCAAAUGCAUUAGAGACCCUGCCC |
| | | UGGUUCACCAAAGGCCAGCGCCACCCGGGGGAGGAUCUGGCGGCGGUGGGUCUGGCGGG |
| | | GGAUCUGGCGGAGGAGGAAGCUUACAGAACUGGGUGAAUGUAAUAAGUGAUUUGAAAAA |
| | | AAUUGAAGAUCUUAUUCAAUCUAUGCAUAUUGAUGCUACUUUAUAUACGGAAAGUGAUG |
| | | UUCACCCCAGUUGCAAAGUAACAGCAAUGAAGUGCUUUCUCUUGGAGUUACAAGUUAUU |
| | | UCACUUGAGUCCGGAGAUGCAAGUAUUCAUGAUACAGUAGAAAAUCUGAUCAUCCUAGC |
| | | AAACAACAGUUUGUCUUCUAAUGGGAAUGUAACAGAAUCUGGAUGCAAAGAAUGUGAGG |
| | | AACUGGAGGAAAAAAAUAUUAAAGAAUUUUUGCAGAGUUUUGUACAUAUUGUCCAAAUG |
| | | UUCAUCAACACUUCUUGAUGA |

GM-CSF

| | | |
|---|---|---|
| 24 | Human GM- | MWLQSLLLLGTVACSISAPARSPSPSTOPWEHVNAIQEARRLLNLSRDTAAEMNETVEV |
| | CSF (amino | ISEMEDLQEPTCLQTRLELYKOGLRGSLTKLKGPLTMMASHYKQHCPPTPETSCATQII |
| | acid) | TFESFKENLKDELLVIPFDCWEPVQE |
| 25 | Human GM- | ATGTGGCTCCAGAGCCTGCTGCTCTTGGGCACTGTGGCCTGCTCCATCTCTGCACCCGC |
| | CSF (CDS DNA) | CCGCTCGCCCAGCCCCAGCACGCAGCCCTGGGAGCATGTGAATGCCATCCAGGAGGCCC |
| | | GGCGTCTGCTGAACCTGAGTAGAGACACTGCTGCTGAGATGAATGAAACAGTAGAAGTC |
| | | ATCTCAGAAATGTTTGACCTCCAGGAGCCGACCTGCCTACAGACCCGCCTGGAGCTGTA |
| | | CAAGCAGGGCCTGCGGGGCAGCCTCACCAAGCTCAAGGGCCCCTTGACCATGATGGCCA |
| | | GCCACTACAAGCAGCACTGCCCTCCAACCCCGGAAACTTCCTGTGCAACCCAGATTATC |
| | | ACCTTTGAAAGTTTCAAAGAGAACCTGAAGGACTTTCTGCTTGTCATCCCCTTTGACTG |
| | | CTGGGAGCCAGTCCAGGAGTGATGA |
| 26 | Human GM- | AUGUGGCUCCAGAGCCUGCUGCUCUUGGGCACUGUGGCCUGCUCCAUCUCUGCACCCGC |
| | CSF (RNA | CCGCUCGCCCAGCCCCAGCACGCAGCCCUGGGAGCAUGUGAAUGCCAUCCAGGAGGCCC |
| | encoding CDS) | GGCGUCUGCUGAACCUGAGUAGAGACACUGCUGCUGAGAUGAAUGAAACAGUAGAAGUC |
| | | AUCUCAGAAAUGUUUGACCUCCAGGAGCCGACCUGCCUACAGACCCGCCUGGAGCUGUA |
| | | CAAGCAGGGCCUGCGGGGCAGCCUCACCAAGCUCAAGGGCCCCUUGACCAUGAUGGCCA |
| | | GCCACUACAAGCAGCACUGCCCUCCAACCCCGGAAACUUCCUGUGCAACCCAGAUUAUC |
| | | ACCUUUGAAAGUUUCAAAGAGAACCUGAAGGACUUUCUGCUUGUCAUCCCCUUUGACUG |
| | | CUGGGAGCCAGUCCAGGAGUGAUGA |
| 27 | Exemplary | AAAAAAAAAAAAAAAAAAAAAAAAAAAAAAGCAUAUGACUAAAAAAAAAAAAAAAAAAA |
| | Poly-A | AAAAAAAAAAAAAAAAAAAAAAAAAAAAAAAAAAAAAAAAAAAAAAAAAAA |

### EXAMPLES

### Example 1: Experimental Part

### Preparation of Phosphatidylserine Lipid Nano Particles (PS-LNPs)

Preparation of Lipid Phase: The calculated lipid solution was prepared and placed in an ultrasonic water bath at 40-45° C. until a clear solution was obtained, while avoiding degradation due to over ultrasonication.

Preparation of Aqueous Phase: Aqueous RNA solution was stored at −20° C. and thawed on an ice bath. In a sterile nucleases-free 5 mL eppendorf tube, the calculated amount of RNA, citrate buffer (100 mM, pH 4), Tween20, and nuclease free water was pipetted. This was then mixed by vortexing gently for 1 second.

Preparation of PS-LNPs by Hand Mixing: The aqueous phase (RNA/citrate buffer/tween20) was withdrawn in a 5 mL syringe equipped with a 27G needle and any air bubbles removed. This was injected quickly into the lipid phase, avoiding the injection of air bubbles. This was gently mixed and incubated 10-15 min at room temperature before dialysis.

Preparation of PS-LNPs by NanoAssemblr Mixing: The aqueous phase (RNA/citrate buffer/tween20) was withdrawn into a 5 mL syringe and any air bubbles removed. The acidified ethanolic lipid solution was withdrawn into a 3 mL syringe and any air bubbles removed. The two phases were combined using the NanoAssemblr with a flow rate of 12 mL/min and mixing ratio of 3:1 (lipid:aqueous). After mixing of lipid and aqueous phases, the resulting PS-LNPs were incubated for 10-15 min at room temperature before dialysis.

Dialysis: An appropriate beaker was filled with the calculated 1× D-PBS (250 mL 1×PBS for 1 mL PS-LNP sample), and placed on a magnetic stirrer. The solution was agitated to ensure fluent movement of the dialysis cassette filled with PS-LNP and allowed to dialyze for 2 h.

The dialyzed PS-LNP were recovered from the dialysis cassettes in a nuclease free eppendorf tube and stored at 2-8° C.

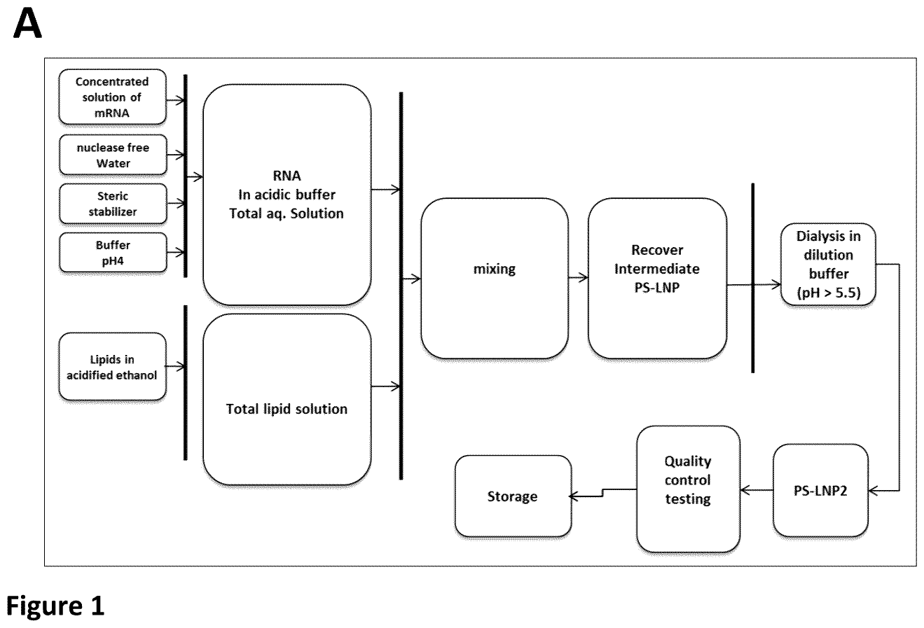

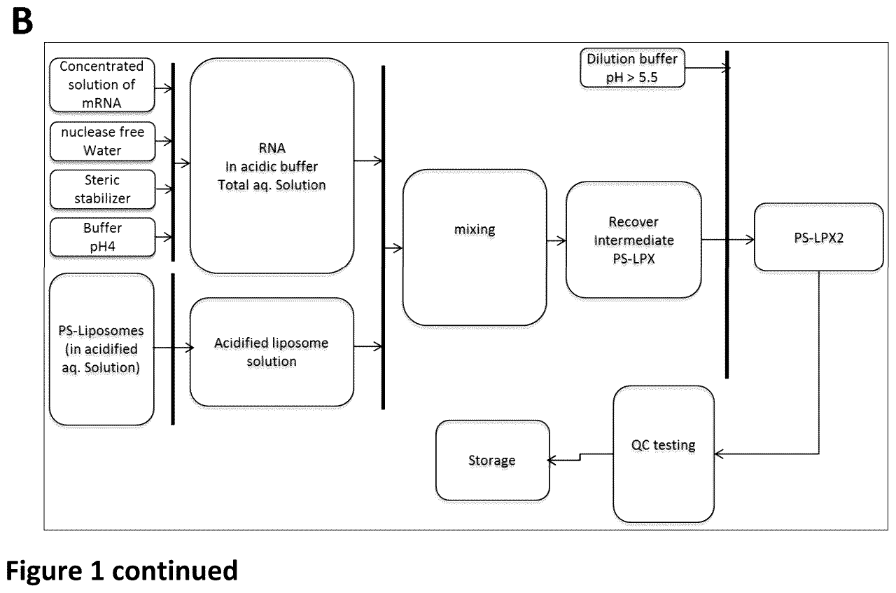

An overview of the PS-LNP Manufacturing Process is shown in FIG. 1A.

### Preparation of Phosphatidylserine Lipoplexes (PS-LPXs)

Prior to preparation of PS-LPXs, PS-liposomes were prepared separately.

### Preparation of DODMA/DOPE/DOPS Liposomes as an Example of PS-Liposomes:

TABLE 1
Lipids required for 60 mM lipid stock solution of PS-Liposomes

| | | |
|---|---|---|
| DODMA lipid liquid | 0.278 | g |
| DOPE lipid powder | 0.056 | g |
| DOPS lipid powder | 0.061 | g |
| HCl_{(aq)} solution 1M | 0.60 | mL |
| Ethanol absolute ad | 10 | mL |

1. To the weighed lipids, absolute ethanol was added until the total volume of the stock solution was 10 mL.
2. The solution was sonicated in a water bath at 40-45° C. for 2-3 minutes.
3. To the lipid/ethanol mixture, the calculated amount of the 1M HCl aqueous solution was added and sonicated in a water bath at 40-45° C. until a clear solution was obtained.
4. 42.50 mL nuclease-free water was transferred into a 100 mL nuclease-free glass beaker.
5. With the aid of a 10 mL syringe equipped with a 20G needle, 7.50 mL of acidified lipid blend ethanol solution was withdrawn and any air bubbles removed.
6. The needle was inserted into the water and 7.50 mL of lipid solution directly injected while stirring at speed of 250 rpm on digital stirrer. The resulting liposomes were stirred for a further 30 min prior to dialysis.

### Dialysis of PS-Liposomes:

The formed liposomes were dialyzed against nuclease-free 5 mM acetic acid (HAC) aqueous solution while constantly stirring (2500 mL of 5 mM acetic acid aqueous solution for each Slide-A-Lyzer™ G2 Dialysis Cassette (3.5 KD MWCO, 15 mL (Thermo Scientific, Catalogue ID 87724) filled with 10 mL PS-liposomes) for 3 hr, and subsequently recovered in a nuclease-free glass bottle. Under Laminar flow clean bench sterile the dialyzed liposomes were filtered using 0.22 μm PES membrane (Merck Millipore Ref number: SLMPL25SS or SLGP033RS depending on volume), then stored at 2-8° C.

### Preparation of Phosphatidylserine Lipoplexes (PS-LPXs):

Preparation of PS-Liposomes Phase: The previously prepared PS-liposomes were diluted with nuclease-free water to adjust the concentration to 3.914 mM DODMA, 0.652 mM DOPE, 0.652 mM DOPS, and 5 mM HAC.

Preparation of RNA Aqueous Phase: Aqueous RNA solution stored at −20° C., was thawed on ice prior to use. In a sterile nuclease-free 5 mL eppendorf tube, the following components were pipetted in a descending order as described in Table 2. The solution was mixed well by pipetting up and down twice while avoiding the formation of air bubbles.

TABLE 2
RNA Aqueous phase mix for PS-LPX formation

| Component | Volume [μl] | Final Concentration | |
|---|---|---|---|
| Nuclease-free water | 910.85 | | |
| RNA (aqueous solution, 2 mg/mL) | 400 | 0.435 | mg/mL |
| Citrate buffer (100 mM, pH 4) | 500 | 27.174 | mM |
| Tween20, 10% (aqueous solution) | 29.15 | 1.304 | mM |

| | |
|---|---|
| Volume (Total) | 1840 |

Preparation of PS-LPXs by Hand Mixing: The aqueous phase (RNA/citrate buffer/tween20) was withdrawn with a 3 mL syringe equipped with a 27G needle and any air bubbles removed. With the syringe needle directly in the lipid phase, the aqueous phase was injected quickly, avoiding the injection of air bubbles. A 1 mL pipette mixed the resulting PS-LPXs by pipetting up and down 3 times which were then incubated for 10-15 min at room temperature prior to dilution with 0.320 mL of 10×PBS and mixed in the same way.

Preparation of PS-LPXs by NanoAssemblr: The aqueous phase (RNA/citrate buffer/tween20) was withdrawn with a 3 mL syringe and any air bubbles removed. In the same way, the PS-Liposomes phase was also withdrawn with a 3 mL syringe and any air bubbles removed. The phases were mixed with the NanoAssemblr through a 1:1 mixing ratio (lipid:aqueous) and flow rate of 12 mL/min and the resulting PS-LPX incubated for 10-15 min at room temperature. Subsequently, with an appropriate pipette, 0.320 mL of 10×PBS was added and mixed by pipetting up and down 3 times.

An overview of the PS-LPX Manufacturing Process is shown in FIG. 1B.

### Physicochemical Characterization:

Particle sizes and polydispersity indices (PDI) were routinely measured by dynamic light scattering with a DynaPro plate reader II from WYATT technology. The samples were diluted with 1× D-PBS buffer to obtain a final mRNA concentration of 2-10 μg/mL. For data analysis, diameter (z-average, calculated from intensity weighted distribution) and PDI ((std. dev./mean)²) of samples are considered.
Zeta potential measurements were performed by using High Resolution Laser Doppler Electrophoretic Mobility Technique using a WALLIS ξ-ZETA POTENTIAL ANALYZER and subsequent data analysed according to Smoluchovski or Huckel theory.
The RNA concentration in Lipid Particles (PS-LNPs and PS-LPXs) was measured through the Ribogreen Assay. This assay also enables the assessment of RNA accessibility in the formulation by comparing the fluorescence signal of the dye in the presence and absence of Triton X100. This is possible as the dye only fluoresces when bound to RNA. The excitation maximum for RiboGreen reagent bound to RNA is ˜500 nm and the emission maximum is ˜525 nm.
The integrity of RNA molecules was tested with an Agilent 2100 Bioanalyzer. The Bioanalyzer operating principle is based upon traditional gel electrophoresis principles, but utilises chip technology. Each chip is composed of several sample wells, gel wells and a well for an external standard (Ladder). The wells are connected to each other by micro-channels which are filled with a polymer and a fluorescence dye. Then, twelve samples, ladder with marker are loaded in each chip. When the wells and the micro-channels are filled the chip is connected via electrodes to a power supply and the RNA molecules are separated according to size, with smaller fragments migrating faster than larger ones. Data is then translated into gel images (bands) and electropherograms (peaks).

### Cryo-Transmission Electron Microscopy Imaging (Cryo-TEM)

Before grid preparation samples were vortexed for 30 sec. Grids were hydrophilized by oxygen plasma (negative surface charge). Each sample was preserved in vitrified ice supported by holey carbon films on 200-mesh copper grids (QuantiFoil® R2/1). Each sample was prepared by applying a 6 μL drop of sample suspension to a cleaned grid, blotting away with filter paper, and immediately proceeding with vitrification in liquid ethane at −180° C. with a Leica EM GP. Grids were stored under liquid Nitrogen until transferred to the electron microscope for imaging. Cryogenic TEM imaging was performed by means of a Zeiss Libra® 120 under liquid N2 cryo conditions on holey carbon-coated copper grids after freezing the solution. The microscope was used at 120 kV acceleration voltage and the images were taken with a Gatan UltraScan® ccd camera. Vitreous ice grids were transferred into the electron microscope using a cryostage that maintains the grids at a temperature below −170° C. Images of each grid were acquired at multiple scales to assess the overall distribution of the specimen.

### Biological Testing:

Administration of PS-LNPs and PS-LPXs into Mice:

Animals were injected with 0.05-0.15 mg/kg doses of mRNA-PS-LNPs and mRNA-PS-LPXs in Dulbecco's Phosphate Buffered Saline (DPBS) by intramuscular (IM, 2×25 μL), intradermal (ID, 2×25 μL), intravenous (IV, 200 μL), subcutaneous (SC, 200 μL), intraperitoneal (IP, 200 μL) or intratumoral (IT, 50 μL) routes using 30G 3/10 cc insulin syringes (BD Biosciences). Intravenous injection was performed through retro-orbital sinus of anesthetized mice.

Measurement of plasma EPO in mice following administration of murine EPO-encoding mRNA formulated PS-LNPs and PS-LPXs: For determining the translation of mRNA encoding murine erythropoietin (mEPO) in-vivo, cap1 structure (CleanCap413, TriLink)- and 1-methylpseudouridine (m1ψ)-containing mRNA formulated with PS-LNPs and PS-LPXs was injected by 4 different routes including intramuscularly (3.0 μg), intraperitoneally (1.0 μg), intravenously (3.0 μg) and subcutaneously (3.0 μg) into mice. Blood was collected at 6, 24 and 48 hours post mRNA injection as described in Mahiny and Karik6 (2016, Methods in Mol Biol 1428: 297) to avoid impact of the sampling on the hematological parameters of the animals.

In brief, eighteen microliter of blood was drawn by puncture of the tail vein, mixed with 2 μL 0.5 M EDTA (pH 7.2) and centrifuged in 20 μL Drummond microcaps glass microcapillary tubes (Sigma-Aldrich). After snapping the microcapillary tubes, the plasma was recovered for the measurement of plasma EPO levels using the mEPO DuoSet ELISA Development kit (R&D Systems, Minneapolis, MN) according to the manufacturer instructions. Briefly, flat-bottom 96-well plates were pre-coated with capture antibody (100 μl/well in PBS) and incubated at room temperature (RT) overnight. The plates were washed with PBS containing 0.05% Tween-20 and incubated with 1% bovine albumin serum (BSA) at RT for 2 hours to prevent non-specific binding of the antibody and washed again. Plasma samples diluted in 1% BSA-PBS using different dilution factors (1:20 for i.m. and s.c., 1:150 for i.v. and i.p.) were added to appropriate wells and the plates were incubated at RT for 2 hours. Following this, anti-EPO monoclonal detection antibody diluted in 1% BSA-PBS (1:180) was added to each well and the plates were incubated at RT for 2 hours. After washing, the plates incubated with horseradish peroxidase (HRP)-conjugated secondary antibody (1:200) at RT for 20 min and then substrate solution (KPL TMB 2-Component Microwell Peroxidase Substrate System) was added to each well (100 μl/well). After 5 min incubation, 2 M sulfuric acid was added (50 μl/well) to stop the reaction and absorbance was measured at 450 nm using an Infinite 200 Pro plate reader (Tecan, Mannedorf, Switzerland).

Detection of luciferase activity in mice following administration of firefly luciferase-encoding mRNA formulated with PS-LNPs and PS-LPXs: For determining the translation of mRNA encoding firefly luciferase in vivo, cap1 structure (CleanCap413, TriLink)- and 1-methylpseudouridine (m1ψ)-containing mRNA formulated with PS-LNPs or PS-LPXs were injected by 4 different routes including intradermally, intramuscularly, intravenously and subcutaneously into mice. In-vivo imaging of luciferase expression was performed at 6, 24, 48, 72 hours and at day 6 post-delivery of PS-LNPs using an IVIS Spectrum In-Vivo Imaging System (PerkinElmer, Rodgau, Germany). To determine the differences in bioluminescence, mice were administered D-luciferin (bioluminescent substrate) at a dose of 150 mg/kg intraperitoneally. Mice were anesthetized after receiving D-luciferin in a chamber replenished with 2.5% isoflurane. After placing the mice on the imaging platform, animals were imaged at 5 minutes after injection of D-luciferin using 1 min exposure time. The acquired signal was within effective detection range (4, 8 or 16 bin) below CCD camera saturation limit.

### Example 2: Formulation Examples

The amount of each lipid (e.g., the amount of cationic or cationically ionizable lipid, phosphatidyl serine, and surfactant) reported to be present in the LNPs and LPXs of the following Formulation Examples is the amount used to produce the respective LNP or LPX. For each formulated LNP and LPX described in the Examples herein, it is believed that the amount of each lipid (e.g., the amount of cationic or cationically ionizable lipid, phosphatidyl serine, and surfactant) that is used to produce a formulation is the same as the amount in the formulation that is produced.

### Formulation Example 1: PS-LNP1

The preparation of PS-LNP1 and the quality control testing were done as described in the experimental part. The lipid composition of PS-LNP1 was DODMA/CHOL/DOPS/Tween20 (57/30/10/3 mol %). The positive/negative (cationic lipid/RNA nucleotide) ratio was 3.

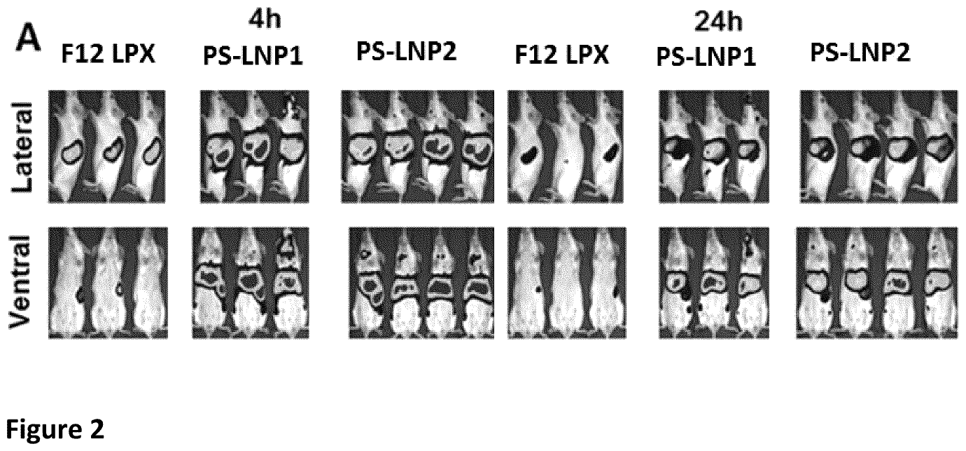

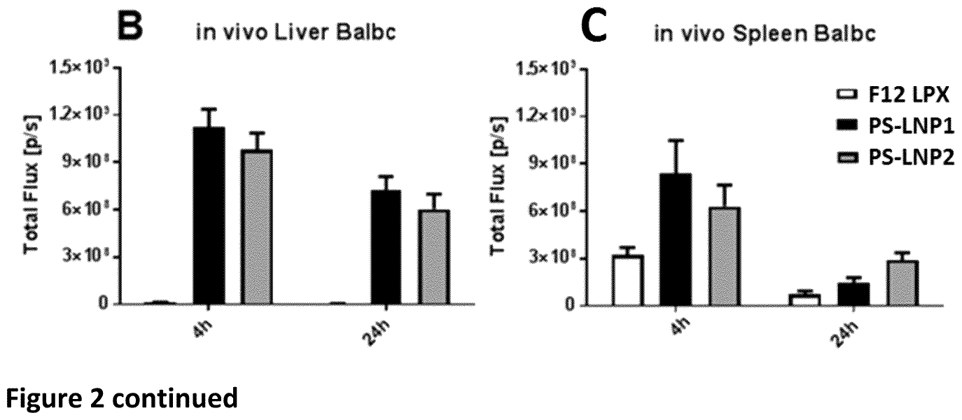

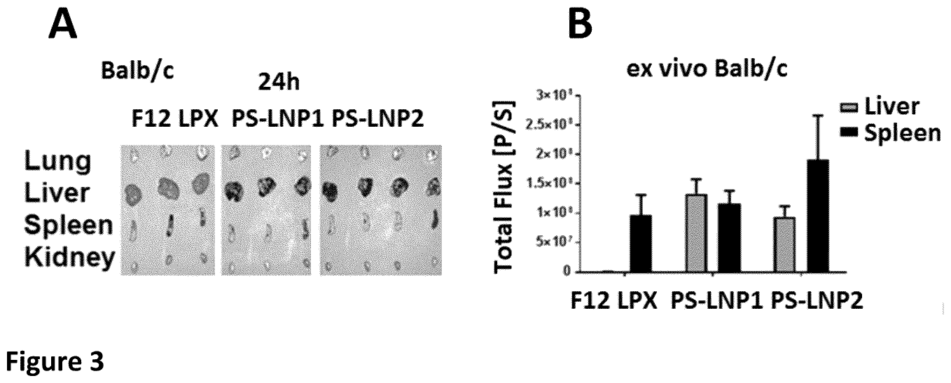

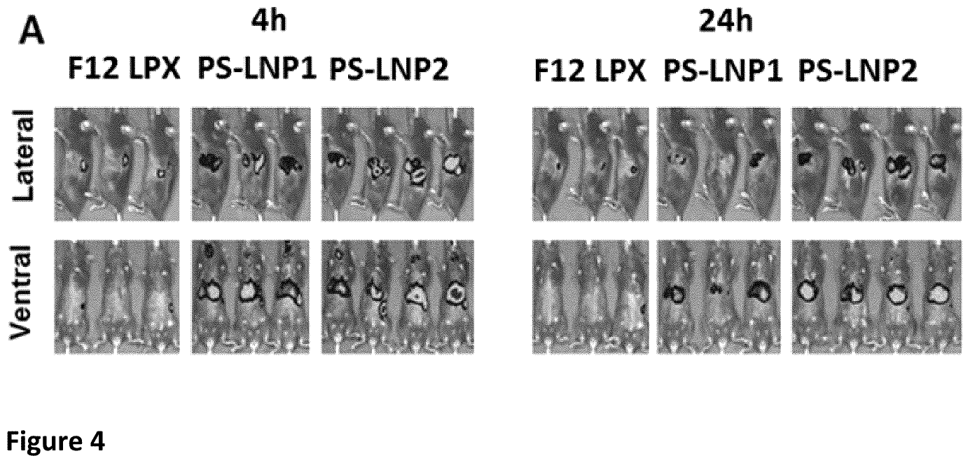

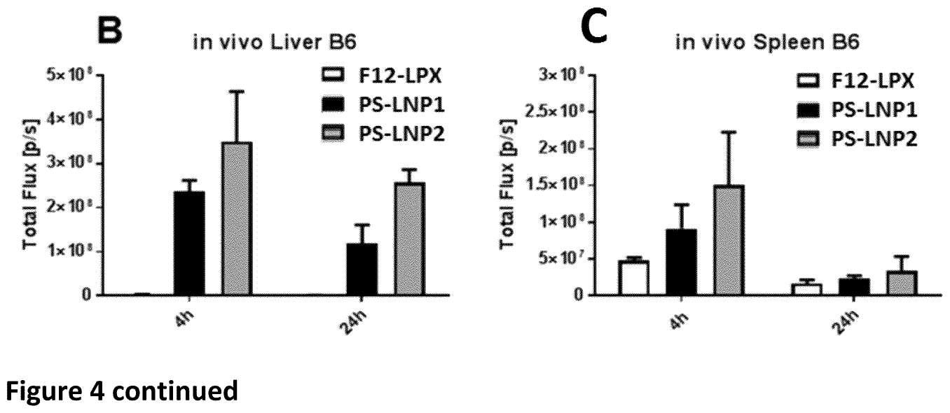

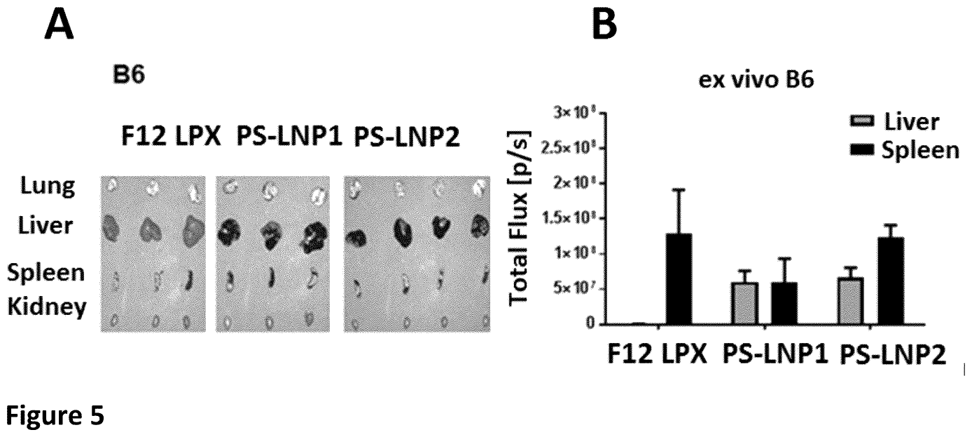

In in vivo experiments (FIGS. 2-5), in vitro transcribed (IVT) firefly luciferase-encoding mRNA was formulated with PS-LNP1 and F12 lipoplex (Kranz et al. 2016, Nature 534: 396, as an internal benchmark). The particle size, PDI, zeta potential, and EE % for PS-LNP1 was 413 nm, 0.20, −3.57 mV, and 86.7, respectively. Each mouse received 200 μL intravenous (IV) injection containing 20 μg of formulated mRNA. Luciferin, the substrate of luciferase was injected intraperitoneally 4 and 24 hours following delivery of the formulated mRNA and luciferase activity detected using an in vivo imaging system (IVIS). The in vivo and ex vivo data of PS-LNP1 demonstrated high spleen and liver tissue transfection, leading to in-vivo highly detectable bioluminescence signal.

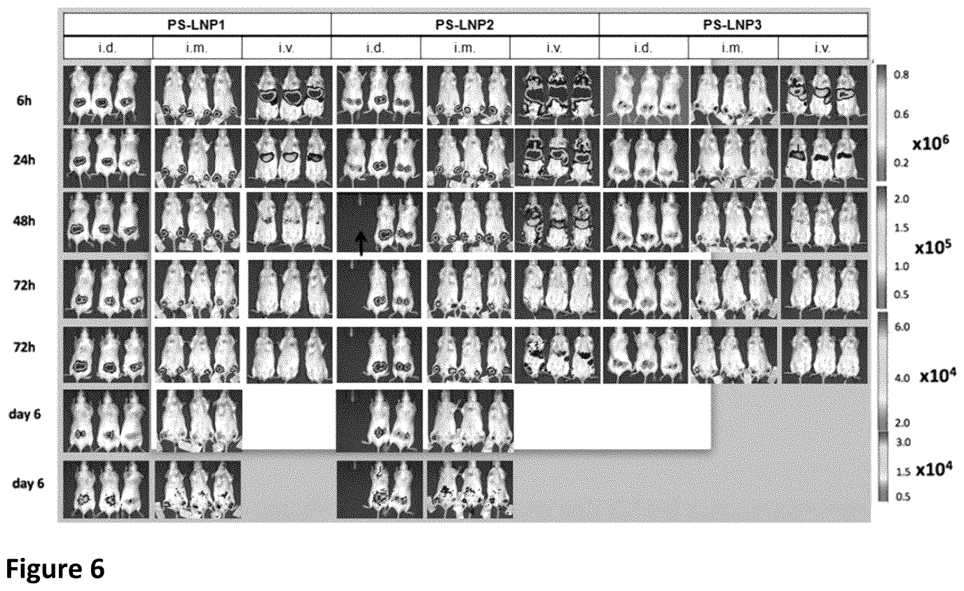

In another in vivo experiment (FIG. 6), PS-LNP1 was formulated with IVT firefly luciferase-encoding mRNA and delivered in mice via diverse application routes. This batch of PS-LNP1 demonstrated a particle size, PDI and zeta potential of 348 nm, 0.34 and −26.29 mV, respectively. Each mouse received 3 μg of formulated RNA by intradermal (ID), intramuscular (IM) or intravenous (IV) routes. The in vivo results show that PS-LNP1 demonstrated high local signal upon i.d. and i.m. administration and spleen and liver tissue transfection upon i.v. application. This in vivo highly detectable bioluminescence signal proves clearly the high efficiency and functionality of PS-LNP1 nanoparticle formulation.

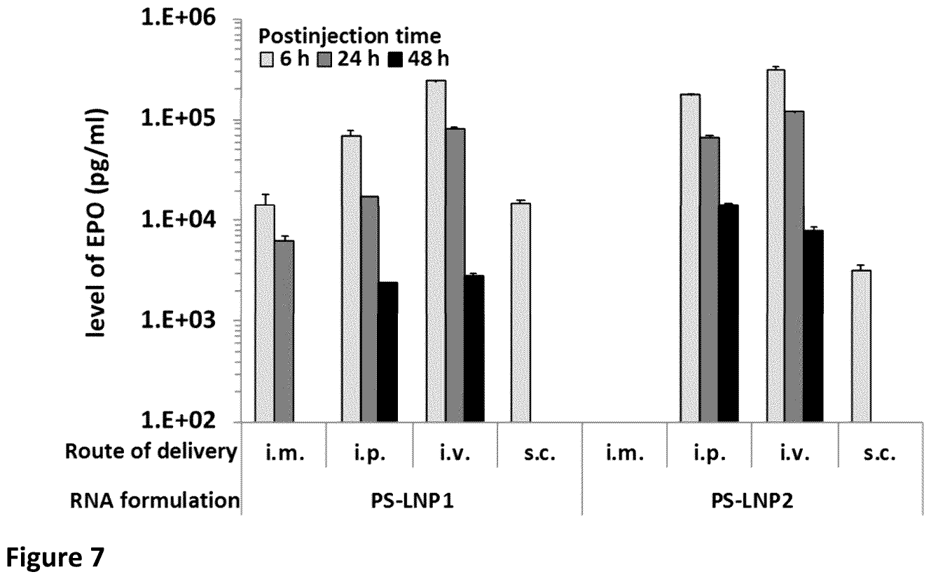

In another in vivo experiment (FIG. 7), PS-LNP1 was formulated with IVT murine EPO (mEPO) encoding mRNA and delivered in mice via diverse application routes. This formulation of PS-LNP1 demonstrated a particle size, PDI, and zeta potential of 243 nm, 0.22, −8.78 mV, and 308 nm, 0.19, −26.89 mV, respectively. Each mouse received either 1 μg (IP) or 3 μg (IM, IV, SC) of formulated RNA by intramuscular (IM), intraperitoneal (IP), intravenous (IV) or subcutaneous (SC) routes. Blood was drawn at 6, 24, and 48 hours post injection and plasma murine EPO levels measured by ELISA as described in Mahiny, A. J. and Karik6, K. (2016) (“Measuring Hematocrit in Mice Injected with In Vitro-Transcribed Erythropoietin mRNA. Methods in Mol Biol 1428: 297-306). The in-vivo data of PS-LNP1 demonstrated significant increase of mEPO in blood with all routes of administration. The increase in mEPO blood level was ranked according to the route of administration as follows: i.v.>i.p.>s.c.>i.m.

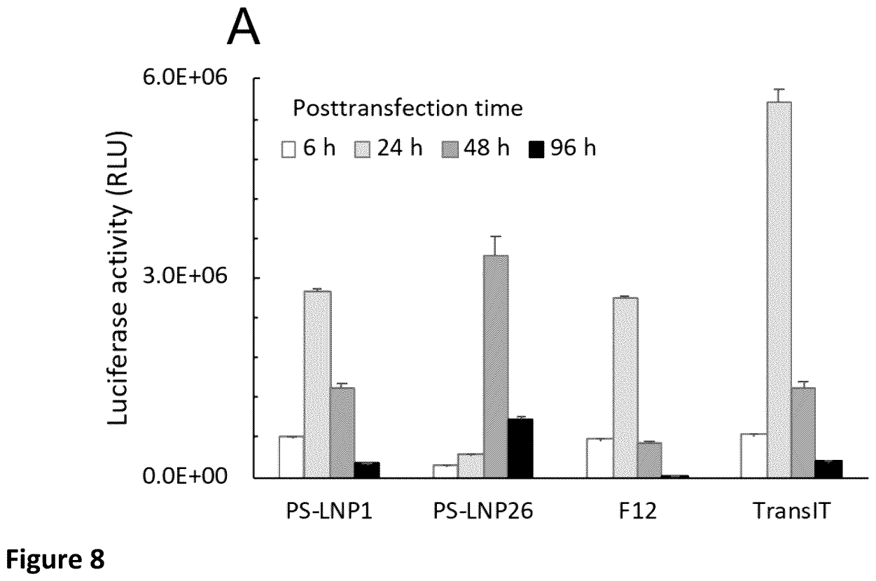

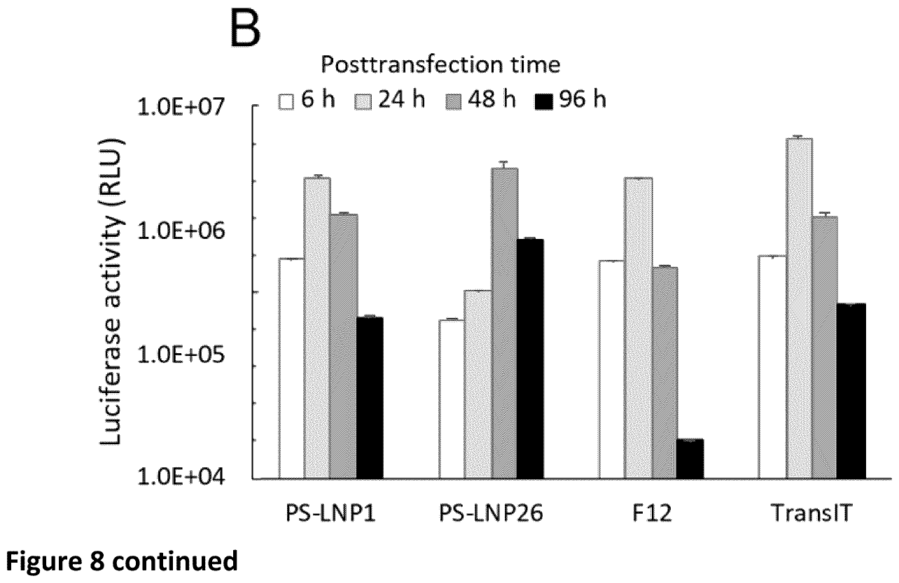

In an in vitro experiment (FIG. 8), PS-LNP1 F12 lipoplex as an internal benchmark (Kranz et al. 2016, Nature 534: 396), and TransIT mRNA (Mirus Bio) was formulated with IVT firefly luciferase-encoding mRNA and transfected into HepG2 cells. This formulation of PS-LNP1 demonstrated a particle size, PDI, and zeta potential of 303.5 nm, 0.29, −23.26 mV, respectively. One day prior to transfection, HepG2 cells were seeded in a 96 well-plate (25,000 cells/well) with DMEM culture medium (200 μL/well) supplemented with 10% fetal calf serum. For transfection, the culture medium was removed from the cells and 50 μL formulated mRNA (0.2 μg/well) was added to the cells. Cells were incubated and subsequently lysed at the indicated time using 100 μL 1× cell lysis buffer (Promega). The lysates were kept frozen at −20° C. until measuring luciferase activity using Promega's standard procedure. FIG. 8 shows that PS-LNP1 demonstrated high transfection efficiency, leading to in vitro highly detectable bioluminescence signal.

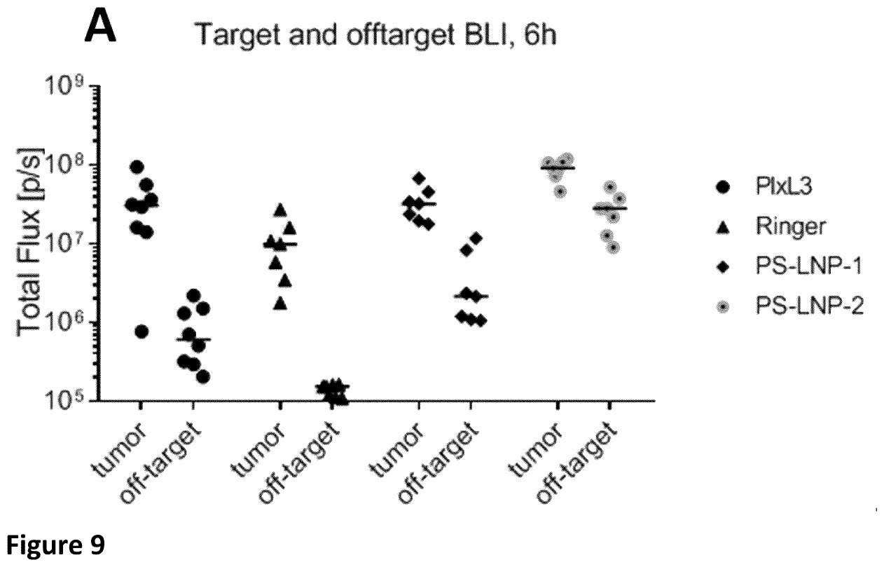

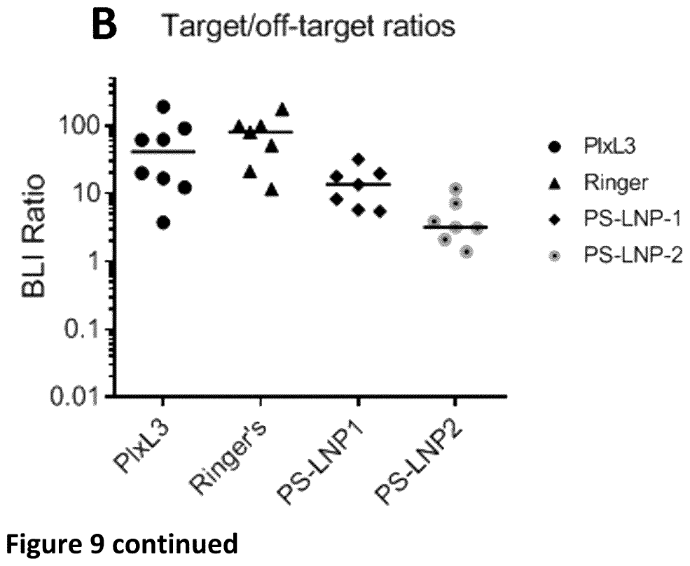

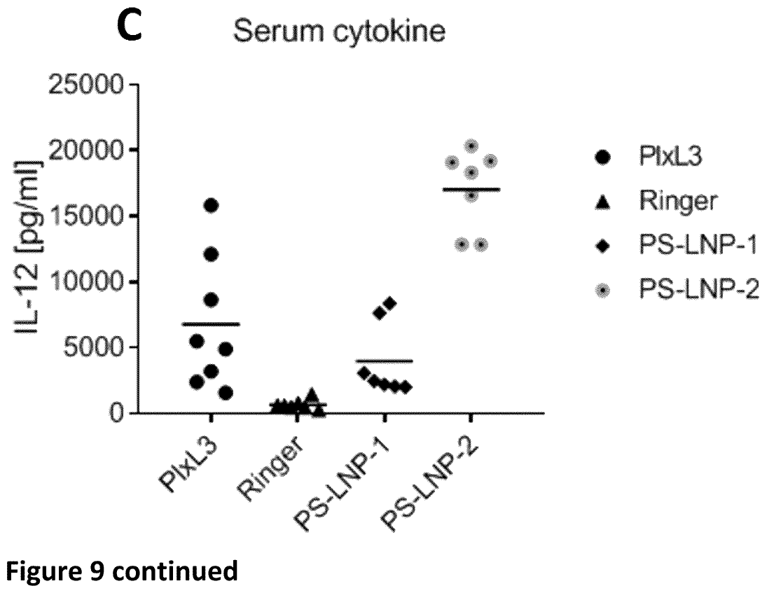

In four different in vivo experiments, (FIGS. 9-12), the IVT mRNA was formulated with PS-LNP1. Mice were injected intratumorally with preparations of two different mRNAs encoding for firefly luciferase and IL-12, respectively, in the indicated formulations to facilitate transfection and target translation. Luciferase protein acitivity was evaluated 6 h p.i. by bioluminescent imaging at the target anatomical site (tumor) as well as off-target locations, the latter reflecting signals from spleen and liver. A value around 1×10⁵ p/s can be considered base-line. Target-to-off target ratios indicate the extent of specific delivery to the tumor by means of formulation. IL-12 is a secreted cytokine and was determined by MSD ELISA in serum withdrawn at 6 h p.i.. The data shows that PS-LNP1 facilitated target mRNA translation and thus protein abundance or enzyme activity at a higher rate than Ringers salt solution and the internal benchmark, depending on the preparation. However, off-target translation was also increased above internal controls.

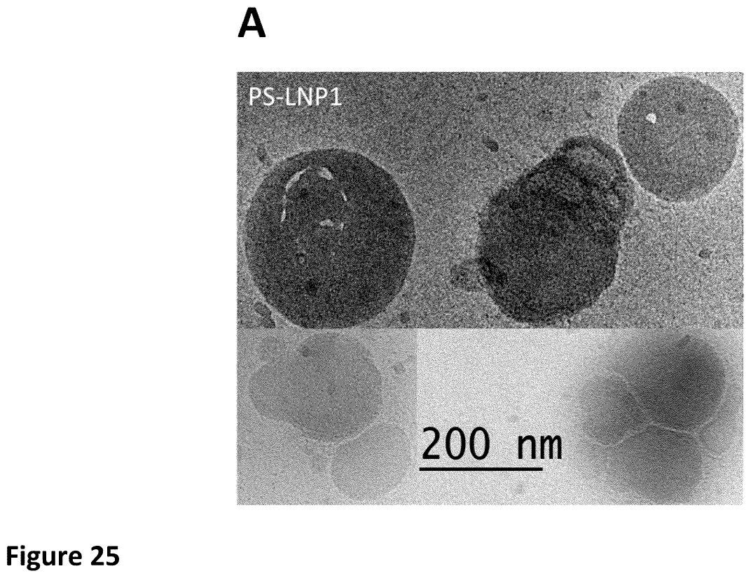

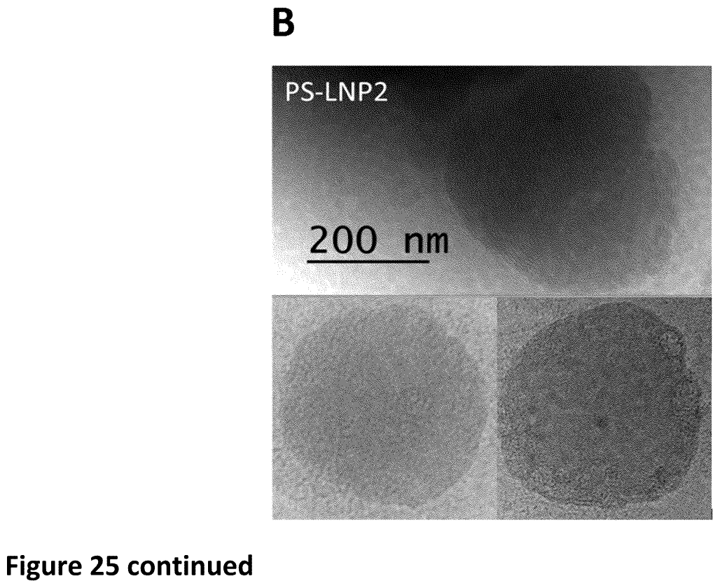

In CryoTEM imaging, as shown in FIG. 25, PS-LNP1 showed vesicular structure with oligo and multilamellar particles. Some of particles are very compact and show size range of 50 to 500 nm.

### Formulation Example 2: PS-LNP2

The preparation of PS-LNP2 and the quality control testing were done as described in the experimental part. The lipid composition of PS-LNP2 was DODMA/DOPE/DOPS/Tween20 (77/10/10/3 mol %). The positive/negative (cationic lipid/RNA nucleotide) ratio was 3.

In one in vivo experiment (FIGS. 2-5), PS-LNP2, and F12 lipoplex as an internal benchmark (Kranz et al. 2016, Nature 534: 396) were formulated with IVT mRNA encoding for firefly luciferase and injected into mice as described in Formulation Example 1. This formulation of PS-LNP2 demonstrated a particle size, PDI, zeta potential, and EE % of 596.5 nm, 0.31, −7.89 mV, and 56.9, respectively. The in vivo and ex vivo data of PS-LNP2 demonstrated high spleen and liver tissue transfection, leading to in vivo highly detectable bioluminescence signal.

In another in vivo experiment (FIG. 6), PS-LNP2 was tested in the same experimental setting as described in Formulation Example 1. This formulation of PS-LNP2 demonstrated a particle size, PDI and zeta potential of 406.6 nm, 0.28 and −26.89 mV, respectively. The in vivo results show that PS-LNP2 demonstrated high local signal upon ID and IM administration and spleen and liver tissue transfection upon i.v. application. This in vivo highly detectable bioluminescence signal shows clearly high functionality of PS-LNP2 nanoparticle formulation.

In another in vivo experiment (FIG. 7), PS-LNP2 was tested in the same experimental setting as described in Formulation Example 1. This batch of formulation PS-LNP2 demonstrated a particle size, PDI, and zeta potential of 308 nm, 0.19, and −26.89 mV, respectively. As shown in FIG. 7, the in vivo results of PS-LNP2 demonstrated significant increase of mEPO in blood stream with all routes of administration. The increase in mEPO blood level was ranked according to the route of administration as follows: i.v.>i.p.>s.c.>i.m.

In three different in vivo experiments as shown in FIGS. 9-11, mice were injected intratumorally following the same experimental setting as described in Formulation Example 1. The data shows that PS-LNP2 facilitated target mRNA translation and thus protein abundance or enzyme activity at a higher rate than Ringers salt solution and the internal benchmark. However, off-target translation was also increased above internal controls.

CryoTEM imaging shows (FIG. 25) that PS-LNP2 showed vesicular nanoparticles, double and multiwalled nanoparticles. Nanoparticles without or with irregular lamellar structure from 50 to 500 nm and multilamellar nanoparticles in the size range of 50 to 400 nm are shown.

### Formulation Example 3: PS-LNP3

The preparation of PS-LNP3 and the quality control testing were done as described in the experimental part. The lipid composition of PS-LNP3 was DODMA/DOPS/Tween20 (87/10/3 mol %) and the positive/negative (cationic lipid/RNA nucleotide) ratio was 3.

In an in vivo experiment (FIG. 6), PS-LNP3 was tested in the same experimental setting as described in Formulation Examples 1 and 2. This batch of PS-LNP3 formulation demonstrated a particle size, PDI and zeta potential of 367.26 nm, 0.31 and −23.84 mV, respectively. The in vivo data shows that PS-LNP3 demonstrated moderate local signal upon ID and IM administration and spleen and liver tissue transfection upon i.v. application. This in vivo detectable bioluminescence signal proves clearly functionality of PS-LNP3 nanoparticle formulation.

### Formulation Example 4: PS-LNP8

The lipid composition of the PS-LNP8 was DODMA/DOPE/DOPS/Tween20 (62/25/10/3 mol %). The formulation was designed to increase the DOPE mol % and decrease the DODMA mol % which might alleviate the liver signal. The positive/negative (cationic lipid/RNA nucleotide) ratio was 4. The formulation was prepared and characterized as described in the experimental part.

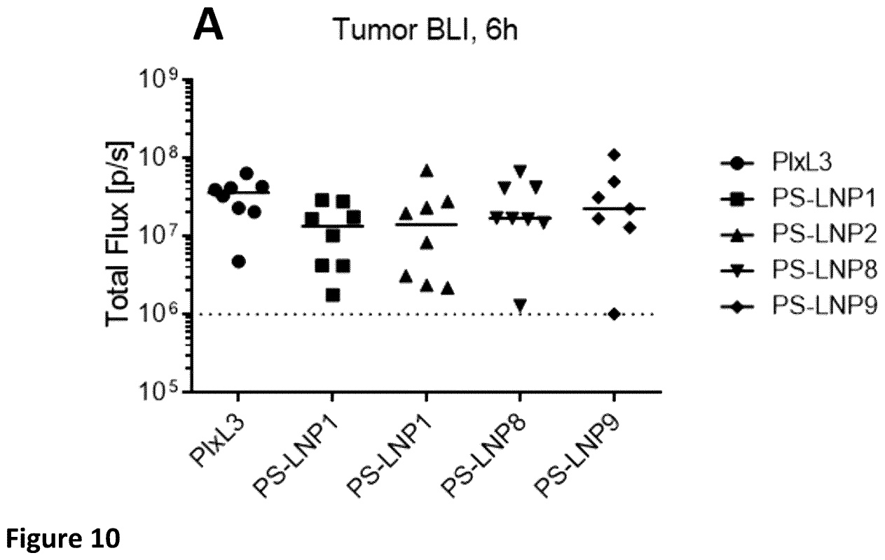

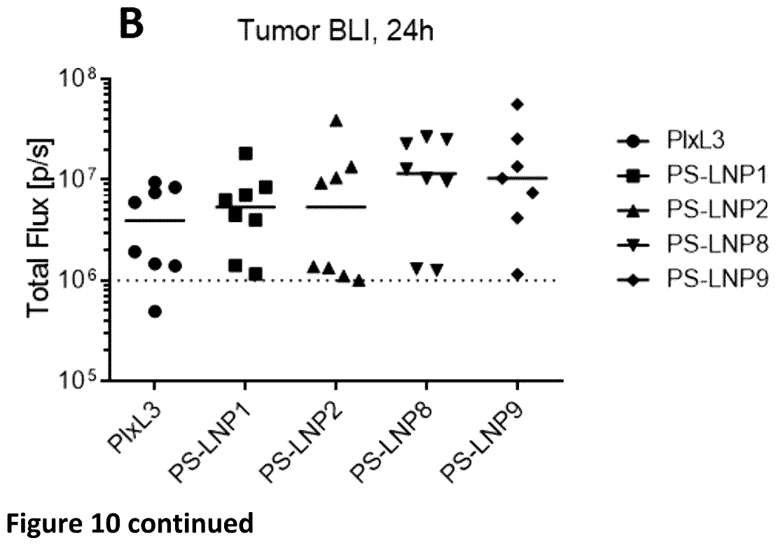

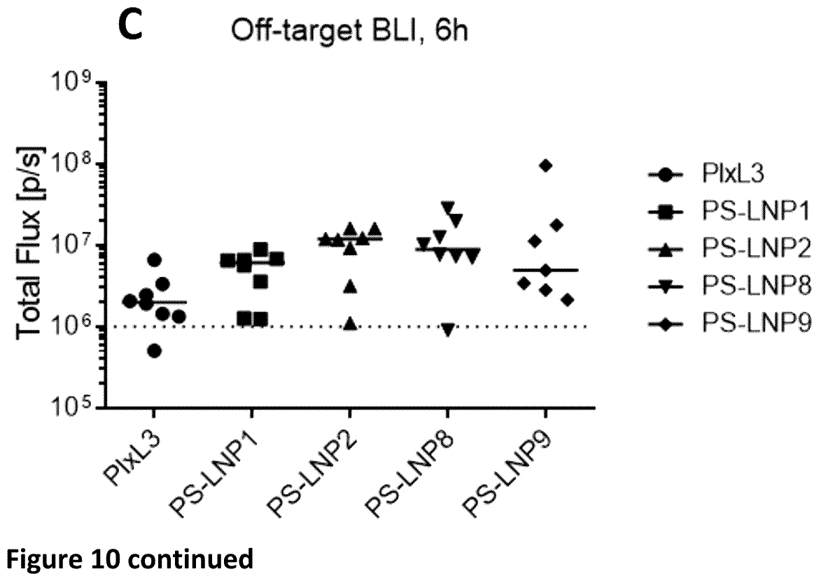

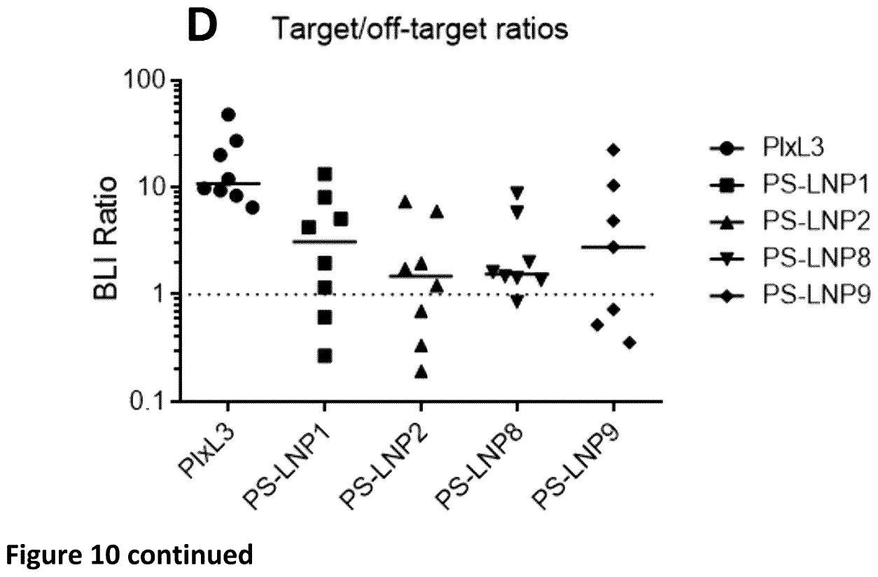

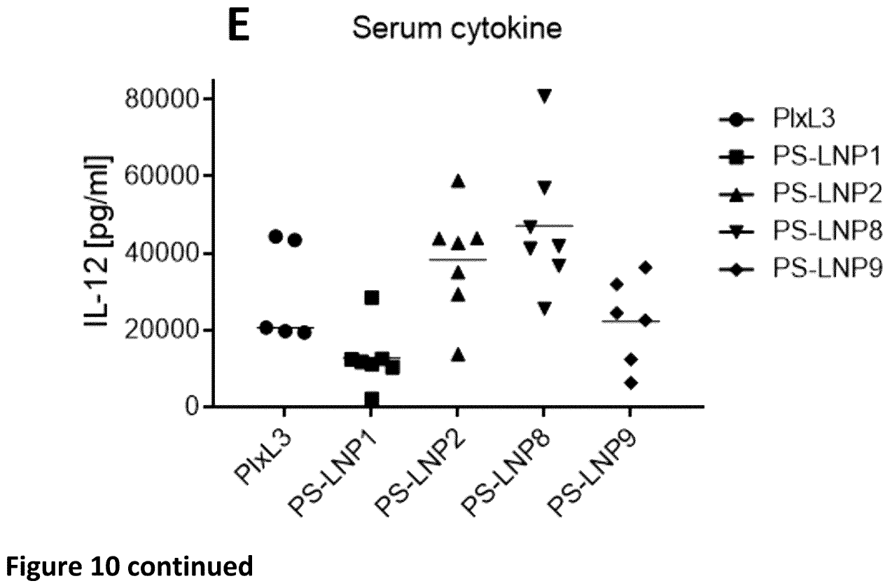

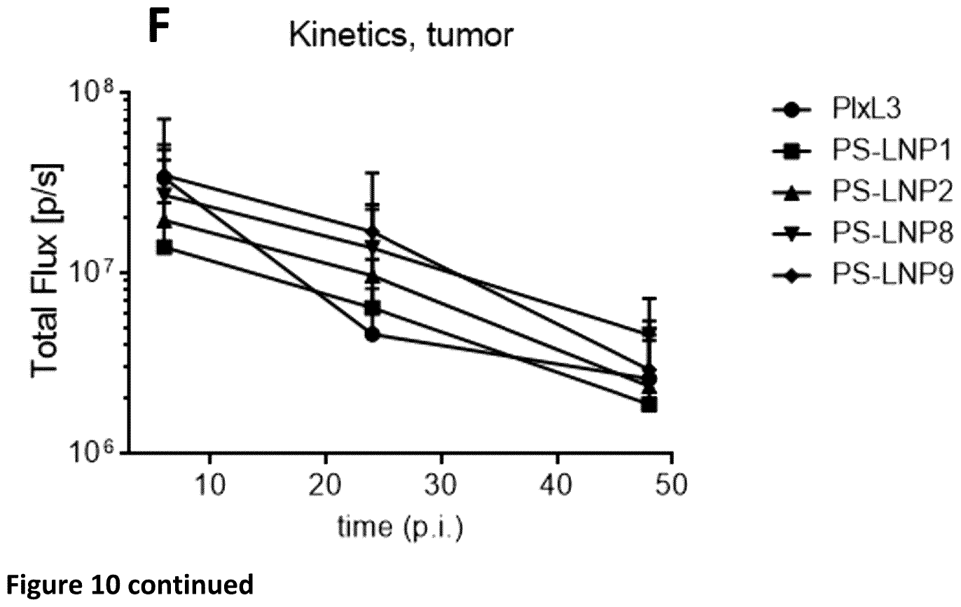

In an intratumoral in-vivo experiment (FIG. 10), PS-LNP8 was formulated with IVT mRNA. This formulation of PS-LNP8 demonstrated a particle size, PDI, zeta potential, and EE % of 243.3 nm, 0.31, −2.87 mV, and 97.2 respectively. Mice were injected intratumorally in the same experimental setting as described in Formulation Examples 1 and 2. Like all Phosphatidylserine-containing LNPs, PS-LNP8 facilitated target mRNA translation in the range of the internal benchmark, but with lower specificity, with PS-LNP8 showing a trend to higher values for both aspects. So, the increase in the DOPE mol % and decrease in the DODMA mol % within the tested range does not show a decrease in liver signal.

### Formulation Example 5: PS-LNP9

The lipid composition of the PS-LNP9 was DODMA/DOPE/DOPS/Tween20, (70/10/10/10 mol %). The formulation was designed to increase the tween20 mol % and decrease DODMA mol % which might decrease the stability of the particles by forming softer particles in blood stream consequently alleviating the liver signal. The positive/negative (cationic lipid/RNA nucleotide) ratio was 4. The formulation was prepared and characterized as described in the experimental part.

In an in-vivo experiment (FIG. 10), PS-LNP9 was formulated with IVT mRNA. This formulation of PS-LNP9 demonstrated a particle size, PDI, zeta potential, and EE % of 200.9 nm, 0.18, −14.35 mV, and 69.70, respectively. Mice were injected intratumorally as described in Formulation Examples 1 and 2. From comparison of PS-LNP2 (3 mol % tween 20) and PS-LNP9 (10 mol % tween 20), it is clear that both formulations facilitated target mRNA translation in the range of the internal benchmark, but with lower specificity, with PS-LNP9 showing a trend to higher values for both aspects (target and off-target). However, increasing the tween20 mol % from 3 to 10 mol % and decreasing the DODMA concentration from 77 to 70 mol % (PS-LNP9) in this experimental setting slightly improves the target/off target ratio (compare PS-LNP2 with PS-LNP9).

### Formulation Example 6: PS-LNP16

The lipid composition of the PS-LNP16 was DODMA/DOPE/DOPS/Tween20, (27/60/10/3 mol %). PS-LNP16 was designed to have high mol % of DOPE (60 mol %) and to decrease the pH sensitive lipid mol % which might lead to a decrease of off target, particularly liver, signal. The formulation was prepared and characterized as described in the experimental part.

### Formulation Example 7: PS-LNP19

Cholesterol (CHOL) is a commonly used ingredient in liposomes and LNPs as a component that stabilizes lipid bilayers by filling in gaps between phospholipids. Inclusion of CHOL contributes to greater stability of LNPs in the presence of serum proteins. In addition, CHOL has been shown to promote membrane fusion. As a co-lipid in LNP formulations for gene delivery in vivo, CHOL generally outperformed DOPE despite its lower fusogenicity. When present at high percentages, CHOL seems to enhance the activity of cationic lipids and promote gene transfer, possibly by promoting bilayer destabilization. The presence of CHOL along with PC results in stable lipid bilayers and CHOL is commonly used in oligonucleotide LNP formulations (Xinwei Cheng, Robert J. Lee, Advanced Drug Delivery Reviews 2017, p 127-137). Therefore, the conventional composition of LNPs contains 40-50 mol % of cholesterol. PS-LNP19 (DODMA/CHOL/DOPS/Tween20 (77/10/10/3 mol %) was designed to have low mol % of CHOL (10 mol %) which might lead to less stable particles in the blood stream which in turn leads to a decrease of off target, particularly liver, signal. As the total mol % is 100, the decrease in cholesterol mol % was compensated by an increase in DODMA mol %. The positive/negative (cationic lipid/RNA nucleotide) ratio was 3. The formulation was prepared and characterized as described in the experimental part.

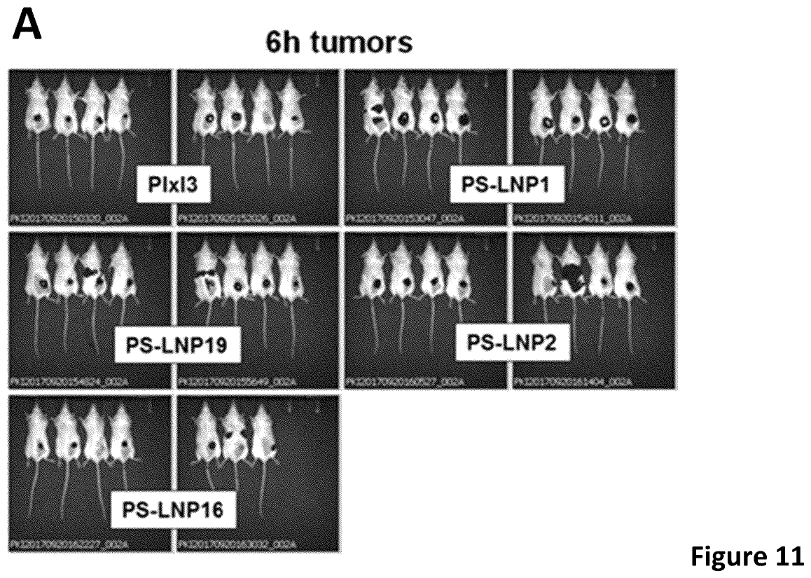

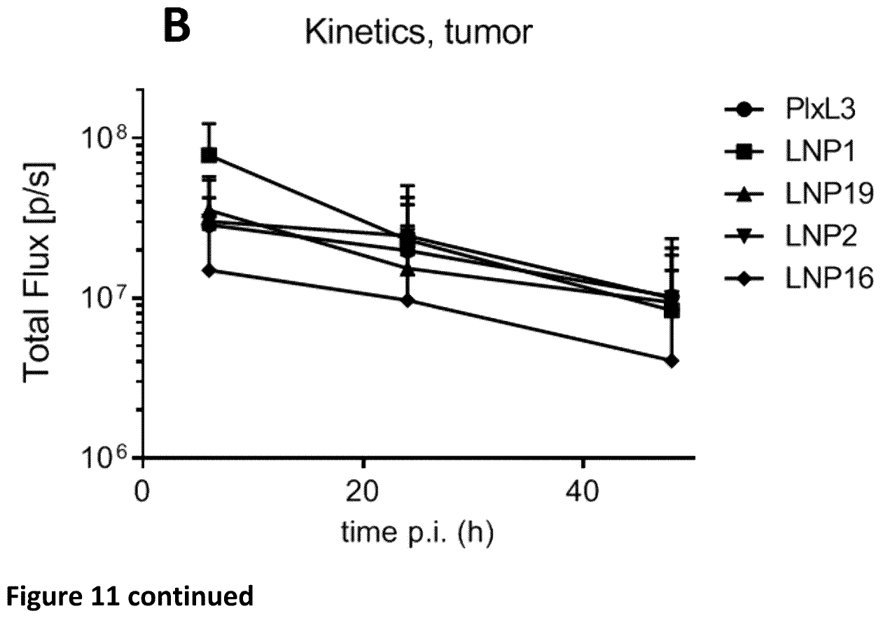

In an in-vivo experiment (FIG. 11), PS-LNP16 and PS-LNP19 were formulated with IVT mRNA. Formulation PS-LNP16 demonstrated a particle size, PDI, zeta potential, and EE % of 243.3 nm, 0.31, -2.87 mV, and 97.2 respectively. Formulation PS-LNP19 demonstrated a particle size, PDI and zeta potential of 235.6 nm, 0.24 and −7.89 mV, respectively. Mice were injected intratumorally following the same experimental setting as described in Formulation Examples 1 and 2. PS-LNP16 and PS-LNP19 consistently mediate equal or better on-target protein translation with lower specificity. Increasing the DOPE mole % (PS-LNP16) and decreasing the CHOL mol % (PS-LNP19) did not improve the on target/off target mRNA translation compared to the respective parental PS-LNP formulations, PS-LNP2 and PS-LNP1, respectively. In addition, it is evident that increasing DOPE to 60 mol % and decreasing DODMA to 27 mol % (PS-LNP16) leads to a decrease in the overall signal.

### Formulation Example 8: PS-LNP23

The lipid composition of the PS-LNP23 was DMRIE/CHOL/DOPE/DOPS/Tween20, (47/30/10/10/3 mol %). The formulation was designed to replace the pH sensitive lipid with the permanent cationic lipid DMRIE which showed very low liver signal in other experiments (data not shown). The cationic lipid/RNA nucleotide (positive/negative) ratio was 3. The formulation was prepared and characterized as described in the experimental part.

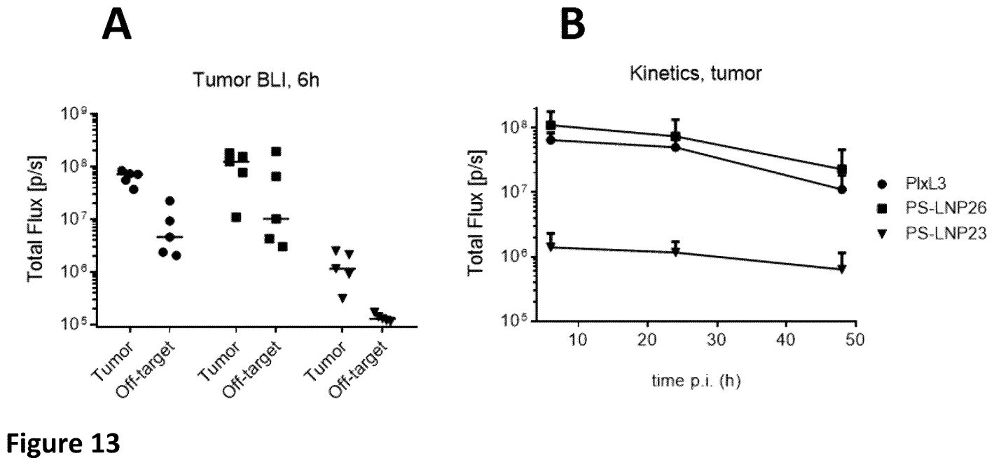

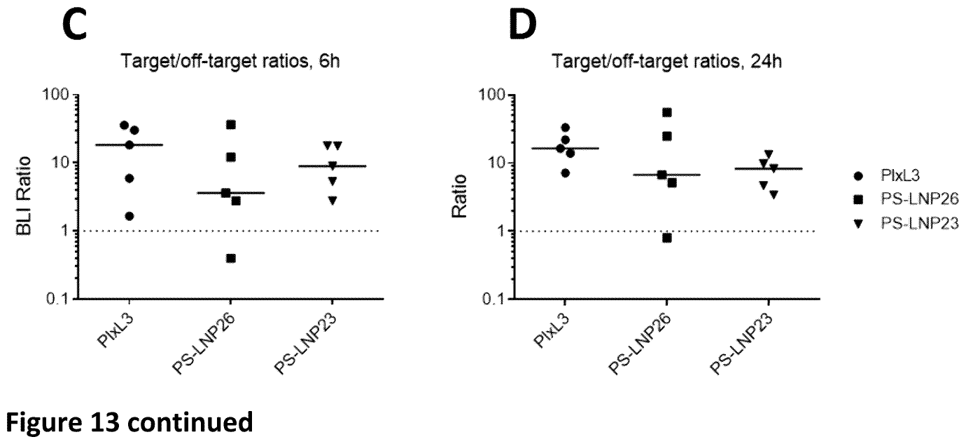

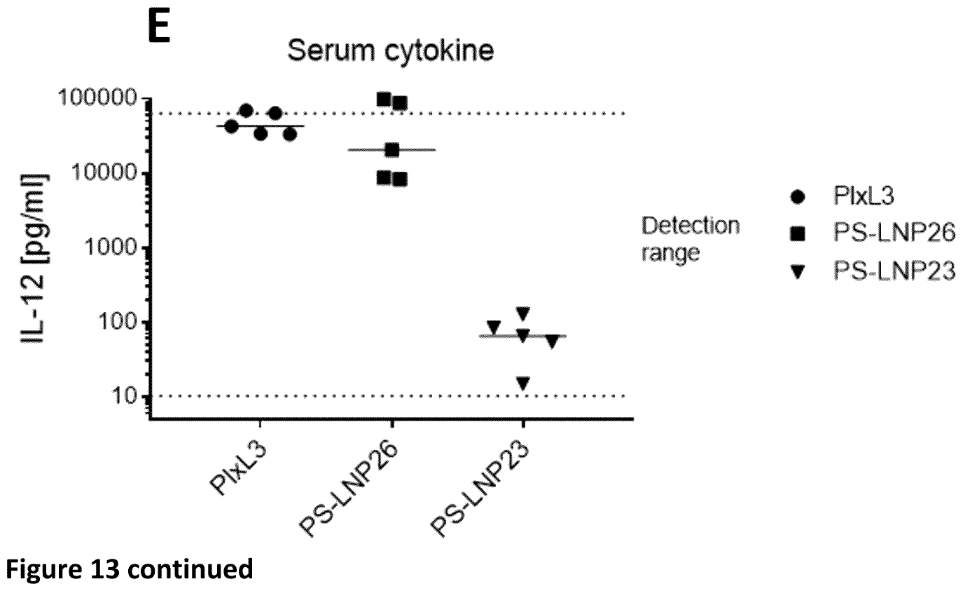

As shown in FIG. 13, the IVT mRNA was formulated with PS-LNP23. Mice were injected intratumorally following the same experimental setting as described in Formulation Examples 1 and 2. The in vivo data shows that PS-LNP23 is able to transfect only tumor tissue without spleen and liver tissues but at low overall signal in comparison to pH sensitive lipid containing PS-LNPs.

### Formulation Examples 9 &10: PS-LNP24 & PS-LNP25

The compositions were designed to test Dlin-KC2-DMA lipid compared to DODMA lipid. The lipid composition of PS-LNP24 was DODMA/CHOL/DOPE/DOPS/Tween20 (47/30/10/10/3 mol %), while the lipid composition of PS-LNP25 was Dlin-KC2/CHOL/DOPE/DOPS/Tween20 (47/30/10/10/3 mol %). The positive/negative (cationic lipid/RNA nucleotide) ratio was 3 for both formulations. The preparation and the quality control testing of PS-LNP24 and PS-LNP25 were done as described in the experimental part.

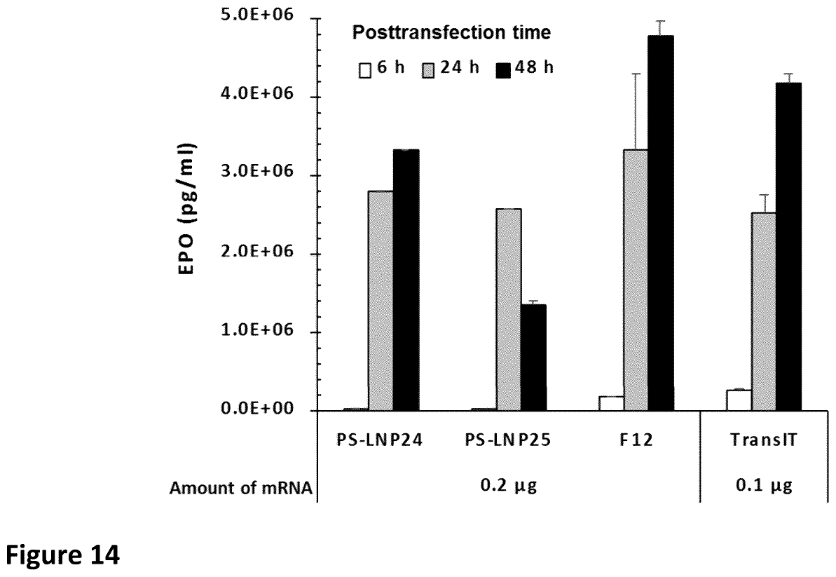

In an in vitro experiment (FIG. 14), PS-LNP24, PS-LNP25, F12 lipoplex as an internal benchmark (Kranz et al. 2016, Nature 534: 396), and TransIT mRNA (Mirus Bio) were formulated with IVT murine EPO mRNA and tranfected into HepG2 cells as described in Formulation Examples 1 and 2. The PS-LNP24 formulation demonstrated a particle size, PDI, and zeta potential of 257.4 nm, 0.21 and −24.38 mV, respectively. PS-LNP25 demonstrated a particle size, PDI, and zeta potential of 235.8 n, 0.20, -22.23 mV, respectively. As shown in FIG. 14, PS-LNP24 and PS-LNP25 demonstrated high transfection efficiency, leading to in vitro highly detectable EPO.

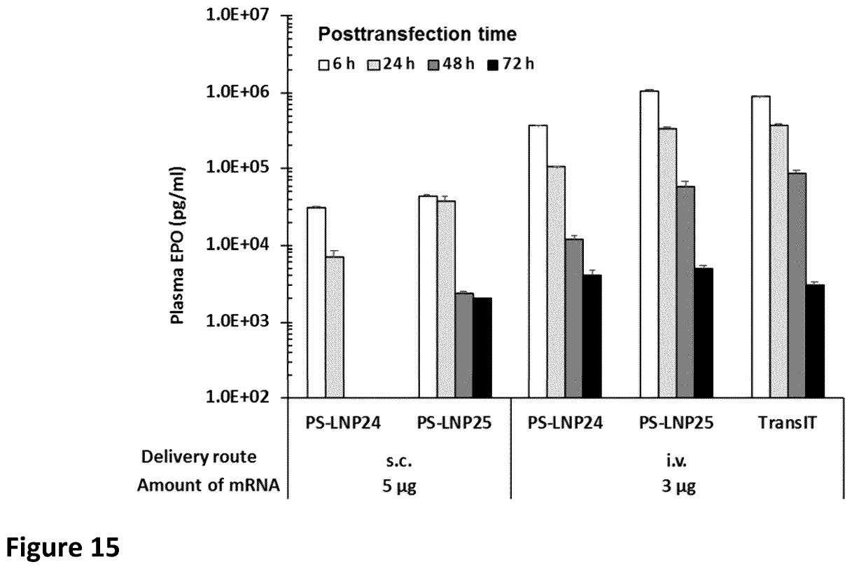

In another in vivo experiment (FIG. 15), PS-LNP24 was formulated along with PS-LNP25 with IVT murine EPO-encoding mRNA (m1ψ-modified CC413 EPO mRNA) and delivered subcutaneously or intravenously to mice. Each mouse received either 5 μg (SC) or 3 μg (IV) of formulated mRNA by intravenous (i.v.) delivery route. Blood was collected 6, 24, 48 and 72 hours post administration of the mRNA and plasma murine EPO levels were measured by ELISA as described in Mahiny and Karik6 (2016, Methods in Mol Biol 1428: 297-306). The results shown in FIG. 15 demonstrated that PS-LNP24 leads to an increase of EPO blood level. IV administration showed higher level of EPO in comparison to SC administration.

### Formulation Example 11: PS-LNP26

The lipid composition of the PS-LNP26 was Dlin-MC3-DMA/CHOL/DOPE/DOPS/Tween20, (47/30/10/10/3 mol %). The formulation was designed to replace the pH sensitive lipid DODMA with the highly efficient pH sensitive lipid Dlin-MC3-DMA as another example of pH sensitive lipids. In addition, the biodistribution profile might be different for each lipid. The cationic lipid/RNA nucleotide (positive/negative) ratio was 3. The formulations were prepared and characterized as described in the experimental part.

PS-LNP26 was tested in the same setting as described in Formulation Example 1 and FIG. 8. This formulation of PS-LNP26 demonstrated a particle size, PDI, and zeta potential of 297.8 n, 0.31 and -21.72 mV, respectively. As shown in FIG. 8, PS-LNP26 demonstrated high transfection efficiency, leading to in vitro highly detectable bioluminescence signal.

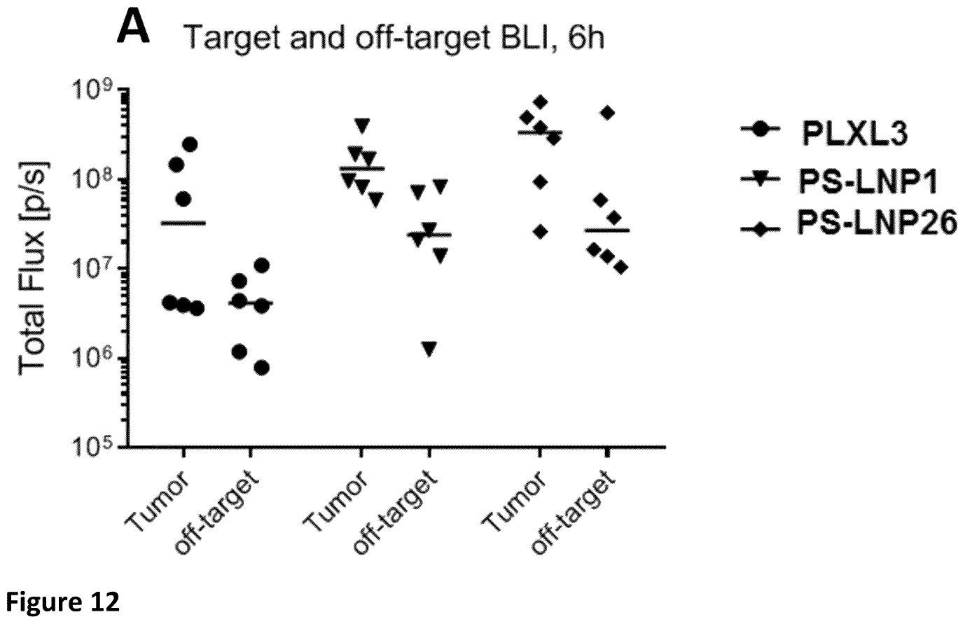

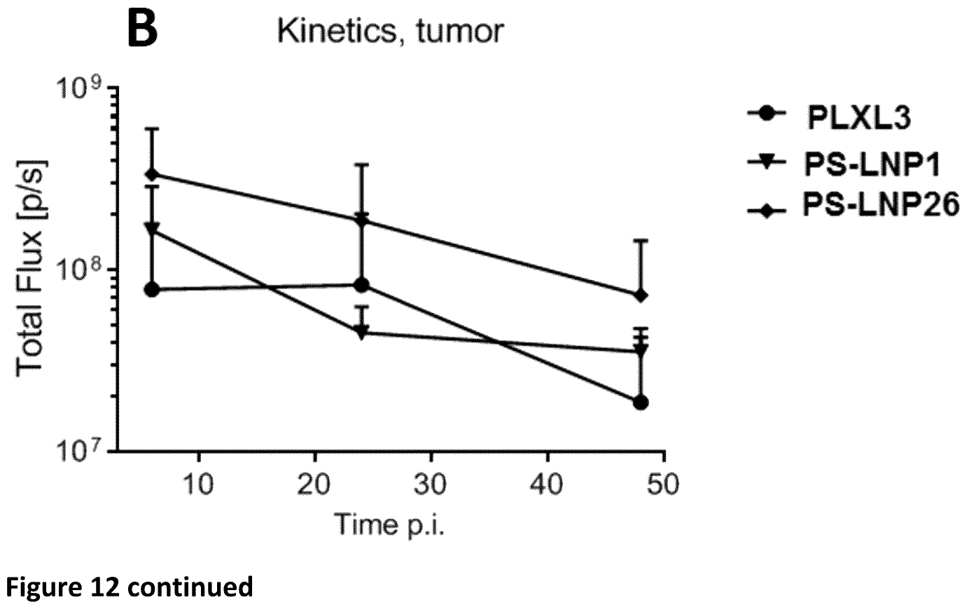

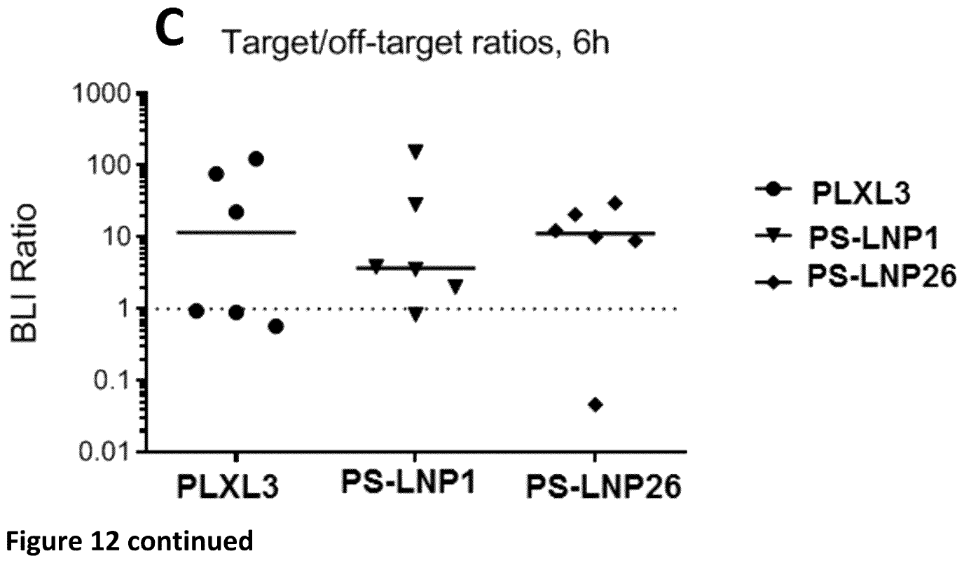

In an in-vivo experiment (FIG. 12), PS-LNP26 was formulated with IVT mRNA and mice were injected intratumorally in the same experimental setting as described in Formulation Examples 1 and 2. As shown in FIG. 12, LNP26 mediated higher luciferase activity than the internal benchmark. The change from DODMA to MC3-DMA improved specific mRNA delivery resulting in similar target-to-off target ratios as the internal benchmark.

In another in-vivo experiment (FIG. 13), PS-LNP26 was tested following the same experimental setting as described above. The in-vivo and ex-vivo data of PS-LNP26 demonstrated high tumor, spleen and liver tissue transfection, leading to in-vivo highly detectable bioluminescence signal.

### Formulation Example 12: PS-LNP49, PS-LNP49-2, PS-LNP49-3, and PS-LNP50. The PS-LNPs

PS-LNP49, PS-LNP49-2, PS-LNP49-3, and PS-LNP50) were all prepared and characterized as described in the experimental part using the lipid ratios listed in Table 3. The formulated PS-LNPs were designed to investigate the influence of the main (ionizable) lipid and Tween20 by varying their molar ratios. In addition, PS-LNP50 does not contain PS lipid.

TABLE 3
PS-LNP49, PS-LNP49-2, PS-LNP49-3, and PS-LNP50, compositions and mol %, buffer, Z-Averages,
PDIs, zeta potential values, and N/P ratio (pH sensitive (cationic) lipid/RNA).

| | | | Z-Average | | Zet. P | |
|---|---|---|---|---|---|---|
| Groups | Compositions | Buffer | (nm) | PDI | (mV) | N/P |
| PS-LNP49 | Dlin-MC3-DMA/DOPE/DOPS/Tween20, | PBS | 288.5 | 0.35 | 19.03 | 3 |
| | (50/10/10/30) | | | | | |
| PS-LNP49-2 | Dlin-MC3-DMA/DOPE/DOPS/Tween20, | PBS | 275.3 | 0.27 | 11.19 | 3 |
| | (55/10/10/25) | | | | | |
| PS-LNP49-3 | Dlin-MC3-DMA/DOPE/DOPS/Tween20, | PBS | 262.2 | 0.226 | 1.71 | 3 |
| | (60/10/10/20) | | | | | |
| PS-LNP50 | Dlin-MC3-DMA/DOPE/Tween20, | PBS | 247.6 | 0.228 | 5.78 | 3 |
| (No DOPS) | (55/20/25) | | | | | |

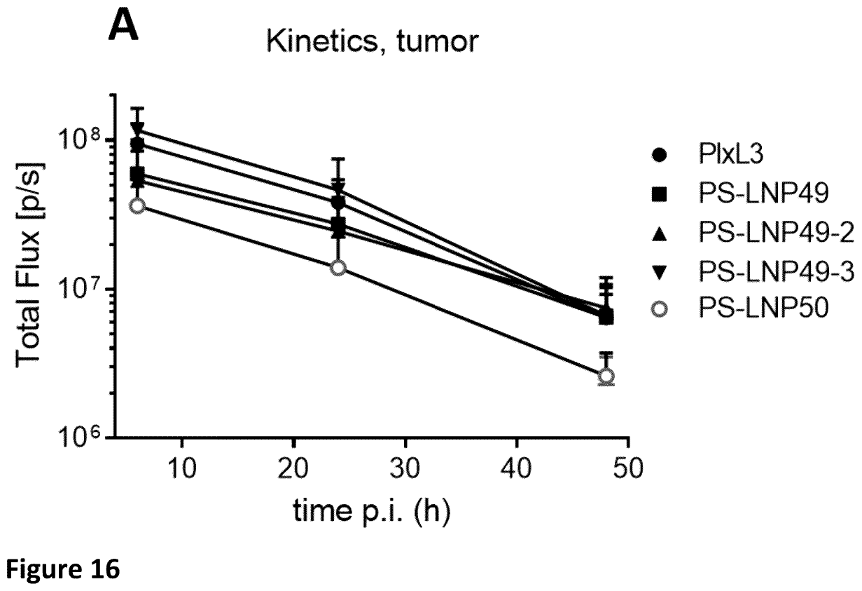

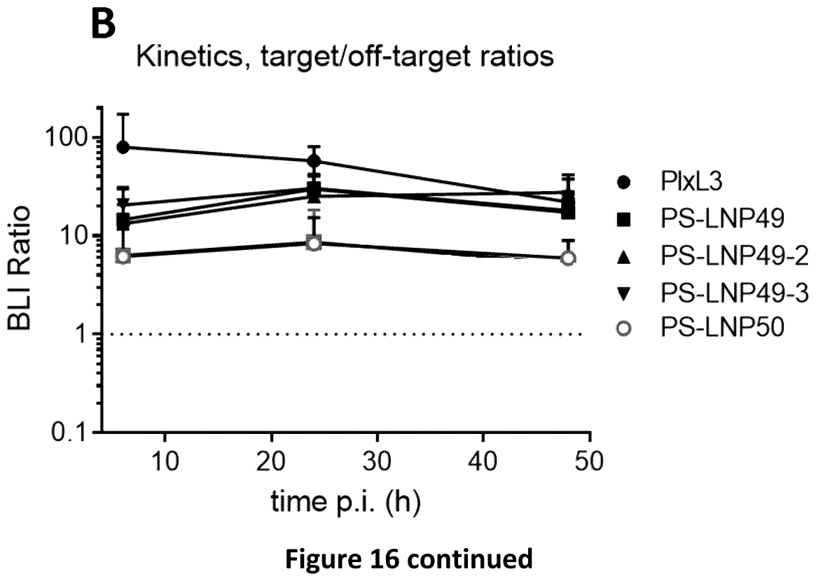

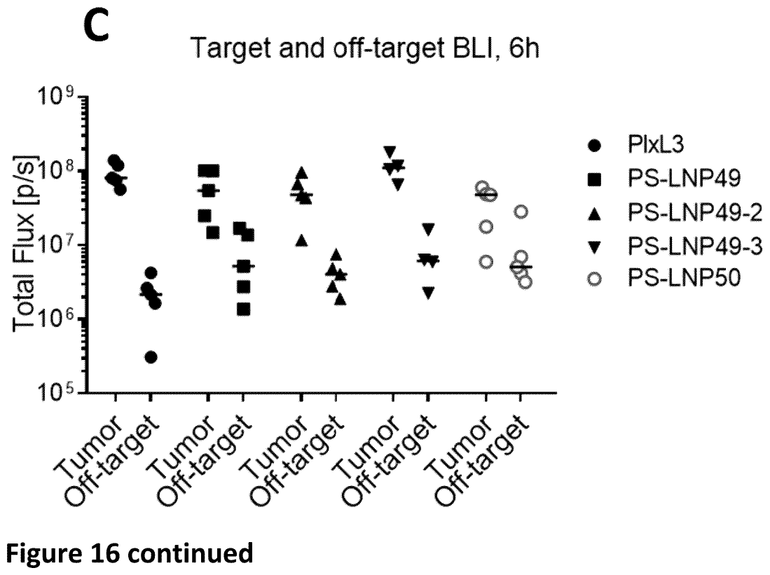

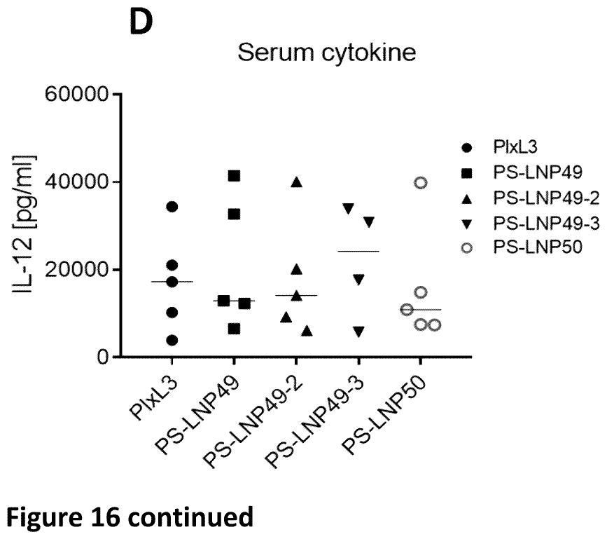

The formulations were tested intratumorally following the experimental setting described in Formulation Example 1. Increasing the molar percentage of the ionizable lipid Dlin-MC3-DMA (from 50 to 60 mol %) and decreasing the molar percentage of Tween20 (from 30 mol % to 20 mol %) improved on-target translation and specificity which leads to an improve in the target/off-target ratio (PS-LNP49-3>PS-LNP49-2>PS-LNP49). Removing phosphatidylserine led to lower activity and a drop in the target-to-off target ratio (PS-LNP50) as shown in FIG. 16A-D.

### Formulation Example 13: PS-LNP49-3 and PS-LNP52

PS-LNP49-3 and PS-LNP52 were prepared and characterized as described in the experimental part using the lipid ratios listed in Table 4. In this experiment, the PS-LNP formulations were designed to create a direct comparison between the ionizable lipids Dlin-MC3-DMA and DODMA. As described in Table 4, PS-LNP49-3 and PS-LNP52 contained Dlin-MC3-DMA and DODMA, respectively.

TABLE 4
PS-LNP49-3 and PS-LNP52 lipid compositions and mol %, buffer, particle sizes,
PDIs, zeta potential values and N/P ratios (pH sensitive (cationic) lipid/RNA).

| | | | Z-Average | | |
|---|---|---|---|---|---|
| Group | Formulation | Buffer | (nm) | PDI | N/P |
| PS-LNP49-3 | Dlin-MC3-DMA/DOPE/DOPS/Tween20 | PBS | 297.9 | 0.155 | 3 |
| | (60/10/10/20) | | | | |
| PS-LNP52 | DODMA/DOPE/DOPS/Tween20 | PBS | 256.6 | 0.257 | 3 |
| | (60/10/10/20) | | | | |

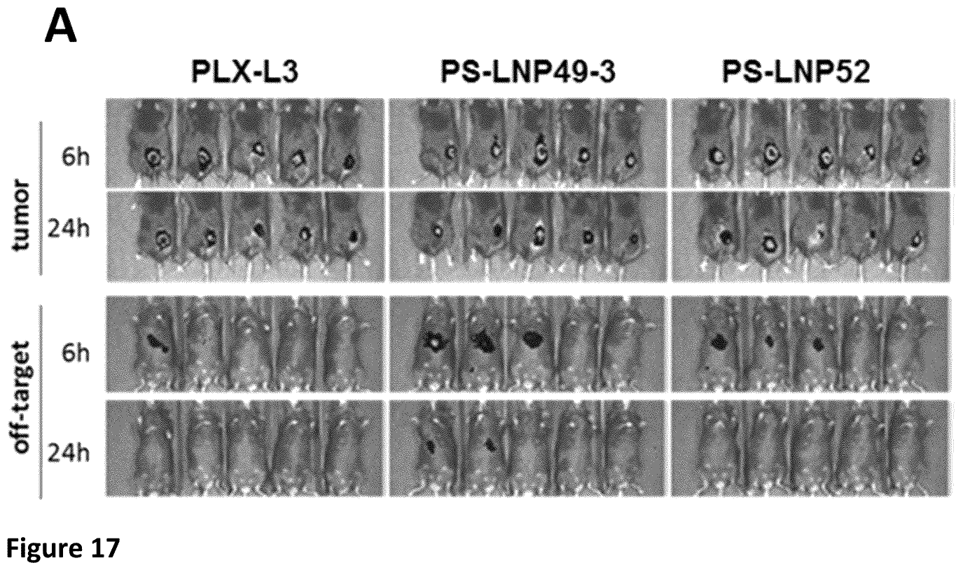

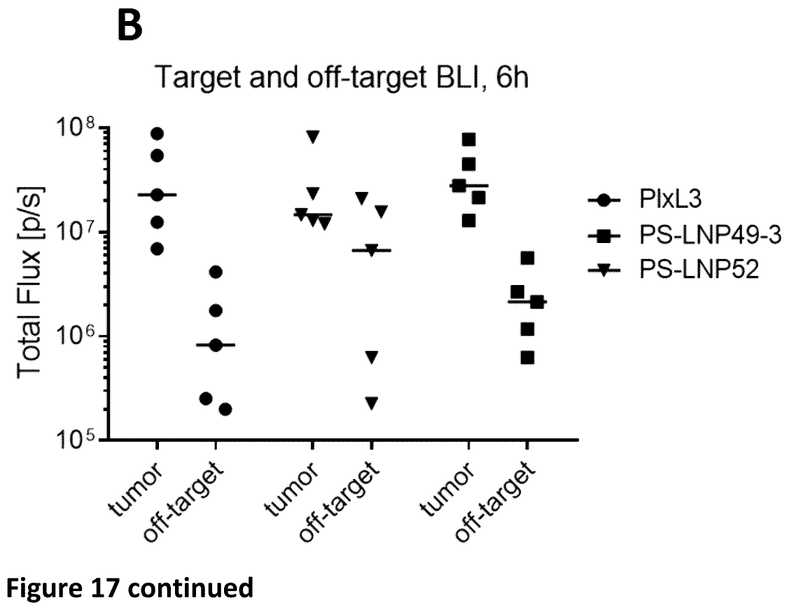

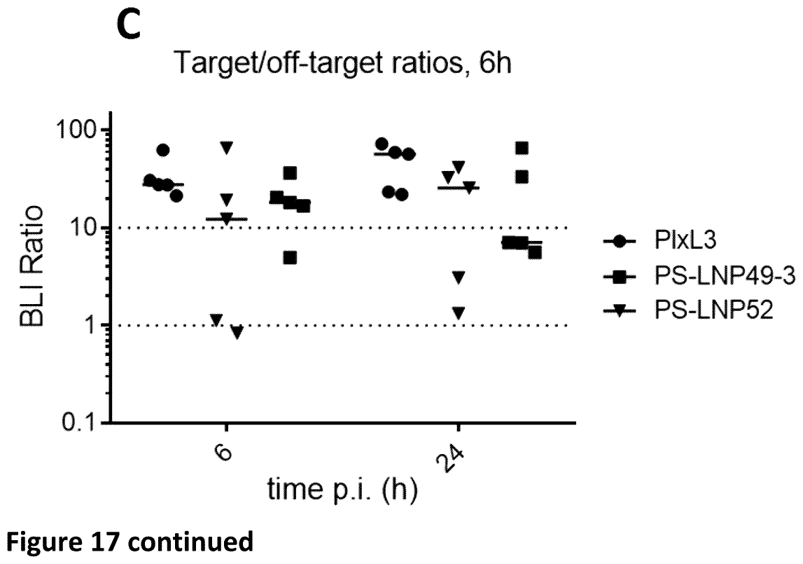

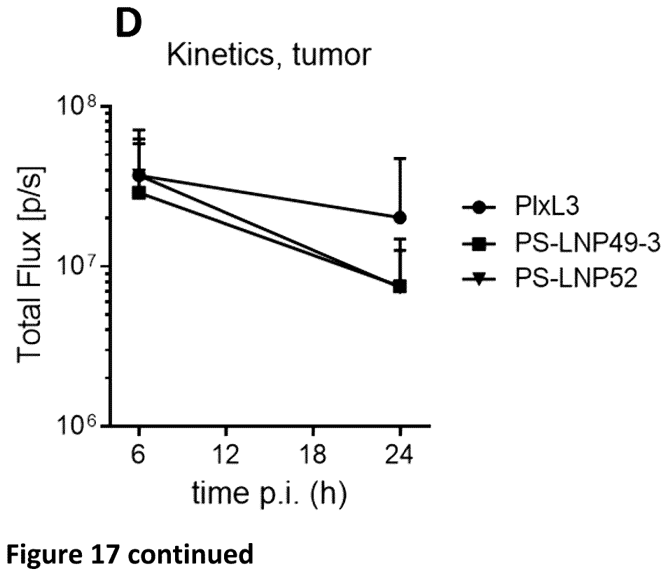

In an in-vivo experiment (FIG. 17), TC1-tumor bearing mice were injected intratumorally with preparations of PS-LNP49-3 and PS-LNP52 formulated with two different mRNAs encoding for firefly luciferase and IL-12. As demonstrated in FIG. 17, it is clearly observed that replacement of Dlin-MC3-DMA with DODMA resulted in very similar on-target translation rates but showed a trend indicating reduced off-targeting, mainly at 24 hours with respect to the liver in TC1-tumor model.

### Formulation Example 14: PS-LNP25, PS-LNP49-3, PS-LNP52

PS-LNP25, PS-LNP49-3, PS-LNP52 were prepared and characterized as described in the experimental part using the lipid ratios listed in Table 5. In this experiment, the PS-LNPs formulations were designed to confirm the results obtained in Formulation Example 13 with another tumor model. As described in Table 5, PS-LNP25, PS-LNP49-3, and PS-LNP52 contain the ionisable lipids Dlin-KC2-DMA, Dlin-MC3-DMA and DODMA, respectively.

TABLE 5
PS-LNP25, PS-LNP49-3, and PS-LNP52 lipid compositions and mol %, buffer,
Z-Averages, PDIs and N/P ratios (pH sensitive (cationic) lipid/RNA).

| | | | Z-Average | | N/P |
|---|---|---|---|---|---|
| Formulations | Compositions | Buffer | (nm) | PDI | ratio |
| PS-LNP25 | Dlin-KC2-DMA/CHOL/DOPE/DOPS/Tween20 | PBS | 135.4 | Multi | 3 |
| | (47/30/10/10/3) | | | modal | |
| PS-LNP49-3 | Dlin-MC3-DMA/DOPE/DOPS/Tween20 | PBS | 266.6 | 0.221 | 3 |
| | (60/10/10/20) | | | | |
| PS-LNP52 | DODMA/DOPE/DOPS/Tween20 | PBS | 160.0 | 0.199 | 3 |
| | (60/10/10/20) | | | | |

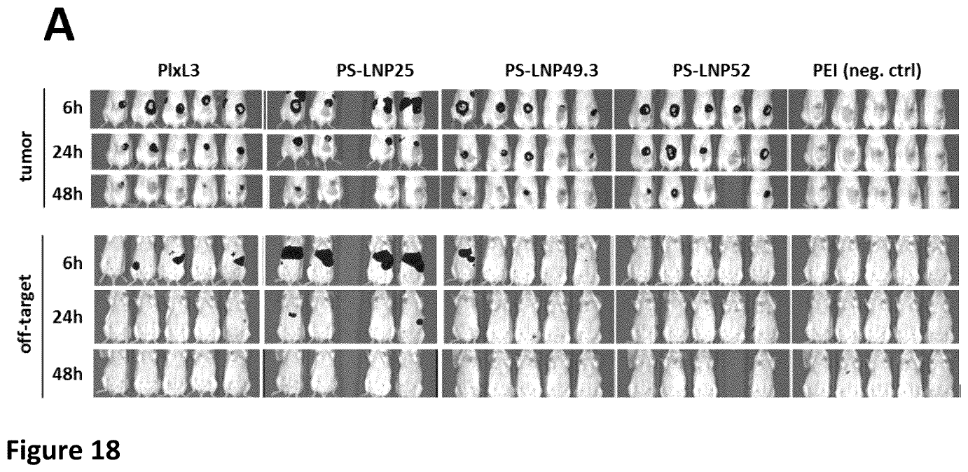

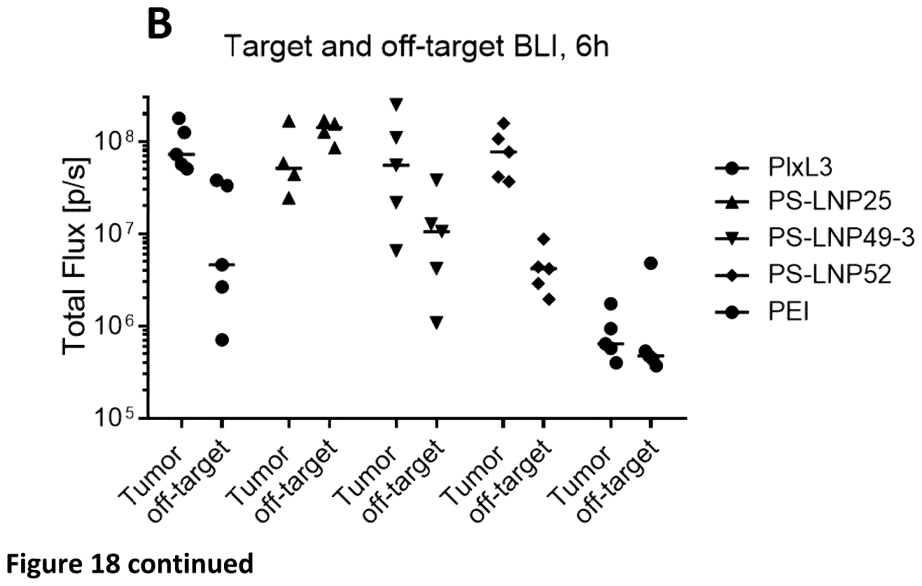

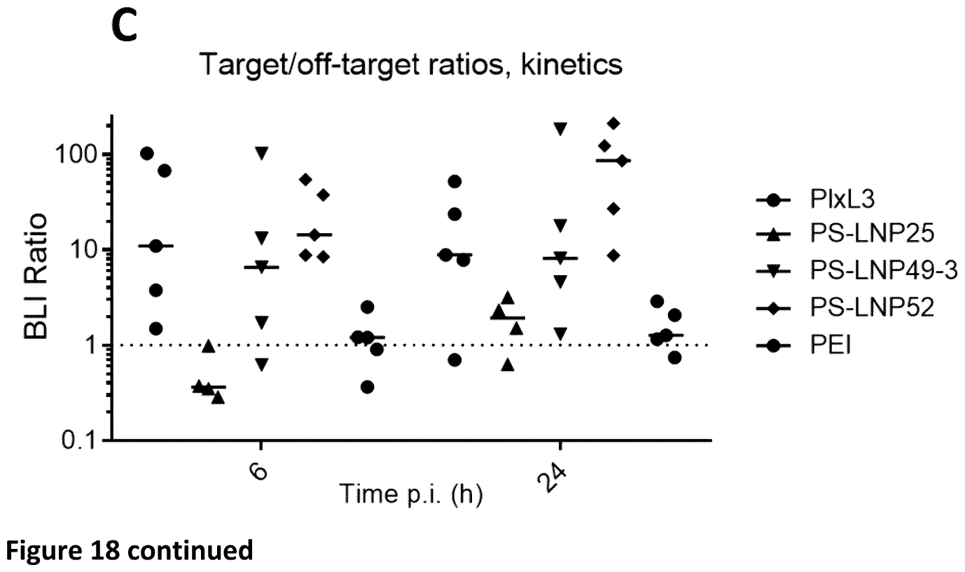

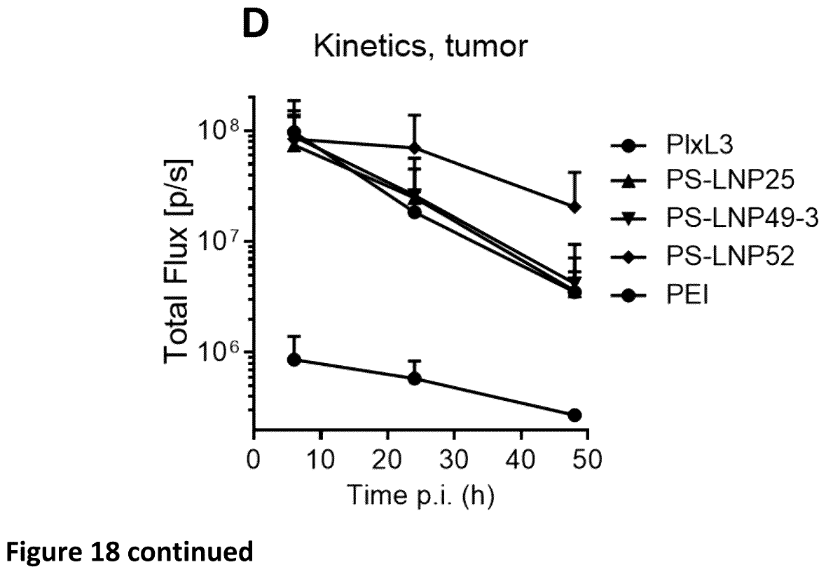

As shown in FIG. 18, in an in vivo experiment 4T1 tumor-bearing mice were injected intra-lesionally with PS-LNPs following the same experimental setting as described in Formulation Example 13. It was demonstrated that the PS-LNP formulations have various biodistribution profiles depending on the ionizable lipid used, in terms of target:off-target ratio. As the target:off-target ratio is an arbitrating parameter for drug specificity, the tested PS-LNPs can be ranked as follows: PS-LNP25<PS-LNP49-3<PS-LNP52.

### Formulation Example 15: PS-LNP52, PS-LNP52-2, PS-LNP52-3, PS-LNP52-4 and PS-LNP52-5

PS-LNP52, PS-LNP52-2, PS-LNP52-3, PS-LNP52-4, PS-LNP52-5 were prepared and characterized as described in the experimental part using the lipid ratios listed in Table 6. In this experiment, the PS-LNPs formulations were designed to investigate the influence of varying the N/P ratio from 3 to 5 and varying the Tween20 concentration from 20 to 5 mol %. As the total mol % is 100, decreasing the tween mol % was compensated by increasing the DOPS concentration from 10 to 30 mol %.

TABLE 6
PS-LNP52, PS-LNP52-2, PS-LNP52-3, PS-LNP52-4, and PS-LNP52-5 lipid
compositions and mol %, buffer, particle sizes, PDI, Zeta-Potential
values, and N/P ratios (pH sensitive (cationic) lipid/RNA).

| | | Z-Average | | Zeta | |
|---|---|---|---|---|---|
| Sample | Lipid Composition | (nm) PBS | PDI | (0.1 PBS) | N/P |
| PS-LNP52 | DODMA/DOPE/DOPS/Tween20 | 188.5 | 0.232 | −32.27 | 3 |
| | (60/10/10/20) | | | | |
| PS-LNP52-2 | DODMA/DOPE/DOPS/Tween20 | 240.3 | 0.243 | −9.71 | 4 |
| | (60/10/10/20) | | | | |
| PS-LNP52-3 | DODMA/DOPE/DOPS/Tween20 | 271.6 | 0.21 | −4.43 | 5 |
| | (60/10/10/20) | | | | |
| PS-LNP52-4 | DODMA/DOPE/DOPS/Tween20 | 242.3 | 0.237 | −27.15 | 3 |
| | (60/10/20/10) | | | | |
| PS-LNP52-5 | DODMA/DOPE/DOPS/Tween20 | 232.4 | 0-219 | −35.25 | 3 |
| | (55/10/30/5) | | | | |

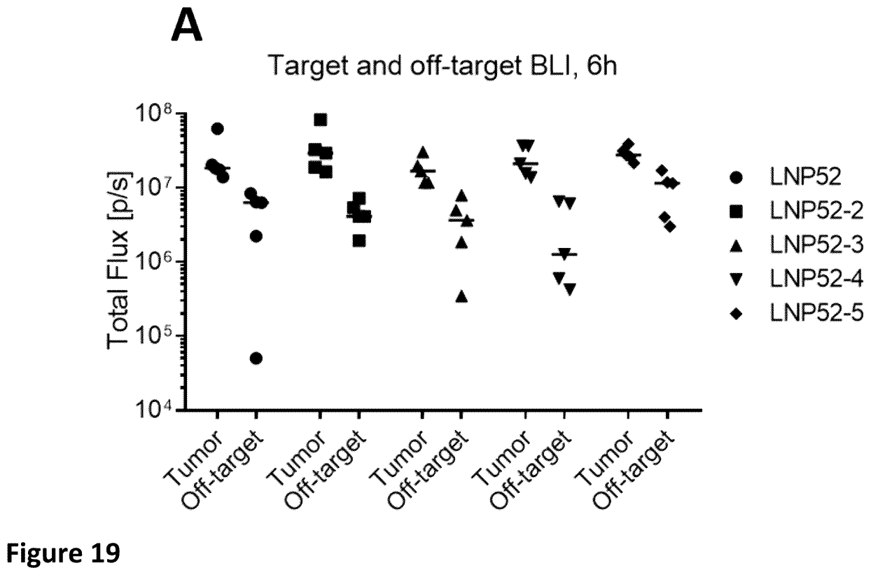

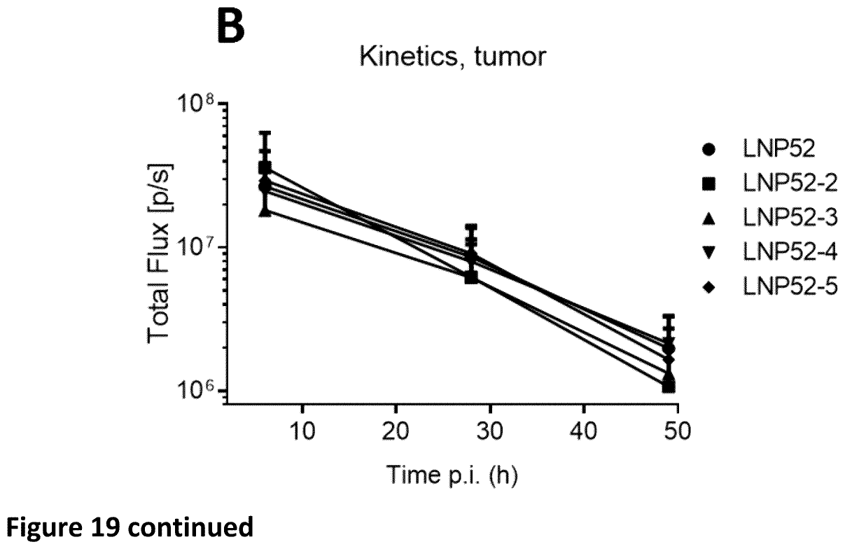

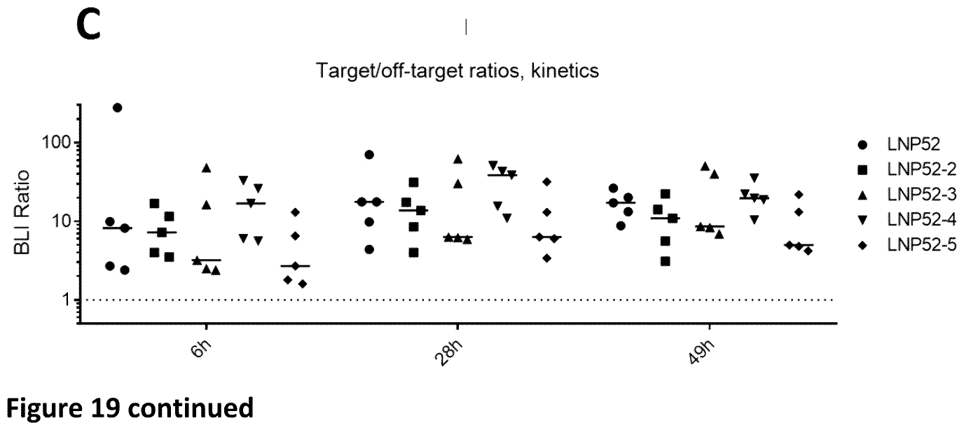

As shown in FIG. 19, mice were injected intratumorally with preparations of the PS-LNPs formulated with two different mRNAs encoding for firefly luciferase and IL-12 following the same experimental setting as described in Formulation Examples 1 and 2. It was demonstrated that increasing the N/P ratio neither substantially changed the organ specificity nor on-target translation of the formulated RNA. It was clearly indicated that replenishing the reduced amount of tween 20 by DOPS does not show a clear impact on target/off-target ratio (FIG. 19).

### Formulation Example 16: PS-LNP52

PS-LNP52 was prepared and characterized as described in the experimental part. The lipid composition and physicochemical data are listed in Table 7.

TABLE 7
PS-LNP52 lipid composition and mol %, buffer, particle sizes, polydispersity
indices (PDI), and N/P ratio (pH sensitive (cationic) lipid/RNA).

| | | | Z-Average | | | |
|---|---|---|---|---|---|---|
| Group | Lipid Composition | Buffer | (nm) | PDI | N/P | pH |
| LNP52 | DODMA/DOPE/DOPS/Tween20 | PBS | 216.1 | 0.169 | 3 | 7 |
| | (60/10/10/20) | | | | | |

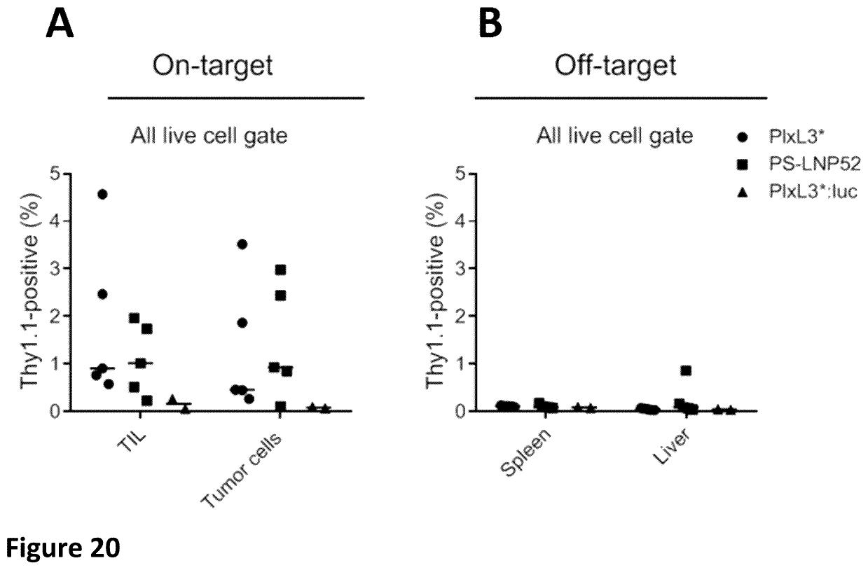

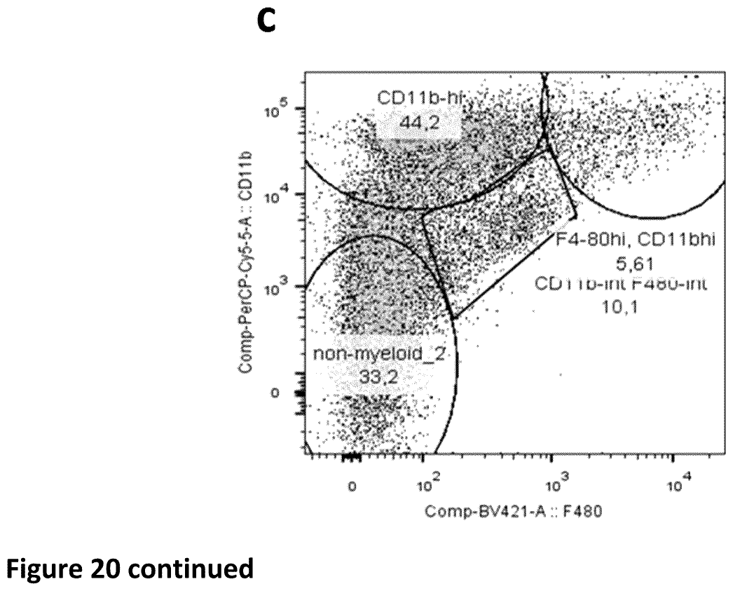

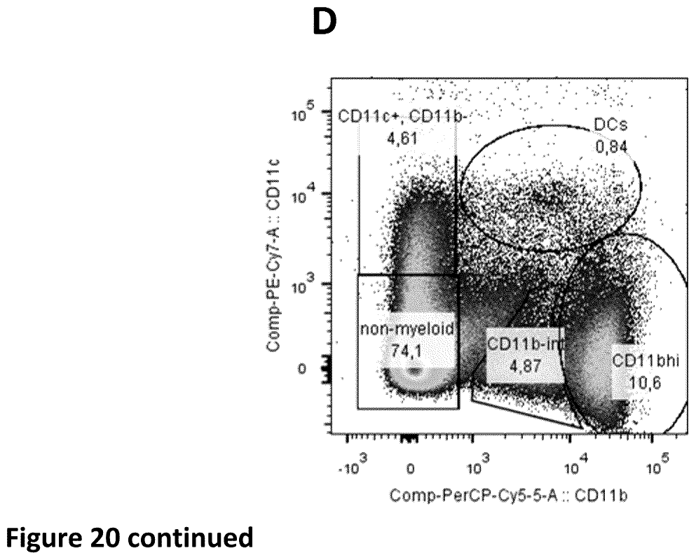

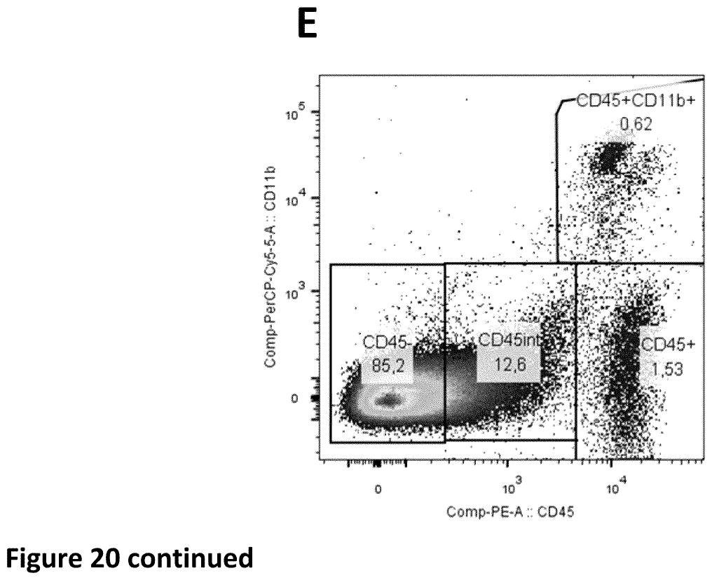

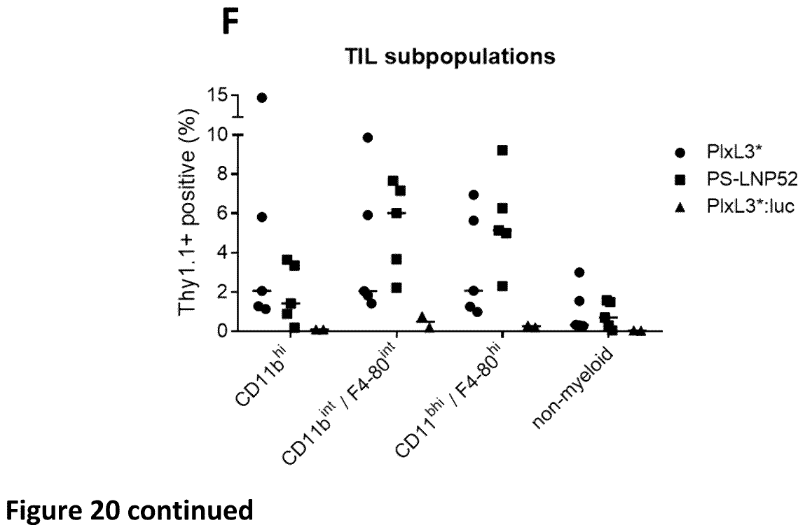

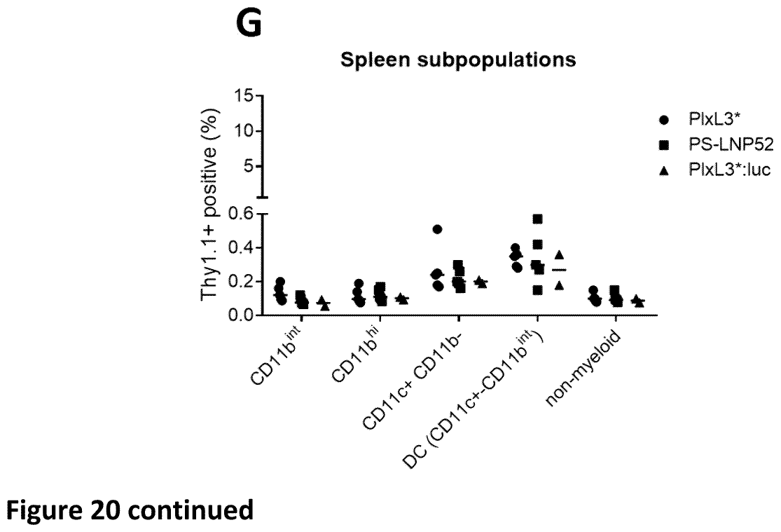

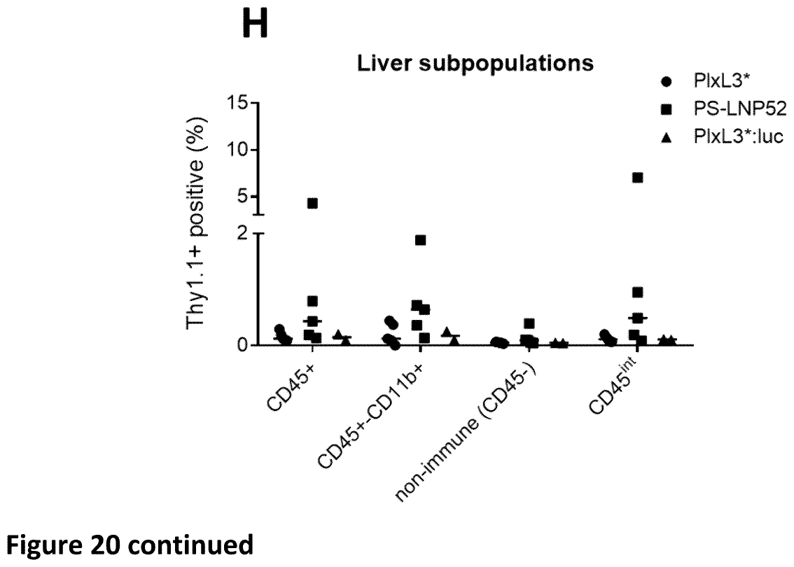

In an in-vivo experiment (FIGS. 20 A&B), PS-LNP52 was formulated with mRNA encoding for Thy 1.1 and control mRNA and injected intratumorally into CT26 tumor-bearing mice. 20 hours post injection, the organs were harvested and flow cytometric analysis completed to analyse Thy1.1 expression. It was demonstrated that the LNP52 formulations tested and the internal benchmark facilitated transfection equally between mRNA tumor-infiltrating leukocytes (TIL) and tumor cells. The off-target translation in spleen and liver cells was low. FIG. 20B showed immune cell type-specific expression of Thy1.1 after intratumoral injection of formulated RNA as analyzed by flow cytometry. It was found that within TIL, meyloid cells are predominantly targeted by the formulated mRNA. Transfection rates were generally low in off-target organs, being mostly confined to myeloid and other immune cells. Hepatocytes (the major liver cell type) are most likely not targeted by the formulated mRNA, as non-immune cells demonstrated barely any reporter expression above background levels.

### Formulation Example 17: PS-LPX52 with Different Tween20 Concentrations Independent from Lipid Concentration as Tween20 Added to Previously Prepared Liposomes

PS-liposomes and PS-LPX52 were prepared as described in the experimental part. The PS-liposomes showed mean particles sizes of 50-90 nm, PDI<0.5 and zeta potential of +30-60 mV when measured in 15 mM NaCl aqueous solution.

TABLE 8
Lipid Composition, N/P ratio, Z-Average, PDI, pH, reporter
RNA and total formulated RNA dose/mouse of PS-LPX52.

| | | | | | | | | Total |
|---|---|---|---|---|---|---|---|---|
| | | | | | | | | dose |
| | | Compositions/mol | | Size | | | | μg/ |
| | Form | %/concentration mM | N/P | (nm) | PDI | pH | Reporter | mouse |
| A | PS- | (DODMA/DOPE/DOPS/Tween20, | 3 | 233.9 | 0.237 | 5-6 | IL12-LUC | 10 |
| | LPX52 | 60/10/10/20, | | | | | | |
| | | 1.8/0.3/0.3/0.6 | | | | | | |
| B | PS- | DODMA/DOPE/DOPS/Tween20, | 3 | 189.8 | 0.231 | 5-6 | IL12-LUC | 10 |
| | LPX52 | 66.67/11.11/11.11/11.11, | | | | | | |
| | | 1.8/0.3/0.3/0.3 | | | | | | |
| C | PS- | DODMA/DOPE/DOPS/Tween20, | 3 | 259.3 | 0.216 | 5-6 | IL12-LUC | 10 |
| | LPX52 | 70.59/11.76/11.76/5.89, | | | | | | |
| | | 1.8/0.3/0.3/0.15 | | | | | | |

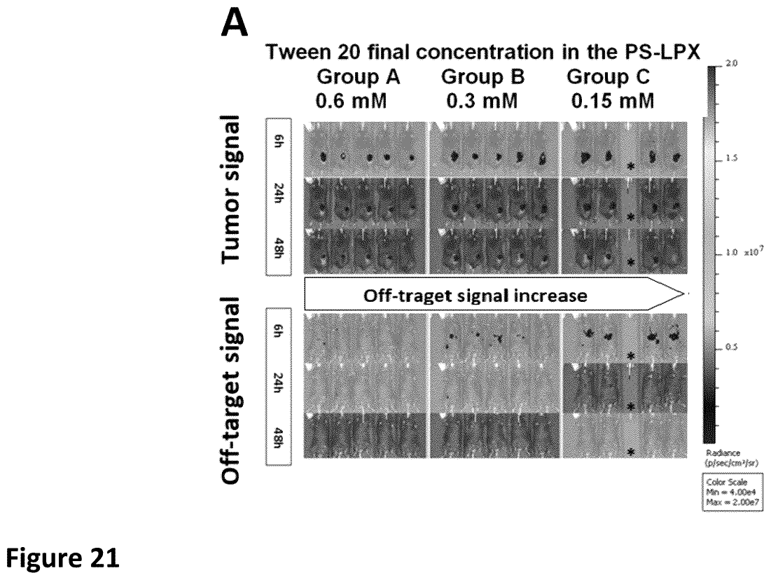

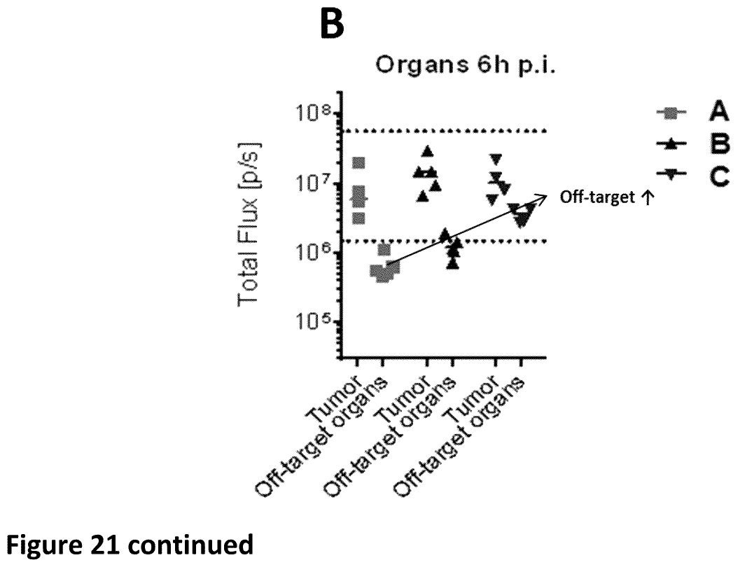

In an in-vivo experiment (FIG. 21), PS-LPX52 with varying Tween20 concentrations was formulated with mRNA encoding for firefly luciferase and injected intratumorally into mice with the experimental aim to facilitate transfection and target translation: PS-LPX52 Group A: DODMA/DOPE/DOPS/Tween20, 60/10/10/20 (1.8/0.3/0.3/0.6 mM), PS-LPX52 Group B: DODMA/DOPE/DOPS/Tween20, 66.67/11.11/11.11/11.11 (1.8/0.3/0.3/0.3 mM), PS-LPX52 Group C: DODMA/DOPE/DOPS/Tween20, 70.59/11.76/11.76/5.89 (1.8/0.3/0.3/0.15 mM). All formulations showed in-range particle sizes and PDI values (Table 8). All formulations of PS-LPX52 mediated high luciferase activity and demonstrated that decreasing Tween20 from 0.6 mM to 0.15 mM leads to a decrease in target/off-target ratio (group A>group B>Group C).

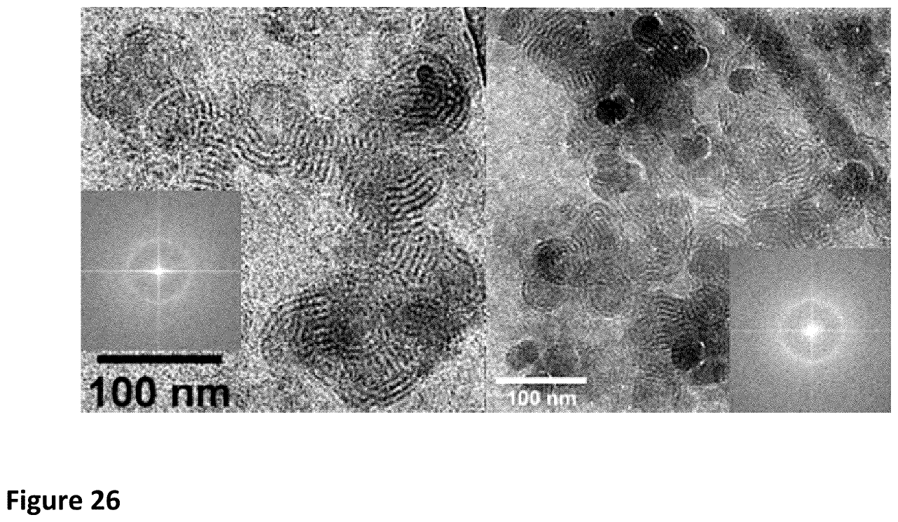

In CryoTEM imaging of the PS-LPX as shown in FIG. 26, PS-LPX52 showed vesicular nanoparticles, oligo and multilamellar lipoplexes in the size range of 50 to 200 nm

### Formulation Example 18: Therapeutic Efficacy of PS-LNP25 and PS-LNP49-3

This experiment was designed to test the therapeutic efficacy of PS-LNP25 (that demonstrates high target/off-target ratio as shown in Formulation Example 14, FIG. 18) and PS-LNP49-3 (that demonstrates reasonable improvement in target/off-target ratio as shown in Formulation Example 14, FIG. 18). PS-LNP25 and PS-LNP49-3 were prepared and characterized as described in the experimental part using the lipid ratios listed in Table 9.

TABLE 9
PS-LNP25, PS-LNP49-3, and Ringer compositions and mol %, buffer, zeta potential
values, N/P ratio (pH sensitive (cationic) lipid/RNA), and RNA used.

| | | | Zeta. | | |
|---|---|---|---|---|---|
| | | | Pot. | | |
| Groups | Compositions | Buffer | [mV] | N/P | RNA |
| PS-LNP25 | Dlin-KC2-DMA/CHOL/DOPE/DOPS/Tween20 | PBS | −4.96 | 3 | RNA |
| | (47/30/10/10/3) | | | | Cytokine |
| | | | | | Mix |
| PS-LNP49-3 | Dlin-MC3-DMA/DOPE/DOPS/Tween20 | PBS | −2.48 | 3 | RNA |
| | (60/10/10/20) | | | | Cytokine |
| | | | | | Mix |
| Ringer | Ringer | Ringer | | — | RNA |
| | | | | | Cytokine |
| | | | | | Mix |
| PS-LNP25 | Dlin-KC2-DMA/CHOL/DOPE/DOPS/Tween20 | PBS | −6.45 | 3 | LUC |
| | (47/30/10/10/3) | | | | |
| PS-LNP49-3 | Dlin-MC3-DMA/DOPE/DOPS/Tween20 | PBS | −2.98 | 3 | LUC |
| | (60/10/10/20) | | | | |
| Ringer | Ringer | Ringer | | — | LUC |

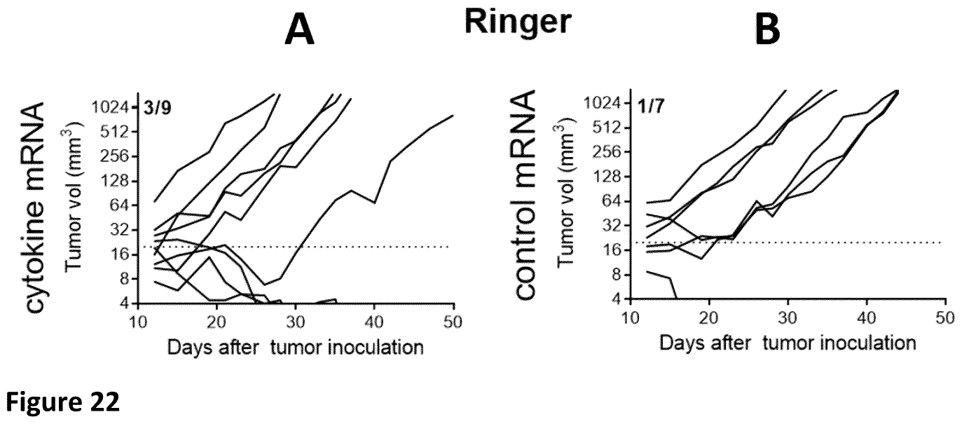

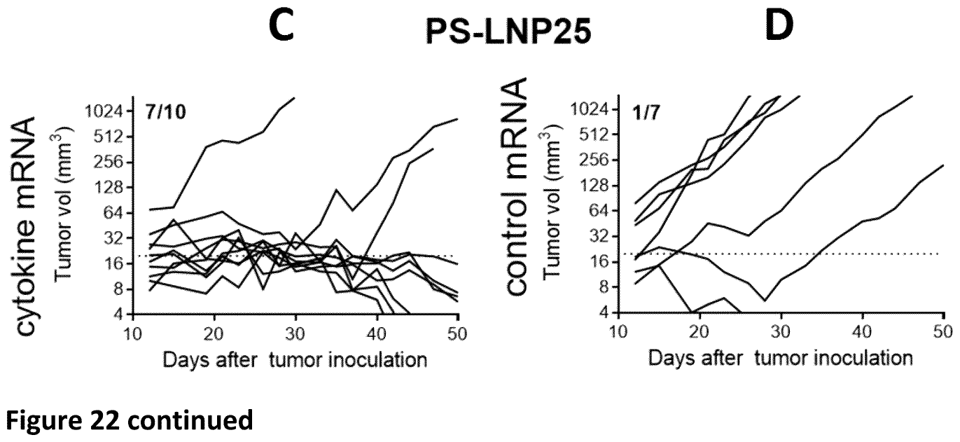

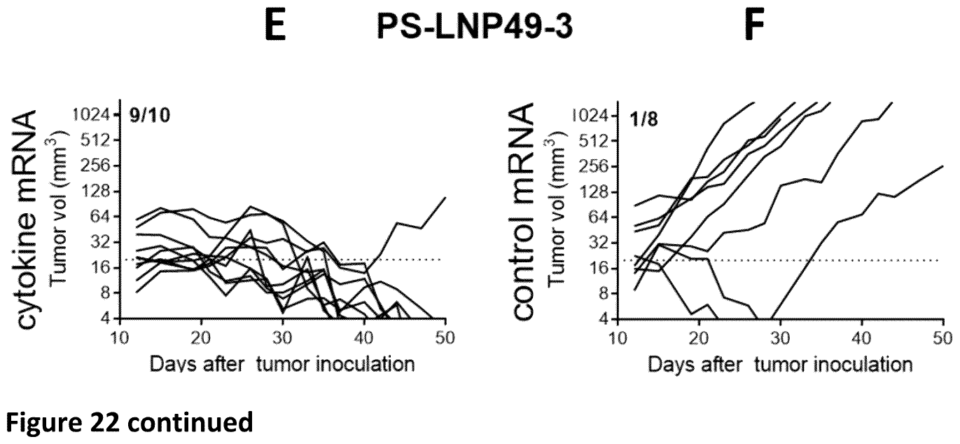

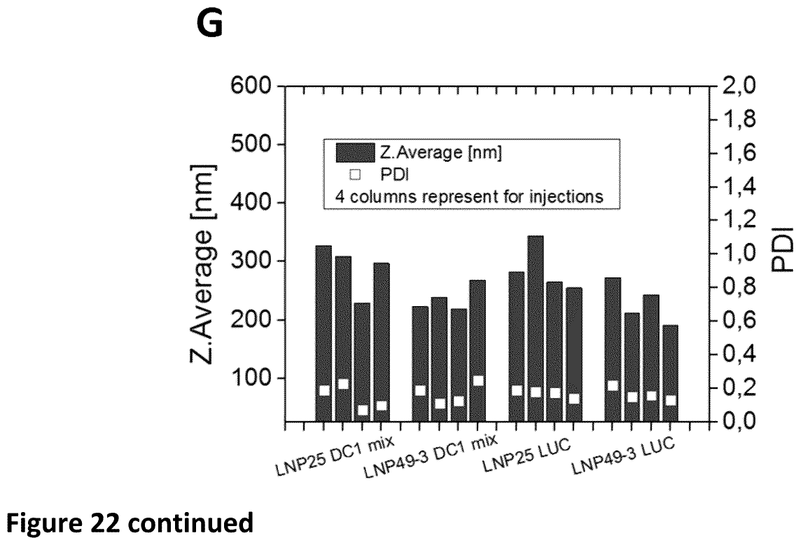

As shown in FIG. 22, mice bearing established CT26 tumors were injected intratumorally with a mixture of cytokine-encoding mRNAs (RNA Cytokine Mix) or control (LUC) mRNA complexed with the indicated formulations. Particle sizes and PDI values of four different batches are depicted in FIG. 22 B. As shown in FIG. 22 A, compared to naked RNA in Ringer solution, all formulations increased the complete response rate, with LNP49-3 curing 90% of treated mice.

### Formulation Example 19: Therapeutic Efficacy of PS-LPX52 vs Ringer

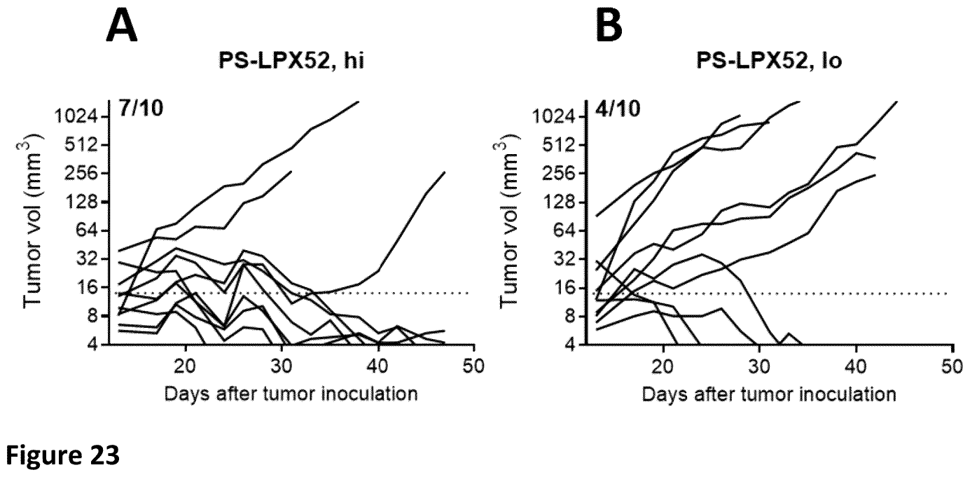

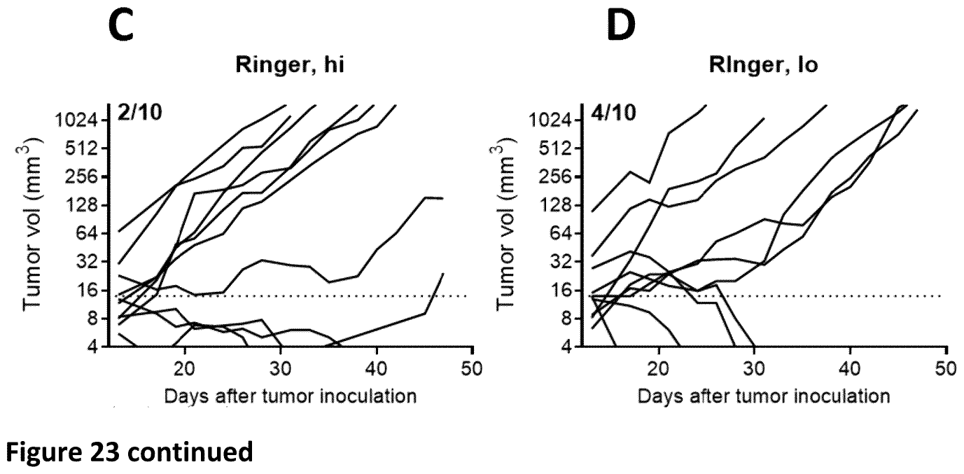

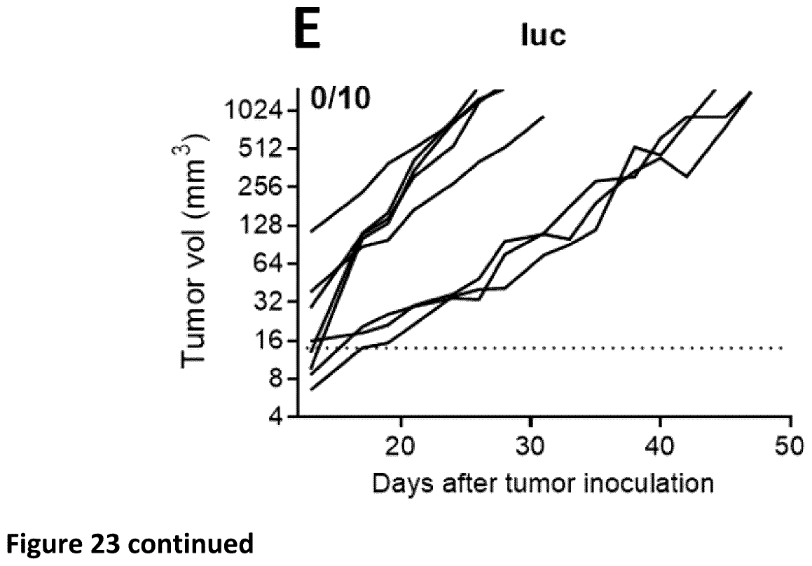

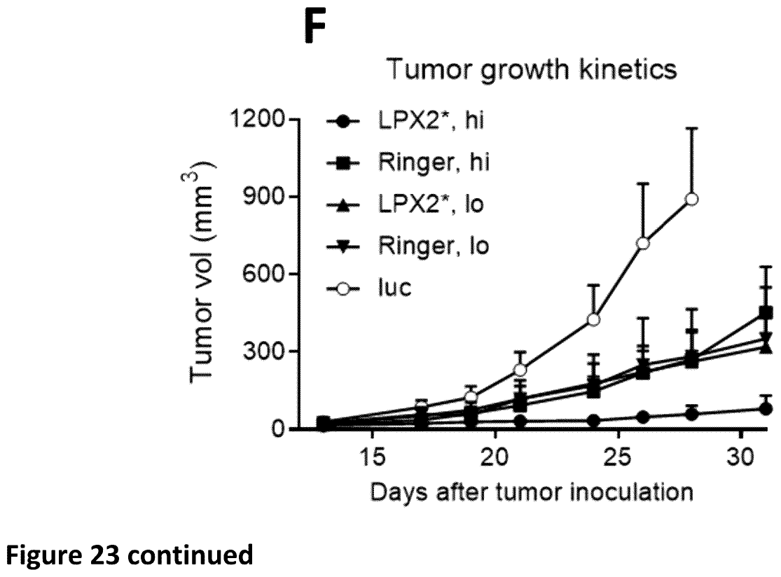

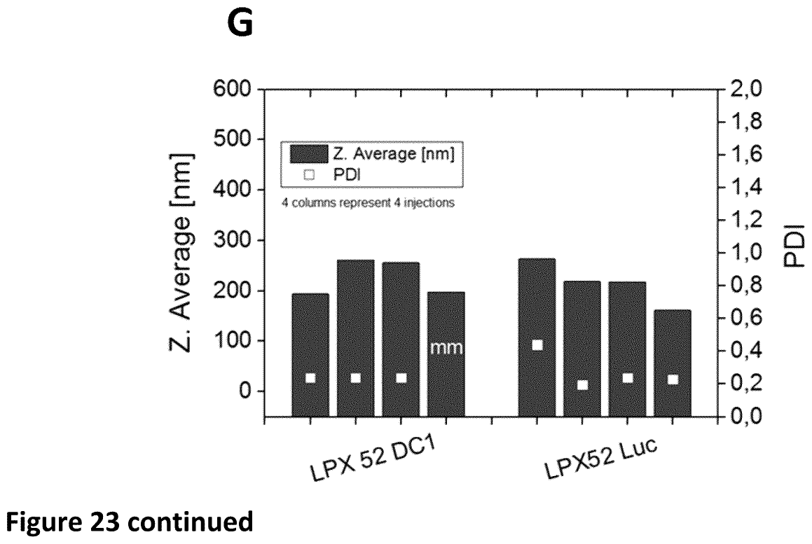

In this experiment, PS-LPX52 (DODMA/DOPE/DOPS/Tween20/RNA) and RNA ringer solution were tested concurrently to prove the superiority of the PS-LPX-formulated RNA over the RNA-Ringer solution. PS-LPX52 was prepared and characterized as described in the experimental part while the Ringer/RNA solution was prepared by simple mixing of both Ringer and aqueous RNA solutions. As depicted in FIG. 23B, all particle sizes values were in the expected range (200-280 nm). Mice bearing established CT26 tumors were treated intratumorally once a week for 4 weeks with a mixture of cytokine-encoding mRNAs or control LUC mRNA complexed with either PS-LPX52 or in plain Ringers salt solution in two doses each. It was demonstrated that the cytokine mRNA formulated with PS-LPX52 in the highest dose was most efficient in suppressing tumor growth and cured 7 out of 10 treated mice (FIG. 23 A).

TABLE 10
PS-LPX52 and Ringer Compositions, N/P ratio (pH sensitive (cationic)
lipid/RNA), dose/target/mouse, total RNA per mouse, and RNA used.

| | | N/P | Dose/Target/ | Total RNA/ | |
|---|---|---|---|---|---|
| Formulations | Compositions | ratio | Mouse [μg] | Mouse [μg] | RNA |
| PS-LPX52 | DODMA/DOPE/DOPS/Tween20 | 3 | 2.5 | 10 | RNA |
| | (60/10/10/20) | | | | Cytokine Mix |
| Ringer | | — | | 10 | RNA |
| | | | | | Cytokine Mix |
| PS-LPX52 | DODMA/DOPE/DOPS/Tween20 | 3 | 1 | 4 | RNA |
| | (60/10/10/20) | | | | Cytokine Mix |
| Ringer | | — | | 4 | RNA |
| | | | | | Cytokine Mix |
| PS-LPX52 | DODMA/DOPE/DOPS/Tween20 | 3 | 10 | 10 | LUC |
| | (60/10/10/20) | | | | |

### Formulation Example 20: Effect of Long Term Frozen Storage of PS-LPX2*It.IV Vs Ringer

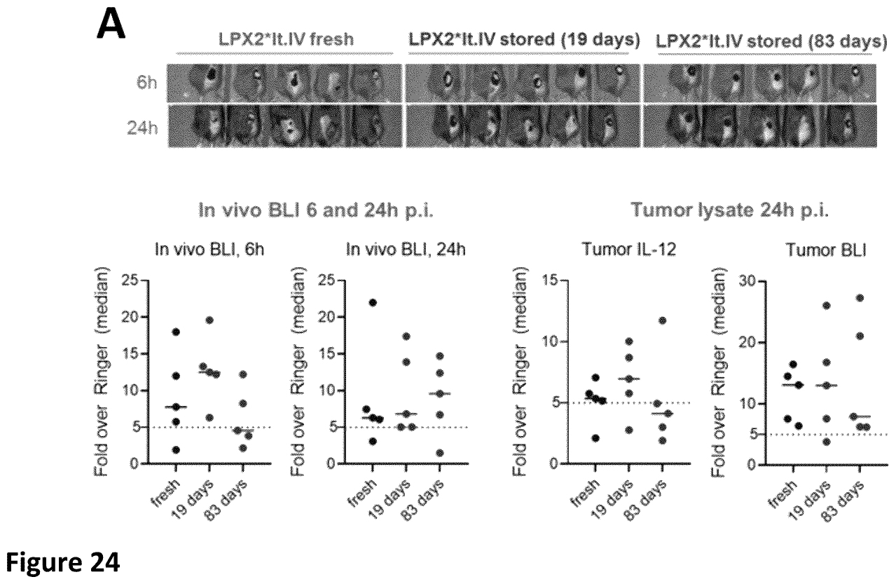

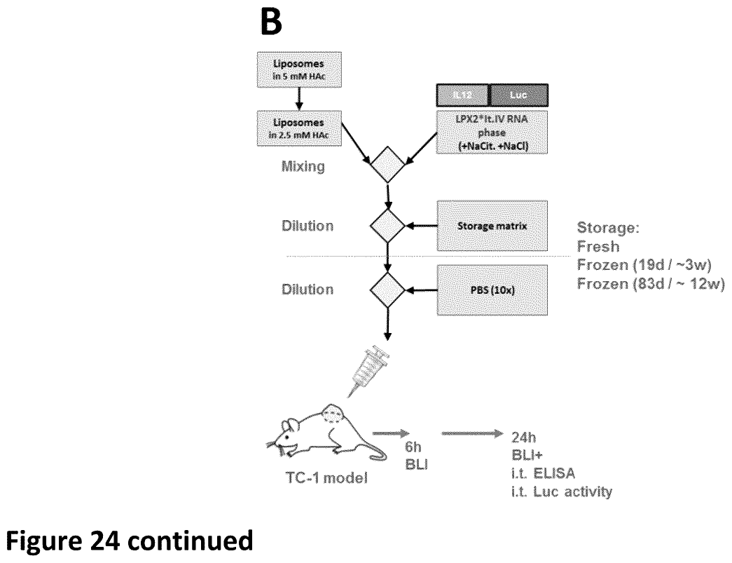

In this experiment PS-LPX2* It.IV (DODMA/DOPE/DOPS/Tween20, 60/10/10/20 mol %, PS-LPX52 with storage additives) was formulated in a manner which made it compatible with extended frozen storage at −20° C. and manufactured using a modified protocol adapted for fluid path. In brief, PS-LPX2*It.IV was prepared by combining two equal volume phases of RNA and liposomal precursor through a fluid path, the RNA phase containing some buffering salt (disodium citrate and sodium chloride). The lipoplexed RNA was then collected, and a cryoprotectant-containing Storage Matrix (Sodium acetate and sucrose) was added, enabling the PS-LPX2*It.IV material to be frozen. On the day of the below referenced in vivo experiment, aliquots of the PS-LPX2*It.IV were removed and brought to room temperature; 10×DPBS was then added to each aliquot to reach 1×PBS concentration and the solution was gently mixed. The PS-LPX2*It.IV material was then diluted with the bulk phase to the appropriate concentration (0.1 mg/mL), corresponding to the intended dose of 5 μg total RNA. A quantitative description of the excipients within PS-LPX2*It.IV is given in Table 11. Summarised physicochemical data for this experiment is presented in Table 12. Following QC measurements, the material was administered intratumorally into mice bearing TC-1 model tumors. No effect of extended storage of the PS-LPX2*It.IV was observed and both the fresh and aged samples produced comparably enhanced cytokine expressions relative to the Ringer control, marked by the dashed line in FIG. 24.

TABLE 11
Composition of PS-LPX2*It.IV (PS-LPX52 with storage
additives) during storage and at injection

| | | Concentration | | Concentration | |
|---|---|---|---|---|---|
| Product attribute | Function | during storage | | At injection | |
| Drug Substance | Active Substance | 0.22 | mg/mL | 0.20 | mg/mL |
| DODMA | Functional lipid | 1.24 | mg/mL | 1.12 | mg/mL |
| DOPE | Structural lipid | 0.25 | mg/mL | 0.225 | mg/mL |
| DOPS | Structural lipid | 0.27 | mg/mL | 0.24 | mg/mL |
| TWEEN20 | Surfactant | 0.82 | mg/mL | 0.74 | mg/mL |
| HAc | Buffer | 0.03 | mg/mL | 0.027 | mg/mL |
| Sucrose | Cryoprotectant | 100.00 | mg/mL | 90.00 | mg/mL |
| NaOCit. | Buffer | 0.26 | mg/mL | 0.23 | mg/mL |
| NaOAc. | Buffer | 0.41 | mg/mL | 0.37 | mg/mL |
| NaCl | Buffer | 0.13 | mg/mL | 8.14 | mg/mL |
| KCl | Buffer | 0.00 | mg/mL | 0.20 | mg/mL |
| Na₂HPO₄ | Buffer | 0.00 | mg/mL | 1.42 | mg/mL |
| KH₂PO₄ | Buffer | 0.00 | mg/mL | 0.24 | mg/mL |

| | | | |
|---|---|---|---|
| Water for | Solvent/Vehicle | quantum satis | quantum satis |
| injection | | | |

TABLE 12
Table of summarised physicochemical parameters for the PS-LPX2*It.IV
DP corresponding to the in vivo data presented in FIG. 24

| Time | | | | | | | | |
|---|---|---|---|---|---|---|---|---|
| stored | Osmolality | | SVP count | SVP count | SVP count | zeta | size/z. ave | |
| at −20° C. | (Osmol/kg) | pH | (>1 μm) | (>10 μm) | (>25 μm) | (mV) | (nm) | PDI |
| ,,fresh“ | 0.610 | 6.23 | 13977 | 22 | 0 | −31.25 | 214.20 | 0.238 |
| 1 day | | | | | | | | |
| 19 days | 0.637 | 6.77 | 6270 | 30 | 2 | −33.38 | 235.50 | 0.229 |
| 83 days | 0.615 | 6.78 | 316 | 1 | 0 | −18.68 | 203.13 | 0.238 |

### Formulation Example 21: Generation of RNA Lipid Nanoparticles Comprising Phosphatidylserine in the Presence of Different Types of Non-Ionic Surfactants

This experiment aimed to compare the physiochemical characteristics of PS-LNPs and PS-LPXs formulated with RNA in the presence of different types of non-ionic surfactants.

As shown in Table 13, all particles had a well-defined particle size and a neutral to negative zeta potential.

TABLE 13
Physicochemical characterization of PS-LNPs and PS-
LPXs stabilized by different non-ionic surfactants.

| | | | | Zeta P. (mV) | | |
|---|---|---|---|---|---|---|
| | | Size | | | 15 mM | N/P |
| | Lipid Composition (mol %) | (nm) | PDI | water | NaCl | ratio |
| PS-LNP- | DODMA/DOPS/TPGS, (87/10/3) | 151.7 | 0.09 | −31.19 | −4.78 | 3 |
| TPGS-1 | | | | | | |
| PS-LNP- | DODMA/DOPE/DOPS/TPGS, | 171.9 | 0.10 | −29.76 | −3.98 | 3 |
| TPGS-2 | (77/10/10/3) | | | | | |
| PS-LNP- | DODMA/CHOL/DOPS/TPGS, | 115.5 | 0.02 | −4.12 | 7.2 | 3 |
| TPGS-3 | (57/30/10/3) | | | | | |
| PS-LNP- | DODMA/CHOL/DOPS/Solutol, | 245.7 | 0.10 | −16.13 | 7.1 | 3 |
| Solutol | (57/30/10/3) | | | | | |
| PS-LNP- | DODMA/CHOL/DOPS/Tween80, | 194.3 | 0.10 | −2.87 | 2.87 | 3 |
| Tween80 | (57/30/10/3) | | | | | |
| PS-LNP- | DODMA/DOPS/Myrj52, 87/10/3 | 229.3 | 0.24 | −16.31 | −7.17 | 3 |
| Myrj52 | | | | | | |
| PS-LPX- | Dlin-MC3-DMA/DOPE/DOPS/Tween20 | 420.1 | 0.363 | NA | −38.21 | 3 |
| Dlin-MC3 | (60/10/10/20) | | | | | |
| PS-LPX- | DlinDMA/DOPE/DOPS/Tween20 | 449.5 | 0.45 | NA | NA | 3 |
| DlinDMA | (60/10/10/20) | | | | | |

TABLE 14
Summary table for all PS-LNPs and PS-LPXs

| Formulation | |
|---|---|
| name | Lipid Composition (mol %) |
| PS-LNP1 | DODMA/CHOL/DOPS/Tween20, (57/30/10/3) |
| PS-LNP2 | DODMA/DOPE/DOPS/Tween20, (77/10/10/3) |
| PS-LNP3 | DODMA/DOPS/Tween 20, (87/10/3) |
| PS-LNP8 | DODMA/DOPE/DOPS/Tween20, (62/25/10/3) |
| PS-LNP9 | DODMA/DOPE/DOPS/Tween20, (70/10/10/10) |
| PS-LNP16 | DODMA/DOPE/DOPS/Tween20, (27/60/10/3) |
| PS-LNP19 | DODMA/CHOL/DOPS/Tween20, (77/10/10/3) |
| PS-LNP23 | DMRIE/CHOL/DOPE/DOPS/Tween20, (47/30/10/10/3) |
| PS-LNP24 | DODMA/CHOL/DOPE/DOPS/Tween20, (47/30/10/10/3) |
| PS-LNP25 | DlinDMA-KC2/CHOL/DOPE/DOPS/Tween20, (47/30/10/10/3) |
| PS-LNP26 | DlinDMA-MC3/CHOL/DOPE/DOPS/Tween20, (47/30/10/10/3) |
| PS-LNP49 | Dlin-MC3/DOPE/DOPS/Tween20, (50/10/10/30) |
| PS-LNP49-2 | Dlin-MC3/DOPE/DOPS/Tween20, (55/10/10/25) |
| PS-LNP49-3 | Dlin-MC3/DOPE/DOPS/Tween20, (60/10/10/20) |
| PS-LNP50 | Dlin-MC3/DOPE/Tween20, (55/20/25) |
| PS-LNP52 | DODMA/DOPE/DOPS/Tween20, (60/10/10/20) |
| PS-LNP52-4 | DODMA/DOPE/DOPS/Tween20, (60/10/20/10) |
| PS-LNP52-5 | DODMA/DOPE/DOPS/Tween20, (55/10/30/5) |
| PS-LPX52 | (DODMA/DOPE/DOPS/Tween20, 60/10/10/20 (1.8/0.3/0.3/0.6 mM) |
| PS-LPX52 | DODMA/DOPE/DOPS/Tween20, 66.67/11.11/11.11/11.11 |
| | (1.8/0.3/0.3/0.3 mM) |
| PS-LPX52 | DODMA/DOPE/DOPS/Tween20, 70.59/11.76/11.76/5.89 |
| | (1.8/0.3/0.3/0.15) |
| PS-LNP | DODMA/DOPS/TPGS, (87/10/3) |
| PS-LNP | DODMA/DOPE/DOPS/TPGS, (77/10/10/3) |
| PS-LNP | DODMA/CHOL/DOPS/TPGS, (57/30/10/3) |
| PS-LNP | DODMA/CHOL/DOPS/Solutol, (57/30/10/3) |
| PS-LNP | DODMA/DOPS/Myrj52, 87/10/3 |
| PS-LPX | Dlin-MC3-DMA/DOPE/DOPS/Tween20 (60/10/10/20) |
| PS-LPX | DlinDMA/DOPE/DOPS/Tween20 (60/10/10/20) |

### Example 3: Further Optimisation of PS-LPX Formulation

Additional experiments were performed to further optimize an RNA-lipoplex formulation comprising a mixture of four chemically modified RNAs encoding human cytokines in a 1:1:1:1 weight/volume ratio. The RNAs were single-stranded, 5′-capped RNAs produced by in vitro transcription, and provided as 2 mg/mL solution in 10 mM HEPES/0.1 mM EDTA (NaOH, pH 7.0) buffer. The RNA mixture was complexed with a liposome precursor comprising 1,2-dioleyloxy-3-dimethylaminopropane (DODMA), 1,2-dioleoyl-sn-glycero-3-phosphoethanolamine (DOPE), 1,2-dioleoyl-sn-glycero-3-phosphoserine (DOPS), Polysorbate 20 and provided in a sodium acetate buffer supplemented with sucrose as cryoprotectant.

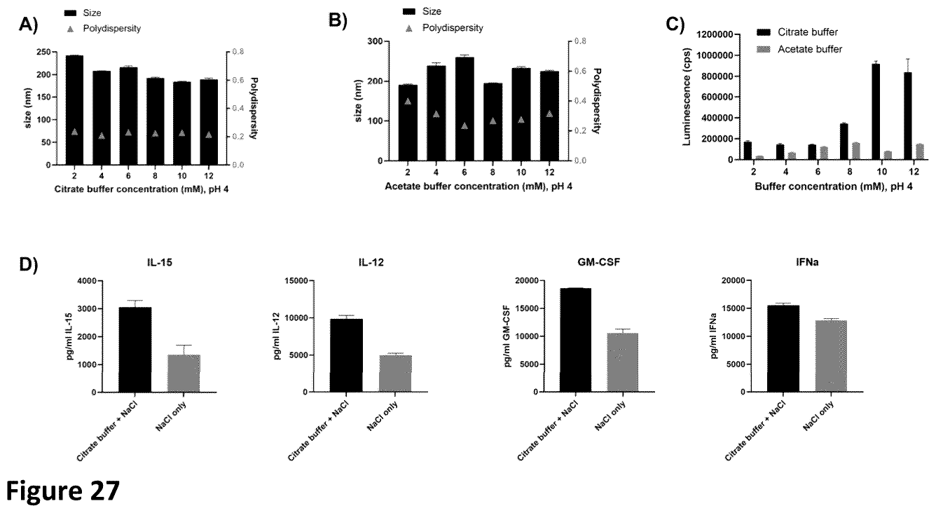

Lipoplexes (LPX) formulated with luciferase RNA, with citrate buffer as acidifier were relatively smaller in particle size as compared to that prepared with acetate buffer at tested concentrations (FIG. 27A,B). Furthermore, lipoplexes prepared with citrate buffer showed higher levels of in vitro transfection as compared to those prepared with acetate buffer (FIG. 27C). Higher citrate buffer concentrations lead to higher luciferase expression, but the corresponding higher ionic load negatively impacted the colloidal stability of the LPX in a frozen state. As a balance between biological activity and colloidal stability, a citrate buffer concentration of 4.5 mM was selected. Additionally, an increased in vitro transfection was noted for all four cytokines, when the RNA phase was supplemented with a combination of citrate buffer and sodium chloride (FIG. 27D).

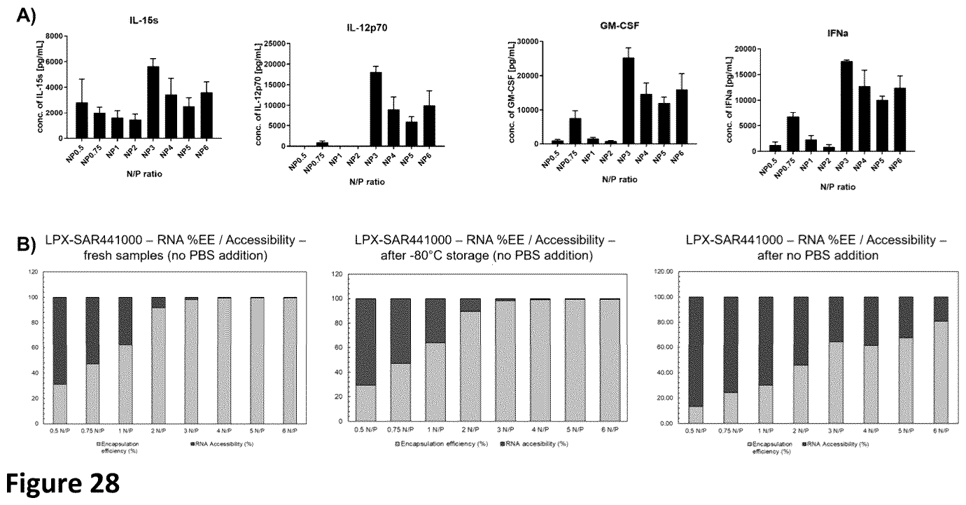

The ratio of complexing cationic lipid to RNA (N/P ratio, as further described herein) was investigated. It was found that in vitro protein expression of the 4-cytokine RNA mixture from PS-LPX was lower at N/P ratios <3 (FIG. 28A). Additionally, the encapsulation efficiency of the RNA in the PS-LPX was assessed, and accessible RNA was also measured. It was found that improved RNA encapsulation was observed at N/P ratios of 3 or more (FIG. 28B). Significant improvements in either protein expression or encapsulation efficiency were not observed for increasingly higher N/P ratios. Therefore, an N/P ratio of 3 was selected for the 4-cytokine RNA drug product.

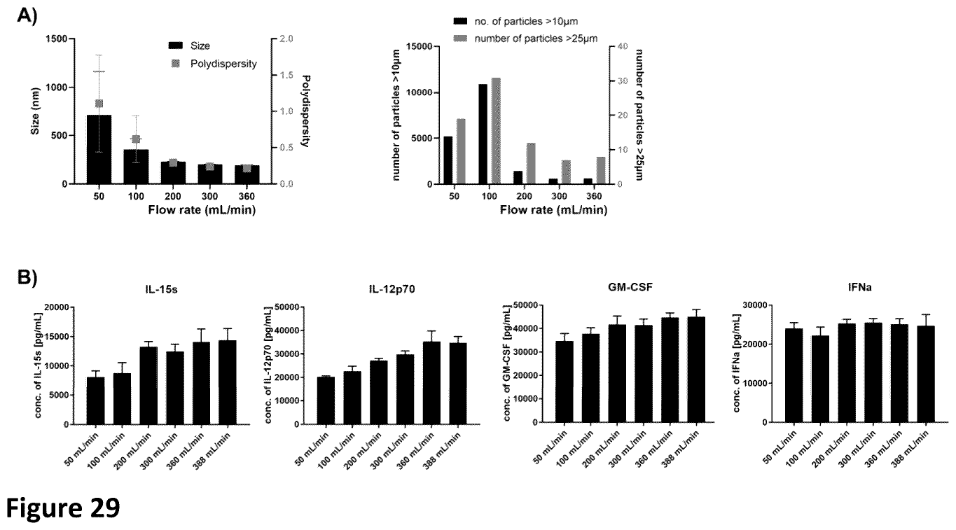

LPX formation was performed using a 1:1 mixing of the RNA and liposome phase using a Y-type mixing element with tubing length of 5 cm for the liposome and RNA phases channels, and 20 cm for the mixing channel, and a tubing diameter of either 1.6 or 2.4 mm. LPX were prepared at varying total flow rates from 50-388 mL/min. LPX particles formed at flow rates <200 mL/min had relatively higher sub-visible particles counts (FIG. 29A). Particle size and polydispersity was higher for samples prepared at flow rates <100 mL/min (FIG. 29A). Samples prepared at both 300 and 360 mL/min were comparable in terms of particle size, polydispersity and sub-visible count (FIG. 29A). A trend of increasing protein expression with increasing flow rate is observed (FIG. 29B). A flow total rate of 360 mL/min was selected for LPX manufacturing.

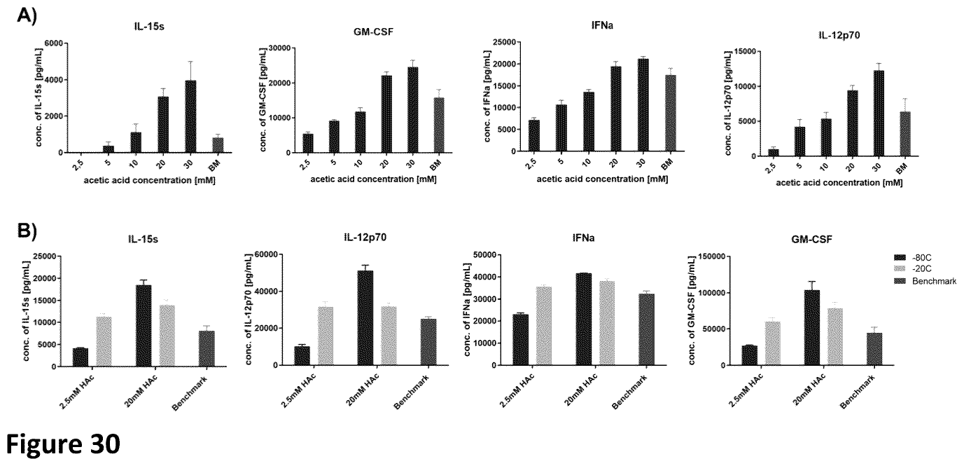

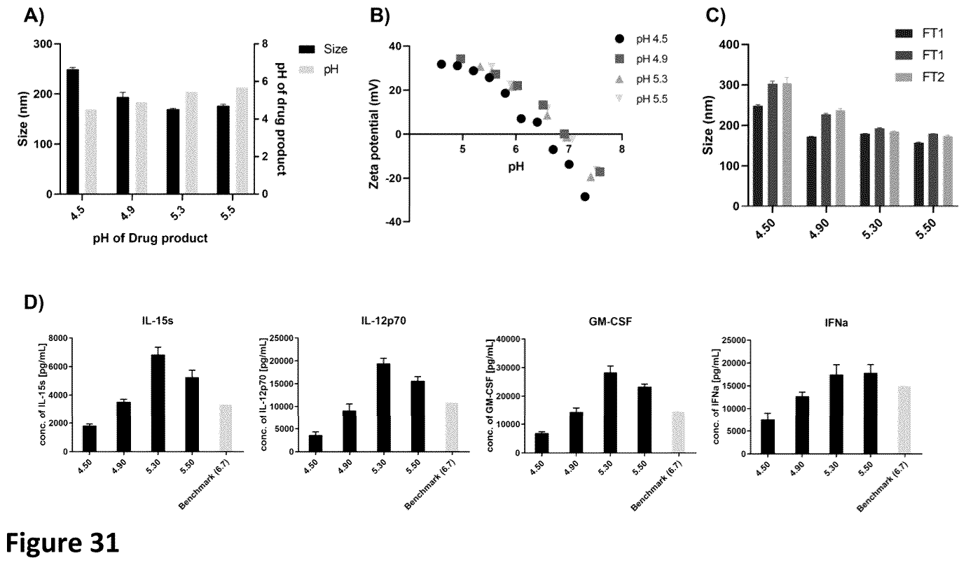

Optimizations to the PS-LPX manufacturing process were also investigated and an improved method of manufacturing PS-LPX was developed. Described is a method of producing RNA lipoplex (RNA-LPX) particles, the method comprising: (i) preparing an acidified aqueous solution comprising liposomes, wherein the acidified aqueous solution comprising liposomes comprises from 10 to 30 mM acetic acid; and (ii) mixing the acidified aqueous solution comprising liposomes with an aqueous solution comprising RNA; thereby producing a solution comprising RNA-LPX particles; wherein the RNA-LPX particles and the liposomes comprise dioleoylphosphatidylserine (DOPS); N,N-dimethyl-2,3-dioleyloxypropylamine (DODMA); and dioleoylphosphatidylethanolamine (DOPE) (DODMA/DOPE/DOPS RNA-LPX). The effects of the tested acetic acid concentrations on in vitro protein expression of the 4-cytokine RNA mixture from the DODMA/DOPE/DOPS RNA-LPX are shown (FIG. 30A,B). The aqueous solution comprising the RNA did not comprise sodium chloride (NaCl). The concentration of RNA in the aqueous solution comprising RNA, at the point of mixing with the solution comprising liposomes, was 0.5 mg/mL or less. The method further comprises adding a storage/dilution buffer to the formed DODMA/DOPE/DOPS RNA-LPX, wherein the storage/dilution buffer comprises 30% sucrose and 20 mM histidine buffer at pH 5.5 or 6.5. The pH of the DODMA/DOPE/DOPS RNA-LPX comprising the storage/dilution buffer was 5.30 or 5.50. Improved storage stability and higher protein expression from DODMA/DOPE/DOPS RNA-LPX at pH 5.30 or pH 5.5 was observed (FIG. 31A-D).

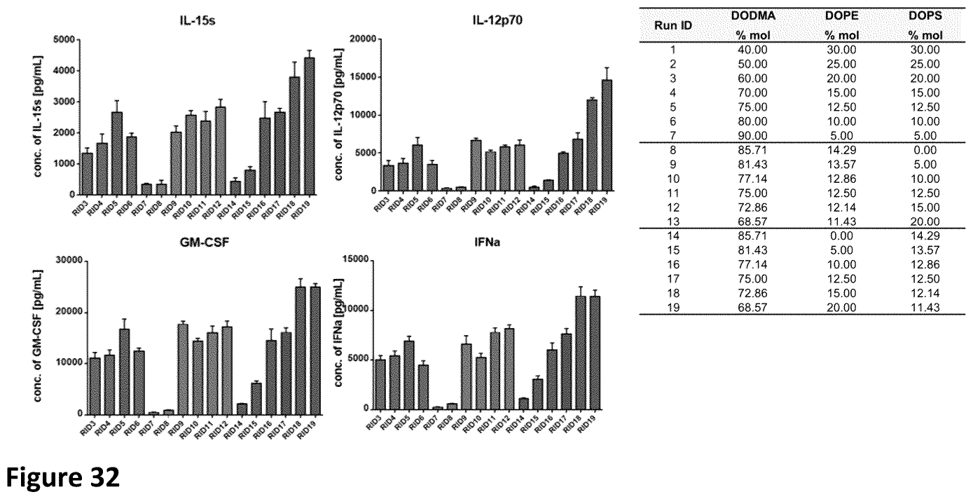

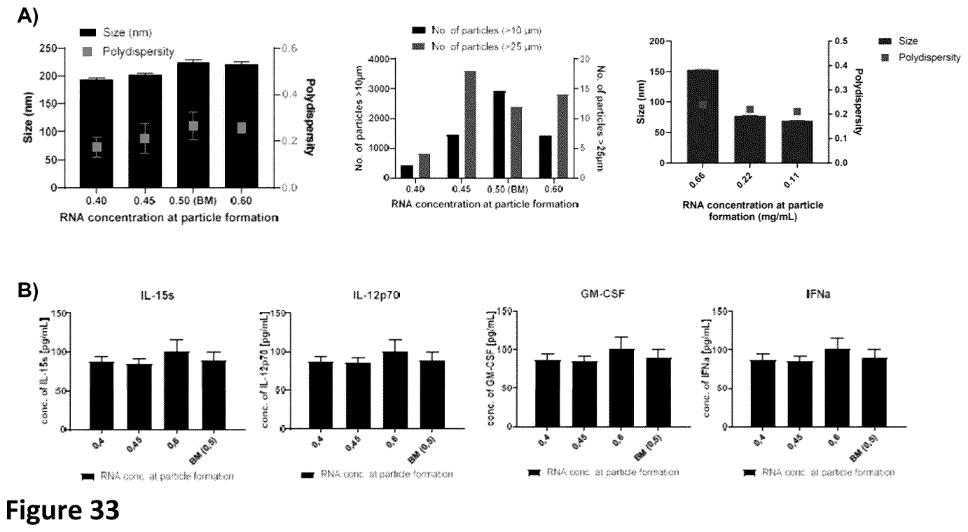

In order to assess the impact of the different lipid components and their ratios on protein expression of the 4-cytokine RNA mixture, LPX particles were prepared containing 19 different % mol ratios of the three key lipid components: DODMA, DOPE and DOPS. The in vitro protein expression was measured for each of the four cytokines and the lipid composition was observed to have an effect of protein expression of the drug product (FIG. 32). Run ID numbers 18 and 19 appeared to result in higher protein expression in vitro. The concentration of RNA at particle formation typically used during PS-LPX manufacturing as described herein was 0.50 mg/mL, following subsequent dilution with cryoprotectant a target final RNA concentration of 0.22 mg/mL was used. Different RNA concentrations were tested to assess the impact on physiochemical properties and protein expression levels. A trend of increasing particle size and polydispersity with increasing RNA concentration is observed (FIG. 33A). Additionally, LPX manufactured at RNA concentrations >0.40 mg/mL showed higher numbers of sub-visible particles of size >25 m (FIG. 33A). Protein expression from LPX manufactured at different RNA concentrations appeared not to change significantly.

The composition of the formulation (shown in Table 15) was fixed after the described further optimization of excipients and parameters.

TABLE 15
Composition of PS-LPX-A.

| | | | | Composition | |
|---|---|---|---|---|---|
| | | | | (per 1.35 mL | |
| Components | Function | Concentration | | drug product vial) | |
| RNA | Drug substance | 0.22 | mg/mL^{[b]} | 0.30 | mg |
| DODMA | Ionizable lipid | 1.23 | mg/mL | 1.66 | mg |
| DOPE | Helper lipid | 0.25 | mg/mL | 0.33 | mg |
| DOPS | Helper lipid | 0.27 | mg/mL | 0.36 | mg |
| Polysorbate 20 | Surfactant | 0.81 | mg/mL | 1.09 | mg |
| Acetic acid | Buffer component | 0.03 | mg/mL | 0.04 | mg |
| Sucrose | Cryoprotectant | 100 | mg/mL | 135 | mg |
| Sodium citrate | Buffer component | 0.12 | mg/mL | 0.16 | mg |
| Citric acid | Buffer component | 0.12 | mg/mL | 0.16 | mg |
| NaCl | Tonicity modifier | 0.13 | mg/mL | 0.17 | mg |
| HEPES | Buffer component | 0.26 | mg/mL | 0.35 | mg |
| EDTA | Chelating agent | 0.003 | mg/mL | 0.004 | mg |
| Sodium acetate | Buffer component | 0.41 | mg/mL | 0.55 | mg |

| | | | |
|---|---|---|---|
| Water for | Solvent | q.s. 1 mL | q.s. 1.35 mL |
| injection | | | |

Note:
q.s.—quantum satis;
[a] When it is referred to a Pharmacopeia, this means that the current edition of this Pharmacopoeia is applied.
^{[b]}total RNA (ratio of each: RNA drug substance is 1:1:1:1 by weight)

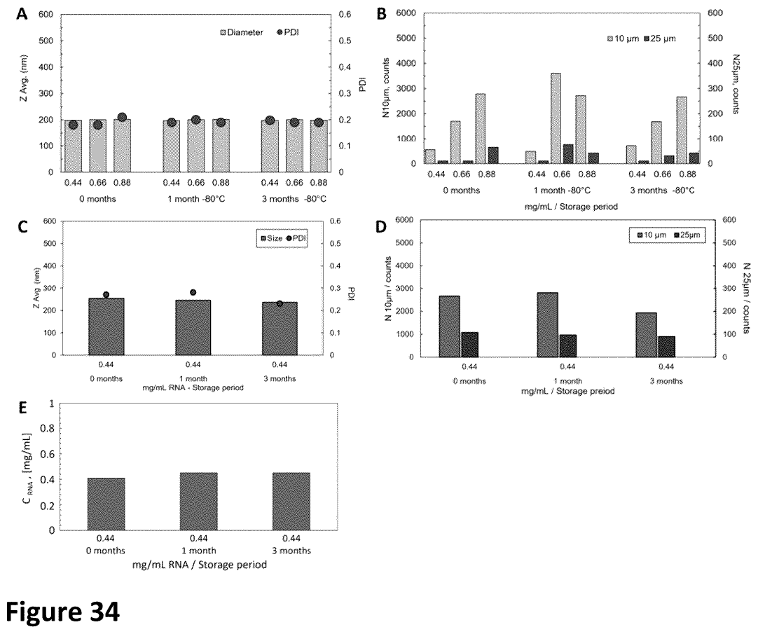

Additionally, up-concentrated versions of the PS-LPX formulation were generated by 5 ultrafiltration/diafiltration (UF/DF) and it was shown that stable particles could be formed. The up-concentrated PS-LPX formulation comprising 0.44 mg/mL RNA was found to be most stable whilst providing a higher RNA concentration (FIG. 34). The composition of this PS-LPX formulation is shown below in Table 16.

TABLE 16
Comparison of the composition of PS-
LPX and up-concentrated PS-LPX.

| | | |
|---|---|---|
| Components | Concentration | Concentration |

| | | | | |
|---|---|---|---|---|
| RNA | 0.22 | mg/mL | 0.44 | mg/mL |
| DODMA | 1.23 | mg/mL | 2.48 | mg/mL |
| DOPE | 0.25 | mg/mL | 0.50 | mg/mL |
| DOPS | 0.27 | mg/mL | 0.54 | mg/mL |
| Polysorbate 20 | 0.81 | mg/mL | 1.64 | mg/mL |
| Acetic acid | 0.03 | mg/mL | 0.05 | mg/mL |
| Sucrose | 100 | mg/mL | 100 | mg/mL |
| TriSodium citrate dihydrate | 0.12 | mg/mL | 0.18 | mg/mL |
| Citric acid monohydrate | 0.13 | mg/mL | 0.19 | mg/mL |
| NaCl | 0.13 | mg/mL | 0.19 | mg/mL |
| HEPES | 0.26 | mg/mL | 0.53 | mg/mL |
| EDTA | 0.003 | mg/mL | 0.006 | mg/mL |
| Sodium acetate trihydrate | 0.68 | mg/mL | 0.68 | mg/mL |

1. An RNA particle comprising:
(i) RNA,
(ii) at least one cationic or cationically ionizable lipid, and
(iii) at least one phosphatidylserine.

2. The RNA particle of claim 1, wherein the cationic or cationically ionizable lipid comprises a head group which includes at least one nitrogen atom which is positively charged or capable of being protonated, preferably under physiological conditions.

3. The RNA particle of claim 1 or 2, wherein the cationic or cationically ionizable lipid is selected from the group consisting of N,N-dimethyl-2,3-dioleyloxypropylamine (DODMA), 1,2-dimyristyloxypropyl-3-dimethyl-hydroxy ethyl ammonium bromide (DMRIE), N,N-dioleyl-N,N-dimethylammonium chloride (DODAC), 1,2-dioleoyl-3-dimethylammonium-propane (DODAP), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), (6Z,9Z,28Z,31Z)-heptatriaconta-6,9,28,31-tetraen-19-yl 4-(dimethylamino)butanoate (D-Lin-MC3-DMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), and 4-((di((9Z,12Z)-octadeca-9,12-dien-1-yl)amino)oxy)-N,N-dimethyl-4-oxobutan-1-amine (DPL-14) or a mixture thereof.

4. The RNA particle of any one of claims 1 to 3, wherein the phosphatidylserine comprises dioleoylphosphatidylserine (DOPS), 1,2-dioctanoyl-sn-glycero-3-phospho-L-serine (08:0 PS), 1,2-didecanoyl-sn-glycero-3-phospho-L-serine (10:0 PS), 1,2-dilauroyl-sn-glycero-3-phospho-L-serine (12:0 PS), dimyristoylphosphatidylserine (DMPS), dipalmitoylphosphatidylserine (DPPS), 1,2-diheptadecanoyl-sn-glycero-3-phospho-L-serine (17:0 PS), 1,2-distearoyl-sn-glycero-3-phospho-L-serine (18:0 PS), 1-palmitoyl-2-oleoyl-sn-glycero-3-phospho-L-serine (POPS), 1-stearoyl-2-oleoyl-sn-glycero-3-phospho-L-serine (SOPS), 1-palmitoyl-2-linoleoyl-sn-glycero-3-phospho-L-serine (PLenPS), 1-palmitoyl-2-arachidonoyl-sn-glycero-3-phospho-L-serine (16:0-20:4 PS), 1-palmitoyl-2-docosahexaenoyl-sn-glycero-3-phospho-L-serineor (16:0-22:6 PS) 1-stearoyl-2-linoleoyl-sn-glycero-3-phospho-L-serine (18:0-18:2 PS), 1-stearoyl-2-arachidonoyl-sn-glycero-3-phospho-L-serine (18:0-20:4 PS), 1-stearoyl-2-docosahexaenoyl-sn-glycero-3-phospho-L-serine (18:0-22:6 PS), 1,2-dilinoleoyl-sn-glycero-3-phospho-L-serine (18:2 PS), 1,2-diarachidonoyl-sn-glycero-3-phospho-L-serine (20:4 PS), 1,2-didocosahexaenoyl-sn-glycero-3-phospho-L-serine (22:6 PS), 1-tridecanoyl-sn-glycero-3-phospho-L-serine (13:0 Lyso PS), 1-(10Z-heptadecenoyl)-2-hydroxy-sn-glycero-3-[phospho-L-serine] (17:1 Lyso PS), 1-palmitoyl-2-hydroxy-sn-glycero-3-phospho-L-serine (16:0 Lyso PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), brain PS, brain lyso PS, soy PS, or a mixture thereof.

5. The RNA particle of any one of claims 1 to 4, which further comprises at least one additional lipid.

6. The RNA particle of claim 5, wherein the additional lipid is selected from the group consisting of neutral lipids, steroids, and combinations thereof.

7. The RNA particle of claim 5 or 6, wherein the additional lipid comprises a neutral lipid.

8. The RNA particle of any one of claims 5 to 7, wherein the additional lipid comprises a phospholipid.

9. The RNA particle of claim 8, wherein the phospholipid is selected from the group consisting of phosphatidylcholines, phosphatidylethanolamines, phosphatidylglycerols, phosphatidic acids, and sphingomyelins.

10. The RNA particle of claim 8 or 9, wherein the phospholipid is selected from the group consisting of dioleoylphosphatidylethanolamine (DOPE), distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).

11. The RNA particle of any one of claims 5 to 10, wherein the additional lipid comprises cholesterol or a cholesterol derivative.

12. The RNA particle of any one of claims 5 to 11, wherein the additional lipid comprises a mixture of a phospholipid and cholesterol or a cholesterol derivative.

13. The RNA particle of any one of claims 5 to 12, wherein the additional lipid comprises DOPE, cholesterol, or a mixture of DOPE and cholesterol.

14. The RNA particle of any one of claims 1 to 13, wherein the cationic or cationically ionizable lipid comprises from about 20 mol % to about 95 mol % of the total lipid present in the particle.

15. The RNA particle of any one of claims 1 to 14, wherein the phosphatidylserine comprises from about 2 mol % to about 70 mol % of the total lipid present in the particle.

16. The RNA particle of any one of claims 5 to 15, wherein the additional lipid comprises from about 0 mol % to about 80 mol % of the total lipid present in the particle.

17. The RNA particle of any one of claims 1 to 16, which comprises a cationic or cationically ionizable lipid, a phosphatidylserine, and an additional lipid.

18. The RNA particle of claim 17, wherein the additional lipid comprises a neutral lipid, a steroid or a mixture thereof.

19. The RNA particle of any one of claims 5 to 18, wherein the molar ratio of cationic or cationically ionizable lipid, phosphatidylserine, and additional lipid is 2-9.5:0.2-7:0.2-8.

20. The RNA particle of any one of claims 5 to 19, wherein the molar ratio of cationic or cationically ionizable lipid, phosphatidylserine, and additional lipid is about 6:about 1:about 1.

21. The RNA particle of any one of claims 5 to 19, wherein the molar ratio of cationic or cationically ionizable lipid, phosphatidylserine, and additional lipid is about 6:about 1:about 1.2-1.8, such as about 6:about 1:about 1.2, about 6:about 1:about 1.7, or about 6:about 1:about 1.8.

22. The RNA particle of any one of claims 1 to 21, which comprises a cationic or cationically ionizable lipid, DOPS, and DOPE.

23. The RNA particle of claim 22, wherein the molar ratio of cationic or cationically ionizable lipid, DOPS, and DOPE is 2-9.5:0.2-7:0.2-8.

24. The RNA particle of claim 22 or 23, wherein the molar ratio of cationic or cationically ionizable lipid, DOPS, and DOPE is about 6:about 1:about 1.

25. The RNA particle of claim 22 or 23, wherein the molar ratio of cationic or cationically ionizable lipid, DOPS, and DOPE is about 6:about 1:about 1.2-1.8, such as about 6:about 1:about 1.2, about 6:about 1:about 1.7, or about 6:about 1:about 1.8.

26. The RNA particle of any one of claims 1 to 22, which comprises DODMA, DOPS, and DOPE.

27. The RNA particle of claim 26, wherein the molar ratio of DODMA, DOPS, and DOPE is 2-9.5:0.2-7:0.2-8.

28. The RNA particle of claim 26 or 27, wherein the molar ratio of DODMA, DOPS, and DOPE is about 6:about 1:about 1.

29. The RNA particle of any one of claims 26 to 28, wherein the molar ratio of DODMA, DOPS, and DOPE is about 6:about 1:about 1.2-1.8, such as about 6:about 1:about 1.2, about 6:about 1:about 1.7, or about 6:about 1:about 1.8.

30. An RNA particle comprising:
(i) RNA,
(ii) at least one cationic or cationically ionizable lipid,
(iii) a first phospholipid which is a phosphatidylserine, and
(iv) a second phospholipid.

31. The RNA particle of claim 30, wherein the cationic or cationically ionizable lipid comprises DODMA.

32. The RNA particle of claim 30 or 31, wherein the phosphatidylserine comprises DOPS.

33. The RNA particle of any one of claims 30 to 32, wherein the second phospholipid comprises DOPE.

34. The RNA particle of any one of claims 30 to 33, wherein the cationic or cationically ionizable lipid comprises from about 20 mol % to about 95 mol % of the total lipid present in the particle.

35. The RNA particle of any one of claims 30 to 34, wherein the phosphatidylserine comprises from about 2 mol % to about 70 mol % of the total lipid present in the particle.

36. The RNA particle of any one of claims 30 to 35, wherein the second phospholipid comprises from about 2 mol % to about 80 mol % of the total lipid present in the particle.

37. The RNA particle of any one of claims 30 to 36, wherein the molar ratio of cationic or cationically ionizable lipid, phosphatidylserine, and second phospholipid is 2-9.5:0.2-7:0.2-8.

38. The RNA particle of any one of claims 30 to 37, wherein the molar ratio of cationic or cationically ionizable lipid, phosphatidylserine, and second phospholipid is about 6:about 1:about 1.

39. The RNA particle of any one of claims 30 to 37, wherein the molar ratio of cationic or cationically ionizable lipid, phosphatidylserine, and second phospholipid is about 6:about 1:about 1.2-1.8, such as about 6:about 1:about 1.2, or about 6:about 1:about 1.7, or about 6:about 1:about 1.8.

40. An RNA particle comprising (i) RNA, (ii) N,N-dimethyl-2,3-dioleyloxypropylamine (DODMA) and (iii) dioleoylphosphatidylserine (DOPS).

41. The RNA particle of claim 40, which further comprises dioleoylphosphatidylethanolamine (DOPE).

42. The RNA particle of claim 40 or 41, wherein the DODMA comprises from about 20 mol % to about 95 mol % of the total lipid present in the particle.

43. The RNA particle of any one of claims 40 to 42, wherein the DOPS comprises from about 2 mol % to about 70 mol % of the total lipid present in the particle.

44. The RNA particle of any one of claims 41 to 43, wherein the DOPE comprises from about 2 mol % to about 80 mol % of the total lipid present in the particle.

45. The RNA particle of any one of claims 41 to 44, wherein the molar ratio of DODMA:DOPE:DOPS is 2-9.5:0.2-8:0.2-7.

46. The RNA particle of any one of claims 41 to 45, wherein the molar ratio of DODMA:DOPE:DOPS is 5.5-6.5:0.9-1.1:0.9-1.1.

47. The RNA particle of any one of claims 41 to 46, wherein the molar ratio of DODMA:DOPE:DOPS is about 6:about 1:about 1.

48. The RNA particle of any one of claims 41 to 45, wherein the molar ratio of DODMA:DOPE:DOPS is about 6:about 1.2-1.8:about 1, such as about 6:about 1.2:about 1, or about 6:about 1.7:about 1, or about 6:about 1.8:about 1.

49. The RNA particle of any one of claims 1 to 48, which comprises a non-ionic amphiphilic organic compound.

50. The RNA particle of claim 49, wherein the non-ionic amphiphilic organic compound is a surfactant.

51. The RNA particle of claim 49 or 50, wherein the non-ionic amphiphilic organic compound is a poly(ethylene glycol) (PEG) surfactant.

52. The RNA particle of any one of claims 49 to 51, wherein the non-ionic amphiphilic organic compound comprises a poly(ethylene glycol) (PEG) chain linked to a single hydrophobic chain.

53. The RNA particle of any one of claims 49 to 52, wherein the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof.

54. The RNA particle of any one of claims 1 to 53, which comprises a polyoxyethylene sorbitan fatty acid ester.

55. The RNA particle of any one of claims 1 to 54, which comprises a polysorbate.

56. The RNA particle of claim 55, wherein the polysorbate is polysorbate 20.

57. The RNA particle of any one of claims 1 to 10, and 13 to 56, which does not comprise cholesterol.

58. The RNA particle of any one of claims 1 to 10, and 13 to 57, which does not comprise a steroid.

59. The RNA particle of any one of claims 1 to 58, wherein the RNA is mRNA or saRNA.

60. The RNA particle of any one of claims 1 to 59, wherein the particle is a lipid nanoparticle (LNP) or a lipoplex particle (LPX).

61. The RNA particle of any one of claims 1 to 60, wherein the particle has a size of from about 30 nm to about 500 nm.

62. The RNA particle of any one of claims 1 to 61, wherein the particle is a non-viral particle.

63. The RNA particle of any one of claims 1 to 62, wherein the RNA is of unimolecular or multimolecular species.

64. The RNA particle of any one of claims 1 to 63, wherein the RNA is coding RNA.

65. The RNA particle of any one of claims 1 to 64, wherein the RNA comprises a mixture of mRNAs that encode different peptides or polypeptides.

66. The RNA particle of any one of claims 1 to 65, wherein the RNA encodes one or more peptides or polypeptides selected from the group consisting of cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

67. The RNA particle of any one of claims 1 to 66, wherein the RNA comprises RNA encoding an IL-12sc protein, RNA encoding an IL-15 sushi protein, RNA encoding an IFNα protein, and RNA encoding a GM-CSF protein.

68. The RNA particle of any one of claims 65 to 67, wherein
viii. each RNA is in a 1:1:1:1 (w/w/w/w) ratio; and/or
ix. each RNA comprises a modified nucleoside in place of each uridine; and/or
x. each RNA comprises a modified nucleoside in place of each uridine, wherein the modified nucleoside is independently selected from pseudouridine (W), Ni-methyl-pseudouridine (m1ψ), and 5-methyl-uridine (m5U); and/or
xi. each RNA comprises the 5′ cap m₂^{7,3′-O}Gppp(m₁^{2′-O})ApG or 3′-O-Me-m⁷G(5′)ppp(5′)G; and/or
xii. each RNA comprises a 5′ UTR comprising a nucleotide sequence selected from the group consisting of SEQ ID NOs: 2 and 4, or a nucleotide sequence having at least 99%, 98%, 97%, 96%, 95%, 90%, 85%, or 80% identity to a nucleotide sequence selected from the group consisting of SEQ ID NOs: 2 and 4; and/or
xiii. each RNA comprises a 3′ UTR comprising the nucleotide sequence of SEQ ID NO: 6, or a nucleotide sequence having at least 99%, 98%, 97%, 96%, 95%, 90%, 85%, or 80% identity to the nucleotide sequence of SEQ ID NO: 6; and/or
xiv. each RNA comprises a poly-A tail, wherein the poly-A tail comprises at least 100 nucleotides, and wherein the poly-A tail comprises or consists of the poly-A tail shown in SEQ ID NO: 27.

69. The RNA particle of claim 67 or 68, wherein
i. the RNA encoding IL-12sc protein comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7; and/or
ii. the RNA encoding an IL-15 sushi protein comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8; and/or
iii. the RNA encoding an IFNα protein comprises the nucleotide sequence of SEQ ID NO: 9, or a nucleotide sequence having at least 99%, 98%, 97%, 96%, 95%, 90%, 85%, or 80% identity to the nucleotide sequence of SEQ ID NO: 9; and/or
iv. the RNA encoding a GM-CSF protein comprises the nucleotide sequence of SEQ ID NO: 10, or a nucleotide sequence having at least 99%, 98%, 97%, 96%, 95%, 90%, 85%, or 80% identity to the nucleotide sequence of SEQ ID NO: 10.

70. The RNA particle of any one of claims 67 to 69, wherein
i. the IL-12sc protein comprises the amino acid sequence of SEQ ID NO: 11, or an amino acid sequence having at least 99%, 98%, 97%, 96%, 95%, 90%, 85%, or 80% identity to the amino acid sequence of SEQ ID NO: 11; and/or
ii. the IL-15 sushi protein comprises the amino acid sequence of SEQ ID NO: 21, or an amino acid sequence having at least 99%, 98%, 97%, 96%, 95%, 90%, 85%, or 80% identity to the amino acid sequence of SEQ ID NO: 21; and/or
iii. the IFNα protein comprises the amino acid sequence of SEQ ID NO: 16, or an amino acid sequence having at least 99%, 98%, 97%, 96%, 95%, 90%, 85%, or 80% identity to the amino acid sequence of SEQ ID NO: 16; and/or
iv. the GM-CSF protein comprises the amino acid sequence of SEQ ID NO: 24, or an amino acid sequence having at least 99%, 98%, 97%, 96%, 95%, 90%, 85%, or 80% identity to the amino acid sequence of SEQ ID NO: 24.

71. The RNA particle of any one of claims 1 to 70, which is obtainable by a method comprising the steps:
(i) preparing an acidified lipid solution in an organic solvent;
(ii) adding the acidified lipid solution to an aqueous phase to produce a liposome colloid; and
(iii) adding the liposome colloid to an aqueous solution comprising RNA and a non-ionic amphiphilic organic compound to produce the RNA lipid particle.

72. The RNA particle of claim 71, wherein the particle is a lipoplex particle (LPX).

73. The RNA particle of any one of claims 1 to 70, which is obtainable by a method comprising the steps:
(i) preparing an acidified lipid solution in an organic solvent; and
(ii) adding the acidified lipid solution to an aqueous solution comprising RNA and a non-ionic amphiphilic organic compound to produce the RNA lipid particle.

74. The RNA particle of claim 73, wherein the particle is a lipid nanoparticle (LNP).

75. The RNA particle of any one of claims 71 to 74, wherein the organic solvent comprises an alcohol.

76. The RNA particle of claim 75, wherein the alcohol is ethanol.

77. The RNA particle of any one of claims 71 to 76, wherein the non-ionic amphiphilic organic compound comprises from about 0.1 mol % to about 30 mol % of the total lipid.

78. The RNA particle of any one of claims 71 to 77, wherein the non-ionic amphiphilic organic compound comprises more than about 5 mol % of the total lipid.

79. The RNA particle of any one of claims 71 to 78, wherein the non-ionic amphiphilic organic compound comprises from about 5 mol % to about 25 mol % of the total lipid.

80. A composition comprising one or more RNA particles or a plurality of RNA particles according to any one of claims 1 to 79.

81. The composition of claim 80, which comprises an aqueous solution.

82. The composition of claim 81, wherein the particles and/or the aqueous solution comprises a non-ionic amphiphilic organic compound.

83. The composition of claim 82, wherein the non-ionic amphiphilic organic compound is a surfactant.

84. The composition of claim 82 or 83, wherein the non-ionic amphiphilic organic compound is a poly(ethylene glycol) (PEG) surfactant.

85. The composition of any one of claims 82 to 84, wherein the non-ionic amphiphilic organic compound comprises a poly(ethylene glycol) (PEG) chain linked to a single hydrophobic chain.

86. The composition of any one of claims 82 to 85, wherein the non-ionic amphiphilic organic compound comprises a polyoxyethylene sorbitan ester, D-α-tocopheryl polyethylene glycol-succinate (TPGS), a polyoxyethylene mono ester of a saturated C10 to C22 hydroxy fatty acid, a polyoxyethylene fatty acid ester, a polyoxyethylene alkyl ether, or a combination thereof.

87. The composition of any one of claims 80 to 86, which comprises a polyoxyethylene sorbitan fatty acid ester.

88. The composition of any one of claims 80 to 87, which comprises a polysorbate.

89. The composition of claim 88, wherein the polysorbate is polysorbate 20.

90. The composition of any one of claims 82 to 89, wherein the non-ionic amphiphilic organic compound comprises from about 0.1 mol % to about 30 mol % of the total lipid present in the composition.

91. The composition of any one of claims 82 to 90, wherein the non-ionic amphiphilic organic compound comprises more than about 5 mol % of the total lipid present in the composition.

92. The composition of any one of claims 82 to 91, wherein the non-ionic amphiphilic organic compound comprises from about 5 mol % to about 25 mol % of the total lipid present in the composition.

93. The composition of any one of claims 80 to 92, comprising DODMA, DOPE, DOPS, and polysorbate 20.

94. The composition of any one of claims 80 to 93, comprising DODMA, DOPE, DOPS, and polysorbate 20, wherein the molar ratio of DODMA:DOPE:DOPS:polysorbate 20 is 5.5-6.5:0.9-1.1:0.9-1.1:1.5-2.5.

95. The composition of claim 93 or 94, wherein the molar ratio of DODMA:DOPE:DOPS:polysorbate 20 is about 6:about 1:about 1:about 2, or the molar ratio of DODMA:DOPE:DOPS:polysorbate 20 is about 6:about 1.2-1.8:about 1:about 2, such as about 6:about 1.2:about 1:about 2, or about 6:about 1.7:about 1:about 2, or about 6:about 1.8:about 1:about 2.

96. The composition of any one of claims 80 to 95, comprising a non-ionic amphiphilic organic compound at a level of at least about 0.6 mM.

97. The composition of any one of claims 80 to 96, comprising DODMA at a level of at least about 1.8 mM, DOPE at a level of at least about 0.3 mM, and DOPS at a level of at least about 0.3 mM.

98. The composition of any one of claims 80 to 97, wherein the composition does not comprise cholesterol.

99. The composition of any one of claims 80 to 98, wherein the composition does not comprise a steroid.

100. The composition of any one of claims 80 to 99, wherein the lipids in the composition consist of DODMA, DOPE, DOPS, and polysorbate 20.

101. The composition of any one of claims 80 to 100, wherein the particles are LPX particles.

102. The composition of claim 101, wherein the LPX particles have an N/P ratio from about 3 to about 5, and wherein the pH of the composition is from about 7 to about 7.5.

103. The composition of claim 101, wherein the LPX particles have an N/P ratio from about 3 to about 5, and wherein the pH of the composition is from about 5.0 to about 6.0, optionally from about 5.2 to about 5.8, optionally about 5.5.

104. The composition of any one of claims 101 to 103, wherein the LPX particles have an N/P ratio of about 3.

105. The composition of any one of claims 80 to 104, wherein the RNA comprises RNA encoding an IL-12sc protein, RNA encoding an IL-15 sushi protein, RNA encoding an IFNα protein, and RNA encoding a GM-CSF protein.

106. The composition of any one of claims 80 to 105, wherein the composition comprises citrate buffer, optionally citrate buffer and sodium chloride.

107. A method for delivering RNA to cells of a subject, the method comprising administering to a subject one or more RNA particles or a plurality of RNA particles according to any one of claims 1 to 79 or the composition according to any one of claims 80 to 106.

108. A method for delivering a therapeutic or prophylactic peptide or polypeptide to a subject, the method comprising administering to a subject one or more RNA particles or a plurality of RNA particles according to any one of claims 1 to 79 or the composition according to any one of claims 80 to 106, wherein the RNA encodes the therapeutic or prophylactic peptide or polypeptide.

109. A method for treating or preventing a disease in a subject, the method comprising administering to a subject a pharmaceutically effective amount of one or more RNA particles or a plurality of RNA particles according to any one of claims 1 to 79 or the composition according to any one of claims 80 to 106, wherein delivering the RNA to cells of the subject is beneficial in treating or preventing the disease.

110. A method for treating or preventing a disease in a subject, the method comprising administering to a subject a pharmaceutically effective amount of one or more RNA particles or a plurality of RNA particles according to any one of claims 1 to 79 or the composition according to any one of claims 80 to 106, wherein the RNA encodes a therapeutic peptide or polypeptide and wherein delivering the therapeutic peptide or polypeptide to the subject is beneficial in treating or preventing the disease.

111. The method of any one of claims 107 to 110, wherein administration is by intravenous, intratumoral, or peritumoral injection.

112. The method of any one of claims 107 to 111, wherein the subject is a mammal.

113. The method of claim 112, wherein the mammal is a human.

114. A method of producing RNA lipid particles comprising at least one phosphatidylserine, the method comprising the step of preparing a lipid solution in an organic solvent comprising the at least one phosphatidylserine, wherein the lipid solution is acidified.

115. The method of claim 114, wherein the organic solvent comprises an alcohol.

116. The method of claim 115, wherein the alcohol is ethanol.

117. The method of any one of claims 114 to 116, wherein the lipid solution has an acidic pH so as to render the at least one phosphatidylserine electroneutral.

118. The method of any one of claims 114 to 117, wherein the lipid solution further comprises a cationically ionizable lipid and the acidic pH of the lipid solution renders the cationically ionizable lipid protonated.

119. The method of any one of claims 114 to 118 further comprising adding the lipid solution to an aqueous phase to produce a liposome colloid.

120. The method of claim 119 further comprising adding the liposome colloid to an aqueous solution comprising the RNA to produce the RNA lipid particles.

121. The method of claim 120, wherein the aqueous solution comprising the RNA comprises citrate buffer, optionally citrate buffer and sodium chloride.

122. The method of any one of claims 114 to 121, wherein the RNA lipid particles are lipoplex particles (LPX).

123. The method of any one of claims 120 to 122, wherein the liposome colloid and the aqueous solution comprising the RNA are mixed at a flow rate of at least 200 mL/min, optionally at a flow rate of 300-400 mL/min, preferably at a flow rate of 300-360 mL/min.

124. The method of any one of claims 114 to 118 further comprising mixing the lipid solution with an aqueous solution comprising the RNA to produce the RNA lipid particles.

125. The method of any one of claims 114 to 118, and 124, wherein the RNA lipid particles are lipid nanoparticles (LNP).

126. The method of claim 124 or 125, wherein the lipid solution and the aqueous solution comprising the RNA are mixed at a flow rate of about 2 to about 12 mL/min, optionally at a flow rate of about 4 to about 10 mL/min, preferably at a flow rate of about 6 to about 10 mL/min.

127. The method of any one of claims 114 to 126, wherein the lipid solution and/or the RNA solution comprises a non-ionic amphiphilic organic compound.

128. The method of any one of claims 114 to 127, the method further comprising freezing the RNA lipid particles at −80° C. for storage.

129. A method of producing RNA lipid particles, the method comprising the step of preparing a lipid solution in an organic solvent and mixing the lipid solution with an aqueous solution comprising the RNA to produce the RNA lipid particles, wherein a non-ionic amphiphilic organic compound is present in the lipid solution or in the RNA solution, or both.

130. The method of claim 129, wherein the organic solvent comprises an alcohol.

131. The method of claim 130, wherein the alcohol is ethanol.

132. The method of any one of claims 127 to 131, wherein the lipid solution and/or the RNA solution comprises a mol fraction of non-ionic amphiphilic organic compound from about 0.1 mol % to about 30 mol % of the total lipid.

133. The method of any one of claims 127 to 132, wherein the non-ionic amphiphilic organic compound comprises more than about 5 mol % of the total lipid.

134. The method of any one of claims 127 to 133, wherein the non-ionic amphiphilic organic compound comprises from about 5 mol % to about 25 mol % of the total lipid.

135. A method of producing a composition comprising RNA lipoplex particles comprising at least one phosphatidylserine, the method comprising:
i) preparing an acidified aqueous colloid comprising liposomes, wherein the liposomes comprise at least one phosphatidylserine, wherein the acidified aqueous colloid comprising liposomes comprises from 10 to 30 mM acetic acid; and
ii) mixing the acidified aqueous colloid comprising liposomes with an aqueous solution comprising RNA;
thereby producing the composition comprising RNA lipoplex particles,
wherein the RNA lipoplex particles and the liposomes comprise dioleoylphosphatidylserine (DOPS); N,N-dimethyl-2,3-dioleyloxypropylamine (DODMA); and dioleoylphosphatidylethanolamine (DOPE).


SEQUENCE LISTING (27 sequences)

SEQ ID NO: 1 - DNA, 145 bp, synthetic construct
  misc_feature 1..145: note = 5' UTR (DNA)
  source 1..145: mol_type = other DNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 2 - RNA, 145 nt, synthetic construct
  misc_feature 1..145: note = 5' UTR (RNA)
  source 1..145: mol_type = other RNA
ggaataaactagtctcaacacaacatatacaaaacaaacgaatctcaagcaatcaagcattctacttctattgcagcaatttaaatcatttcttttaaagcaaaagcaattttctgaaaattttcaccatttacgaacgatagcc

SEQ ID NO: 3 - DNA, 52 bp, synthetic construct
  misc_feature 1..52: note = Mod 5' UTR (DNA)
  source 1..52: mol_type = other DNA
agacgaactagtattcttctggtccccacagactcagagagaacccgccacc

SEQ ID NO: 4 - RNA, 52 nt, synthetic construct
  misc_feature 1..52: note = Mod 5' UTR (RNA)
  source 1..52: mol_type = other RNA
agacgaactagtattcttctggtccccacagactcagagagaacccgccacc

SEQ ID NO: 5 - DNA, 317 bp, synthetic construct
  misc_feature 1..317: note = 3' UTR (DNA)
  source 1..317: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 6 - RNA, 317 nt, synthetic construct
  misc_feature 1..317: note = 3' UTR (RNA)
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 7 - RNA, 2102 nt, synthetic construct
  misc_feature 1..2102: note = mRNA encoding IL-12sc
  modified_base 1..2102: mod_base = OTHER; note = n is N1-methylpseudouridine
  source 1..2102: mol_type = other RNA
agacgaacnagnanncnncnggnccccacagacncagagagaacccgccaccangngncaccagcagcnggngancncanggnncncccnggnannncnggcancnccncnngncgcaancngggaacngaagaaagacgngnangncgnngagcncgacnggnanccggangcgccnggcgaganggnggngcngaccngngacaccccagaggaggangggancacnnggacccnngancaanccnccgaagngcncgggncnggcaagacncngaccanacaagngaaagagnnnggcgangccgggcagnacacnngccanaagggcggagaagnncngncccacncacngcngcngcngcacaagaaagaggacggaannnggagnaccgananccngaaagancagaaagagcccaagaacaaaaccnncnngcggngcgaagccaagaacnacncagggagannnacnngnnggnggcngacgacgancagcaccgancngacnnncnccgngaaancaagnaggggancancngacccncaaggagncacangnggagcggcnacncngagcgcngaacgcgnaagaggggacaanaaggagnacgagnanagcgnngagngccaagagganagcgcangccccgccgccgaagaancanngcccanngaagnganggnggangcngnacacaagcngaagnangagaacnacacaagcnccnncnncanccgngacancancaaaccaganccnccnaagaaccnccagcnnaaaccncngaagaacncnagacaggnggaagngncnngggagnancccgacaccnggncnacaccacannccnacnncagncncacanncngcgnncaggnacagggcaagnccaaaagggagaagaaggancgggncnnnacaganaaaacaagngccaccgnnanangccggaagaangccncnannncngngcgngcgcaggacaganacnanagcagcncnnggagngaangggccagngncccangnncagggncanccggnggnggcggcagccccggaggcggnagcnccagaaancncccngnggcnacaccnganccaggcangnnncccngnnngcaccanagccaaaaccnccngagagcagncagcaacangcnccagaaagcnagacaaacacnggaanncnacccangcaccnccgaggaaanagancacgagganancacnaaggacaaaacaagcacngncgaagcangccnncccnnggaacngacaaagaacgagagnngccnnaanncaagagaaacancnnncannacaaacggnagcngcnnggcaagcagaaaaacancnnnnanganggcccnnngncngagcagnannnangaggancncaaaangnaccaggnggagnnnaagaccangaangccaagcngcnganggacccaaagagacagannnnccncgancagaanangcnggcngnganngangaacngangcaggccnngaannncaacagcgaaaccgnnccccagaaaagcagncnngaagaaccngacnnnnanaagaccaagancaaacngngnanncnccngcangccnnnagaancagagcagncacnanaganagagngangnccnaccngaangcnnccngangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacaccgagaccnggnccagagncgcnagccgcgncgcnaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

SEQ ID NO: 8 - RNA, 1208 nt, synthetic construct
  misc_feature 1..1208: note = mRNA encoding IL-15 sushi
  modified_base 1..1208: mod_base = OTHER; note = n is N1-methylpseudouridine
  source 1..1208: mol_type = other RNA
agacgaacnagnanncnncnggnccccacagacncagagagaacccgccaccanggccccgcggcgggcgcgcggcngccggacccncggncncccggcgcngcnacngcngcngcngcnccggccgccggcgacgcggggcancacgngcccnccccccangnccgnggaacacgcagacancngggncaagagcnacagcnngnacnccagggagcggnacannngnaacncnggnnncaagcgnaaagccggcacgnccagccngacggagngcgngnngaacaaggccacgaangncgcccacnggacaacccccagncncaaangcannagagacccngcccnggnncaccaaaggccagcgccacccgggggaggancnggcggcggngggncnggcgggggancnggcggaggaggaagcnnacagaacngggngaangnaanaagngannngaaaaaaanngaagancnnanncaancnangcananngangcnacnnnananacggaaagngangnncaccccagnngcaaagnaacagcaangaagngcnnncncnnggagnnacaagnnannncacnngagnccggagangcaagnanncanganacagnagaaaancngancanccnagcaaacaacagnnngncnncnaangggaangnaacagaancnggangcaaagaangngaggaacnggaggaaaaaaanannaaagaannnnngcagagnnnngnacananngnccaaangnncancaacacnncnngangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacaccgagaccnggnccagagncgcnagccgcgncgcnaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

SEQ ID NO: 9 - RNA, 1049 nt, synthetic construct
  misc_feature 1..1049: note = mRNA encoding IFNalpha2b
  modified_base 1..1049: mod_base = OTHER; note = n is N1-methylpseudouridine
  source 1..1049: mol_type = other RNA
agacgaacnagnanncnncnggnccccacagacncagagagaacccgccaccanggcccngacnnnngcccnncncgnggcnnngnnggngcngagnngcaaancnnccngnagngncggangngancngccncaaacccacagncngggancnaggagaacacngangcngnnggcacagangaggagaannagccncnnnnccngccngaagganagacangacnncggcnnnccccaagaggagnnnggcaancagnnccagaaagcggaaacganncccgnncngcacgaganganccagcagancnncaaccncnnnncaaccaaagacagcncagcagccngggangagacacngcnggacaaanncnacacagaacngnancagcagcnnaacgancnggaggcangcgnganccaaggggnnggngngacngaaacnccgcnnangaaggaggacnccanncnggcngnacggaagnacnnccagagaanaacccncnancngaaggagaagaagnacncaccangngcnngggaagncgngagagccgaaancangaganccnncagccnnagcaccaancnccaggaancncngagaagcaaagagngangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacaccgagaccnggnccagagncgcnagccgcgncgcnaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

SEQ ID NO: 10 - RNA, 917 nt, synthetic construct
  misc_feature 1..917: note = mRNA encoding GM-CSF
  modified_base 1..917: mod_base = OTHER; note = n is N1-methylpseudouridine
  source 1..917: mol_type = other RNA
agacgaacnagnanncnncnggnccccacagacncagagagaacccgccaccangnggcnccagagccngcngcncnngggcacngnggccngcnccancncngcacccgcccgcncgcccagccccagcacgcagcccngggagcangngaangccanccaggaggcccggcgncngcngaaccngagnagagacacngcngcngagangaangaaacagnagaagncancncagaaangnnngaccnccaggagccgaccngccnacagacccgccnggagcngnacaagcagggccngcggggcagccncaccaagcncaagggccccnngaccanganggccagccacnacaagcagcacngcccnccaaccccggaaacnnccngngcaacccagannancaccnnngaaagnnncaaagagaaccngaaggacnnncngcnngncanccccnnngacngcngggagccagnccaggagngangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacaccgagaccnggnccagagncgcnagccgcgncgcnaaaaaaaaaaaaaaaaaaaaaaaaaaaaaagcanangacnaaaaaaaaaaaaaaaaaaaaaaaaaaaaaaaaaaaaaaaaaaaaaaaaaaaaaaaaaaaaaaaaaaaaaa

SEQ ID NO: 11 - protein, 539 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSSGGGGSPGGGSSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - DNA, 1623 bp, Homo sapiens
  source 1..1623: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagagggtcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggctctagcggagggggaggctctcctggcgggggatctagcagaaacctccccgtggccactccagacccaggaatgttcccatgccttcaccactcccaaaacctgctgagggccgtcagcaacatgctccagaaggccagacaaactctagaattttacccttgcacttctgaggaaattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctgatga

SEQ ID NO: 13 - DNA, 1623 bp, synthetic construct
  misc_feature 1..1623: note = Human optimized IL-12sc (CDS DNA)
  source 1..1623: mol_type = other DNA
atgtgtcaccagcagctggtgatctcatggttctccctggtatttctggcatctcctcttgtcgcaatctgggaactgaagaaagacgtgtatgtcgttgagctcgactggtatccggatgcgcctggcgagatggtggtgctgacctgtgacaccccagaggaggatgggatcacttggacccttgatcaatcctccgaagtgctcgggtctggcaagactctgaccatacaagtgaaagagtttggcgatgccgggcagtacacttgccataagggcggagaagttctgtcccactcactgctgctgctgcacaagaaagaggacggaatttggagtaccgatatcctgaaagatcagaaagagcccaagaacaaaaccttcttgcggtgcgaagccaagaactactcagggagatttacttgttggtggctgacgacgatcagcaccgatctgactttctccgtgaaatcaagtaggggatcatctgaccctcaaggagtcacatgtggagcggctactctgagcgctgaacgcgtaagaggggacaataaggagtacgagtatagcgttgagtgccaagaggatagcgcatgccccgccgccgaagaatcattgcccattgaagtgatggtggatgctgtacacaagctgaagtatgagaactacacaagctccttcttcatccgtgacatcatcaaaccagatcctcctaagaacctccagcttaaacctctgaagaactctagacaggtggaagtgtcttgggagtatcccgacacctggtctacaccacattcctacttcagtctcacattctgcgttcaggtacagggcaagtccaaaagggagaagaaggatcgggtctttacagataaaacaagtgccaccgttatatgccggaagaatgcctctatttctgtgcgtgcgcaggacagatactatagcagctcttggagtgaatgggccagtgtcccatgttcagggtcatccggtggtggcggcagccccggaggcggtagctccagaaatctccctgtggctacacctgatccaggcatgtttccctgtttgcaccatagccaaaacctcctgagagcagtcagcaacatgctccagaaagctagacaaacactggaattctacccatgcacctccgaggaaatagatcacgaggatatcactaaggacaaaacaagcactgtcgaagcatgccttcccttggaactgacaaagaacgagagttgccttaattcaagagaaacatctttcattacaaacggtagctgcttggcaagcagaaaaacatcttttatgatggccctttgtctgagcagtatttatgaggatctcaaaatgtaccaggtggagtttaagaccatgaatgccaagctgctgatggacccaaagagacagattttcctcgatcagaatatgctggctgtgattgatgaactgatgcaggccttgaatttcaacagcgaaaccgttccccagaaaagcagtcttgaagaacctgacttttataagaccaagatcaaactgtgtattctcctgcatgcctttagaatcagagcagtcactatagatagagtgatgtcctacctgaatgcttcctgatga

SEQ ID NO: 14 - RNA, 1623 nt, Homo sapiens
  source 1..1623: mol_type = other RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagagggtcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggctctagcggagggggaggctctcctggcgggggatctagcagaaacctccccgtggccactccagacccaggaatgttcccatgccttcaccactcccaaaacctgctgagggccgtcagcaacatgctccagaaggccagacaaactctagaattttacccttgcacttctgaggaaattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctgatga

SEQ ID NO: 15 - RNA, 1623 nt, synthetic construct
  misc_feature 1..1623: note = Human optimized IL-12sc (RNA encoding CDS)
  source 1..1623: mol_type = other RNA
atgtgtcaccagcagctggtgatctcatggttctccctggtatttctggcatctcctcttgtcgcaatctgggaactgaagaaagacgtgtatgtcgttgagctcgactggtatccggatgcgcctggcgagatggtggtgctgacctgtgacaccccagaggaggatgggatcacttggacccttgatcaatcctccgaagtgctcgggtctggcaagactctgaccatacaagtgaaagagtttggcgatgccgggcagtacacttgccataagggcggagaagttctgtcccactcactgctgctgctgcacaagaaagaggacggaatttggagtaccgatatcctgaaagatcagaaagagcccaagaacaaaaccttcttgcggtgcgaagccaagaactactcagggagatttacttgttggtggctgacgacgatcagcaccgatctgactttctccgtgaaatcaagtaggggatcatctgaccctcaaggagtcacatgtggagcggctactctgagcgctgaacgcgtaagaggggacaataaggagtacgagtatagcgttgagtgccaagaggatagcgcatgccccgccgccgaagaatcattgcccattgaagtgatggtggatgctgtacacaagctgaagtatgagaactacacaagctccttcttcatccgtgacatcatcaaaccagatcctcctaagaacctccagcttaaacctctgaagaactctagacaggtggaagtgtcttgggagtatcccgacacctggtctacaccacattcctacttcagtctcacattctgcgttcaggtacagggcaagtccaaaagggagaagaaggatcgggtctttacagataaaacaagtgccaccgttatatgccggaagaatgcctctatttctgtgcgtgcgcaggacagatactatagcagctcttggagtgaatgggccagtgtcccatgttcagggtcatccggtggtggcggcagccccggaggcggtagctccagaaatctccctgtggctacacctgatccaggcatgtttccctgtttgcaccatagccaaaacctcctgagagcagtcagcaacatgctccagaaagctagacaaacactggaattctacccatgcacctccgaggaaatagatcacgaggatatcactaaggacaaaacaagcactgtcgaagcatgccttcccttggaactgacaaagaacgagagttgccttaattcaagagaaacatctttcattacaaacggtagctgcttggcaagcagaaaaacatcttttatgatggccctttgtctgagcagtatttatgaggatctcaaaatgtaccaggtggagtttaagaccatgaatgccaagctgctgatggacccaaagagacagattttcctcgatcagaatatgctggctgtgattgatgaactgatgcaggccttgaatttcaacagcgaaaccgttccccagaaaagcagtcttgaagaacctgacttttataagaccaagatcaaactgtgtattctcctgcatgcctttagaatcagagcagtcactatagatagagtgatgtcctacctgaatgcttcctgatga

SEQ ID NO: 16 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 17 - DNA, 570 bp, Homo sapiens
  source 1..570: mol_type = other DNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaaccttttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgatga

SEQ ID NO: 18 - DNA, 570 bp, synthetic construct
  misc_feature 1..570: note = Human optimized IFNalpha2b (CDS DNA)
  source 1..570: mol_type = other DNA
atggccctgacttttgcccttctcgtggctttgttggtgctgagttgcaaatcttcctgtagtgtcggatgtgatctgcctcaaacccacagtctgggatctaggagaacactgatgctgttggcacagatgaggagaattagcctcttttcctgcctgaaggatagacatgacttcggctttccccaagaggagtttggcaatcagttccagaaagcggaaacgattcccgttctgcacgagatgatccagcagatcttcaacctcttttcaaccaaagacagctcagcagcctgggatgagacactgctggacaaattctacacagaactgtatcagcagcttaacgatctggaggcatgcgtgatccaaggggttggtgtgactgaaactccgcttatgaaggaggactccattctggctgtacggaagtacttccagagaataaccctctatctgaaggagaagaagtactcaccatgtgcttgggaagtcgtgagagccgaaatcatgagatccttcagccttagcaccaatctccaggaatctctgagaagcaaagagtgatga

SEQ ID NO: 19 - RNA, 570 nt, Homo sapiens
  source 1..570: mol_type = other RNA
atggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaaccttttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgatga

SEQ ID NO: 20 - RNA, 570 nt, synthetic construct
  misc_feature 1..570: note = Human optimized IFNalpha2b (RNA encoding CDS)
  source 1..570: mol_type = other RNA
atggccctgacttttgcccttctcgtggctttgttggtgctgagttgcaaatcttcctgtagtgtcggatgtgatctgcctcaaacccacagtctgggatctaggagaacactgatgctgttggcacagatgaggagaattagcctcttttcctgcctgaaggatagacatgacttcggctttccccaagaggagtttggcaatcagttccagaaagcggaaacgattcccgttctgcacgagatgatccagcagatcttcaacctcttttcaaccaaagacagctcagcagcctgggatgagacactgctggacaaattctacacagaactgtatcagcagcttaacgatctggaggcatgcgtgatccaaggggttggtgtgactgaaactccgcttatgaaggaggactccattctggctgtacggaagtacttccagagaataaccctctatctgaaggagaagaagtactcaccatgtgcttgggaagtcgtgagagccgaaatcatgagatccttcagccttagcaccaatctccaggaatctctgagaagcaaagagtgatga

SEQ ID NO: 21 - protein, 241 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPGGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 22 - DNA, 729 bp, Homo sapiens
  source 1..729: mol_type = other DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccgggggaggatctggcggcggtgggtctggcgggggatctggcggaggaggaagcttacagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatga

SEQ ID NO: 23 - RNA, 729 nt, Homo sapiens
  source 1..729: mol_type = other RNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccacccgggggaggatctggcggcggtgggtctggcgggggatctggcggaggaggaagcttacagaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgatga

SEQ ID NO: 24 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 25 - DNA, 438 bp, Homo sapiens
  source 1..438: mol_type = other DNA
atgtggctccagagcctgctgctcttgggcactgtggcctgctccatctctgcacccgcccgctcgcccagccccagcacgcagccctgggagcatgtgaatgccatccaggaggcccggcgtctgctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgatga

SEQ ID NO: 26 - RNA, 438 nt, Homo sapiens
  source 1..438: mol_type = other RNA
atgtggctccagagcctgctgctcttgggcactgtggcctgctccatctctgcacccgcccgctcgcccagccccagcacgcagccctgggagcatgtgaatgccatccaggaggcccggcgtctgctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgatga

SEQ ID NO: 27 - RNA, 110 nt, synthetic construct
  misc_feature 1..110: note = Exemplary Poly-A
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa